(19) United States
(12) Patent Application Publication
(10) Pub. No.: US 2025/0340539 A1
(43) Pub. Date: Nov. 6, 2025

(54) IMIDAZOLONE DERIVATIVES AS INHIBITORS OF PROTEIN KINASES IN PARTICULAR DYRK1A, CLK1, AND/OR CLK4

(71) Applicant: PERHA PHARMACEUTICALS, Roscoff (FR)

(72) Inventors: Emmanuel DEAU, Roscoff (FR); Pascal GEORGE, Longvilliers (FR); Laurent MEIJER, Roscoff (FR); Frédéric MIEGE, Lyon (FR)

(21) Appl. No.: 18/704,280

(22) PCT Filed: Oct. 25, 2022
(86) PCT No.: PCT/EP2022/079838

§ 371 Date: Apr. 24, 2024

(30) Foreign Application Priority Data
Oct. 26, 2021 (EP) 21306489.2

(51) Int. Cl. C07D 403/06; C07D 417/14

(52) U.S. Cl. CPC C07D 403/06; C07D 417/14

(57) ABSTRACT

The present invention relates to a compound of formula (I) wherein R¹ represents a (C₃-C₈)cycloalkyl group, a bridged (C₆-C₁₀)cycloalkyl group, a fused phenyl group, a substituted phenyl group, a R′-L- group, wherein L is either a single bond or a (C₁-C₃)alkanediyl group, and R′ represents, a (C₃-C₈)heterocycloalkyl group, or a (C₃-C₈)heteroaryl u group, or a R″-L- group wherein L is a (C₁-C₃)alkanediyl group, and R″ is an optionally substituted phenyl group; R² is selected from the group consisting of a hydrogen atom and a (C₁-C₃)alkyl group; R⁵ represents a hydrogen atom, a (C₁-C₄)alkyl group or a (C₃-C₆)cycloalkyl group or any of its pharmaceutically acceptable salt. The present invention further relates to a composition comprising a compound of formula (I) and a process for manufacturing said compound as well as its synthesis intermediates. It also relates to said compound for use as a medicament, in particular in the treatment and/or prevention of cognitive deficits and neuroinflammation associated with Down syndrome; Alzheimer's disease; dementia; tauopathies; Parkinson's disease; CDKL5 Deficiency Disorder; Phelan-McDermid syndrome; autism; type 1 and type 2 diabetes; abnormal folate and methionine metabolism; osteoarthritis and tendinopathy; Duchenne muscular dystrophy; cancers and leukemias; neuroinflammation, anemia, infections caused by unicellular parasites, viral infections and for regulating body temperature.

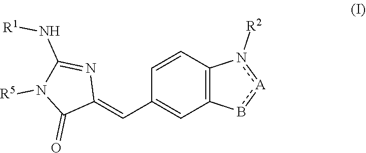

### FIELD OF THE INVENTION

The present invention relates to Leucettinibs, a class of new compounds useful as a medicament. Said new compounds are in particular useful as kinase inhibitors, and even more particularly as inhibitors of DYRK1A and/or CLK1 and/or CLK4. They are efficient for treating and/or preventing cognitive deficits associated with Down syndrome; Alzheimer's disease and related diseases; dementia; tauopathies; Parkinson's disease; other neurodegenerative diseases; CDKL5 Deficiency Disorder; type 1 and type 2 diabetes; abnormal folate and methionine metabolism; osteoarthritis and tendinopathy; Duchenne muscular dystrophy; several cancers and leukemias; viral infections and for regulating body temperature.

Some of said compounds are further inhibitors of other kinases and namely other DYRKs (DYRK1B, 2, 3, 4) and the closely related cdc2-like kinases (CLKs) (CLK 2, 3, 4). Said compounds may then further be efficient for treating and/or preventing Phelan-McDermid syndrome; autism; viral infections, cancers, neuroinflammation, anemia and infections caused by unicellular parasites.

It further relates to the pharmaceutical compositions containing said new compounds and to the chemical synthesis processes for obtaining them.

### BACKGROUND

The DYRK and CLK kinase families belong to the CMGC group of kinases which also includes the mitogen-activated protein kinases (MAPK), cyclin-dependent kinases (CDKs) and glycogen synthase kinase-3 (GSK-3). They phosphorylate many substrates involved in signaling pathways. DYRKs and CLKs play key roles in mRNA splicing, chromatin transcription, DNA damage repair, cell survival, cell cycle, differentiation, homocysteine/methionine/folate regulation, endocytosis, neuronal development and functions, synaptic plasticity (review in Lindberg, M. and Meijer, L., 2021. Dual-specificity, tyrosine phosphorylation-regulated kinases (DYRKs) and cdc2-like kinases (CLKs) in human disease, an overview. Internat. J. Mol. Sci. 22, 6047).

### DYRK1A and Down Syndrome (DS)

The gene encoding DYRK1A is located on chromosome 21, in particular in the “Down syndrome critical region” (DSCR), the triploidy of which is responsible for most DS-associated deficiencies. There is considerable genetic and pharmacological evidence showing that the mere 1.5-fold overexpression of DYRK1A is responsible for most cognitive deficits, especially memory and learning deficits, observed in DS patients (Feki, A., Hibaoui, Y., 2018. DYRK1A protein, a promising therapeutic target to improve cognitive deficits in Down syndrome. Brain Sci. 8, 187; Rueda N et al., 2020. Translational validity and implications of pharmacotherapies in preclinical models of Down syndrome. Prog Brain Res 251, 245). Pharmacological or genetical normalization of DYRK1A levels restores cognitive functions (Nguyen T L et al., 2017. Dual-specificity tyrosine phosphorylation-regulated kinase 1A (DYRK1A) inhibitors: a survey of recent patent literature. Expert Opin. Ther. Pat. 27, 1183-1199; Nguyen T L et al., 2018. Correction of cognitive deficits in mouse models of Down syndrome by pharmacological inhibitor of DYRK1A. Dis. Model Mech. 11, dmm035634).

### DYRK1A and Alzheimer's Disease (AD), Tauopathies

There is mounting evidence for a role of DYRK1A in the onset of AD. DYRK1A phosphorylates key substrates involved in AD and dementia: Tau, septin 4, amyloid precursor protein (APP), presenilin 1, neprilysin, Munc18-1, α-synuclein, RCAN1, s-Tubulin. There is evidence for abnormal expression and post-translational modifications of DYRK1A in AD. By modulating alternative splicing of exon 10, DYRK1A favors the production of the 3R-Tau splice isoform (characteristic for DS/AD/tauopathy) over the normal 4R-Tau isoform. DYRK1A inhibition promotes autophagy which could counterbalance the autophagy deficit seen in AD. There is a clear association of AD with DS (Fortea J. et al., 2021. Alzheimer's disease associated with Down syndrome: a genetic form of dementia. The Lancet 20, 930-942): most DS patients show AD neuropathology at an early age (40's) and high prevalence of dementia in later age (>60).

### DYRK1A and Parkinson's Disease (PD) and Pick Disease

GWAS studies have revealed that DYRK1A is a risk factor for PD (Nalls M A et al., 2019. Identification of novel risk loci, causal insights, and heritable risk for Parkinson's disease: a meta-analysis of genome-wide association studies. Lancet Neurol 18, 1091). DYRK1A phosphorylates key factors for PD such as Parkin, septin 4, α-synuclein. Upregulation of micro-RNA specific for PD target DYRK1A expression (Chiu C C et al., 2019. Upregulated expression of microRNA-204-5p leads to the death of dopaminergic cells by targeting DYRK1A-mediated apoptotic signaling cascade. Front Cell Neurosci 13, 399). There is further evidence that DYRK1A expression is increased in PD. DYRK1A is overexpressed in Pick disease.

### DYRK1A and Viral Infections

DYRK1A and DYRK1B are utilized during HCMV placental replication. Inhibition of DYRKs prevent replication of various viruses including Herpes virus, cytomegalovirus and HIV-1.

### DYRK1A and Type 1 and Type 2 Diabetes

DYRK1A inhibitors stimulate the proliferation of pancreatic, insulin-producing β-cells. This constitutes a promising approach to diabetes, both type 1 (which displays low number of β-cells) and type 2 (where the β-cell mass is reduced by half) (Ackeifi C et al., 2020. Pharmacologic and genetic approaches define human pancreatic P cell mitogenic targets of DYRK1A inhibitors. JCI Insight 5, e132594; Kumar K et al., 2021. DYRK1A inhibitors as potential therapeutics for β-cell regeneration for diabetes. J Med Chem 64, 2901-2922. Barzowska A, et al., 2021. DYRK1A kinase inhibitors promote β-cell survival and insulin homeostasis. Cells. 10, 2263; Wang P et al., 2021. Human beta cell regenerative drug therapy for diabetes: past achievements and future challenges. Front Endocrinol 12, 671946). Besides direct application to diabetic patients, DYRK1A inhibitors could be applied to stimulate β-cell proliferation in vitro or ex vivo to increase β-cell mass prior to grafting.

### DYRK1A, Cancers and Leukemias

There is abundant literature linking DYRK1A with cancer. The most prominent examples are megakaryoblastic leukemia (Malinge S et al., 2012. Increased dosage of the chromosome 21 ortholog Dyrkla promotes megakaryoblastic leukemia in a murine model of Down syndrome. J Clin Invest 122, 948-962), acute lymphoblastic leukemia (Bhansali R S et al., 2021. DYRK1A regulates B cell Acute Lymphoblastic Leukemia through phosphorylation of FOXO1 and STAT3. J Clin Investig, 131, e135937), pancreatic cancer and brain tumor (glioblastoma) (see review in Lindberg and Meijer, 2021. cited above).

Accordingly, abnormalities in DYRK1A dosage are associated with cognitive disorders observed in Down syndrome, and Alzheimer's disease. DYRK1A is a risk factor for Parkinson's disease. Inhibition of DYRK1A additionally triggers the proliferation of pancreatic, insulin-producing β-cells. DYRK1A inhibitors may thus find applications in preventing and/or treating DS, AD, and other Tauopathies (particularly cognitive deficits associated with these pathologies), dementia, PD, Niemann-Pick Type C Disease, CDKL5 deficiency disorder, type 1 and type 2 diabetes, viral infections, several cancers (leukemia, pancreatic cancer, glioblastoma), tendinopathy and osteoarthritis, infections caused by unicellular parasites and for regulating body temperature.

### Other DYRKs and Human Disease

DYRK1B is involved in the replication of various viruses including hepatitis C virus, Chikungunya virus, Dengue virus and SARS coronavirus, cytomegalovirus and human papillomavirus. Like DYRK1A, DYRK1B inhibition leads to the proliferation of pancreatic, insulin-producing β-cells. DYRK1B is involved in neuroinflammation. Targeting DYRK1B provides a new rationale for treatment of various cancers such as liposarcoma or breast cancers.

DYRK2, in association with GSK-3p, regulates neuronal morphogenesis. DYRK2 is involved in various ways in cancer development.

DYRK3 promotes hepatocellular carcinoma. DYRK3 couples stress granule condensation/dissolution to mTORC1 signaling. DYRK3 regulates phase transition of membraneless organelles in mitosis. DYRK3 and DYRK4 are involved in the regulation of cytoskeletal organization and process outgrowth in neurons.

DYRK1A decreases axon growth, DYRK3 and DYRK4 increase dendritic branching and DYRK2 decreases both axon and dendrite growth and branching.

### CLKs and Human Disease

Note that CLK is a confusing abbreviation as it has the following meanings: (a) monooxygenase CLK-1 (human homologue COQ7); (b) Collectin-K1 (CL-K1, or CL-11), a multifunctional Ca(2+)-dependent lectin; (c) MAPK gene of the maize pathogen Curvularia lunata, Clk1; (d) mitochondrial membrane-bound enzyme Clock-1 (CLK-1); (e) Colletotrichum lindemuthianum kinase 1 (clk1).

CLKs play essential functions in alternative splicing. CLKs act as a body-temperature sensor which globally controls alternative splicing and gene expression. The activity of CLKs is indeed highly responsive to physiological temperature changes, which is conferred by structural rearrangements within the kinase activation segment (Haltenhof T et al., 2020. A conserved kinase-based body-temperature sensor globally controls alternative splicing and gene expression. Mol Cell 78, 57).

### CLK1 and Human Disease

CLK1 triggers periodic alternative splicing during the cell division cycle. CLK1 regulates influenza A virus mRNA splicing and its inhibition prevents viral replication. CLK1 and CLK2 also regulates HIV-1 gene expression. CLK1 is an autophagy inducer. CLK1 inhibition may prevent chemoresistance in glioma and CLK1 inhibition by TG693 allows the skipping of mutated exon 31 of the dystrophin gene in Duchenne Muscular Dystrophy.

### Other CLKs and Human Disease

Inhibition of CLK2 has been proposed as a way to improve neuronal functions and combat intellectual disability and autism in Phelan-McDermid syndrome (PMDS). Dual inhibition of CLK2 and DYRK1A by Lorecivivint is a potential disease-modifying approach for knee osteoarthritis. CLK2 inhibition compromises MYC-driven breast tumors, triple negative breast cancer and glioblastoma. Inhibition of CLK2 improves autistic features in Phelan-McDermid syndrome (PMDS). Alternative splicing of Tau exon 10 is regulated by CLK2 and other CLKs, leading to changes in the 3R/4R isoforms ratio and neurodegeneration in sporadic AD. Inhibition of CLK2, CLK3, CLK4 blocks HIV-1 production. By regulating alternative splicing CLKs modulate the balance between pro-apoptotic and anti-apoptotic regulators, and inhibition of CLKs may thus find applications in the treatment of numerous cancers, in particular prostate cancer and hepatocellular carcinoma.

The following Table 1 summarizes the implication of the DYRKs and CLKs kinases in various diseases.

TABLE 1

| Kinase target | Disease |
|---|---|
| DYRK1A | Down syndrome (DS) |
| DYRK1A | Alzheimer's disease (AD) and |
| | other Tauopathies |
| DYRK1A | Parkinson's disease |
| DYRK1A | Pick disease |
| DYRK1A | CDKL5 Deficiency Disorder |
| DYRK1A | Type 1 and Type 2 diabetes |
| DYRK1A | Abnormalities in folate and |
| | methionine metabolism |
| DYRK1A | Glioblastoma |
| DYRK1A | Head and neck squamous cell carcinoma |
| DYRK1A | Pancreatic ductal adenocarcinoma |
| DYRK1A | Megakaryoblastic leukemia |
| DYRK1A | Acute Lymphoblastic Leukemia (ALL) |
| DYRK1A | Knee osteoarthritis, tendinopathy |
| DYRK1A | Human immunodeficiency virus type 1 (HIV-1) |
| DYRK1A, | Human cytomegalovirus (HCMV) |
| DYRK1B | |
| DYRK1B | Hepatitis C virus, Chikungunya virus, Dengue virus |
| | and Severe acute respiratory syndrome coronavirus, |
| | Cytomegalovirus, Human papillomavirus |
| DYRK1B | Type 1 and Type 2 diabetes |
| DYRK1B | Neuroinflammation |
| DYRK1B | Liposarcoma, Breast cancer, |
| | Hedgehog/GLI-dependent cancer |
| DYRK2 | Triple-negative breast cancer (TNBC) |
| | and multiple myeloma (MM) |
| DYRK2 | Glioblastoma |
| DYRK3 | Hepatocellular carcinoma |
| DYRK3 | Influenza virus replication |
| DYRK3 | Anemia |
| DYRKs | Glioblastoma |
| DYRKs | Herpes simplex virus, cytomegalovirus, |
| | varicella-zoster virus |
| LmDYRK1 | Leishmaniasis |
| TbDYRK | Trypanosoma brucei |
| CLK1 | Glioblastoma |
| CLK1 | Duchenne muscular dystrophy |
| CLK1 | Influenza A |
| CLK2 | HIV-1 |
| CLK1/CLK2 | Triple-negative breast cancer |
| CLK2 | Autism, Phelan-McDermid syndrome (PMDS) |
| CLK2 | Knee osteoarthritis, tendinopathy |
| CLK2 | Breast cancer, Triple negative breast |
| | cancer, Glioblastoma |
| CLK2 | Alzheimer's disease (alternative |
| | splicing of Tau exon 10) |
| CLK3 | Hepatocellular carcinoma, Prostate cancer |
| CLKs | Body temperature |
| CLKs | Prostate cancer, Gastrointestinal cancer |
| PfCLKs | Malaria |
| DYRKs/CLKs | Glioblastoma and numerous other cancers |

### DYRKs and CLK Inhibitors

Several DYRK1A inhibitors have been reported in recent years. Most DYRK1A inhibitors also inhibit DYRK1B, 2, 3, 4, as well as the closely related CLK1, 2, 3, 4, with several possible inhibition profiles.

Some imidazolone derivatives, named Leucettines in the text below, were disclosed in WO 2009/050352 as kinase inhibitors and more particularly as inhibitors of the DYRK1A kinase.

WO 2021/114314 and WO 2021/115489 disclose compounds useful in cardiomyocyte proliferation activity for the treatment of heart diseases. However, these documents comprise a very broad range of compounds illustrated in a very partial way and focused on benzothiazoles derivatives (i.e., in the terms of the present invention, wherein A=═C— and B=—S—). In other words, none of the compounds disclosed are comprised in the formula (I) of the present invention. Furthermore, the compounds disclosed in these applications do not have an optimized activity with regards to DYRK1A and CLK1 and are not directed to the PATHOLOGIES as defined herein after.

There is still a need to identify new compounds for treating and/or preventing the diseases as recited above, and particularly through the inhibition, and in particular selective inhibition of DYRK1A, other DYRKs and the related CLKs kinases. Example 13 herein after further provides comparative biological data showing the biological activity superiority of the claimed compounds with respect to the closest compounds (C1 to C5) of WO2021/114314.

### SUMMARY OF THE INVENTION

It has now been found that the compounds as defined in formula (I) herein after are useful in the treatment and/or prevention of a disease selected from cognitive deficits and neuroinflammation associated with Down syndrome, Alzheimer's disease and related diseases, dementia and tauopathies; Parkinson's disease and other neurodegenerative diseases; CDKL5 Deficiency Disorder; Phelan-McDermid syndrome; autism; type 1 and type 2 diabetes; abnormal folate and methionine metabolism; osteoarthritis and tendinopathy; several cancers and leukemias, neuroinflammation, anemia, infections caused by unicellular parasites, viral infections and for regulating body temperature.

The present invention therefore relates to a compound of formula (I), as defined below.

The present invention further relates to a compound of formula (I) as defined below for use as a medicament.

The present invention further relates to a compound of formula (I) as defined below for use in the treatment and/or prevention of a disease selected from cognitive deficits and neuroinflammation associated with Down syndrome, Alzheimer's disease and related diseases, dementia or tauopathies; Parkinson's disease; other neurodegenerative diseases; CDKL5 Deficiency Disorder; Phelan-McDermid syndrome; autism; type 1 and type 2 diabetes; abnormal folate and methionine metabolism; osteoarthritis and tendinopathy; several cancers and leukemias, neuroinflammation, anemia, infections caused by unicellular parasites, viral infections and for regulating body temperature.

The present invention further relates to a pharmaceutical composition comprising it and to a process for manufacturing it.

The present invention at last describes synthetic intermediates of formula (III) and (IX) as defined below and relates to synthetic intermediates of formula (XI) as defined below.

### Definitions

As used herein, the term “patient” refers to either an animal, such as a valuable animal for breeding, company or preservation purposes, or preferably a human or a human child, which is afflicted with, or has the potential to be afflicted with one or more diseases and conditions described herein.

In particular, as used in the present application, the term “patient” refers to a mammal such as a rodent, cat, dog, primate or human, preferably said subject is a human and also extends to birds.

The identification of those patients who are in need of treatment of herein-described diseases and conditions is well within the ability and knowledge of one skilled in the art. A veterinarian or a physician skilled in the art can readily identify, by the use of clinical tests, physical examination, medical/family history or biological and diagnostic tests, those patients who are in need of such treatment.

In the context of the invention, the term “treating” or “treatment”, as used herein, means preventing, reversing, alleviating, inhibiting the progress of, or preventing the disease and its cognitive, motor or metabolic changes resulting from high DYRK1A kinase and/or CLK1 expression and activity, and optionally associated with the abnormalities in other DYRKs (DYRK1B, 2, 3, 4) and the closely related further cdc2-like kinases (CLKs) (CLK 2, 3, 4) and more particularly in connection to the diseases as described herein after in the paragraph “PATHOLOGIES”.

Therefore, the term “treating” or “treatment” encompasses within the framework of the present invention the improvement of medical conditions of patients suffering from the diseases as described herein after in the paragraph “PATHOLOGIES”, related to high expression and activity of any of the DYRK1A and CLK1 kinases, and optionally associated with the abnormalities in other DYRKs (DYRK1B, 2, 3, 4) and the closely related further cdc2-like kinases (CLKs) (CLK 2, 3, 4).

As used herein, an “effective amount” refers to an amount of a compound of the present invention which is effective in preventing, reducing, eliminating, treating or controlling the symptoms of the herein-described diseases and conditions.

The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

The term “effective amount” includes “prophylaxis-effective amount” as well as “treatment-effective amount”.

The term “preventing”, as used herein, means reducing the risk of onset or slowing the occurrence of a given phenomenon, namely in the present invention, a disease resulting from abnormal DYRKs/CLKs kinase activity, in particular DYRK1A kinase activity.

As used herein, “preventing” also encompasses “reducing the likelihood of occurrence” or “reducing the likelihood of reoccurrence”.

The term “prophylaxis-effective amount” refers to a concentration of compound of this invention that is effective in inhibiting, preventing, decreasing the likelihood of anyone of the hereabove described diseases.

Likewise, the term “treatment-effective amount” refers to a concentration of compound that is effective in treating the hereabove described diseases, e.g. leads to a reduction or normalization DYRK1A and/or CLK1 kinase activity, and optionally additionally of DYRKs/CLKs kinase activity in general, following examination when administered after disease has occurred.

As used herein, the term “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

### DETAILED DESCRIPTION OF THE INVENTION

The inventors have surprisingly found that the compounds of formula (I) as disclosed herein after inhibit the DYRK1A, other DYRKs (DYRK1B, DYRK2, DYRK3, DYRK4) and CLKs (CLK1, CLK2, CLK3, CLK4). This assertion is based on data as illustrated in the following examples and more detailed herein after.

According to a first aspect, a subject-matter of the present invention relates to a compound of formula (I)

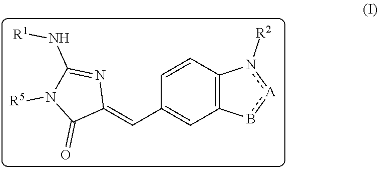

wherein:
A is selected from the group consisting of ═CH—, —N═ and —NR³—,
B is selected from the group consisting of —O—, ═CH—, ═N—, —NH—, —S— and —NR⁴—,
at least one of A and B is ═CH— or —S—,
A and B can not both represent a ═CH—,
R² is selected from the group consisting of a hydrogen atom and a (C₁-C₃)alkyl group or does not exist when the nitrogen atom to which it is attached is —N═,
R³ and R⁴ are independently selected from (C₁-C₃)alkyl groups,
R⁵ represents a hydrogen atom, a (C₁-C₄)alkyl group or a (C₃-C₆)cycloalkyl group, and
when A represents ═CH— and B represents ═N— or —NH—, then R² respectively represents a hydrogen atom or does not exist, and
wherein R¹ represents:
   (i) a (C₄-C₆)alkyl group substituted by a group selected from a hydroxy group, a halogen and a (C₁-C₃)alkoxy group,
   (ii) a (C₃-C₈)cycloalkyl group, optionally substituted by a group selected from a hydroxy group and a (C₁-C₃) alkoxy group,
   (iii) a bridged (C₆-C₁₀)cycloalkyl group, optionally substituted by a group selected from a (C₁-C₄)alkyl group, a (C₁-C₄)alkoxy group, a halogen atom and a hydroxy group,
   (iv) a fused phenyl group, selected from phenyl groups fused with a (C₅-C₆)cycloalkyl, which (C₅-C₆)cycloalkyl is optionally substituted by a hydroxy group,
   (v) a phenyl group, substituted by a (C₄-C₇)heterocycloalkyl group, said (C₄-C₇)heterocycloalkyl group being itself optionally substituted by a (C₁-C₄) alkyl group, or
   (vi) a R′-L- group, wherein L is either a single bond or a (C₁-C₃)alkanediyl group, optionally substituted by a group selected from a hydroxy group and a (C₁-C₃)alkoxy group, and
      R′ represents:
      (vi.1) a (C₃-C₈)heterocycloalkyl group, optionally substituted by one or two groups selected from a hydroxy group, and a (C₁-C₄)alkyl group, or
      (vi.2) a (C₃-C₈)heteroaryl group, optionally substituted by a (C₁-C₄)alkyl group, or
   (vii) a R″-L- group wherein L is a (C₁-C₃)alkanediyl group, optionally substituted by a group selected from a —NR^{b}R^{c} group, a (C₁-C₃)alkoxy group and a hydroxy group, and
   R″ is a phenyl group, optionally substituted by a fluoro(C₁-C₄)alkyl group,
R^{b} and R^{c} independently represent a (C₁-C₆)alkyl group or a hydrogen atom,
or any of its pharmaceutically acceptable salt.

By the expression “at least one of A and B is ═CH— and A and B can not both represent a ═CH—” is meant that only one of A and B can be a ═CH—.

By the expression “R² does not exist” is meant that R² is a lone pair.

The inventors have surprisingly discovered that compounds having the following scaffolds (A) to (J) displayed much reduced kinase inhibitory activities on DYRK1A and other related kinases compared to their homologs (compounds according to the invention): IC₅₀ values were reduced by 10 to 1000-fold factors, and some compounds were completely inactive at the highest dose tested (10 μM).

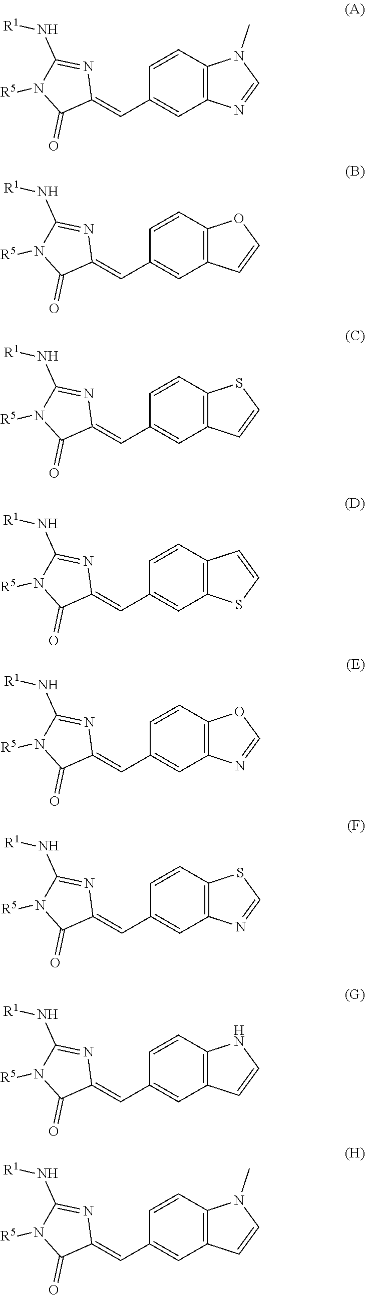

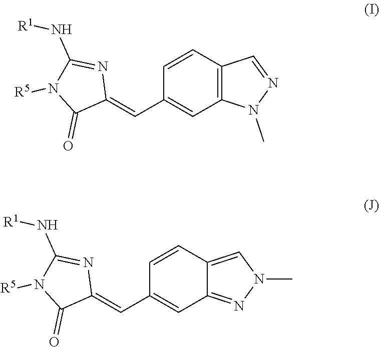

These much-reduced kinase inhibitory activities have been verified, for example, by individual comparison of a compound of formula (I) and of a compound having a scaffold of formula (A) to (J), wherein in both, R′ is selected from the group consisting of cyclohexyl, cycloheptyl, cyclooctyl, 1-(hydroxymethyl)-3-methyl-butyl, 1-(methoxymethyl)-3-methyl-butyl, 2-methoxy-1-phenyl-ethyl, 3-noradamantyl, 1-adamantyl, 3-hydroxy-1-adamantyl, 3-fluoro-1-adamantyl and wherein in both, R⁵ is selected from the group consisting of a methyl group or a hydrogen atom.

According to a particular embodiment, the present invention relates to a compound of formula (I) as defined herein above, wherein R′ represents:
(i) a (C₅-C₆)alkyl group substituted by a group selected from a hydroxy group, a halogen and a (C₁-C₃)alkoxy group,
(ii) a (C₅-C₈)cycloalkyl group, optionally substituted by a group selected from a hydroxy group and a (C₁-C₃) alkoxy group,
(iii) a bridged (C₉-C₁₀)cycloalkyl group, optionally substituted by a group selected from a (C₁-C₄)alkyl group, a (C₁-C₄)alkoxy group, a halogen atom and a hydroxy group,
(iv) a phenyl group fused with a cyclopentyl, which cyclopentyl is optionally substituted by a hydroxy group,
(v) a phenyl group, substituted by a (C₄-C₇)heterocycloalkyl group, said (C₄-C₇)heterocycloalkyl group being itself optionally substituted by a (C₁-C₄) alkyl group, or
(vi) a R′-L- group wherein
   L is either a single bond or a (C₁-C₃)alkanediyl group, optionally substituted by a group selected from a hydroxy group and a (C₁-C₃)alkoxy group, and
   R′ represents:
(vi.1) a (C₆-C₇)heterocycloalkyl group, optionally substituted by one or two groups selected from a hydroxy group and a (C₁-C₄)alkyl group, or
(vi.2) a (C₃-C₈)heteroaryl group, optionally substituted by a (C₁-C₄)alkyl group,
(vi) a R″-L- group wherein
   L is a (C₁-C₃)alkanediyl group, optionally substituted by a group selected from a —NR^{b}R^{c} group, a (C₁-C₃)alkoxy group and a hydroxy group, and
   R″ is a phenyl group, optionally substituted by a fluoro(C₁-C₄)alkyl group, and
R^{b} and R^{c} independently represent a (C₁-C₆)alkyl group or a hydrogen atom,
or any of its pharmaceutically acceptable salt.

According to another particular embodiment, the present invention relates to a compound of formula (I) as defined herein above, wherein R′ represents:
(i) a (C₅-C₆)alkyl group substituted by a group selected from a hydroxy group, a fluorine atom and a methoxy group,
(ii) a (C₅-C₈)cycloalkyl group in particular a cyclopentyl, a cyclohexyl, a cycloheptyl or a cyclooctyl, optionally substituted by a group selected from a hydroxy group and a methoxy group,
(iii) a bridged (C₉-C₁₀)cycloalkyl group in particular an adamantyl or a noradamantyl, optionally substituted by a group selected from a methoxy group, a fluorine atom and a hydroxy group,
(iv) a phenyl group fused with a cyclopentyl, which cyclopentyl is substituted by a hydroxy group,
(v) a phenyl group, substituted by a N-methylpiperazinyl group, or
(vi) a R′-L- group wherein L is either a single bond or a (C₁-C₃)alkanediyl group, optionally substituted by a group chosen from a hydroxy group and a methoxy group,
and R′ is selected from the group consisting of:
   (vi.1) a (C₆-C₇)heterocycloalkyl group, in particular a tetrahydropyranyl or an oxepanyl, optionally substituted by one or two groups selected from a hydroxy group and a methyl group,
   (vi.2) a (C₃-C₈)heteroaryl group, in particular a pyridinyl or a thiazolyl, optionally substituted by a methyl group, or
(vii) a R″-L- group wherein L is a (C₁-C₃)alkanediyl group, optionally substituted by a group selected from the group consisting of a —NH₂ group, a methoxy group, a hydroxy group, a —COOR^{a} group and a halogen atom, in particular a fluorine atom, and
   R″ is a phenyl group, optionally substituted by a trifluoromethyl group,
or any of its pharmaceutically acceptable salts.

According to another particular embodiment, the present invention relates to a compound of formula (I) as defined herein above wherein L is selected from a group consisting of a —CH₂— group, a —CH(CH₂OH)— group, a —CH(CH₂OCH₃)— group, a —CH(OH)—CH₂— group and a —CH(CH₂NH₂)— group, or any of its pharmaceutically acceptable salts.

According to another particular embodiment, the present invention relates to a compound of formula (I) as defined herein above wherein:
(vi.1) when R′ is a (C₅-C₈)heterocycloalkyl group, L is a —CH— group,
(vi.2) when R′ is a phenyl, L is selected from the group consisting of a —CH₂— group, a —CH(CH₂OH)— group, a —CH(CH₂OCH₃)— group, a —CH(OH)—CH₂— group and a —CH(CH₂NH₂)— group,
(vi.3) when R′ is a (C₃-C₈)heteroaryl group, L is a —CH₂— group,
   or any of its pharmaceutically acceptable salts.

According to another particular embodiment, the present invention relates to a compound selected from the following compounds

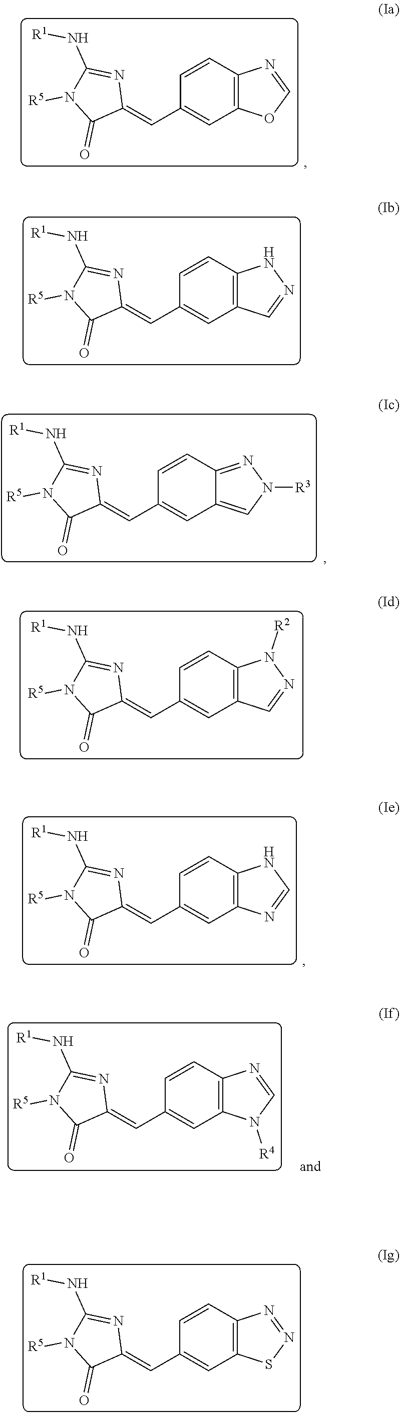

wherein R¹, R², R³, R and R⁵ are as defined above,
or any pharmaceutically acceptable salt thereof.

According to another particular embodiment, the present invention relates to a compound of formula (I) as defined herein above wherein when the compound of formula (I) is chosen from the subformulae (Ib), (Id), (Ie) and (If), R′ represents:
(i) a (C₄-C₆)alkyl group substituted by a group selected from a hydroxy group, a halogen and a (C₁-C₃)alkoxy group,
(ii) a (C₃-C₈)cycloalkyl group, optionally substituted by a group selected from a hydroxy group and a (C₁-C₃) alkoxy group,
(iii) a bridged (C₆-C₁₀)cycloalkyl group, optionally substituted by a group selected from a (C₁-C₄)alkyl group, a (C₁-C₄)alkoxy group, a halogen atom and a hydroxy group, or
(iv) a R′-L- group, wherein L is either a single bond or a (C₁-C₃)alkanediyl group, optionally substituted by a group selected from a hydroxy group and a (C₁-C₃)alkoxy group, and
   R′ represents:
   (iv.1) a (C₃-C₈)heterocycloalkyl group, optionally substituted by one or two groups selected from a hydroxy group, and a (C₁-C₄)alkyl group, or
   (iv.2) a (C₃-C₈)heteroaryl group, optionally substituted by a (C₁-C₄)alkyl group, or
(v) a R″-L- group wherein L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group,
R^{b} and R^{c} independently represent a (C₁-C₆)alkyl group or a hydrogen atom,
or any of its pharmaceutically acceptable salt.

According to another particular embodiment, the present invention relates to a compound of formula (I) as defined herein above wherein R′ represents:
(i) a (C₄-C₆)alkyl group substituted by a group selected from a hydroxy group, a halogen and a (C₁-C₃)alkoxy group,
(ii) a (C₃-C₈)cycloalkyl group, optionally substituted by a group selected from a hydroxy group and a (C₁-C₃) alkoxy group,
(iii) a bridged (C₆-C₁₀)cycloalkyl group, optionally substituted by a group selected from a (C₁-C₄)alkyl group, a (C₁-C₄)alkoxy group, a halogen atom and a hydroxy group, or
(iv) a R′-L- group, wherein L is either a single bond or a (C₁-C₃)alkanediyl group, optionally substituted by a group selected from a hydroxy group and a (C₁-C₃)alkoxy group, and
   R′ represents:
   (iv.1) a (C₃-C₈)heterocycloalkyl group, optionally substituted by one or two groups selected from a hydroxy group, and a (C₁-C₄)alkyl group, or
   (iv.2) a (C₃-C₈)heteroaryl group, optionally substituted by a (C₁-C₄)alkyl group, or
(v) a R″-L- group wherein L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group,
R^{b} and R^{c} independently represent a (C₁-C₆)alkyl group or a hydrogen atom,
or any of its pharmaceutically acceptable salt.

According to another particular embodiment, the present invention relates to a compound of formula (I) as defined herein above wherein R′ represents:
(i) a (C₄-C₆)alkyl group substituted by a group selected from a hydroxy group, a halogen and a (C₁-C₃)alkoxy group,
(ii) a (C₃-C₈)cycloalkyl group, optionally substituted by a group selected from a hydroxy group and a (C₁-C₃) alkoxy group,
(iii) a bridged (C₆-C₁₀)cycloalkyl group, optionally substituted by a group selected from a (C₁-C₄)alkyl group, a (C₁-C₄)alkoxy group, a halogen atom and a hydroxy group, or
(iv) a R′-L- group, wherein L is either a single bond or a (C₁-C₃)alkanediyl group, optionally substituted by a group selected from a hydroxy group and a (C₁-C₃)alkoxy group, and
   R′ represents:
   (iv.1) a (C₃-C₈)heterocycloalkyl group, optionally substituted by one or two groups selected from a hydroxy group, and a (C₁-C₄)alkyl group, or
   (iv.2) a (C₃-C₈)heteroaryl group, optionally substituted by a (C₁-C₄)alkyl group, or
(v) a R″-L- group wherein L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group,
R^{b} and R^{c} independently represent a (C₁-C₆)alkyl group or a hydrogen atom,
or any pharmaceutically acceptable salt thereof.

According to another particular embodiment, the present invention relates to a compound of formula (I) as defined herein above wherein R⁵ represents a hydrogen atom or a (C₁-C₄)alkyl group.

According to another particular embodiment, the present invention relates to a compound of formula (Ia)

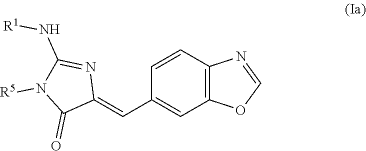

wherein R′ represents:
a (C₅-C₆)alkyl group substituted by a group selected from a hydroxy group and a methoxy group,
a (C₅-C₈)cycloalkyl group in particular a cyclopentyl, a cyclohexyl, a cycloheptyl or a cyclooctyl, optionally substituted by a group selected from a hydroxy group and a methoxy group,
an adamantyl group, optionally substituted by a group selected from a methoxy group and a hydroxy group
a phenyl group fused with a cyclopentyl, which cyclopentyl is optionally substituted by a hydroxy group,
a phenyl group, substituted by a N-methylpiperazinyl group,
a R′-L- group, L is either a single bond or a methylene group, and
R′ is selected from the group consisting of:
   a (C₆-C₇)heterocycloalkyl group, in particular a tetrahydropyranyl or an oxepanyl, optionally substituted by one or two groups selected from a hydroxy group and a methyl group, and
   a (C₃-C₈)heteroaryl group, in particular a pyridyl or a thiazolyl, optionally substituted by a methyl group, or
a R″-L- group wherein
   L is a (C₁-C₂)alkanediyl group, optionally substituted by a group chosen from a hydroxy group and a methoxy group, and
   R″ is a phenyl group, optionally substituted by a trifluoromethyl, and
R⁵ is as defined above,
   or any pharmaceutically acceptable salt thereof.

According to another particular embodiment, the present invention relates to a compound of formula (Ib)

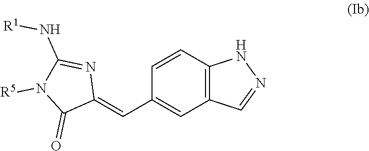

wherein R¹ represents:
a (C₅-C₆)alkyl group substituted by a group selected from a hydroxy group and a methoxy group,
a cyclohexyl group,
an adamantyl group,
a phenyl group, substituted by a N-methylpiperazinyl group, or
a R″-L- group wherein
   L is a methoxymethylmethylene group, and
   R″ is a phenyl group, and
R⁵ is as defined above,
   or any pharmaceutically acceptable salt thereof.

According to another particular embodiment, the present invention relates to a compound of formula (Ic)

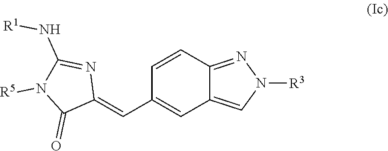

wherein R′ represents:
a (C₅-C₆)alkyl group substituted by a group selected from a hydroxy group and a methoxy group,
a cyclohexyl group,
an adamantyl group, optionally substituted by a group selected from a halogen atom and a hydroxy group, or
a R″-L- group wherein
   L is a methoxymethylmethylene group, and
   R″ is a phenyl group, and
wherein R³ represents a methyl group, and
R⁵ is as defined above,
   or any pharmaceutically acceptable salt thereof.

According to another particular embodiment, the present invention relates to a compound of formula (Id)

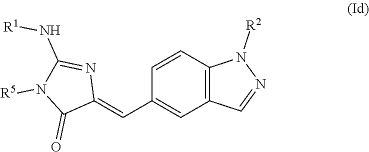

wherein R′ represents:
a (C₅-C₆)alkyl group substituted by a group selected from a hydroxy group, a methoxy group and a fluorine atom,
a (C₅-C₈)cycloalkyl group in particular a cyclopentyl, a cyclohexyl, a cycloheptyl or a cyclooctyl group, optionally substituted by a group selected from a hydroxy group and a methoxy group,
an adamantyl group, optionally substituted by a group selected from a methoxy group, a hydroxy group and a fluorine atom,
a phenyl group fused with a cyclopentyl, which cyclopentyl is optionally substituted by a hydroxy group,
a phenyl group, substituted by a N-methylpiperazinyl group,
a R′-L- group, L is either a single bond or a methylene group, and R′ is selected from the group consisting of:
   a (C₆-C₇)heterocycloalkyl group, in particular a tetrahydropyranyl or an oxepanyl, optionally substituted by one or two groups selected from a hydroxy group and a methyl group, and
   a (C₃-C₈)heteroaryl group, in particular a pyridyl or a thiazolyl group, optionally substituted by a methyl group, or
a R″-L- group wherein
   L is a (C₁-C₂)alkanediyl group, optionally substituted by a group chosen from a hydroxy group, a methoxy group and a —NH₂ group, and
   R″ is a phenyl group, optionally substituted by a trifluoromethyl, and
wherein R² represents a methyl group, and
R′ is as defined above,
   or any pharmaceutically acceptable salt thereof.

According to another particular embodiment, the present invention relates to a compound of formula (Ie)

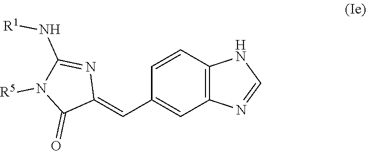

wherein R′ represents:
a (C₅-C₆)alkyl group substituted by a group selected from a hydroxy group, a methoxy group and a fluorine atom,
a (C₅-C₈)cycloalkyl group in particular a cyclopentyl, a cyclohexyl, a cycloheptyl or a cyclooctyl, optionally substituted by a group selected from a hydroxy group and a methoxy group,
a bridged (C₉-C₁₀)cycloalkyl group, in particular an adamantyl or a noradamantyl, optionally substituted by a group selected from a methoxy group, a fluorine atom and a hydroxy group,
a phenyl group fused with a cyclopentyl, which cyclopentyl is optionally substituted by a hydroxy group,
a phenyl group, substituted by a N-methylpiperazinyl group,
a R′-L- group wherein L is either a single bond or a methylene group, and
R′ is selected from the group consisting of:
   a (C₆-C₇)heterocycloalkyl group, in particular a tetrahydropyranyl or an oxepanyl, optionally substituted by a hydroxy group, and
   a (C₃-C₈)heteroaryl group, in particular a pyridinyl or a thiazolyl group, optionally substituted by a methyl group, or
a R″-L- group wherein
   L is a (C₁-C₂)alkanediyl group, optionally substituted by a group chosen from a hydroxy group, a methoxy group and a —NH₂ group, and
   R″ is a phenyl group, optionally substituted by a trifluoromethyl, and
R⁵ is as defined above,
   or any pharmaceutically acceptable salt thereof.

According to another particular embodiment, the present invention relates to a compound of formula (If)

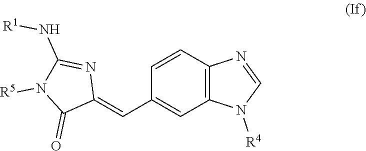

wherein R′ represents:
a (C₅-C₆)alkyl group substituted by a group selected from a hydroxy group, a methoxy group and a fluorine atom,
a (C₆-C₈)cycloalkyl group in particular a cyclohexyl, a cycloheptyl or a cyclooctyl,
a bridged (C₉-C₁₀)cycloalkyl group in particular an adamantyl or a noradamantyl, optionally substituted by a group selected from a methoxy group, a fluorine atom and a hydroxy group,
a phenyl group fused with a cyclopentyl, which cyclopentyl is optionally substituted by a hydroxy group,
a phenyl group, substituted by a N-methylpiperazinyl group,
a R′-L- group wherein L is either a single bond or a methylene group, and R′ is a (C₃-C₈)heteroaryl group, in particular a pyridyl or a thiazolyl, optionally substituted by a methyl group,
a Rₐ′-L- group wherein L is a methylene group and Rₐ′ is a tetrahydropyranyl group, or
a R″-L- group wherein
   L is a (C₁-C₂)alkanediyl group, optionally substituted by a group chosen from a hydroxy group, a methoxy group and a —NH₂ group, and
   R″ is a phenyl group, optionally substituted by a trifluoromethyl, and
wherein R⁴ represents a methyl group, and
R⁵ is as defined above,
or any pharmaceutically acceptable salt thereof.

According to another particular embodiment, the present invention relates to a compound of formula (Ig)

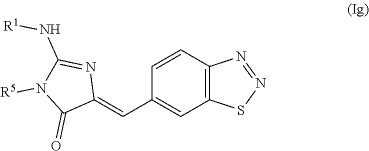

wherein R′ represents:
   a (C₅-C₆)alkyl group substituted by a group selected from a hydroxy group, a methoxy group and a fluorine atom,
   a (C₆-C₈)cycloalkyl group in particular a cyclohexyl or a cycloheptyl,
   a bridged (C₉-C₁₀)cycloalkyl group in particular an adamantyl or a noradamantyl, optionally substituted by a group selected from a methoxy group, a fluorine atom and a hydroxy group.

According to another particular embodiment, the present invention relates to a compound of formula (I) as defined herein above, wherein R′ represents:
a (C₄-C₆)alkyl group substituted by a group selected from a hydroxy group, a halogen and a (C₁-C₃)alkoxy, or
a (C₃-C₈)cycloalkyl group, optionally substituted by a group selected from a hydroxy group and a (C₁-C₃) alkoxy group,
a bridged (C₆-C₁₀)cycloalkyl group, optionally substituted by a group selected from a (C₁-C₄)alkyl group, a (C₁-C₄)alkoxy group, a halogen atom and a hydroxy group,

or any pharmaceutically acceptable salt thereof.

Said sub-group of compounds are gathered under the “A1” type of compounds within the herein after table 1.

Still according to said embodiment, R′ may more particularly represent a cyclohexyl, a cycloheptyl, a cyclooctyl, a 1-adamantyl, a 3-hydroxy-1-adamantyl, a 3-methoxy-1-adamantyl, a 1-(hydroxymethyl)-3-methyl-butyl, a 1-(methoxymethyl)-3-methyl-butyl, a 1-(fluoromethyl)-3-methyl-butyl, a 2-methoxycyclopentyl, a 4-hydroxycycloheptyl, a 3-methoxycycloheptyl, a 4-methoxycycloheptyl, a 3-noradamantyl, 3-fluoro-1-adamantyl.

According to another particular embodiment, the present invention relates to a compound of formula (I) as defined herein above wherein R′ represents:
a fused phenyl group, selected from phenyl groups fused with a (C₅-C₆)cycloalkyl, which (C₅-C₆)cycloalkyl is optionally substituted by a hydroxy group,
a phenyl group, substituted by a (C₄-C₇)heterocycloalkyl group, said (C₄-C₇)heterocycloalkyl group being itself optionally substituted by a (C₁-C₄) alkyl group, or
a R″-L- group, wherein
   L is a (C₁-C₃)alkanediyl group, optionally substituted by a group chosen from a hydroxy group, a (C₁-C₃)alkoxy group and a —NR^{b}R^{c} group, and
   R″ is a phenyl group, optionally substituted by a fluoro(C₁-C₄)alkyl group, wherein R^{b} and R^{c} are independently chosen from (C₁-C₆)alkyl and a hydrogen atom
or any pharmaceutically acceptable salt thereof.

Said sub-group of compounds are gathered under the “A2” and “A5” type of compounds within the herein after table 1.

Still according to said embodiment, R¹ may more particularly represent a [2-(trifluoromethyl)phenyl]methyl, a 2-hydroxyindan-1-yl, a 2-amino-1-phenyl-ethyl, a 2-hydroxy-1-phenyl-ethyl, a 2-methoxy-1-phenyl-ethyl, a 2-hydroxy-2-phenyl-ethyl, a 4-(4-methylpiperazin-1-yl)phenyl.

According to another particular embodiment, the present invention relates to a compound of formula (I) as defined herein above wherein R¹ represents a R′-L- group wherein:
R′ is a (C₃-C₈)heteroaryl group, optionally substituted by a (C₁-C₄)alkyl group and
L is a (C₁-C₃)alkanediyl or a single bond,

or any pharmaceutically acceptable salt thereof.

Said sub-group of compounds are gathered under the “A3” and “A6” type of compounds within the herein after table 1.

Still according to said embodiment, R¹ may more particularly represent a (4-methylthiazol-2-yl)methyl or a 2-pyridyl.

Herein is further provided a compound of formula (I) as defined above wherein R′ represents:
a (C₄-C₆)alkyl group substituted by a group selected from a hydroxy group, a halogen and a (C₁-C₃)alkoxy group,
a bridged (C₆-C₁₀)cycloalkyl group, optionally substituted by a group selected from a (C₁-C₄)alkyl group, a (C₁-C₄)alkoxy group, a halogen atom and a hydroxy group, or
a R″-L- group wherein L is a (C₁-C₃)alkanediyl group, optionally substituted by a group selected from a —NR^{b}R^{c} group, a (C₁-C₃)alkoxy group and a hydroxy group, and R″ is a phenyl group, optionally substituted by a fluoro(C₁-C₄)alkyl group.

According to another particular embodiment, the present invention relates to a compound of formula (I) as defined herein above wherein R¹ represents a R′-L- group wherein:
R′ is a (C₃-C₈)heterocycloalkyl group, optionally substituted by one to two groups selected from a hydroxy group and a (C₁-C₄)alkyl, and
L is a (C₁-C₃)alkanediyl or a single bond,

or any pharmaceutically acceptable salt thereof.

Said sub-group of compounds are gathered under the “A4” and “A7” type of compounds within the herein after table 1.

Still according to said embodiment, R′ may more particularly a tetrahydropyran-4-yl-methyl, a tetrahydropyran-3-yl, a 6,6-dimethyltetrahydropyran-3-yl, a 4-hydroxytetrahydropyran-3-yl, or an oxepan-3-yl.

In the context of the present invention, the term:
“halogen” is understood to mean chlorine, fluorine, bromine, or iodine, and in particular denotes chlorine, fluorine or bromine,
“(Cₓ-C_{y})alkyl”, as used herein, respectively refers to a Cₓ-C_{y} normal, secondary or tertiary monovalent saturated hydrocarbon radical, for example (C₁-C₆)alkyl. Examples are, but are not limited to, methyl, ethyl, propyl, n-propyl, isopropyl, butyl, isobutyl, sec-butyl, tert-butyl, pentyl, isopentyl, hexyl and isohexyl groups, and the like.
“(C₁-C₃)alkanediyl”, as used herein, refers to a divalent saturated hydrocarbon radical which is branched or linear, comprises from 1 to 3 carbon atoms, and more particularly a methylene, ethylene or propylene, such as linear propylene or isopropylene, said alkanediyl may be substituted as it is apparent from the following description.
“(C₃-C₈)cycloalkyl”, as used herein, refers to a cyclic saturated hydrocarbon, from 3 to 8 carbon atoms, saturated or partially unsaturated and unsubstituted or substituted. Examples are, but are not limited to, cyclopropyl, cyclobutyl, cyclopentyl, cyclohexyl, cycloheptyl and cyclooctyl.
“(C₃-C₈)heterocycloalkyl group”, as used herein, refers to a (C₃-C₈)cycloalkyl group wherein one or two of the carbon atoms are replaced with a heteroatom such as oxygen, nitrogen or sulphur, and more particularly such as an oxygen or a nitrogen atom. Such heterocycloalkyl group may be saturated or partially saturated and unsubstituted or substituted. Examples are, but are not limited to, morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl and tetrahydrothiopyranyl, and more particularly piperidinyl and piperazinyl, and even more particularly piperazinyl.
“(C₁-Cₓ)alkoxy”, as used herein, refers to a —O—(C₁-Cₓ)alkyl or —O—(C₃-Cₓ)cycloalkyl moiety, wherein alkyl and cycloalkyl are as defined above, for example (C₁-C₆)alkoxy. Examples are, but are not limited to, methoxy, ethoxy, 1-propoxy, 2-propoxy, cyclopropoxy, butoxy, tert-butoxy and pentoxy.
a “bridged (C₆-C₁₀)cycloalkyl” group, as used herein, refers to a bi- or tricyclic compound where the cycles are cycloalkyls, the rings share three or more atoms and the bridge contains at least one atom, for example 1, 2 or 3 atoms. Such bridged cycloalkyl groups may be substituted by one or more C₁-C₃ alkyl. Examples are, but not limited to adamantyl and noradamantyl.
a “fused phenyl group” refers to a bicyclic radical that contains a phenyl moiety and may be substituted. Said fused phenyl group may be fused to a cycloalkyl or to a heterocycloalkyl and bound to the rest of the molecule either by its phenyl moeity or by said cycloalkyl or heterocycloalkyl. Examples are, but are not limited to indanyl, acetylindolinyl, methylindazolyl, hydroxyindanyl, benzothiazolyl, indolyl, indazolyl, methoxyindanyl and the like.
a (C₃-C₈)heteroaryl group, as used herein, refers to a monocyclic aromatic group where at least one of the ring is aromatic and wherein one to three ring carbon atom is replaced by a heteroatom, such as nitrogen, oxygen or sulphur. By way of examples of (C₃-C₈)heteroaryl groups, mention may be made of, but not limited to: oxazole, isoxazole, pyridine, pyrimidine, pyridazine, triazine, pyrazine, oxadiazole, furane, pyrazole, thiazole, isothiazole, thiadiazole, imidazole, triazole and the like. In the framework of the present invention, the (C₃-C₈)heteroaryl is advantageously pyridine, imidazole, pyrazine, furane, thiazole, pyrazole, thiadiazole, pyridazine and pyrimidine.
an aromatic ring means, according to Hickel's rule, that a molecule has 4n+2 π-electrons.
a (C₁-Cₓ)fluoroalkyl group, as used herein, refers to a (C₁-Cₓ)alkyl as defined herein above in which one or more hydrogen atom(s) have been substituted by fluorines. In one embodiment all the hydrogen atoms are replaced by fluorine atoms, forming perfluoroalkyl groups, such as trifluoromethyl.

In the context of the present invention, the terms “aromatic ring”, and “heteroaryl” include all the positional isomers.

The nomenclature of the following compounds (1) to (271) was generated according to the principles of the International Union of Pure and Applied Chemistry, using Accelrys Draw 4.1 SPI. To avoid any confusion, the “(+)” symbol added to designates a racemic mixture; “cis” and “trans” prefixes were also used to assign the relative stereochemistry of two adjacent chiral centers.

According to a preferred embodiment of the present invention, the compound of formula (I) is chosen from:
(1) (4Z)-4-(1,3-Benzoxazol-6-ylmethylene)-2-(cyclohexylamino)-1H-imidazol-5-one,
(2) (4Z)-4-(1,3-Benzoxazol-6-ylmethylene)-2-(cycloheptylamino)-1H-imidazol-5-one,
(3) (4Z)-4-(1,3-Benzoxazol-6-ylmethylene)-2-(cyclooctylamino)-1H-imidazol-5-one,
(4) (4Z)-4-(1,3-Benzoxazol-6-ylmethylene)-2-[[(1R)-1-(hydroxymethyl)-3-methyl-butyl]amino]-1H-imidazol-5-one,
(5) (4Z)-4-(1,3-Benzoxazol-6-ylmethylene)-2-[[(1R)-1-(methoxymethyl)-3-methyl-butyl]amino]-1H-imidazol-5-one,
(6) (4Z)-4-(1,3-Benzoxazol-6-ylmethylene)-2-[[(1R,2R)-2-methoxycyclopentyl]amino]-1H-imidazol-5-one,
(7) (4Z)-4-(1,3-Benzoxazol-6-ylmethylene)-2-[[(1S,2S)-2-methoxycyclopentyl]amino]-1H-imidazol-5-one,
(8) (±)-(4Z)-4-(1,3-Benzoxazol-6-ylmethylene)-2-[[trans-4-hydroxycycloheptyl]amino]-1H-imidazol-5-one,
(9) (±)-(4Z)-4-(1,3-Benzoxazol-6-ylmethylene)-2-[[trans-4-methoxycycloheptyl]amino]-1H-imidazol-5-one,
(10) (±)-(4Z)-4-(1,3-Benzoxazol-6-ylmethylene)-2-[[cis-3-methoxycycloheptyl]amino]-1H-imidazol-5-one,
(11) (4Z)-2-(1-Adamantylamino)-4-(1,3-benzoxazol-6-ylmethylene)-1H-imidazol-5-one,
(12) (4Z)-4-(1,3-Benzoxazol-6-ylmethylene)-2-[(3-hydroxy-1-adamantyl)amino]-1H-imidazol-5-one,
(13) (4Z)-4-(1,3-Benzoxazol-6-ylmethylene)-2-[(3-methoxy-1-adamantyl)amino]-1H-imidazol-5-one.
(14) (4Z)-4-(1,3-Benzoxazol-6-ylmethylene)-2-[[2-(trifluoromethyl)phenyl]methylamino]-1H-imidazol-5-one,
(15) (4Z)-4-(1,3-Benzoxazol-6-ylmethylene)-2-[[(1S,2S)-2-hydroxyindan-1-yl]amino]-1H-imidazol-5-one,
(16) (4Z)-4-(1,3-Benzoxazol-6-ylmethylene)-2-[[(1R)-2-hydroxy-1-phenyl-ethyl]amino]-1H-imidazol-5-one,
(17) (4Z)-4-(1,3-Benzoxazol-6-ylmethylene)-2-[[(1S)-2-hydroxy-1-phenyl-ethyl]amino]-1H-imidazol-5-one,
(18) (4Z)-4-(1,3-Benzoxazol-6-ylmethylene)-2-[[(1R)-2-methoxy-1-phenyl-ethyl]amino]-1H-imidazol-5-one,
(19) (4Z)-4-(1,3-Benzoxazol-6-ylmethylene)-2-[(4-methylthiazol-2-yl)methylamino]-1H-imidazol-5-one,
(20) (4Z)-4-(1,3-Benzoxazol-6-ylmethylene)-2-(tetrahydropyran-4-ylmethylamino)-1H-imidazol-5-one,
(21) (4Z)-4-(1,3-Benzoxazol-6-ylmethylene)-2-[4-(4-methylpiperazin-1-yl)anilino]-1H-imidazol-5-one,
(22) (4Z)-4-(1,3-Benzoxazol-6-ylmethylene)-2-(2-pyridylamino)-1H-imidazol-5-one,
(23) (±)-(4Z)-4-(1,3-Benzoxazol-6-ylmethylene)-2-[(6,6-dimethyltetrahydropyran-3-yl)amino]-1H-imidazol-5-one.
(24) (4Z)-4-(1,3-Benzoxazol-6-ylmethylene)-2-[[(3S)-tetrahydropyran-3-yl]amino]-1H-imidazol-5-one,
(25) (±)-(4Z)-4-(1,3-Benzoxazol-6-ylmethylene)-2-(oxepan-3-ylamino)-1H-imidazol-5-one,
(26) (4Z)-2-(Cyclohexylamino)-4-(1H-indazol-5-ylmethylene)-1H-imidazol-5-one,
(27) 4Z)-2-(Cycloheptylamino)-4-(1H-indazol-5-ylmethylene)-1H-imidazol-5-one,
(28) 4Z)-2-(Cyclooctylamino)-4-(1H-indazol-5-ylmethylene)-1H-imidazol-5-one,
(29) (4Z)-2-[[(1R)-1-(Hydroxymethyl)-3-methyl-butyl]amino]-4-(1H-indazol-5-ylmethylene)-1H-imidazol-5-one,
(30) (4Z)-4-(1H-Indazol-5-ylmethylene)-2-[[(1R)-1-(methoxymethyl)-3-methyl-butyl]amino]-1H-imidazol-5-one,
(31) (4Z)-2-(1-Adamantylamino)-4-(1H-indazol-5-ylmethylene)-1H-imidazol-5-one,
(32) (4Z)-2-[(3-Hydroxy-1-adamantyl)amino]-4-(1H-indazol-5-ylmethylene)-1H-imidazol-5-one,
(33) (4Z)-4-(1H-Indazol-5-ylmethylene)-2-[[(1R)-2-methoxy-1-phenyl-ethyl]amino]-1H-imidazol-5-one,
(34) (4Z)-2-(Cyclohexylamino)-4-[(2-methylindazol-5-yl)methylene]-1H-imidazol-5-one,
(35) (4Z)-2-(Cycloheptylamino)-4-[(2-methylindazol-5-yl)methylene]-1H-imidazol-5-one,
(36) (4Z)-2-(Cyclooctylamino)-4-[(2-methylindazol-5-yl)methylene]-1H-imidazol-5-one,
(37) (4Z)-2-[[(1R)-1-(Hydroxymethyl)-3-methyl-butyl]amino]-4-[(2-methylindazol-5-yl)methylene]-1H-imidazol-5-one,
(38) (4Z)-2-[[(1R)-1-(Methoxymethyl)-3-methyl-butyl]amino]-4-[(2-methylindazol-5-yl)methylene]-1H-imidazol-5-one,
(39) (4Z)-2-(1-Adamantylamino)-4-[(2-methylindazol-5-yl)methylene]-1H-imidazol-5-one,
(40) (4Z)-2-[(3-Hydroxy-1-adamantyl)amino]-4-[(2-methylindazol-5-yl)methylene]-1H-imidazol-5-one,
(41) (4Z)-2-[(3-Fluoro-1-adamantyl)amino]-4-[(2-methylindazol-5-yl)methylene]-1H-imidazol-5-one,
(42) (4Z)-2-[[(1R)-2-Methoxy-1-phenyl-ethyl]amino]-4-[(2-methylindazol-5-yl)methylene]-1H-imidazol-5-one,
(43) (4Z)-2-(Cyclohexylamino)-4-[(1-methylindazol-5-yl)methylene]-1H-imidazol-5-one,
(44) (4Z)-2-(Cycloheptylamino)-4-[(1-methylindazol-5-yl)methylene]-1H-imidazol-5-one,
(45) (4Z)-2-(Cyclooctylamino)-4-[(1-methylindazol-5-yl)methylene]-1H-imidazol-5-one,
(46) (4Z)-2-[[(1R)-1-(Hydroxymethyl)-3-methyl-butyl]amino]-4-[(1-methylindazol-5-yl)methylene]-1H-imidazol-5-one,
(47) (4Z)-2-[[(1R)-1-(Methoxymethyl)-3-methyl-butyl]amino]-4-[(1-methylindazol-5-yl)methylene]-1H-imidazol-5-one,
(48) (4Z)-2-[[(1R)-1-(Fluoromethyl)-3-methyl-butyl]amino]-4-[(1-methylindazol-5-yl)methylene]-1H-imidazol-5-one,
(49) (4Z)-2-[[(1S)-1-(Fluoromethyl)-3-methyl-butyl]amino]-4-[(1-methylindazol-5-yl)methylene]-1H-imidazol-5-one,
(50) (4Z)-2-[[(1R,2R)-2-Methoxycyclopentyl]amino]-4-[(1-methylindazol-5-yl)methylene]-1H-imidazol-5-one,
(51) (4Z)-2-[[(1S,2S)-2-Methoxycyclopentyl]amino]-4-[(1-methylindazol-5-yl)methylene]-1H-imidazol-5-one,
(52) (±)-(4Z)-2-[[trans-4-Hydroxycycloheptyl]amino]-4-[(1-methylindazol-5-yl)methylene]-1H-imidazol-5-one,
(53) (f)-(4Z)-2-[[trans-4-Methoxycycloheptyl]amino]-4-[(1-methylindazol-5-yl)methylene]-1H-imidazol-5-one,
(54) (f)-(4Z)-2-[[cis-3-Methoxycycloheptyl]amino]-4-[(1-methylindazol-5-yl)methylene]-1H-imidazol-5-one,
(55) (4Z)-2-(1-Adamantylamino)-4-[(1-methylindazol-5-yl)methylene]-1H-imidazol-5-one,
(56) (4Z)-2-[(3-Hydroxy-1-adamantyl)amino]-4-[(1-methylindazol-5-yl)methylene]-1H-imidazol-5-one,
(57) (4Z)-2-[(3-Methoxy-1-adamantyl)amino]-4-[(1-methylindazol-5-yl)methylene]-1H-imidazol-5-one,
(58) (4Z)-2-[(3-Fluoro-1-adamantyl)amino]-4-[(1-methylindazol-5-yl)methylene]-1H-imidazol-5-one,
(59) (4Z)-4-[(I-Methylindazol-5-yl)methylene]-2-[[2-(trifluoromethyl)phenyl]methylamino]-1H-imidazol-5-one,
(60) (4Z)-2-[[(1S,2S)-2-Hydroxyindan-1-yl]amino]-4-[(1-methylindazol-5-yl)methylene]-1H-imidazol-5-one,
(61) (4Z)-2-[[(1R)-2-Hydroxy-1-phenyl-ethyl]amino]-4-[(1-methylindazol-5-yl)methylene]-1H-imidazol-5-one.
(62) (4Z)-2-[[(1S)-2-Hydroxy-1-phenyl-ethyl]amino]-4-[(1-methylindazol-5-yl)methylene]-1H-imidazol-5-one,
(63) (4Z)-2-[[(1R)-2-Methoxy-1-phenyl-ethyl]amino]-4-[(1-methylindazol-5-yl)methylene]-1H-imidazol-5-one,
(64) (4Z)-2-[[(2R)-2-Hydroxy-2-phenyl-ethyl]amino]-4-[(1-methylindazol-5-yl)methylene]-1H-imidazol-5-one,
(65) (4Z)-2-[[(1R)-2-Amino-1-phenyl-ethyl]amino]-4-[(1-methylindazol-5-yl)methylene]-1H-imidazol-5-one dihydrochloride,
(66) (4Z)-2-[[(1S)-2-Amino-1-phenyl-ethyl]amino]-4-[(1-methylindazol-5-yl)methylene]-1H-imidazol-5-one dihydrochloride,
(67) (4Z)-4-[(1-Methylindazol-5-yl)methylene]-2-[(4-methylthiazol-2-yl)methylamino]-1H-imidazol-5-one,
(68) (4Z)-4-[(1-Methylindazol-5-yl)methylene]-2-(tetrahydropyran-4-ylmethylamino)-1H-imidazol-5-one,
(69) (4Z)-4-[(1-Methylindazol-5-yl)methylene]-2-[4-(4-methylpiperazin-1-yl)anilino]-1H-imidazol-5-one,
(70) (4Z)-4-[(1-Methylindazol-5-yl)methylene]-2-(2-pyridylamino)-1H-imidazol-5-one,
(71) (4Z)-2-[(6,6-Dimethyltetrahydropyran-3-yl)amino]-4-[(1-methylindazol-5-yl)methylene]-1H-imidazol-5-one,
(72) (4Z)-4-[(1-Methylindazol-5-yl)methylene]-2-[[(3S)-tetrahydropyran-3-yl]amino]-1H-imidazol-5-one,
(73) (4Z)-2-[[(3R,4R)-4-Hydroxytetrahydropyran-3-yl]amino]-4-[(1-methylindazol-5-yl)methylene]-1H-imidazol-5-one,
(74) (±)-(4Z)-4-[(1-Methylindazol-5-yl)methylene]-2-(oxepan-3-ylamino)-1H-imidazol-5-one,
(75) (4Z)-4-(1H-Benzimidazol-5-ylmethylene)-2-(cyclohexylamino)-1H-imidazol-5-one,
(76) (4Z)-4-(1H-Benzimidazol-5-ylmethylene)-2-(cycloheptylamino)-1H-imidazol-5-one.
(77) (4Z)-4-(1H-Benzimidazol-5-ylmethylene)-2-(cyclooctylamino)-1H-imidazol-5-one,
(78) (4Z)-4-(1H-Benzimidazol-5-ylmethylene)-2-[[(1R)-1-(hydroxymethyl)-3-methyl-butyl]amino]-1H-imidazol-5-one,
(79) (4Z)-4-(1H-Benzimidazol-5-ylmethylene)-2-[[(1R)-1-(methoxymethyl)-3-methyl-butyl]amino]-1H-imidazol-5-one,
(80) (4Z)-4-(1H-Benzimidazol-5-ylmethylene)-2-[[(1R)-1-(fluoromethyl)-3-methyl-butyl]amino]-1H-imidazol-5-one,
(81) (4Z)-4-(1H-Benzimidazol-5-ylmethylene)-2-[[(1S)-1-(fluoromethyl)-3-methyl-butyl]amino]-1H-imidazol-5-one,
(82) (4Z)-4-(1H-Benzimidazol-5-ylmethylene)-2-[[(1R,2R)-2-methoxycyclopentyl]amino]-1H-imidazol-5-one,
(83) (4Z)-2-(3-Noradamantylamino)-4-(1H-benzimidazol-5-ylmethylene)-1H-imidazol-5-one,
(84) (4Z)-2-(1-Adamantylamino)-4-(1H-benzimidazol-5-ylmethylene)-1H-imidazol-5-one,
(85) (4Z)-4-(1H-Benzimidazol-5-ylmethylene)-2-[(3-hydroxy-1-adamantyl)amino]-1H-imidazol-5-one,
(86) (4Z)-4-(1H-Benzimidazol-5-ylmethylene)-2-[(3-methoxy-1-adamantyl)amino]-1H-imidazol-5-one,
(87) (4Z)-4-(1H-benzimidazol-5-ylmethylene)-2-[(3-fluoro-1-adamantyl)amino]-1H-imidazol-5-one,
(88) (4Z)-4-(1H-Benzimidazol-5-ylmethylene)-2-[[2-(trifluoromethyl)phenyl]methylamino]-1H-imidazol-5-one,
(89) (4Z)-4-(1H-Benzimidazol-5-ylmethylene)-2-[[(1S,2S)-2-hydroxyindan-1-yl]amino]-1H-imidazol-5-one,
(90) (4Z)-4-(1H-Benzimidazol-5-ylmethylene)-2-[[(1R)-2-hydroxy-1-phenyl-ethyl]amino]-1H-imidazol-5-one,
(91) (4Z)-4-(1H-Benzimidazol-5-ylmethylene)-2-[[(1S)-2-hydroxy-1-phenyl-ethyl]amino]-1H-imidazol-5-one,
(92) (4Z)-4-(1H-Benzimidazol-5-ylmethylene)-2-[[(1R)-2-methoxy-1-phenyl-ethyl]amino]-1H-imidazol-5-one,
(93) (4Z)-4-(1H-Benzimidazol-5-ylmethylene)-2-[[(2R)-2-hydroxy-2-phenyl-ethyl]amino]-1H-imidazol-5-one,
(94) (4Z)-4-(1H-Benzimidazol-5-ylmethylene)-2-[[(2S)-2-hydroxy-2-phenyl-ethyl]amino]-1H-imidazol-5-one,
(95) (4Z)-2-[[(1R)-2-Amino-1-phenyl-ethyl]amino]-4-(1H-benzimidazol-5-ylmethylene)-1H-imidazol-5-one dihydrochloride,
(96) (4Z)-2-[[(1S)-2-Amino-1-phenyl-ethyl]amino]-4-(1H-benzimidazol-5-ylmethylene)-1H-imidazol-5-one dihydrochloride,
(97) (4Z)-4-(1H-Benzimidazol-5-ylmethylene)-2-[(4-methylthiazol-2-yl)methylamino]-1H-imidazol-5-one,
(98) (4Z)-4-(1H-Benzimidazol-5-ylmethylene)-2-(tetrahydropyran-4-ylmethylamino)-1H-imidazol-5-one,
(99) (4Z)-4-(1H-Benzimidazol-5-ylmethylene)-2-[4-(4-methylpiperazin-1-yl)anilino]-1H-imidazol-5-one,
(100) (4Z)-4-(1H-Benzimidazol-5-ylmethylene)-2-(2-pyridylamino)-1H-imidazol-5-one,
(101) (4Z)-4-(1H-Benzimidazol-5-ylmethylene)-2-[[(3S)-tetrahydropyran-3-yl]amino]-1H-imidazol-5-one,
(102) (4Z)-4-(1H-Benzimidazol-5-ylmethylene)-2-[[(3R,4R)-4-hydroxytetrahydropyran-3-yl]amino]-1H-imidazol-5-one,
(103) (4Z)-4-(1H-Benzimidazol-5-ylmethylene)-2-[[(3S,4S)-4-hydroxytetrahydropyran-3-yl]amino]-1H-imidazol-5-one,
(104) (4Z)-4-(1H-Benzimidazol-5-ylmethylene)-2-(oxepan-3-ylamino)-1H-imidazol-5-one,
(105) (4Z)-2-(Cyclohexylamino)-4-[(3-methylbenzimidazol-5-yl)methylene]-1H-imidazol-5-one,
(106) (4Z)-2-(Cycloheptylamino)-4-[(3-methylbenzimidazol-5-yl)methylene]-1H-imidazol-5-one,
(107) (4Z)-2-(Cyclooctylamino)-4-[(3-methylbenzimidazol-5-yl)methylene]-1H-imidazol-5-one,
(108) (4Z)-2-[[(1R)-1-(Hydroxymethyl)-3-methyl-butyl]amino]-4-[(3-methylbenzimidazol-5-yl)methylene]-1H-imidazol-5-one,
(109) (4Z)-2-[[(1R)-1-(Methoxymethyl)-3-methyl-butyl]amino]-4-[(3-methylbenzimidazol-5-yl)methylene]-1H-imidazol-5-one,
(110) (4Z)-2-[[(1R)-1-(Fluoromethyl)-3-methyl-butyl]amino]-4-[(3-methylbenzimidazol-5-yl)methylene]-1H-imidazol-5-one,
(111) (4Z)-2-[[(1S)-1-(Fluoromethyl)-3-methyl-butyl]amino]-4-[(3-methylbenzimidazol-5-yl)methylene]-1H-imidazol-5-one,
(112) (4Z)-2-[[(1R,2R)-2-Methoxycyclopentyl]amino]-4-[(3-methylbenzimidazol-5-yl)methylene]-1H-imidazol-5-one,
(113) (4Z)-2-[[(1S,2S)-2-Methoxycyclopentyl]amino]-4-[(3-methylbenzimidazol-5-yl)methylene]-1H-imidazol-5-one,
(114) (4Z)-2-(3-Noradamantylamino)-4-[(3-methylbenzimidazol-5-yl)methylene]-1H-imidazol-5-one,
(115) (4Z)-2-(1-Adamantylamino)-4-[(3-methylbenzimidazol-5-yl)methylene]-1H-imidazol-5-one,
(116) (4Z)-2-[(3-Hydroxy-1-adamantyl)amino]-4-[(3-methylbenzimidazol-5-yl)methylene]-1H-imidazol-5-one,
(117) (4Z)-2-[(3-Methoxy-1-adamantyl)amino]-4-[(3-methylbenzimidazol-5-yl)methylene]-1H-imidazol-5-one,
(118) (4Z)-2-[(3-Fluoro-1-adamantyl)amino]-4-[(3-methylbenzimidazol-5-yl)methylene]-1H-imidazol-5-one,
(119) (4Z)-4-[(3-Methylbenzimidazol-5-yl)methylene]-2-[[2-(trifluoromethyl)phenyl]methylamino]-1H-imidazol-5-one,
(120) (4Z)-2-[[(1S,2S)-2-Hydroxyindan-1-yl]amino]-4-[(3-methylbenzimidazol-5-yl)methylene]-1H-imidazol-5-one,
(121) (4Z)-2-[[(1R)-2-Hydroxy-1-phenyl-ethyl]amino]-4-[(3-methylbenzimidazol-5-yl)methylene]-1H-imidazol-5-one,
(122) (4Z)-2-[[(1S)-2-Hydroxy-1-phenyl-ethyl]amino]-4-[(3-methylbenzimidazol-5-yl)methylene]-1H-imidazol-5-one,
(123) (4Z)-2-[[(1R)-2-Methoxy-1-phenyl-ethyl]amino]-4-[(3-methylbenzimidazol-5-yl)methylene]-1H-imidazol-5-one,
(124) (4Z)-2-[[(2R)-2-Hydroxy-2-phenyl-ethyl]amino]-4-[(3-methylbenzimidazol-5-yl)methylene]-1H-imidazol-5-one,
(125) (4Z)-2-[[(2S)-2-Hydroxy-2-phenyl-ethyl]amino]-4-[(3-methylbenzimidazol-5-yl)methylene]-1H-imidazol-5-one,
(126) (4Z)-2-[[(1R)-2-amino-1-phenyl-ethyl]amino]-4-[(3-methylbenzimidazol-5-yl)methylene]-1H-imidazol-5-one dihydrochloride,
(127) (4Z)-2-[[(1S)-2-amino-1-phenyl-ethyl]amino]-4-[(3-methylbenzimidazol-5-yl)methylene]-1H-imidazol-5-one dihydrochloride,
(128) (4Z)-4-[(3-Methylbenzimidazol-5-yl)methylene]-2-[(4-methylthiazol-2-yl)methylamino]-1H-imidazol-5-one,
(129) (4Z)-4-[(3-Methylbenzimidazol-5-yl)methylene]-2-(tetrahydropyran-4-ylmethylamino)-1H-imidazol-5-one,
(130) (4Z)-4-[(3-Methylbenzimidazol-5-yl)methylene]-2-[4-(4-methylpiperazin-1-yl)anilino]-1H-imidazol-5-one,
(131) (4Z)-4-[(3-Methylbenzimidazol-5-yl)methylene]-2-(2-pyridylamino)-1H-imidazol-5-one,
(132) (5Z)-5-(1,3-Benzoxazol-6-ylmethylene)-2-(cyclohexylamino)-3-methyl-imidazol-4-one,
(133) (5Z)-5-(1,3-Benzoxazol-6-ylmethylene)-2-(cyclooctylamino)-3-methyl-imidazol-4-one,
(134) (5Z)-5-(1,3-Benzoxazol-6-ylmethylene)-2-[[(1R)-1-(methoxymethyl)-3-methyl-butyl]amino]-3-methyl-imidazol-4-one,
(135) (5Z)-5-(1,3-Benzoxazol-6-ylmethylene)-2-[[(1R,2R)-2-methoxycyclopentyl]amino]-3-methyl-imidazol-4-one,
(136) (5Z)-5-(1,3-Benzoxazol-6-ylmethylene)-2-[[(1SR,2S)-2-methoxycyclopentyl]amino]-3-methyl-imidazol-4-one,
(137) (5Z)-2-(3-Noradamantylamino)-5-(1,3-benzoxazol-6-ylmethylene)-3-methyl-imidazol-4-one,
(138) (5Z)-2-(1-Adamantylamino)-5-(1,3-benzoxazol-6-ylmethylene)-3-methyl-imidazol-4-one,
(139) (5Z)-5-(1,3-Benzoxazol-6-ylmethylene)-2-[(3-hydroxy-1-adamantyl)amino]-3-methyl-1-4-one,
(140) (5Z)-5-(1,3-Benzoxazol-6-ylmethylene)-2-[(3-methoxy-1-adamantyl)amino]-3-methyl-imidazol-4-one,
(141) (5Z)-5-(1,3-Benzoxazol-6-ylmethylene)-2-[(3-fluoro-1-adamantyl)amino]-3-methyl-imidazol-4-one,
(142) (5Z)-5-(1,3-Benzoxazol-6-ylmethylene)-3-methyl-2-[[2-(trifluoromethyl)phenyl]methylamino]imidazol-4-one,
(143) (5Z)-5-(1,3-Benzoxazol-6-ylmethylene)-2-[[(1R)-2-methoxy-1-phenyl-ethyl]amino]-3-methyl-imidazol-4-one,
(144) (5Z)-5-(1,3-Benzoxazol-6-ylmethylene)-3-methyl-2-[(4-methylthiazol-2-yl)methylamino]imidazol-4-one,
(145) (5Z)-2-(Cyclohexylamino)-5-(1H-indazol-5-ylmethylene)-3-methyl-imidazol-4-one,
(146) (5Z)-2-(Cycloheptylamino)-5-(1H-indazol-5-ylmethylene)-3-methyl-imidazol-4-one,
(147) (5Z)-2-(Cyclooctylamino)-5-(1H-indazol-5-ylmethylene)-3-methyl-imidazol-4-one,
(148) (5Z)-2-[[(1R)-1-(Hydroxymethyl)-3-methyl-butyl]amino]-5-(1H-indazol-5-ylmethylene)-3-methyl-imidazol-4-one,
(149) (5Z)-5-(1H-Indazol-5-ylmethylene)-2-[[(1R)-1-(methoxymethyl)-3-methyl-butyl]amino]-3-methyl-imidazol-4-one,
(150) (5Z)-5-(1H-Indazol-5-ylmethylene)-2-[[(1R,2R)-2-methoxycyclopentyl]amino]-3-methyl-imidazol-4-one,
(151) (5Z)-5-(1H-Indazol-5-ylmethylene)-2-[[(1S,2S)-2-methoxycyclopentyl]amino]-3-methyl-imidazol-4-one,
(152) (5Z)-2-(3-Nordamantylamino)-5-(1H-indazol-5-ylmethylene)-3-methyl-imidazol-4-one,
(153) (5Z)-2-(1-Adamantylamino)-5-(1H-indazol-5-ylmethylene)-3-methyl-imidazol-4-one,
(154) (5Z)-2-[(3-Hydroxy-1-adamantyl)amino]-5-(1H-indazol-5-ylmethylene)-3-methyl-imidazol-4-one,
(155) (5Z)-5-(1H-Indazol-5-ylmethylene)-2-[(3-methoxy-1-adamantyl)amino]-3-methyl-imidazol-4-one,
(156) (5Z)-2-[(3-Fluoro-1-adamantyl)amino]-5-(1H-indazol-5-ylmethylene)-3-methyl-imidazol-4-one,
(157) (5Z)-5-(1H-Indazol-5-ylmethylene)-3-methyl-2-[[2-(trifluoromethyl)phenyl]methylamino]imidazol-4-one,
(158) (5Z)-2-[[(1S,2S)-2-Hydroxyindan-1-yl]amino]-5-(1H-indazol-5-ylmethylene)-3-methyl-imidazol-4-one,
(159) (5Z)-2-[[(1R)-2-Hydroxy-1-phenyl-ethyl]amino]-5-(1H-indazol-5-ylmethylene)-3-methyl-imidazol-4-one,
(160) (5Z)-2-[[(1S)-2-Hydroxy-1-phenyl-ethyl]amino]-5-(1H-indazol-5-ylmethylene)-3-methyl-imidazol-4-one,
(161) (5Z)-5-(1H-Indazol-5-ylmethylene)-2-[[(1R)-2-methoxy-1-phenyl-ethyl]amino]-3-methyl-imidazol-4-one,
(162) (5Z)-2-[[(2R)-2-Hydroxy-2-phenyl-ethyl]amino]-5-(1H-indazol-5-ylmethylene)-3-methyl-imidazol-4-one,
(163) (5Z)-2-[[(1R)-2-Amino-1-phenyl-ethyl]amino]-5-(1H-indazol-5-ylmethylene)-3-methyl-imidazol-4-one dihydrochloride,
(164) (5Z)-5-(1H-Indazol-5-ylmethylene)-3-methyl-2-[(4-methylthiazol-2-yl)methylamino]imidazol-4-one,
(165) (5Z)-5-(1H-Indazol-5-ylmethylene)-3-methyl-2-(tetrahydropyran-4-ylmethylamino)imidazol-4-one,
(166) (5Z)-5-(1H-Indazol-5-ylmethylene)-3-methyl-2-[4-(4-methylpiperazin-1-yl)anilino]imidazol-4-one,
(167) (5Z)-5-(1H-Indazol-5-ylmethylene)-3-methyl-2-(2-pyridylamino)imidazol-4-one,
(168) (5Z)-5-(1H-Indazol-5-ylmethylene)-3-methyl-2-[[(3S)-tetrahydropyran-3-yl]amino]imidazol-4-one,
(169) (5Z)-2-[[(3R,4R)-4-Hydroxytetrahydropyran-3-yl]amino]-5-(1H-indazol-5-ylmethylene)-3-methyl-imidazol-4-one,
(170) (5Z)-2-(Cyclohexylamino)-3-methyl-5-[(2-methylindazol-5-yl)methylene]imidazol-4-one,
(171) (5Z)-2-(Cycloheptylamino)-3-methyl-5-[(2-methylindazol-5-yl)methylene]imidazol-4-one,
(172) (5Z)-2-(Cyclooctylamino)-3-methyl-5-[(2-methylindazol-5-yl)methylene]imidazol-4-one,
(173) (5Z)-2-[[(1R)-1-(Hydroxymethyl)-3-methyl-butyl]amino]-3-methyl-5-[(2-methylindazol-5-yl)methylene]imidazol-4-one,
(174) (5Z)-2-[[(1R)-1-(Methoxymethyl)-3-methyl-butyl]amino]-3-methyl-5-[(2-methylindazol-5-yl)methylene]imidazol-4-one,
(175) (5Z)-2-[[(1R,2R)-2-Methoxycyclopentyl]amino]-3-methyl-5-[(2-methylindazol-5-yl)methylene]imidazol-4-one,
(176) (5Z)-2-[[(1S,2S)-2-Methoxycyclopentyl]amino]-3-methyl-5-[(2-methylindazol-5-yl)methylene]imidazol-4-one,
(177) (5Z)-2-(3-Noradamantylamino)-3-methyl-5-[(2-methylindazol-5-yl)methylene]imidazol-4-one,
(178) (5Z)-2-(1-Adamantylamino)-3-methyl-5-[(2-methylindazol-5-yl)methylene]imidazol-4-one,
(179) (5Z)-2-[(3-Hydroxy-1-adamantyl)amino]-3-methyl-5-[(2-methylindazol-5-yl)methylene]imidazol-4-one,
(180) (5Z)-2-[(3-Methoxy-1-adamantyl)amino]-3-methyl-5-[(2-methylindazol-5-yl)methylene]imidazol-4-one,
(181) (5Z)-2-[(3-Fluoro-1-adamantyl)amino]-3-methyl-5-[(2-methylindazol-5-yl)methylene]imidazol-4-one,
(182) (5Z)-3-Methyl-5-[(2-methylindazol-5-yl)methylene]-2-[[2-(trifluoromethyl)phenyl]methylamino]imidazol-4-one,
(183) (5Z)-2-[[(1S,2S)-2-Hydroxyindan-1-yl]amino]-3-methyl-5-[(2-methylindazol-5-yl)methylene]imidazol-4-one,
(184) (5Z)-2-[[(1R)-2-Hydroxy-1-phenyl-ethyl]amino]-3-methyl-5-[(2-methylindazol-5-yl)methylene]imidazol-4-one,
(185) (5Z)-2-[[(1S)-2-Hydroxy-1-phenyl-ethyl]amino]-3-methyl-5-[(2-methylindazol-5-yl)methylene]imidazol-4-one,
(186) (5Z)-2-[[(1R)-2-Methoxy-1-phenyl-ethyl]amino]-3-methyl-5-[(2-methylindazol-5-yl)methylene]imidazol-4-one,
(187) (5Z)-2-[[(2R)-2-Hydroxy-2-phenyl-ethyl]amino]-3-methyl-5-[(2-methylindazol-5-yl)methylene]imidazol-4-one,
(188) (5Z)-2-[[(1R)-2-Amino-1-phenyl-ethyl]amino]-3-methyl-5-[(2-methylindazol-5-yl)methylene]imidazol-4-one dihydrochloride,
(189) (5Z)-3-Methyl-5-[(2-methylindazol-5-yl)methylene]-2-[(4-methylthiazol-2-yl)methylamino]imidazol-4-one,
(190) (5Z)-3-Methyl-5-[(2-methylindazol-5-yl)methylene]-2-(tetrahydropyran-4-ylmethylamino)imidazol-4-one,
(191) (5Z)-3-Methyl-5-[(2-methylindazol-5-yl)methylene]-2-[4-(4-methylpiperazin-1-yl)anilino]imidazol-4-one,
(192) (5Z)-3-Methyl-5-[(2-methylindazol-5-yl)methylene]-2-(2-pyridylamino)imidazol-4-one,
(193) (5Z)-3-Methyl-5-[(2-methylindazol-5-yl)methylene]-2-[[(3S)-tetrahydropyran-3-yl]amino]imidazol-4-one,
(194) (5Z)-2-[[(3R,4R)-4-Hydroxytetrahydropyran-3-yl]amino]-3-methyl-5-[(2-methylindazol-5-yl)methylene]imidazol-4-one,
(195) (5Z)-2-(Cyclohexylamino)-3-methyl-5-[(1-methylindazol-5-yl)methylene]imidazol-4-one,
(196) (5Z)-2-(Cycloheptylamino)-3-methyl-5-[(1-methylindazol-5-yl)methylene]imidazol-4-one,
(197) (5Z)-2-(Cyclooctylamino)-3-methyl-5-[(1-methylindazol-5-yl)methylene]imidazol-4-one,
(198) (5Z)-2-[[(1R)-1-(Hydroxymethyl)-3-methyl-butyl]amino]-3-methyl-5-[(1-methylindazol-5-yl)methylene]imidazol-4-one,
(199) (5Z)-2-[[(1R)-1-(Methoxymethyl)-3-methyl-butyl]amino]-3-methyl-5-[(1-methylindazol-5-yl)methylene]imidazol-4-one,
(200) (5Z)-2-[[(1R,2R)-2-Methoxycyclopentyl]amino]-3-methyl-5-[(1-methylindazol-5-yl)methylene]imidazol-4-one,
(201) (5Z)-2-[[(1S,2S)-2-Methoxycyclopentyl]amino]-3-methyl-5-[(1-methylindazol-5-yl)methylene]imidazol-4-one,
(202) (5Z)-2-(3-Noradamantylamino)-3-methyl-5-[(1-methylindazol-5-yl)methylene]imidazol-4-one,
(203) (5Z)-2-(1-Adamantylamino)-3-methyl-5-[(1-methylindazol-5-yl)methylene]imidazol-4-one,
(204) (5Z)-2-[(3-Hydroxy-1-adamantyl)amino]-3-methyl-5-[(1-methylindazol-5-yl)methylene]imidazol-4-one,
(205) (5Z)-2-[(3-Methoxy-1-adamantyl)amino]-3-methyl-5-[(1-methylindazol-5-yl)methylene]imidazol-4-one,
(206) (5Z)-2-[(3-Fluoro-1-adamantyl)amino]-3-methyl-5-[(1-methylindazol-5-yl)methylene]imidazol-4-one,
(207) (5Z)-3-Methyl-5-[(1-methylindazol-5-yl)methylene]-2-[[2-(trifluoromethyl)phenyl]methylamino]imidazol-4-one,
(208) (5Z)-2-[[(1S,2S)-2-Hydroxyindan-1-yl]amino]-3-methyl-5-[(1-methylindazol-5-yl)methylene]imidazol-4-one,
(209) (5Z)-2-[[(1R)-2-Hydroxy-1-phenyl-ethyl]amino]-3-methyl-5-[(1-methylindazol-5-yl)methylene]imidazol-4-one,
(210) (5Z)-2-[[(1R)-2-Hydroxy-1-phenyl-ethyl]amino]-3-methyl-5-[(1-methylindazol-5-yl)methylene]imidazol-4-one,
(211) (5Z)-2-[[(1R)-2-Methoxy-1-phenyl-ethyl]amino]-3-methyl-5-[(1-methylindazol-5-yl)methylene]imidazol-4-one,
(212) (5Z)-2-[[(2R)-2-Hydroxy-2-phenyl-ethyl]amino]-3-methyl-5-[(1-methylindazol-5-yl)methylene]imidazol-4-one,
(213) (5Z)-2-[[(1R)-2-Amino-1-phenyl-ethyl]amino]-3-methyl-5-[(1-methylindazol-5-yl)methylene]imidazol-4-one dihydrochloride,
(214) (5Z)-3-Methyl-5-[(1-methylindazol-5-yl)methylene]-2-[(4-methylthiazol-2-yl)methylamino]imidazol-4-one,
(215) (5Z)-3-Methyl-5-[(1-methylindazol-5-yl)methylene]-2-(tetrahydropyran-4-ylmethylamino)imidazol-4-one,
(216) (5Z)-3-Methyl-5-[(1-methylindazol-5-yl)methylene]-2-[4-(4-methylpiperazin-1-yl)anilino]imidazol-4-one,
(217) (5Z)-3-Methyl-5-[(1-methylindazol-5-yl)methylene]-2-(2-pyridylamino)imidazol-4-one,
(218) (5Z)-3-Methyl-5-[(1-methylindazol-5-yl)methylene]-2-[[(3S)-tetrahydropyran-3-yl]amino]imidazol-4-one,
(219) (5Z)-2-[[(3R,4R)-4-Hydroxytetrahydropyran-3-yl]amino]-3-methyl-5-[(1-methylindazol-5-yl)methylene]imidazol-4-one,
(220) (5Z)-5-(1H-Benzimidazol-5-ylmethylene)-2-(cyclohexylamino)-3-methyl-imidazol-4-one,
(221) (5Z)-5-(1H-Benzimidazol-5-ylmethylene)-2-(cycloheptylamino)-3-methyl-imidazol-4-one,
(222) (5Z)-5-(1H-Benzimidazol-5-ylmethylene)-2-(cyclooctylamino)-3-methyl-imidazol-4-one,
(223) (5Z)-5-(1H-Benzimidazol-5-ylmethylene)-2-[[(1R)-1-(hydroxymethyl)-3-methyl-butyl]amino]-3-methyl-imidazol-4-one,
(224) (5Z)-5-(1H-Benzimidazol-5-ylmethylene)-2-[[(1R)-1-(methoxymethyl)-3-methyl-butyl]amino]-3-methyl-imidazol-4-one,
(225) (5Z)-5-(1H-Benzimidazol-5-ylmethylene)-2-[[(1R,2R)-2-methoxycyclopentyl]amino]-3-methyl-imidazol-4-one,
(226) (5Z)-5-(1H-Benzimidazol-5-ylmethylene)-2-[[(1S,2S)-2-methoxycyclopentyl]amino]-3-methyl-imidazol-4-one,
(227) (5Z)-2-(3-Nordamantylamino)-5-(1H-benzimidazol-5-ylmethylene)-3-methyl-imidazol-4-one,
(228) (5Z)-2-(1-Adamantylamino)-5-(1H-benzimidazol-5-ylmethylene)-3-methyl-imidazol-4-one,
(229) (5Z)-5-(1H-Benzimidazol-5-ylmethylene)-2-[(3-hydroxy-1-adamantyl)amino]-3-methyl-imidazol-4-one,
(230) (5Z)-5-(1H-Benzimidazol-5-ylmethylene)-2-[(3-methoxy-1-adamantyl)amino]-3-methyl-imidazol-4-one,
(231) (5Z)-5-(1H-Benzimidazol-5-ylmethylene)-2-[(3-fluoro-1-adamantyl)amino]-3-methyl-imidazol-4-one,
(232) (5Z)-5-(1H-Benzimidazol-5-ylmethylene)-3-methyl-2-[[2-(trifluoromethyl)phenyl]methylamino]imidazol-4-one,
(233) (5Z)-5-(1H-Benzimidazol-5-ylmethylene)-2-[[(1S,2S)-2-hydroxyindan-1-yl]amino]-3-methyl-imidazol-4-one,

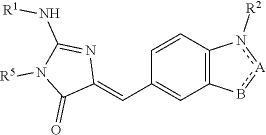

| Cpd N° | R¹—NH— | R5 | 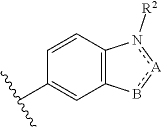 | Class | Formula | MW | Stereo- chemistry |
|---|---|---|---|---|---|---|---|
| 252 | 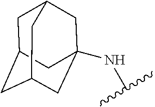 [SMILES: *NC12CC3CC(CC(C3)C1)C2] | Me | 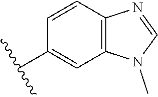 [SMILES: *c1ccc2ncn(C)c2c1] | A1 | C₂₃H₂₇N₅O | 389.50 | N/A |
| 253 | 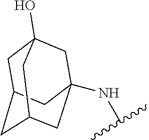 [SMILES: *NC12CC3CC(CC(O)(C3)C1)C2] | Me | 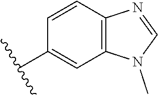 [SMILES: *c1ccc2ncn(C)c2c1] | A1 | C₂₃H₂₇N₅O₂ | 405.50 | N/A |
| 254 | 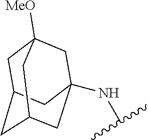 [SMILES: *NC12CC3CC(C1)CC(OC)(C3)C2] | Me | 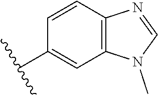 [SMILES: *c1ccc2ncn(C)c2c1] | A1 | C₂₄H₂₉N₅O₂ | 419.53 | N/A |
| 255 | 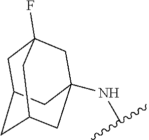 [SMILES: *NC12CC3CC(CC(F)(C3)C1)C2] | Me | 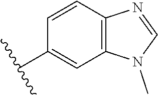 [SMILES: *c1ccc2ncn(C)c2c1] | A1 | C₂₃H₂₆FN₅O | 407.49 | N/A |
| 256 | 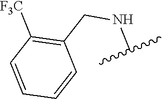 [SMILES: *NCc1ccccc1C(F)(F)F] | Me | 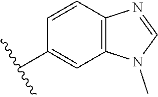 [SMILES: *c1ccc2ncn(C)c2c1] | A2 | C₂₁H₁₈F₃N₅O | 413.40 | N/A |
| 257 | 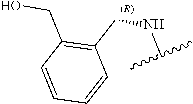 | Me | 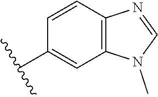 [SMILES: *c1ccc2ncn(C)c2c1] | A2 | C₂₁H₂₁N₅O₂ | 375.43 | (1R) |
| 258 | 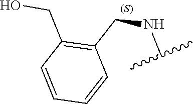 | Me | 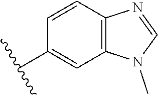 [SMILES: *c1ccc2ncn(C)c2c1] | A2 | C₂₁H₂₁N₅O₂ | 375.43 | (1R) |
| 259 | 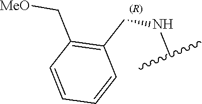 | Me | 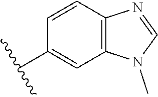 [SMILES: *c1ccc2ncn(C)c2c1] | A2 | C₂₂H₂₃N₅O₂ | 389.46 | (1R) |
| 260 | 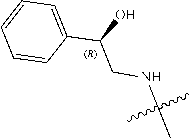 | Me | 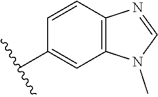 [SMILES: *c1ccc2ncn(C)c2c1] | A2 | C₂₁H₂₁N₅O₂ | 375.43 | (2R) |
| 261 | 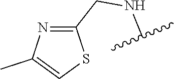 [SMILES: *NCc1nc(C)cs1] | Me | 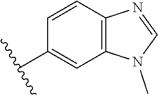 [SMILES: *c1ccc2ncn(C)c2c1] | A3 | C₁₈H₁₈N₆OS | 366.44 | N/A |
| 262 | 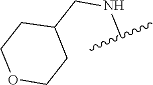 [SMILES: *NCC1CCOCC1] | Me | 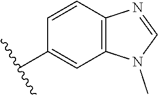 [SMILES: *c1ccc2ncn(C)c2c1] | A4 | C₁₉H₂₃N₅O₂ | 353.43 | N/A |
| 263 | 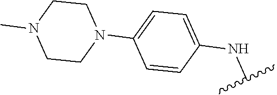 [SMILES: *Nc1ccc(N2CCN(C)CC2)cc1] | Me | 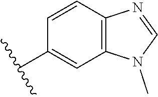 [SMILES: *c1ccc2ncn(C)c2c1] | A5 | C₂₄H₂₇N₇O | 429.53 | N/A |
| 264 | 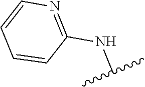 [SMILES: *Nc1ccccn1] | Me | 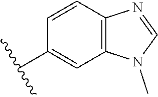 [SMILES: *c1ccc2ncn(C)c2c1] | A6 | C₁₈H₁₆N₆O | 332.37 | N/A |
| 265 | 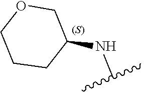 | Me | 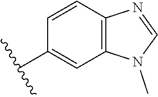 [SMILES: *c1ccc2ncn(C)c2c1] | A7 | C₁₈H₂₁N₅O₂ | 339.40 | (3S) |
| 266 | 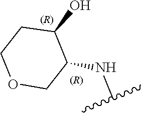 | Me | 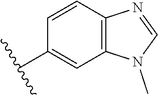 [SMILES: *c1ccc2ncn(C)c2c1] | A7 | C₁₈H₂₁N₅O₃ | 355.40 | (3R,4R) |
| 267 | 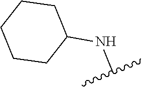 [SMILES: *NC1CCCCC1] | H | 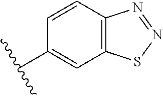 [SMILES: *c1ccc2nnsc2c1] | A1 | C₁₆H₁₇N₅OS | 327.41 | N/A |
| 268 | 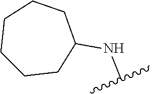 [SMILES: *NC1CCCCCC1] | H | 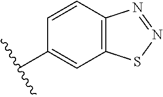 [SMILES: *c1ccc2nnsc2c1] | A1 | C₁₇H₁₉N₅OS | 341.43 | N/A |
| 269 | 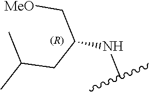 | H | 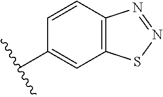 [SMILES: *c1ccc2nnsc2c1] | A1 | C₁₇H₂₁N₅O₂S | 359.45 | (1R) |
| 270 | 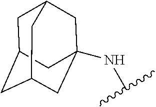 [SMILES: *NC12CC3CC(CC(C3)C1)C2] | H | 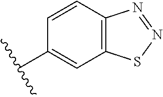 [SMILES: *c1ccc2nnsc2c1] | A1 | C₂₀H₂₁N₅OS | 379.48 | N/A |
| 271 | 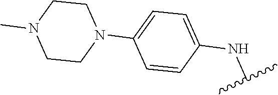 [SMILES: *Nc1ccc(N2CCN(C)CC2)cc1] | H | 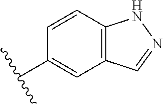 [SMILES: *c1ccc2[nH]ncc2c1] | A5 | C₂₂H₂₃N₇O | 401.47 | N/A |

The below Table 3 describes the analytical and spectroscopic data of the compounds introduced in Table 2.

′ H NMR analyses (400 or 500 MHz) and ¹³C NMR spectra (101 MHz) were recorded with a Bruker ULTRASHIELD 500 or 400 spectrometer. Processing and analyses of the spectra were performed with MestReNova. Data appear in the following order: chemical shifts in ppm which were referenced to the internal solvent signal, multiplicity, number of protons, and coupling constant Jin Hertz.

Reversed-phase HPLC/MS analyses were carried out with a Waters Alliance 2795 HPLC equipped with an autosampler, an inline membrane degasser, a column oven (T°=45° C.), a UV detector, and a ZQ quadrupole mass detector working in ionization electrospray mode. Compounds (0.1 to 0.3 mg) were solubilized in a minimum amount of DMSO completed with acetonitrile (Vₜₒₜₐₗ: 1 mL). Standard analytical parameters: flow rate: 1 mL/min, V_{inj.}: 5 μL.
Acidic conditions: Waters XSelect CSH C18 column (3.5 μm, 2.1×50 mm). Gradient: (H₂O+0.04% v/v HCOOH (10 mM))/ACN from 95/5 to 0/100 in 18.5 min.
Alkaline conditions: Waters Xbridge C18 column (3.5 μm, 2.1×50 mm). Gradient: (H₂O+0.06% v/v NH_{3(aq)} (10 mM))/ACN from 95/5 to 0/100 in 18.5 min.

TABLE 3
Spectroscopic and analytical characterization of compounds (1) to (271)

| Cpd | | | | |
|---|---|---|---|---|
| Nº | Structure | Formula | MW | HPLC |
| 1 | 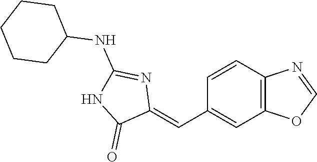 [SMILES: O=C1NC(NC2CCCCC2)=N/C1=C\c1ccc2ncoc2c1] | C₁₇H₁₈N₄O₂ | 310.36 | 97% |
| 2 | 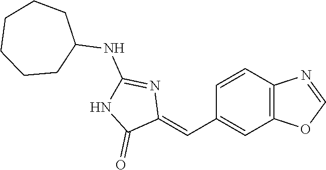 [SMILES: O=C1NC(NC2CCCCCC2)=N/C1=C\c1ccc2ncoc2c1] | C₁₈H₂₀N₄O₂ | 324.38 | >95% |
| 3 | 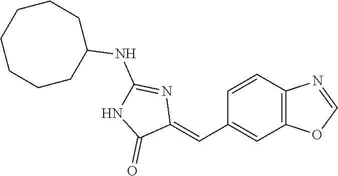 [SMILES: O=C1NC(NC2CCCCCCC2)=N/C1=C\c1ccc2ncoc2c1] | C₁₉H₂₂N₄O₂ | 338.41 | >98% |
| 4 | 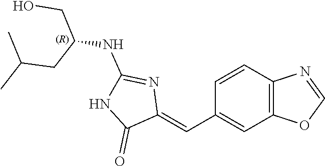 | C₁₇H₂₀N₄O₃ | 328.37 | 96% |
| 5 | 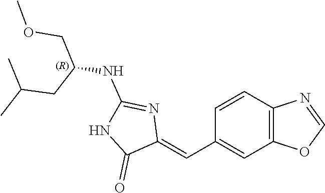 | C₁₈H₂₂N₄O₃ | 342.40 | >98% |
| 6 | 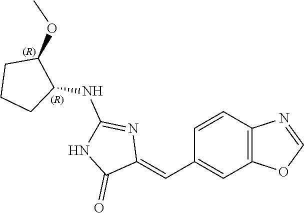 | C₁₇H₁₈N₄O₃ | 326.36 | >98% |
| 7 | 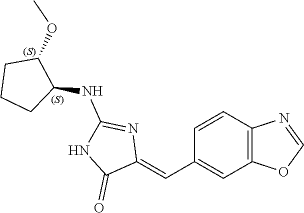 | C₁₇H₁₈N₄O₃ | 326.36 | >98% |
| 8 | 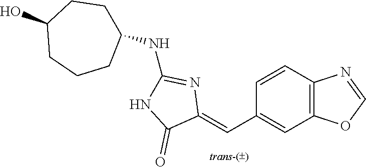 [SMILES: O=C1NC(N[C@@H]2CCC[C@@H](O)CC2)=N/C1=C\c1ccc2ncoc2c1] | C₁₈H₂₀N₄O₃ | 340.38 | 98% |
| 9 | 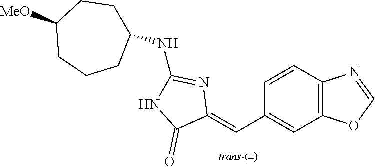 [SMILES: CO[C@@H]1CCC[C@@H](NC2=N/C(=C\c3ccc4ncoc4c3)C(=O)N2)CC1] | C₁₉H₂₂N₄O₃ | 354.41 | >98% |
| 10 | 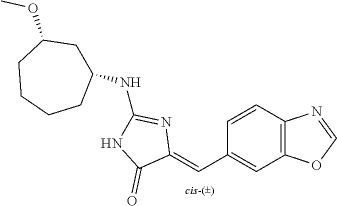 [SMILES: CO[C@H]1CCCC[C@@H](NC2=N/C(=C\c3ccc4ncoc4c3)C(=O)N2)C1] | C₁₉H₂₂N₄O₃ | 354.41 | >98% |
| 11 | 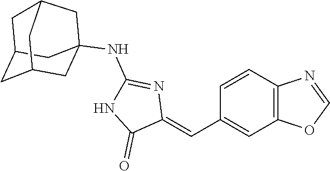 [SMILES: O=C1NC(NC23CC4CC(CC(C4)C2)C3)=N/C1=C\c1ccc2ncoc2c1] | C₂₁H₂₂N₄O₂ | 362.43 | >98% |
| 12 | 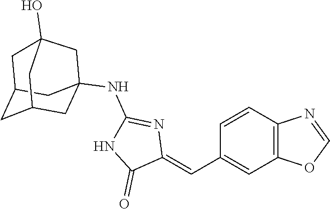 [SMILES: O=C1NC(NC23CC4CC(CC(O)(C4)C2)C3)=N/C1=C\c1ccc2ncoc2c1] | C₂₁H₂₂N₄O₃ | 378.43 | 97% |
| 13 | 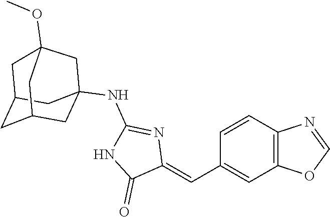 [SMILES: COC12CC3CC(CC(NC4=N/C(=C\c5ccc6ncoc6c5)C(=O)N4)(C3)C1)C2] | C₂₂H₂₄N₄O₃ | 392.46 | >98% |
| 14 | 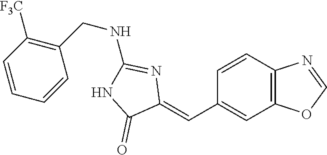 [SMILES: O=C1NC(NCc2ccccc2C(F)(F)F)=N/C1=C\c1ccc2ncoc2c1] | C₁₉H₁₃F₃N₄O₂ | 386.33 | >98% |
| 15 | 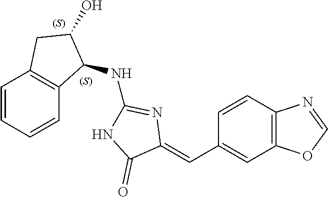 | C₂₀H₁₆N₄O₃ | 360.37 | 93% |
| 16 | 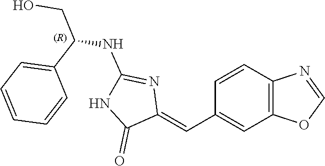 | C₁₉H₁₆N₄O₃ | 348.36 | >98% |
| 17 | 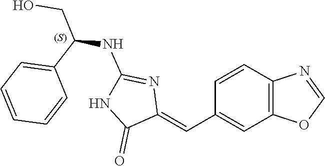 | C₁₉H₁₆N₄O₃ | 348.36 | 96% |
| 18 | 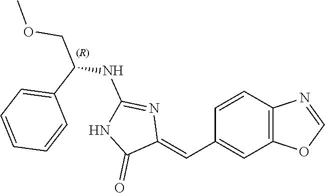 | C₂₀H₁₈N₄O₃ | 362.39 | >98% |
| 19 | 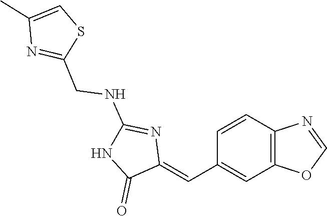 [SMILES: Cc1csc(CNC2=N/C(=C\c3ccc4ncoc4c3)C(=O)N2)n1] | C₁₆H₁₃N₅O₂S | 339.37 | >98% |
| 20 | 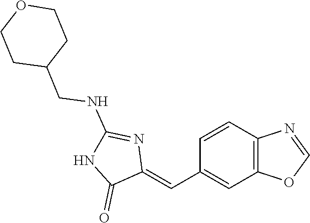 [SMILES: O=C1NC(NCC2CCOCC2)=N/C1=C\c1ccc2ncoc2c1] | C₁₇H₁₈N₄O₃ | 326.36 | >98% |
| 21 | 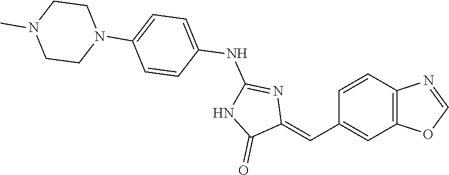 [SMILES: CN1CCN(c2ccc(NC3=N/C(=C\c4ccc5ncoc5c4)C(=O)N3)cc2)CC1] | C₂₂H₂₂N₆O₂ | 402.46 | >98% (¹H NMR) |
| 22 | 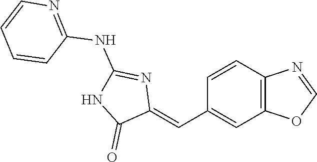 [SMILES: O=C1NC(Nc2ccccn2)=N/C1=C\c1ccc2ncoc2c1] | C₁₆H₁₁N₅O₂ | 305.30 | >95% |
| 23 | 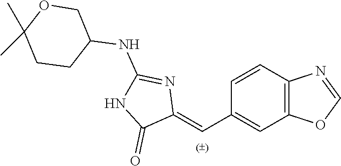 [SMILES: CC1(C)CCC(NC2=N/C(=C\c3ccc4ncoc4c3)C(=O)N2)CO1] | C₁₈H₂₀N₄O₃ | 340.38 | >98% |
| 24 | 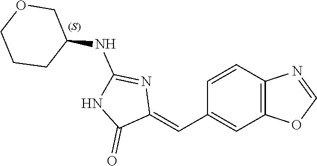 | C₁₆H₁₆N₄O₃ | 312.33 | >98% |
| 25 | 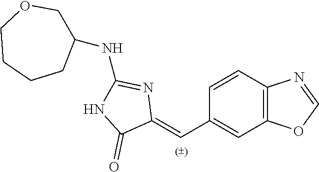 [SMILES: O=C1NC(NC2CCCCOC2)=N/C1=C\c1ccc2ncoc2c1] | C₁₇H₁₈N₄O₃ | 326.36 | >98% |
| 26 | 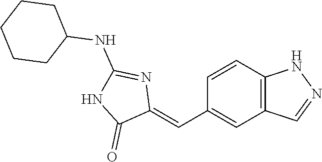 [SMILES: O=C1NC(NC2CCCCC2)=N/C1=C\c1ccc2[nH]ncc2c1] | C₁₇H₁₉N₅O | 309.37 | >98% |
| 27 | 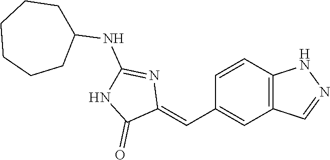 [SMILES: O=C1NC(NC2CCCCCC2)=N/C1=C\c1ccc2[nH]ncc2c1] | C₁₈H₂₁N₅O | 323.40 | >98% |
| 28 | 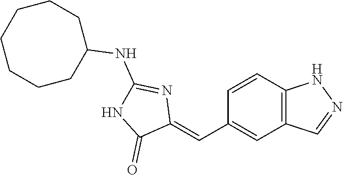 [SMILES: O=C1NC(NC2CCCCCCC2)=N/C1=C\c1ccc2[nH]ncc2c1] | C₁₉H₂₃N₅O | 337.43 | >98% |
| 29 | 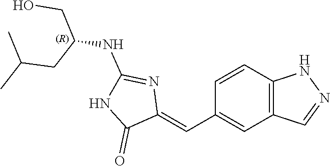 | C₁₇H₂₁N₅O₂ | 327.39 | >98% |
| 30 | 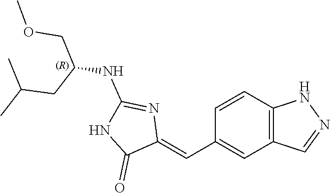 | C₁₈H₂₃N₅O₂ | 341.42 | >98% |
| 31 | 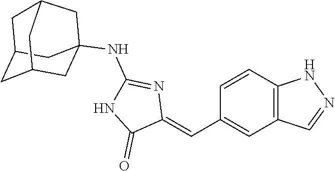 [SMILES: O=C1NC(NC23CC4CC(CC(C4)C2)C3)=N/C1=C\c1ccc2[nH]ncc2c1] | C₂₁H₂₃N₅O | 361.45 | 97% |
| 32 | 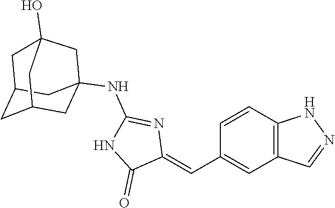 [SMILES: O=C1NC(NC23CC4CC(CC(O)(C4)C2)C3)=N/C1=C\c1ccc2[nH]ncc2c1] | C₂₁H₂₃N₅O₂ | 377.45 | >98% |
| 33 | 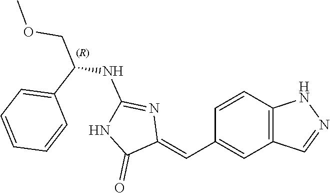 | C₂₀H₁₉N₅O₂ | 361.41 | 97% |
| 34 | 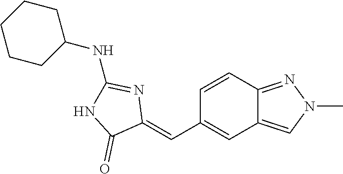 [SMILES: Cn1cc2cc(/C=C3\N=C(NC4CCCCC4)NC3=O)ccc2n1] | C₁₈H₂₁N₅O | 323.40 | >98% |
| 35 | 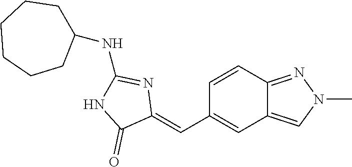 [SMILES: Cn1cc2cc(/C=C3\N=C(NC4CCCCCC4)NC3=O)ccc2n1] | C₁₉H₂₃N₅O | 337.43 | 98% |
| 36 | 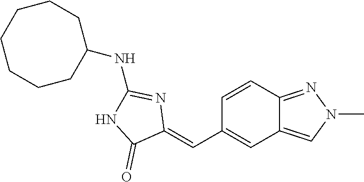 [SMILES: Cn1cc2cc(/C=C3\N=C(NC4CCCCCCC4)NC3=O)ccc2n1] | C₂₀H₂₅N₅O | 351.45 | >98% |
| 37 | 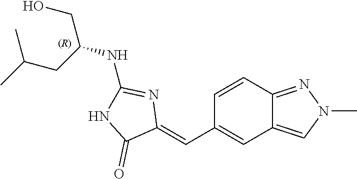 | C₁₈H₂₃N₅O₂ | 341.42 | >98% |
| 38 | 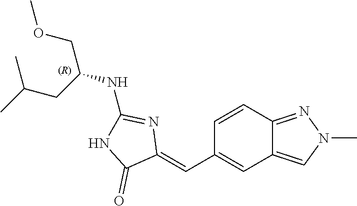 | C₁₉H₂₅N₅O₂ | 355.44 | >98% |
| 39 | 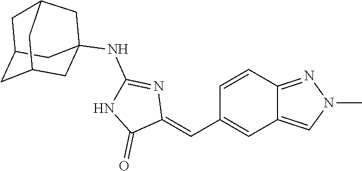 [SMILES: Cn1cc2cc(/C=C3\N=C(NC45CC6CC(CC(C6)C4)C5)NC3=O)ccc2n1] | C₂₂H₂₅N₅O | 375.48 | >98% |
| 40 | 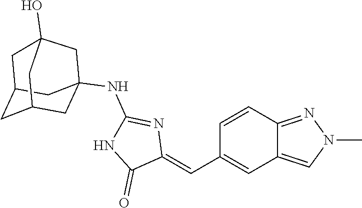 [SMILES: Cn1cc2cc(/C=C3\N=C(NC45CC6CC(CC(O)(C6)C4)C5)NC3=O)ccc2n1] | C₂₂H₂₅N₅O₂ | 391.48 | >98% |
| 41 | 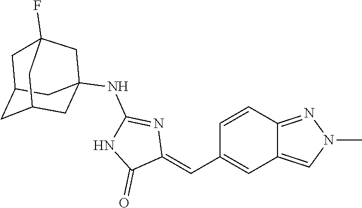 [SMILES: Cn1cc2cc(/C=C3\N=C(NC45CC6CC(CC(F)(C6)C4)C5)NC3=O)ccc2n1] | C₂₂H₂₄FN₅O | 393.47 | >98% |
| 42 | 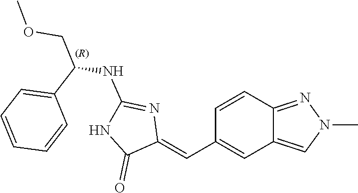 | C₂₁H₂₁N₅O₂ | 375.43 | >98% |
| 43 | 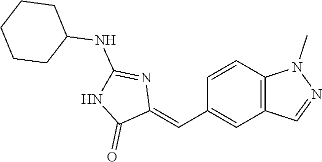 [SMILES: Cn1ncc2cc(/C=C3\N=C(NC4CCCCC4)NC3=O)ccc21] | C₁₈H₂₁N₅O | 323.40 | >98% |
| 44 | 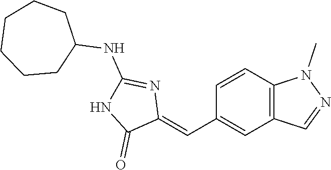 [SMILES: Cn1ncc2cc(/C=C3\N=C(NC4CCCCCC4)NC3=O)ccc21] | C₁₉H₂₃NO | 337.43 | >98% |
| 45 | 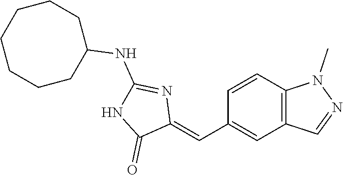 [SMILES: Cn1ncc2cc(/C=C3\N=C(NC4CCCCCCC4)NC3=O)ccc21] | C₂₀H₂₅N₅O | 351.45 | 98% |
| 46 | 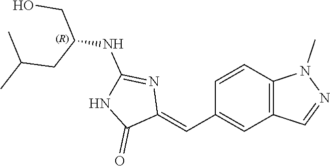 | C₁₈H₂₃N₅O₂ | 341.42 | >98% |
| 47 | 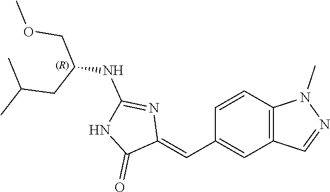 | C₁₉H₂₅N₅O₂ | 355.44 | >98% |
| 48 | 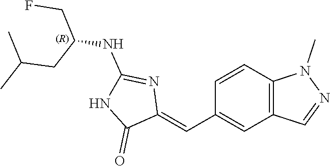 | C₁₈H₂₂FN₅O | 343.41 | 97% |
| 49 | 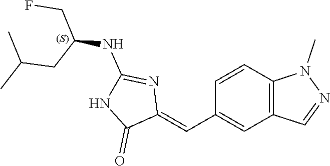 | C₁₈H₂₂FN₅O | 343.41 | 97% |
| 50 | 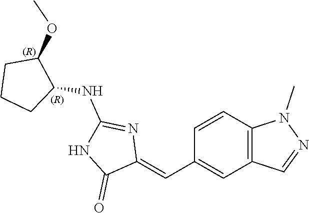 | C₁₈H₂₁N₅O₂ | 339.40 | 98% |
| 51 | 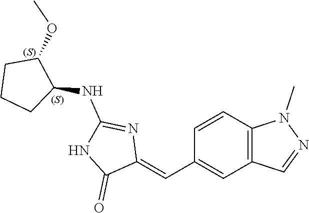 | C₁₈H₂₁N₅O₂ | 339.40 | >98% |
| 52 | 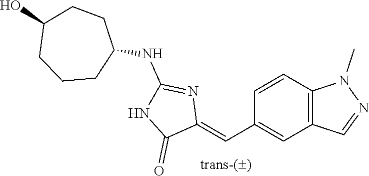 [SMILES: Cn1ncc2cc(/C=C3\N=C(N[C@@H]4CCC[C@@H](O)CC4)NC3=O)ccc21] | C₁₉H₂₃N₅O₂ | 353.43 | >98% |
| 53 | 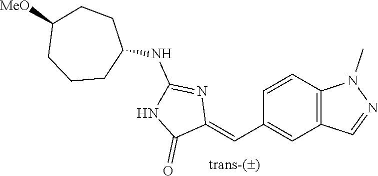 [SMILES: CO[C@@H]1CCC[C@@H](NC2=N/C(=C\c3ccc4c(cnn4C)c3)C(=O)N2)CC1] | C₂₀H₂₅N₅O₂ | 367.45 | >98% |
| 54 | 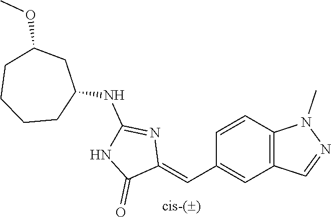 [SMILES: CO[C@H]1CCCC[C@@H](NC2=N/C(=C\c3ccc4c(cnn4C)c3)C(=O)N2)C1] | C₂₀H₂₅N₅O₂ | 367.45 | >98% |
| 55 | 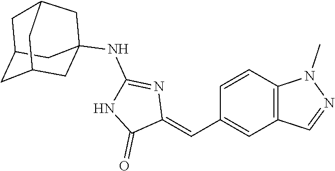 [SMILES: Cn1ncc2cc(/C=C3\N=C(NC45CC6CC(CC(C6)C4)C5)NC3=O)ccc21] | C₂₂H₂₅N₅O | 375.48 | >98% |
| 56 | 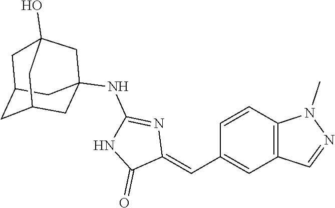 [SMILES: Cn1ncc2cc(/C=C3\N=C(NC45CC6CC(CC(O)(C6)C4)C5)NC3=O)ccc21] | C₂₂H₂₅N₅O₂ | 391.48 | >98% |
| 57 | 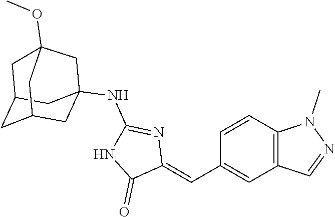 [SMILES: COC12CC3CC(CC(NC4=N/C(=C\c5ccc6c(cnn6C)c5)C(=O)N4)(C3)C1)C2] | C₂₃H₂₇N₅O₂ | 405.50 | >98% |
| 58 | 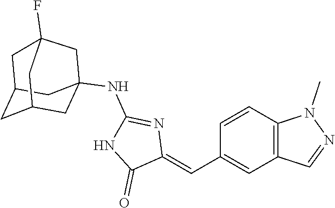 [SMILES: Cn1ncc2cc(/C=C3\N=C(NC45CC6CC(CC(F)(C6)C4)C5)NC3=O)ccc21] | C₂₂H₂₄FN₅O | 393.47 | >98% |
| 59 | 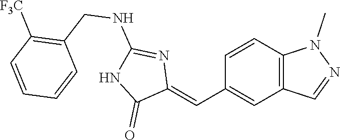 [SMILES: Cn1ncc2cc(/C=C3\N=C(NCc4ccccc4C(F)(F)F)NC3=O)ccc21] | C₂₀H₁₆F₃N₅O | 399.38 | >98% |
| 60 | 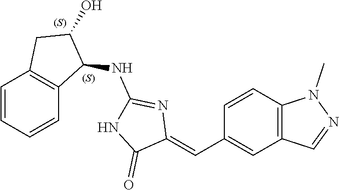 | C₂₁H₁₉N₅O₂ | 373.42 | >98% |
| 61 | 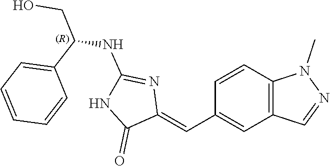 | C₂₀H₁₉N₅O₂ | 361.41 | >98% |
| 62 | 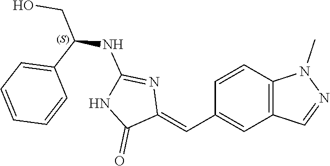 | C₂₀H₁₉N₅O₂ | 361.41 | >98% |
| 63 | 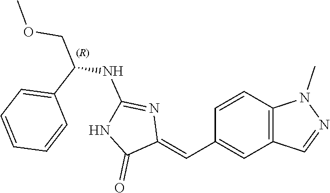 | C₂₁H₂₁N₅O₂ | 375.43 | >98% |
| 64 | 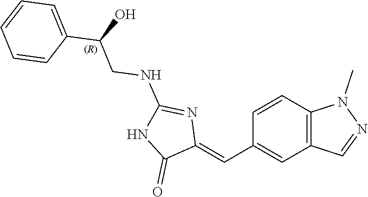 | C₂₀H₁₉N₅O₂ | 361.41 | 95% |
| 65 | 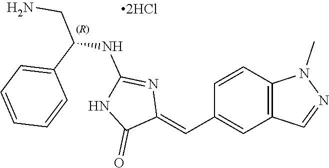 | C₂₀H₂₂C₁₂N₆O | 433.34 | 95% |
| 66 | 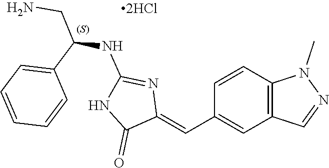 | C₂₀H₂₂C₁₂N₆O | 433.34 | 94% |
| 67 | 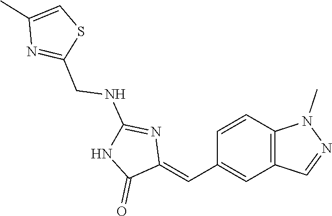 [SMILES: Cc1csc(CNC2=N/C(=C\c3ccc4c(cnn4C)c3)C(=O)N2)n1] | C₁₇H₁₆N₆OS | 352.42 | >98% |
| 68 | 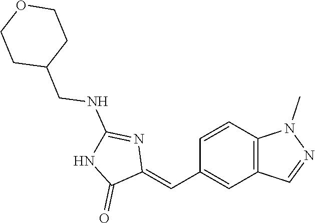 [SMILES: Cn1ncc2cc(/C=C3\N=C(NCC4CCOCC4)NC3=O)ccc21] | C₁₈H₂₁N₅O₂ | 339.40 | >98% |
| 69 | 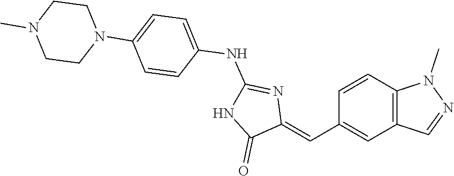 [SMILES: CN1CCN(c2ccc(NC3=N/C(=C\c4ccc5c(cnn5C)c4)C(=O)N3)cc2)CC1] | C₂₃H₂₅N₇O | 415.50 | >98% |
| 70 | 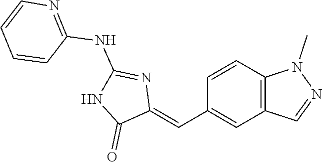 [SMILES: Cn1ncc2cc(/C=C3\N=C(Nc4ccccn4)NC3=O)ccc21] | C₁₇H₁₄N₆O | 318.34 | >98% |
| 71 | 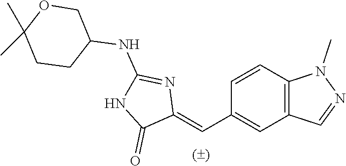 [SMILES: Cn1ncc2cc(/C=C3\N=C(NC4CCC(C)(C)OC4)NC3=O)ccc21] | C₁₉H₂₃N₅O₂ | 353.43 | >98% |
| 72 | 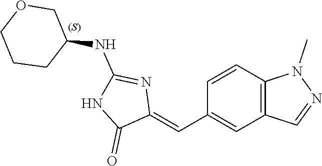 | C₁₇H₁₉N₅O₂ | 325.37 | >98% |
| 73 | 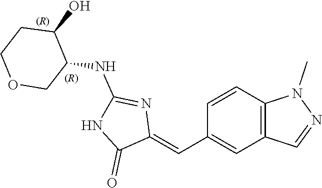 | C₁₇H₁₉N₅O₃ | 341.37 | >98% |
| 74 | 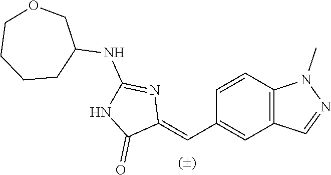 [SMILES: Cn1ncc2cc(/C=C3\N=C(NC4CCCCOC4)NC3=O)ccc21] | C₁₈H₂₁N₅O₂ | 339.40 | 97% |
| 75 | 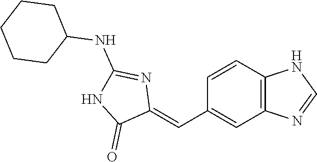 [SMILES: O=C1NC(NC2CCCCC2)=N/C1=C\c1ccc2[nH]cnc2c1] | C₁₇H₁₉N₅O | 309.37 | >98% |
| 76 | 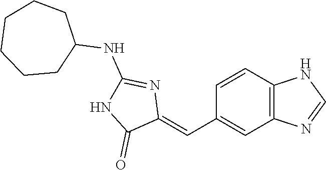 [SMILES: O=C1NC(NC2CCCCCC2)=N/C1=C\c1ccc2[nH]cnc2c1] | C₁₈H₂₁N₅O | 323.4 | >98% |
| 77 | 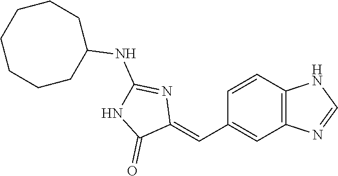 [SMILES: O=C1NC(NC2CCCCCCC2)=N/C1=C\c1ccc2[nH]cnc2c1] | C₁₉H₂₃N₅O | 337.43 | >98% |
| 78 | 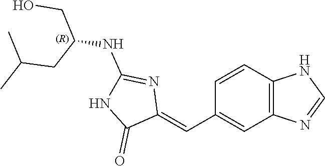 | C₁₇H₂₁N₅O₂ | 327.39 | 95% |
| 79 | 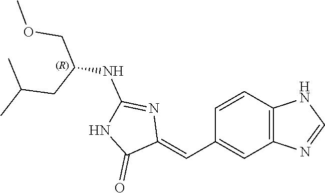 | C₁₈H₂₃N₅O₂ | 341.42 | 97% |
| 80 | 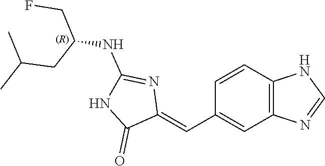 | C₁₇H₂₀FN₅O | 329.38 | 95% |
| 81 | 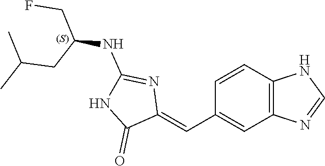 | C₁₇H₂₀FN₅O | 329.38 | 90% |
| 82 | 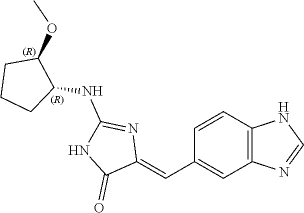 | C₁₇H₁₉N₅O₂ | 325.37 | 95% |
| 83 | 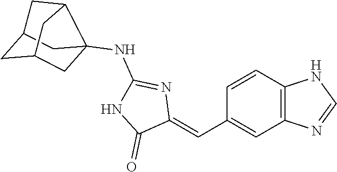 [SMILES: O=C1NC(NC23CC4CC(CC2C4)C3)=N/C1=C\c1ccc2[nH]cnc2c1] | C₂₀H₂₁N₅O | 347.42 | >98% |
| 84 | 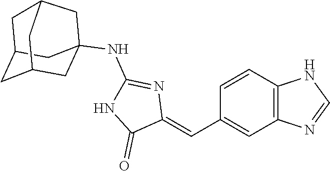 [SMILES: O=C1NC(NC23CC4CC(CC(C4)C2)C3)=N/C1=C\c1ccc2[nH]cnc2c1] | C₂₁H₂₃N₅O | 361.45 | >98% |
| 85 | 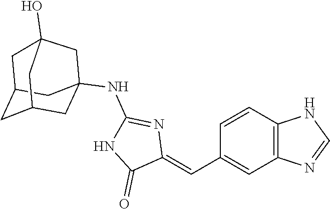 [SMILES: O=C1NC(NC23CC4CC(CC(O)(C4)C2)C3)=N/C1=C\c1ccc2[nH]cnc2c1] | C₂₁H₂₃N₅O₂ | 377.45 | 97% |
| 86 | 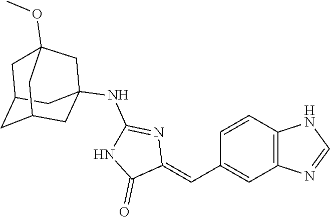 [SMILES: COC12CC3CC(CC(NC4=N/C(=C\c5ccc6[nH]cnc6c5)C(=O)N4)(C3)C1)C2] | C₂₂H₂₅N₅O₂ | 391.48 | 97% |
| 87 | 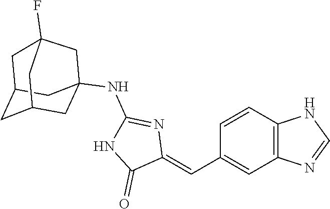 [SMILES: O=C1NC(NC23CC4CC(CC(F)(C4)C2)C3)=N/C1=C\c1ccc2[nH]cnc2c1] | C₂₁H₂₂FN₅O | 379.44 | >98% |
| 88 | 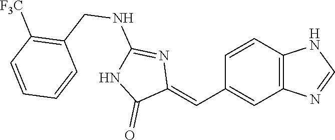 [SMILES: O=C1NC(NCc2ccccc2C(F)(F)F)=N/C1=C\c1ccc2[nH]cnc2c1] | C₁₉H₁₄F₃N₅O | 385.35 | >98% |
| 89 | 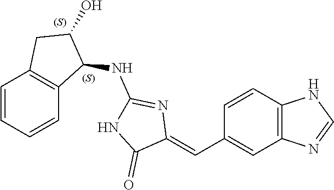 | C₂₀H₁₇N₅O₂ | 359.39 | 96% |
| 90 | 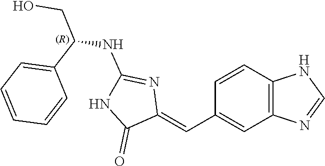 | C₁₉H₁₇N₅O₂ | 347.38 | >98% |
| 91 | 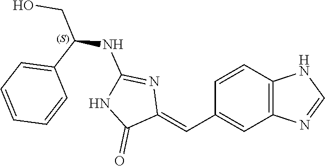 | C₁₉H₁₇N₅O₂ | 347.38 | >98% |
| 92 | 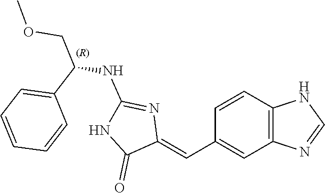 | C₂₀H₁₉N₅O₂ | 361.41 | >98% |
| 93 | 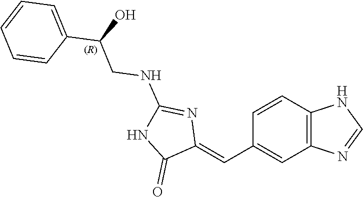 | C₁₉H₁₇N₅O₂ | 347.38 | 96% |
| 94 | 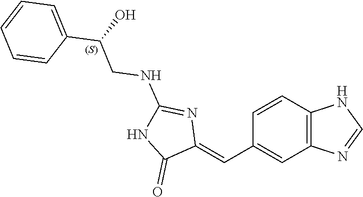 | C₁₉H₁₇N₅O₂ | 347.38 | >98% |
| 95 | 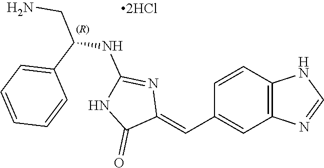 | C₁₉H₂₀C₁₂N₆O | 419.31 | 92% |
| 96 | 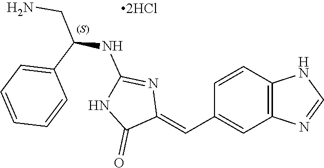 | C₁₉H₂₀C₁₂N₆O | 419.31 | 98% |
| 97 | 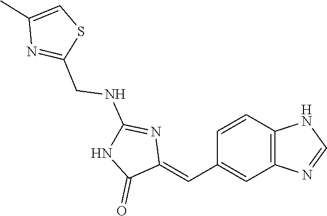 [SMILES: Cc1csc(CNC2=N/C(=C\c3ccc4[nH]cnc4c3)C(=O)N2)n1] | C₁₆H₁₄N₆OS | 338.39 | 97% |
| 98 | 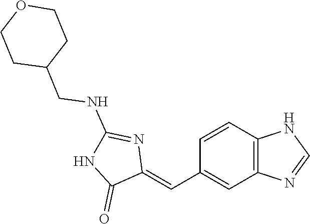 [SMILES: O=C1NC(NCC2CCOCC2)=N/C1=C\c1ccc2[nH]cnc2c1] | C₁₇H₁₉N₅O₂ | 325.37 | >98% |
| 99 | 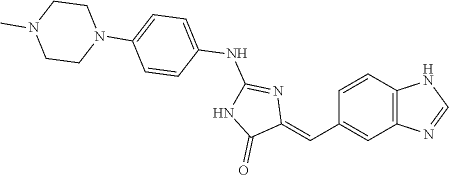 [SMILES: CN1CCN(c2ccc(NC3=N/C(=C\c4ccc5[nH]cnc5c4)C(=O)N3)cc2)CC1] | C₂₂H₂₃N₇O | 401.47 | >98% |
| 100 | 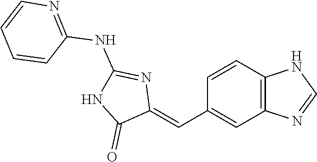 [SMILES: O=C1NC(Nc2ccccn2)=N/C1=C\c1ccc2[nH]cnc2c1] | C₁₆H₁₂N₆O | 304.31 | 95% |
| 101 | 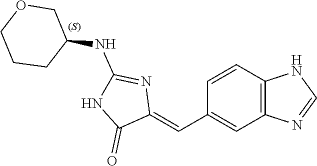 | C₁₆H₁₇N₅O₂ | 311.35 | >98% |
| 102 | 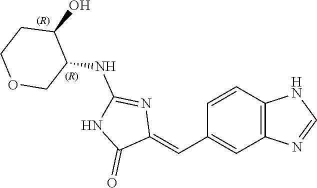 | C₁₆H₁₇N₅O₃ | 327.34 | >98% |
| 103 | 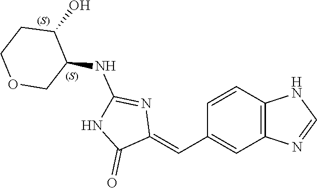 | C₁₆H₁₇N₅O₃ | 327.34 | >98% |
| 104 | 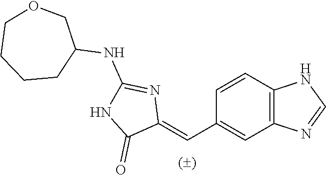 [SMILES: O=C1NC(NC2CCCCOC2)=N/C1=C\c1ccc2[nH]cnc2c1] | C₁₇H₁₉N₅O₂ | 325.37 | >98% |
| 105 | 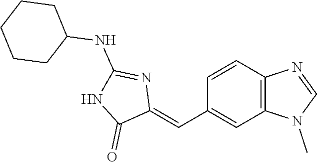 [SMILES: Cn1cnc2ccc(/C=C3\N=C(NC4CCCCC4)NC3=O)cc21] | C₁₈H₂₁N₅O | 323.40 | 97% |
| 106 | 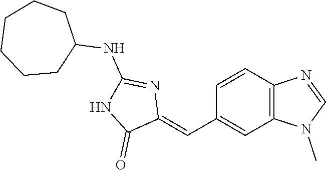 [SMILES: Cn1cnc2ccc(/C=C3\N=C(NC4CCCCCC4)NC3=O)cc21] | C₁₉H₂₃N₅O | 337.43 | >98% |
| 107 | 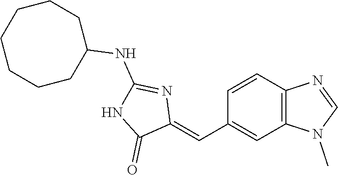 [SMILES: Cn1cnc2ccc(/C=C3\N=C(NC4CCCCCCC4)NC3=O)cc21] | C₂₀H₂₅N₅O | 351.45 | 95% |
| 108 | 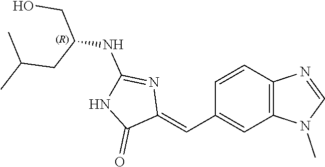 | C₁₈H₂₃N₅O₂ | 341.42 | >98% |
| 109 | 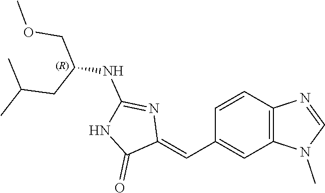 | C₁₉H₂₅N₅O₂ | 355.44 | >98% |
| 110 | 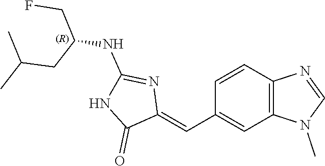 | C₁₈H₂₂FN₅O | 343.41 | 98% |
| 111 | 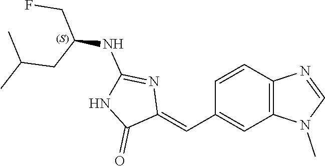 | C₁₈H₂₂FN₅O | 343.41 | >98% |
| 112 | 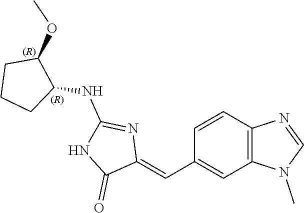 | C₁₈H₂₁N₅O₂ | 339.40 | 95% |
| 113 | 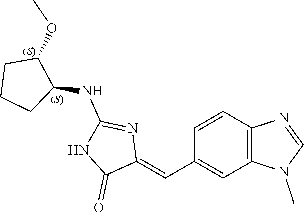 | C₁₈H₂₁N₅O₂ | 339.40 | 95% |
| 114 | 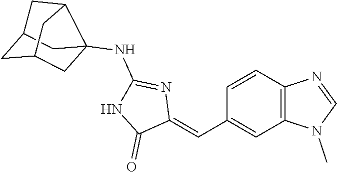 [SMILES: Cn1cnc2ccc(/C=C3\N=C(NC45CC6CC(CC4C6)C5)NC3=O)cc21] | C₂₁H₂₃N₅O | 361.45 | 95% |
| 115 | 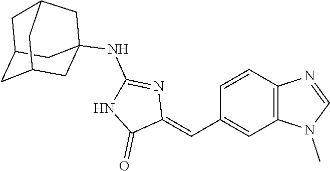 [SMILES: Cn1cnc2ccc(/C=C3\N=C(NC45CC6CC(CC(C6)C4)C5)NC3=O)cc21] | C₂₂H₂₅N₅O | 375.48 | >98% |
| 116 | 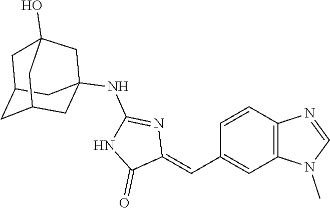 [SMILES: Cn1cnc2ccc(/C=C3\N=C(NC45CC6CC(CC(O)(C6)C4)C5)NC3=O)cc21] | C₂₂H₂₅N₅O₂ | 391.48 | 98% |
| 117 | 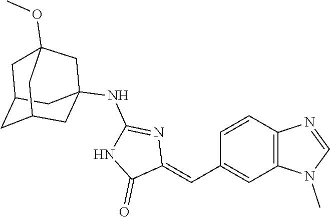 [SMILES: COC12CC3CC(CC(NC4=N/C(=C\c5ccc6ncn(C)c6c5)C(=O)N4)(C3)C1)C2] | C₂₃H₂₇N₅O₂ | 405.50 | 98% (¹H NMR) |
| 118 | 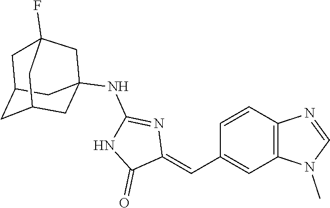 [SMILES: Cn1cnc2ccc(/C=C3\N=C(NC45CC6CC(CC(F)(C6)C4)C5)NC3=O)cc21] | C₂₂H₂₄FN₅O | 393.47 | 96% |
| 119 | 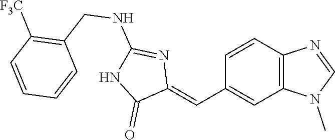 [SMILES: Cn1cnc2ccc(/C=C3\N=C(NCc4ccccc4C(F)(F)F)NC3=O)cc21] | C₂₀H₁₆F₃N₅O | 399.38 | 96% |
| 120 | 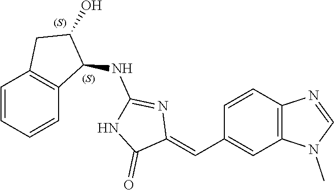 | C₂₁H₁₉N₅O₂ | 373.42 | 89% |
| 121 | 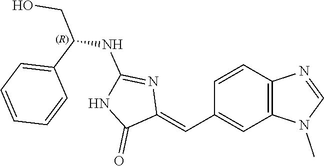 | C₂₀H₁₉N₅O₂ | 361.41 | 97% |
| 122 | 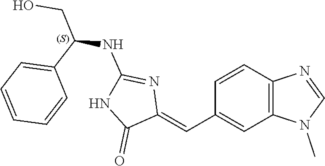 | C₂₀H₁₉N₅O₂ | 361.41 | 93% |
| 123 | 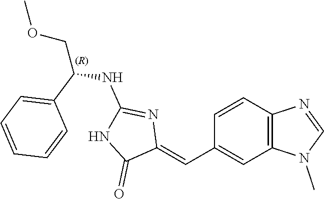 | C₂₁H₂₁N₅O₂ | 375.43 | 91% (Z) + 6% (E) |
| 124 | 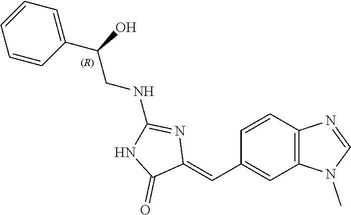 | C₂₀H₁₉N₅O₂ | 361.41 | >98% |
| 125 | 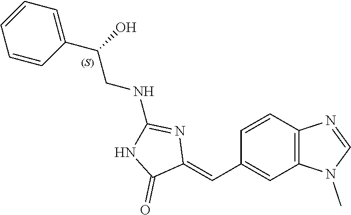 | C₂₀H₁₉N₅O₂ | 361.41 | >98% |
| 126 | 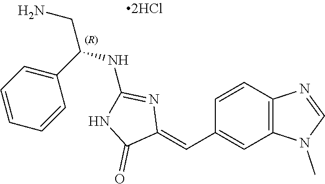 | C₂₀H₂₂C₁₂N₆0 | 433.34 | 97% |
| 127 | 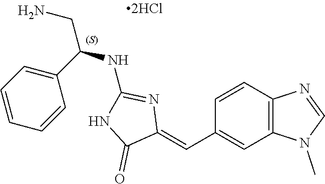 | C₂₀H₂₂Cl₂N₆O | 433.34 | 95% |
| 128 | 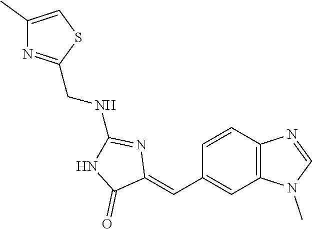 [SMILES: Cc1csc(CNC2=N/C(=C\c3ccc4ncn(C)c4c3)C(=O)N2)n1] | C₁₇H₁₆N₆OS | 352.42 | 96% |
| 129 | 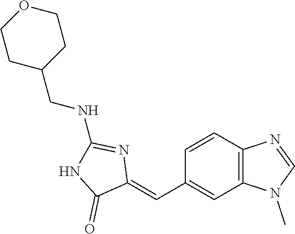 [SMILES: Cn1cnc2ccc(/C=C3\N=C(NCC4CCOCC4)NC3=O)cc21] | C₁₈H₂₁N₅O₂ | 339.40 | >98% |
| 130 | 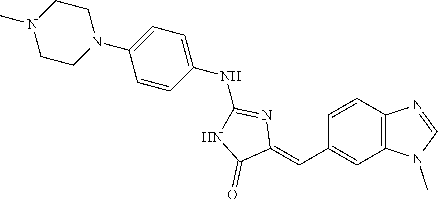 [SMILES: CN1CCN(c2ccc(NC3=N/C(=C\c4ccc5ncn(C)c5c4)C(=O)N3)cc2)CC1] | C₂₃H₂₅N₇O | 415.50 | >98% |
| 131 | 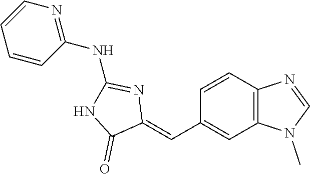 [SMILES: Cn1cnc2ccc(/C=C3\N=C(Nc4ccccn4)NC3=O)cc21] | C₁₇H₁₄N₆O | 318.34 | 96% |
| 132 | 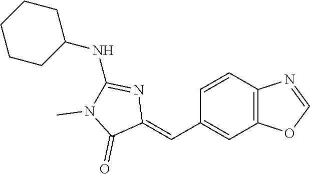 [SMILES: CN1C(=O)/C(=C/c2ccc3ncoc3c2)N=C1NC1CCCCC1] | C₁₈H₂₀N₄O₂ | 324.38 | >98% |
| 133 | 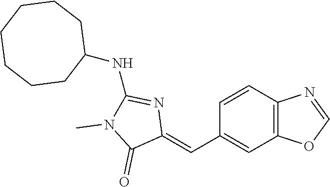 [SMILES: CN1C(=O)/C(=C/c2ccc3ncoc3c2)N=C1NC1CCCCCCC1] | C₂₀H₂₄N₄O₂ | 352.44 | >96% |
| 134 | 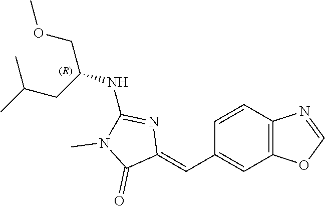 | C₁₉H₂₄N₄O₃ | 356.43 | >98% |
| 135 | 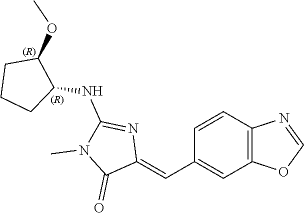 | C₁₈H₂₀N₄O₃ | 340.38 | >98% |
| 136 | 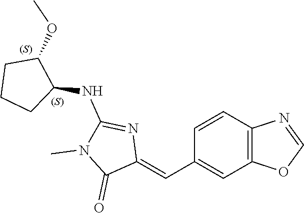 | C₁₈H₂₀N₄O₃ | 340.38 | >98% |
| 137 | 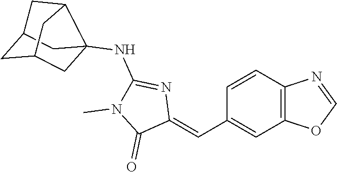 [SMILES: CN1C(=O)/C(=C/c2ccc3ncoc3c2)N=C1NC12CC3CC(CC1C3)C2] | C₂₁H₂₂N₄O₂ | 362.43 | 97% |
| 138 | 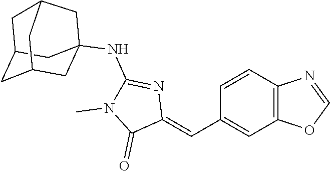 [SMILES: CN1C(=O)/C(=C/c2ccc3ncoc3c2)N=C1NC12CC3CC(CC(C3)C1)C2] | C₂₂H₂₄N₄O₂ | 376.46 | >98% |
| 139 | 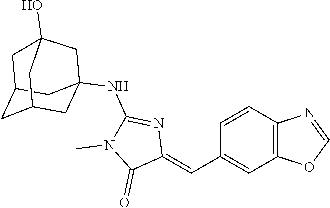 [SMILES: CN1C(=O)/C(=C/c2ccc3ncoc3c2)N=C1NC12CC3CC(CC(O)(C3)C1)C2] | C₂₂H₂₄N₄O₃ | 392.46 | >98% (Z) ⁺ (E) |
| 140 | 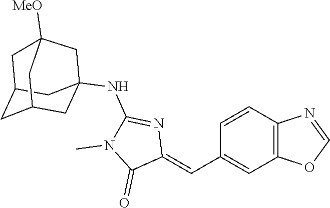 [SMILES: COC12CC3CC(CC(NC4=N/C(=C\c5ccc6ncoc6c5)C(=O)N4C)(C3)C1)C2] | C₂₃H₂₆N₄O₃ | 406.49 | >98% |
| 141 | 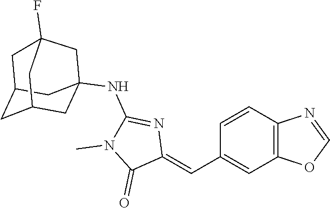 [SMILES: CN1C(=O)/C(=C/c2ccc3ncoc3c2)N=C1NC12CC3CC(CC(F)(C3)C1)C2] | C₂₂H₂₃FN₄O₂ | 394.45 | >98% |
| 142 | 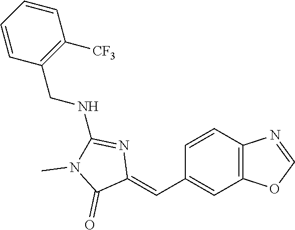 [SMILES: CN1C(=O)/C(=C/c2ccc3ncoc3c2)N=C1NCc1ccccc1C(F)(F)F] | C₂₀H₁₅F₃N₄O₂ | 400.36 | >98% |
| 143 | 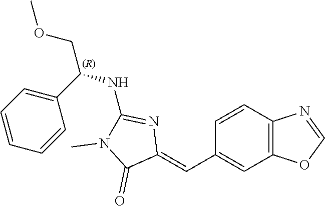 | C₂₁H₂₀N₄O₃ | 376.42 | >98% |
| 144 | 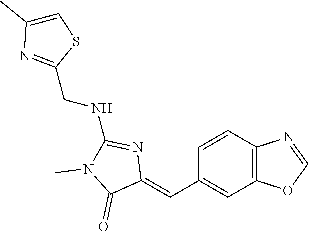 [SMILES: Cc1csc(CNC2=N/C(=C\c3ccc4ncoc4c3)C(=O)N2C)n1] | C₁₇H₁₅N₅O₂S | 353.40 | >98% |
| 145 | 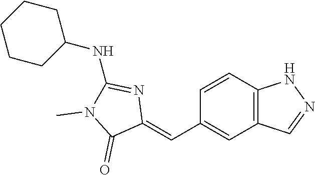 [SMILES: CN1C(=O)/C(=C/c2ccc3[nH]ncc3c2)N=C1NC1CCCCC1] | C₁₈H₂₁N₅O | 323.40 | 95% |
| 146 | 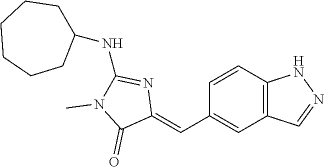 [SMILES: CN1C(=O)/C(=C/c2ccc3[nH]ncc3c2)N=C1NC1CCCCCC1] | C₁₉H₂₃N₅O | 337.43 | >98% |
| 147 | 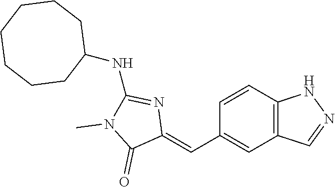 [SMILES: CN1C(=O)/C(=C/c2ccc3[nH]ncc3c2)N=C1NC1CCCCCCC1] | C₂₀H₂₅N₅O | 351.45 | >98% |
| 148 | 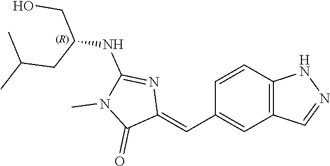 | C₁₈H₂₃N₅O₂ | 341.42 | >98% |
| 149 | 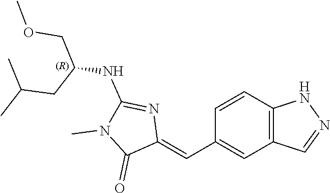 | C₁₉H₂₅N₅O₂ | 355.44 | >98% |
| 150 | 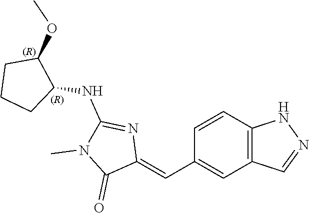 | C₁₈H₂₁N₅O₂ | 339.40 | >98% |
| 151 | 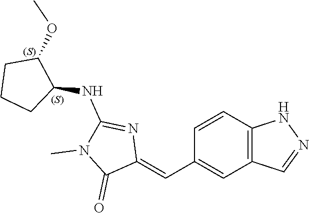 | C₁₈H₂₁N₅O₂ | 339.40 | 96% |
| 152 | 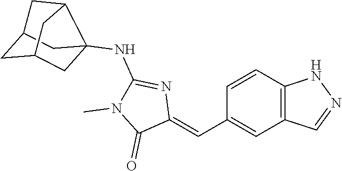 [SMILES: CN1C(=O)/C(=C/c2ccc3[nH]ncc3c2)N=C1NC12CC3CC(CC1C3)C2] | C₂₁H₂₁N₅O₂ | 361.45 | >98% |
| 153 | 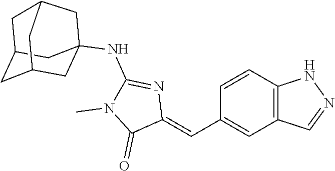 [SMILES: CN1C(=O)/C(=C/c2ccc3[nH]ncc3c2)N=C1NC12CC3CC(CC(C3)C1)C2] | C₂₂H₂₅N₅O | 375.48 | >98% |
| 154 | 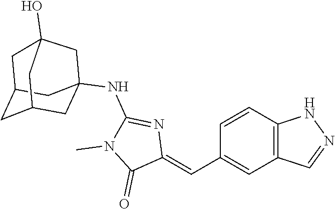 [SMILES: CN1C(=O)/C(=C/c2ccc3[nH]ncc3c2)N=C1NC12CC3CC(CC(O)(C3)C1)C2] | C₂₂H₂₅N₅O₂ | 391.48 | >98% |
| 155 | 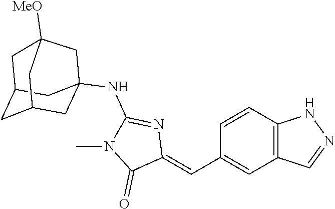 [SMILES: COC12CC3CC(CC(NC4=N/C(=C\c5ccc6[nH]ncc6c5)C(=O)N4C)(C3)C1)C2] | C₂₃H₂₇N₅O₂ | 405.50 | >98% |
| 156 | 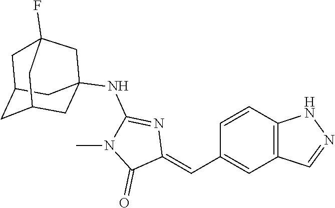 [SMILES: CN1C(=O)/C(=C/c2ccc3[nH]ncc3c2)N=C1NC12CC3CC(CC(F)(C3)C1)C2] | C₂₂H₂₄FN₅O | 393.47 | >98% |
| 157 | 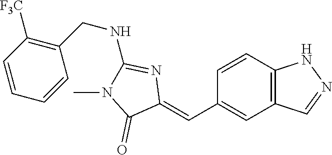 [SMILES: CN1C(=O)/C(=C/c2ccc3[nH]ncc3c2)N=C1NCc1ccccc1C(F)(F)F] | C₂₀H₁₆F₃N₅O | 399.38 | 97% |
| 158 | 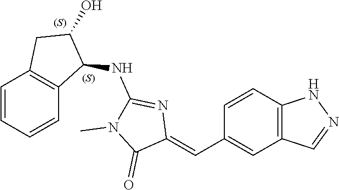 | C₂₁H₁₉N₅O₂ | 373.42 | >98% |
| 159 | 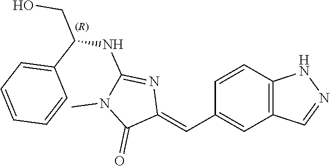 | C₂₀H₁₉N₅O₂ | 361.41 | >98% |
| 160 | 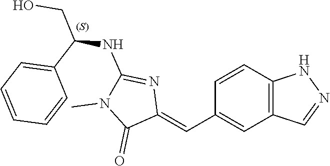 | C₂₀H₁₉N₅O₂ | 361.41 | >98% |
| 161 | 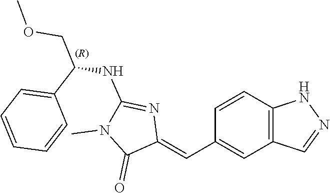 | C₂₁H₂₁N₅O₂ | 375.43 | >98% |
| 162 | 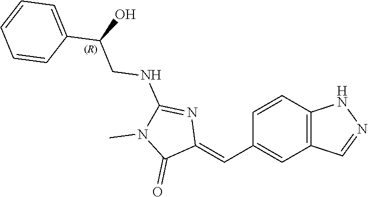 | C₂₀H₁₉N₅O₂ | 361.41 | >98% |
| 163 | 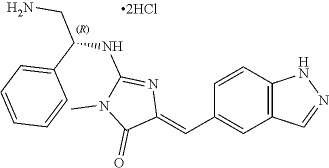 | C₂₀H₂₂C₁₂N₆O | 433.34 | 88% |
| 164 | 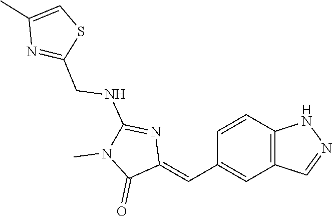 [SMILES: Cc1csc(CNC2=N/C(=C\c3ccc4[nH]ncc4c3)C(=O)N2C)n1] | C₁₇H₁₆N₆OS | 352.42 | >98% |
| 165 | 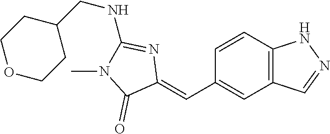 [SMILES: CN1C(=O)/C(=C/c2ccc3[nH]ncc3c2)N=C1NCC1CCOCC1] | C₁₈H₂₁N₅O₂ | 339.40 | >98% |
| 166 | 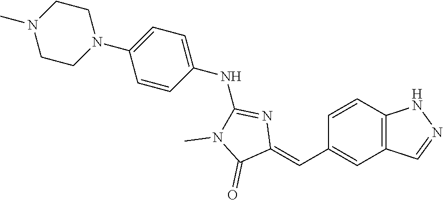 [SMILES: CN1CCN(c2ccc(NC3=N/C(=C\c4ccc5[nH]ncc5c4)C(=O)N3C)cc2)CC1] | C₂₃H₂₅NO | 415.50 | >98% |
| 167 | 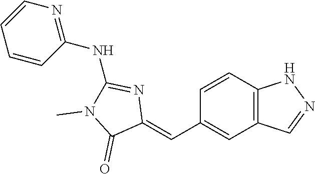 [SMILES: CN1C(=O)/C(=C/c2ccc3[nH]ncc3c2)N=C1Nc1ccccn1] | C₁₇H₁₄N₆O | 318.34 | >98% |
| 168 | 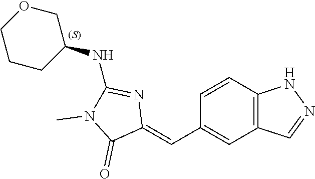 | C₁₇H₁₉N₅O₂ | 325.37 | >98% |
| 169 | 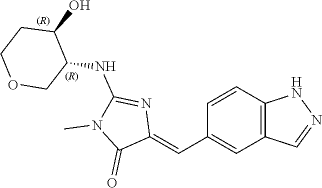 | C₁₇H₁₉N₅O₃ | 341.37 | >98% |
| 170 | 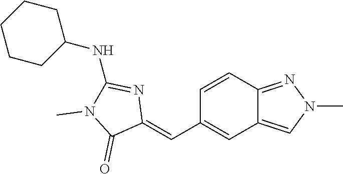 [SMILES: CN1C(=O)/C(=C/c2ccc3nn(C)cc3c2)N=C1NC1CCCCC1] | C₁₉H₂₃N₅O | 337.43 | >98% |
| 171 | 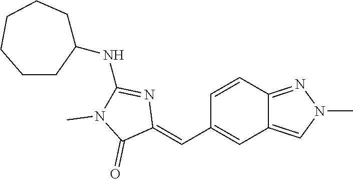 [SMILES: CN1C(=O)/C(=C/c2ccc3nn(C)cc3c2)N=C1NC1CCCCCC1] | C₂₀H₂₅N₅O | 351.45 | >98% |
| 172 | 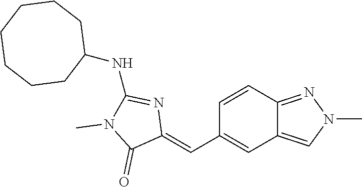 [SMILES: CN1C(=O)/C(=C/c2ccc3nn(C)cc3c2)N=C1NC1CCCCCCC1] | C₂₁H₂₇N₅O | 365.48 | >98% |
| 173 | 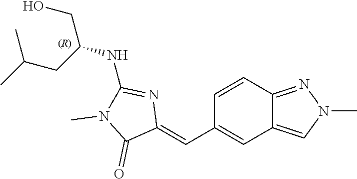 | C₁₉H₂₅N₅O₂ | 355.44 | 96% |
| 174 | 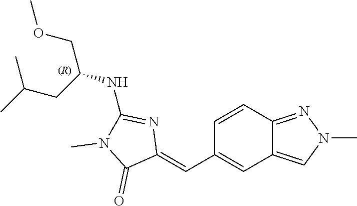 | C₂₀H₂₇N₅O₂ | 369.47 | >98% |
| 175 | 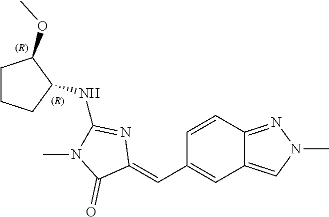 | C₁₉H₂₃N₅O₂ | 353.43 | >98% |
| 176 | 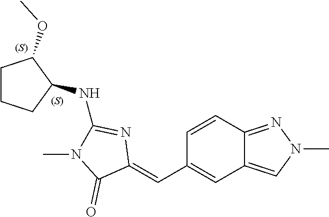 | C₁₉H₂₃N₅O₂ | 353.43 | >98% |
| 177 | 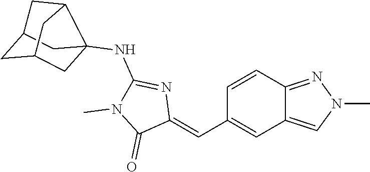 [SMILES: CN1C(=O)/C(=C/c2ccc3nn(C)cc3c2)N=C1NC12CC3CC(CC1C3)C2] | C₂₂H₂₅N₅O | 375.48 | >98% |
| 178 | 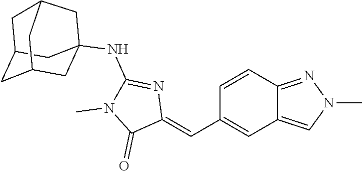 [SMILES: CN1C(=O)/C(=C/c2ccc3nn(C)cc3c2)N=C1NC12CC3CC(CC(C3)C1)C2] | C₂₃H₂₇N₅O | 389.50 | >98% |
| 179 | 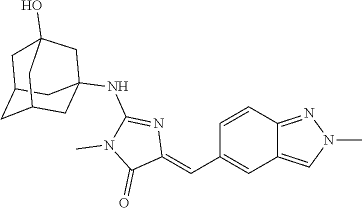 [SMILES: CN1C(=O)/C(=C/c2ccc3nn(C)cc3c2)N=C1NC12CC3CC(CC(O)(C3)C1)C2] | C₂₃H₂₇N₅O₂ | 405.50 | >98% |
| 180 | 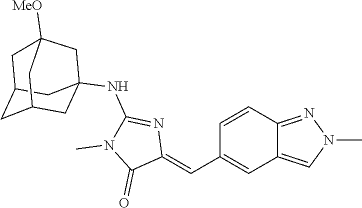 [SMILES: COC12CC3CC(CC(NC4=N/C(=C\c5ccc6nn(C)cc6c5)C(=O)N4C)(C3)C1)C2] | C₂₄H₂₉N₅O₂ | 419.53 | >98% |
| 181 | 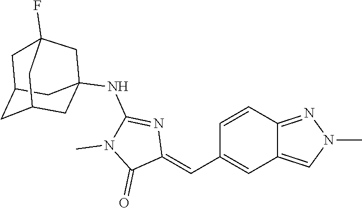 [SMILES: CN1C(=O)/C(=C/c2ccc3nn(C)cc3c2)N=C1NC12CC3CC(CC(F)(C3)C1)C2] | C₂₃H₂₆FN₅O | 407.49 | >98% |
| 182 | 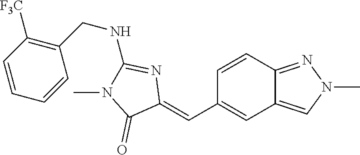 [SMILES: CN1C(=O)/C(=C/c2ccc3nn(C)cc3c2)N=C1NCc1ccccc1C(F)(F)F] | C₂₁H₁₈F₃N₅O | 413.40 | >98% |
| 183 | 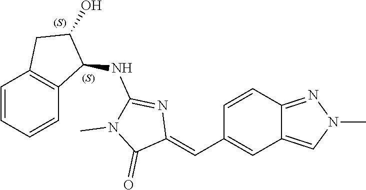 | C₂₂H₂₁N₅O₂ | 387.44 | 97% |
| 184 | 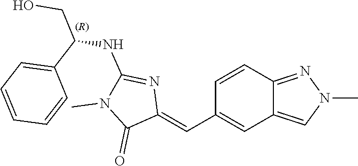 | C₂₁H₂₁N₅O₂ | 375.43 | >98% |
| 185 | 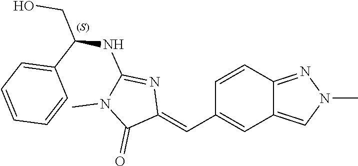 | C₂₁H₂₁N₅O₂ | 375.43 | >98% |
| 186 | 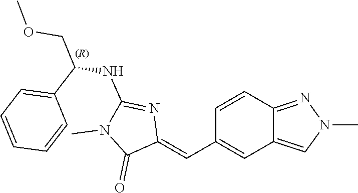 | C₂₂H₂₃N₅O₂ | 389.46 | >98% |
| 187 | 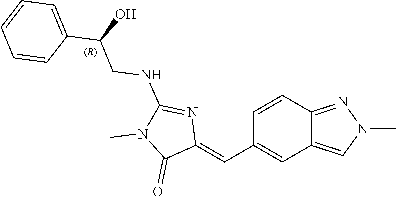 | C₂₁H₂₁N₅O₂ | 375.43 | >98% |
| 188 | 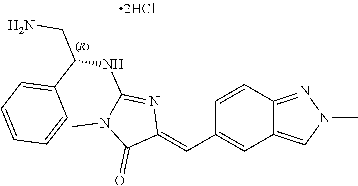 | C₂₁H₂₄Cl₂N₆O | 447.36 | 89% |
| 189 | 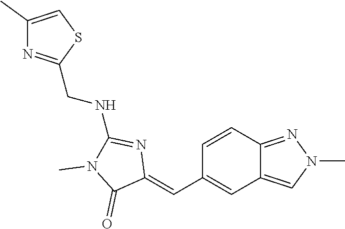 | C₁₈H₁₈N₆OS | 366.44 | >98% |
| 190 | 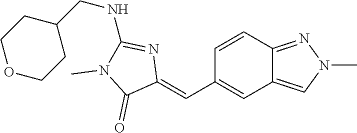 [SMILES: CN1C(=O)/C(=C/c2ccc3nn(C)cc3c2)N=C1NCC1CCOCC1] | C₁₉H₂₃N₅O₂ | 353.43 | >98% |
| 191 | 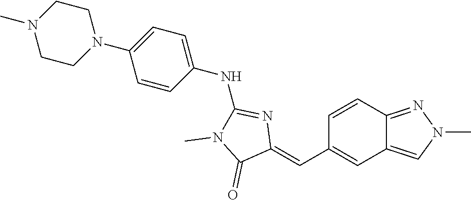 | C₂₄H₂₇N₇O | 429.53 | >98% |
| 192 | 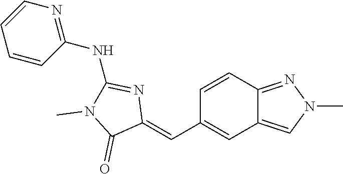 [SMILES: CN1C(=O)/C(=C/c2ccc3nn(C)cc3c2)N=C1Nc1ccccn1] | C₁₈H₁₆N₆O | 332.37 | >98% |
| 193 | 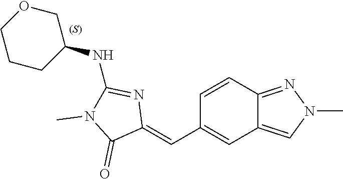 | C₁₈H₂₁N₅O₂ | 339.40 | 94% |
| 194 | 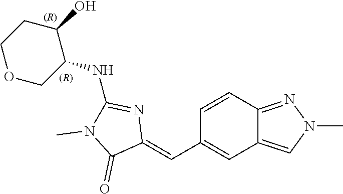 | C₁₈H₂₁N₅O₃ | 355.40 | >98% |
| 195 | 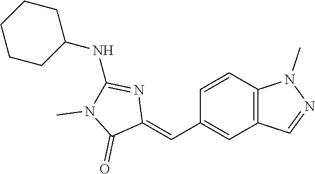 [SMILES: CN1C(=O)/C(=C/c2ccc3c(cnn3C)c2)N=C1NC1CCCCC1] | C₁₉H₂₃N₅O | 337.43 | >98% |
| 196 | 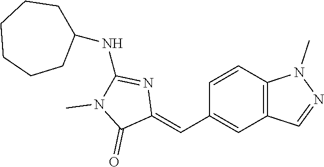 [SMILES: CN1C(=O)/C(=C/c2ccc3c(cnn3C)c2)N=C1NC1CCCCCC1] | C₂₀H₂₅N₅O | 351.45 | >98% |
| 197 | 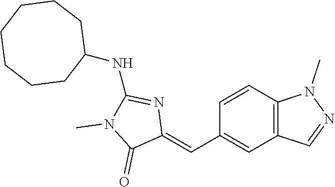 [SMILES: CN1C(=O)/C(=C/c2ccc3c(cnn3C)c2)N=C1NC1CCCCCCC1] | C₂₁H₂₇N₅O | 365.48 | >98% |
| 198 | 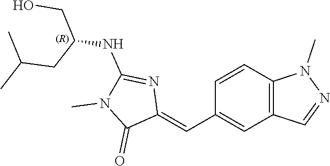 | C₁₉H₂₅N₅O₂ | 355.44 | 98% |
| 199 | 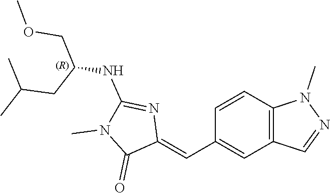 | C₂₀H₂₇N₅O₂ | 369.47 | >98% |
| 200 | 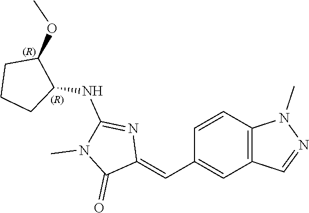 | C₁₉H₂₃N₅O₂ | 353.43 | >98% |
| 201 | 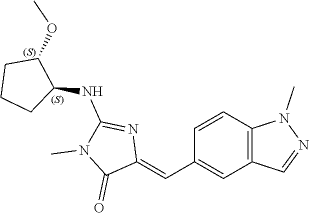 | C₁₉H₂₃N₅O₂ | 353.43 | >98% |
| 202 | 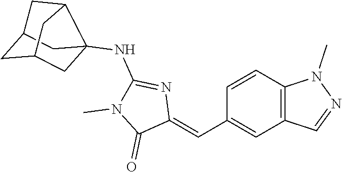 [SMILES: CN1C(=O)/C(=C/c2ccc3c(cnn3C)c2)N=C1NC12CC3CC(CC1C3)C2] | C₂₂H₂₅N₅O | 375.48 | >98% |
| 203 | 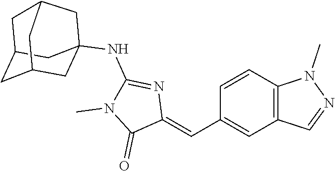 [SMILES: CN1C(=O)/C(=C/c2ccc3c(cnn3C)c2)N=C1NC12CC3CC(CC(C3)C1)C2] | C₂₃H₂₇N₅O | 389.50 | >98% |
| 204 | 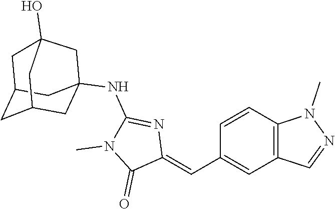 [SMILES: CN1C(=O)/C(=C/c2ccc3c(cnn3C)c2)N=C1NC12CC3CC(CC(O)(C3)C1)C2] | C₂₃H₂₇N₅O₂ | 405.50 | >98% |
| 205 | 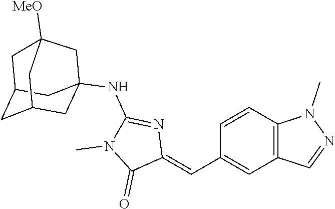 [SMILES: COC12CC3CC(CC(NC4=N/C(=C\c5ccc6c(cnn6C)c5)C(=O)N4C)(C3)C1)C2] | C₂₄H₂₉N₅O₂ | 419.53 | >98% |
| 206 | 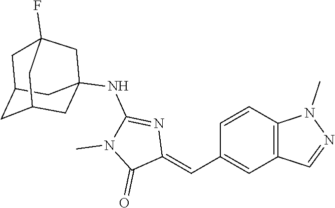 [SMILES: CN1C(=O)/C(=C/c2ccc3c(cnn3C)c2)N=C1NC12CC3CC(CC(F)(C3)C1)C2] | C₂₃H₂₆FN₅O | 407.49 | >98% |
| 207 | 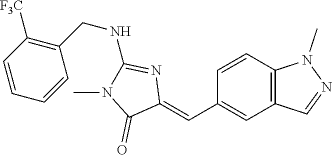 [SMILES: CN1C(=O)/C(=C/c2ccc3c(cnn3C)c2)N=C1NCc1ccccc1C(F)(F)F] | C₂₁H₁₈F₃N₅O | 413.40 | >98% |
| 208 | 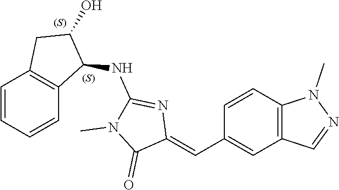 | C₂₂H₂₁N₅O₂ | 387.44 | >98% |
| 209 | 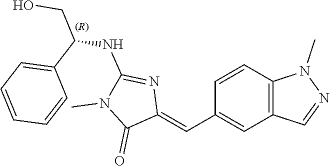 | C₂₁H₂₁N₅O₂ | 375.43 | >98% |
| 210 | 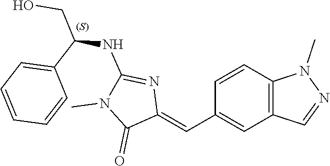 | C₂₁H₂₁N₅O₂ | 375.43 | >98% |
| 211 | 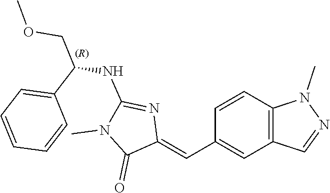 | C₂₂H₂₃N₅O₂ | 389.46 | >98% |
| 212 | 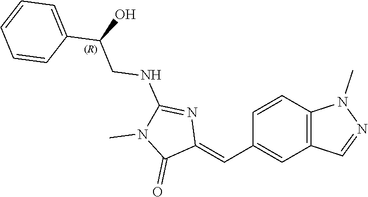 | C₂₁H₂₁N₅O₂ | 375.43 | >98% |
| 213 | 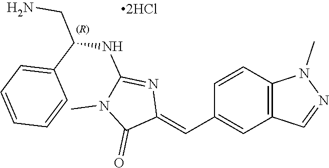 | C₂₁H₂₄Cl₂N₆O | 447.36 | 96% |
| 214 | 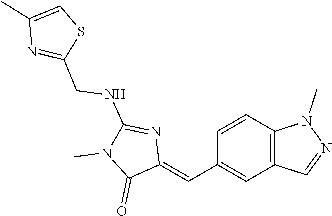 [SMILES: Cc1csc(CNC2=N/C(=C\c3ccc4c(cnn4C)c3)C(=O)N2C)n1] | C₁₈H₁₈N₆OS | 366.44 | >98% |
| 215 | 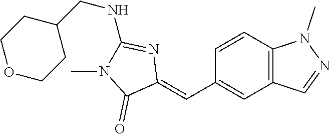 [SMILES: CN1C(=O)/C(=C/c2ccc3c(cnn3C)c2)N=C1NCC1CCOCC1] | C₁₉H₂₃N₅O₂ | 353.43 | >98% |
| 216 | 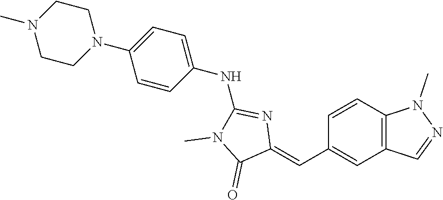 [SMILES: CN1CCN(c2ccc(NC3=N/C(=C\c4ccc5c(cnn5C)c4)C(=O)N3C)cc2)CC1] | C₂₄H₂₇N₇O | 429.53 | >98% |
| 217 | 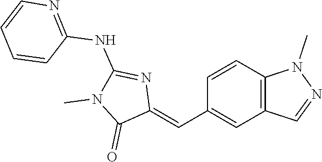 [SMILES: CN1C(=O)/C(=C/c2ccc3c(cnn3C)c2)N=C1Nc1ccccn1] | C₁₈H₁₆N₆O | 332.37 | >98% |
| 218 | 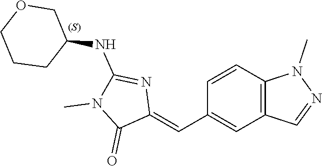 | C₁₈H₂₁N₅O₂ | 339.40 | >98% |
| 219 | 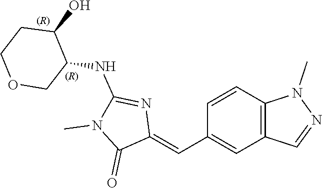 | C₁₈H₂₁N₅O₃ | 355.40 | >98% |
| 220 | 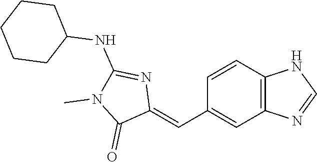 [SMILES: CN1C(=O)/C(=C/c2ccc3[nH]cnc3c2)N=C1NC1CCCCC1] | C₁₈H₂₁N₅O | 323.40 | >98% |
| 221 | 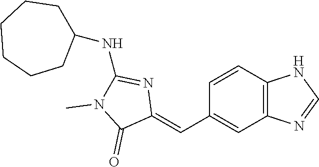 [SMILES: CN1C(=O)/C(=C/c2ccc3[nH]cnc3c2)N=C1NC1CCCCCC1] | C₁₉H₂₃N₅O | 337.43 | >98% |
| 222 | 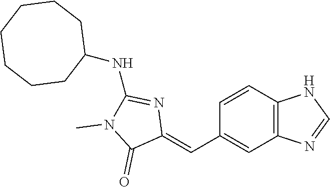 [SMILES: CN1C(=O)/C(=C/c2ccc3[nH]cnc3c2)N=C1NC1CCCCCCC1] | C₂₀H₂₅N₅O | 351.45 | >98% |
| 223 | 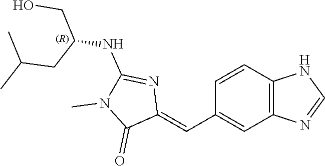 | C₁₈H₂₃N₅O₂ | 341.42 | >98 |
| 224 | 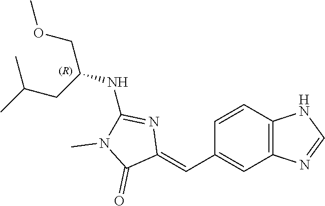 | C₁₉H₂₅N₅O₂ | 355.44 | 94% |
| 225 | 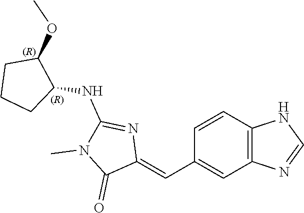 | C₁₈H₂₁N₅O₂ | 339.40 | 98% (¹H NMR) |
| 226 | 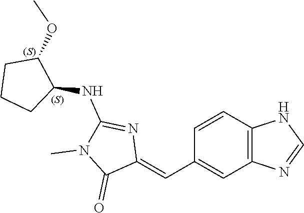 | C₁₈H₂₁N₅O₂ | 339.40 | 98% (¹H NMR) |
| 227 | 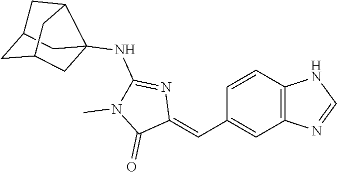 [SMILES: CN1C(=O)/C(=C/c2ccc3[nH]cnc3c2)N=C1NC12CC3CC(CC1C3)C2] | C₂₁H₂₃N₅O | 361.45 | 97% |
| 228 | 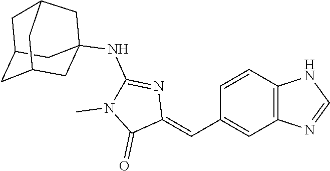 [SMILES: CN1C(=O)/C(=C/c2ccc3[nH]cnc3c2)N=C1NC12CC3CC(CC(C3)C1)C2] | C₂₂H₂₅N₅O | 375.48 | >98% |
| 229 | 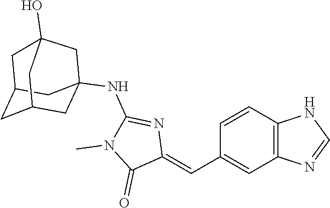 [SMILES: CN1C(=O)/C(=C/c2ccc3[nH]cnc3c2)N=C1NC12CC3CC(CC(O)(C3)C1)C2] | C₂₂H₂₅N₅O₂ | 391.48 | >98% |
| 230 | 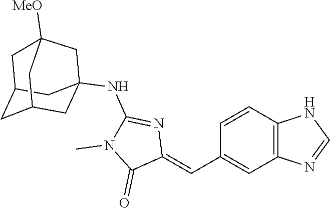 [SMILES: COC12CC3CC(CC(NC4=N/C(=C\c5ccc6[nH]cnc6c5)C(=O)N4C)(C3)C1)C2] | C₂₃H₂₇N₅O₂ | 405.50 | >98% |
| 231 | 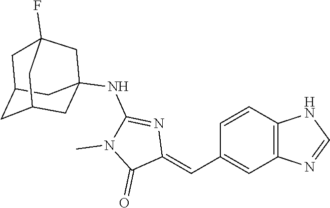 [SMILES: CN1C(=O)/C(=C/c2ccc3[nH]cnc3c2)N=C1NC12CC3CC(CC(F)(C3)C1)C2] | C₂₂H₂₄FN₅O | 393.47 | >98% |
| 232 | 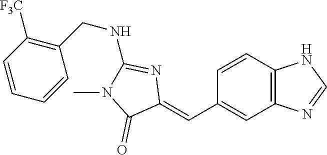 [SMILES: CN1C(=O)/C(=C/c2ccc3[nH]cnc3c2)N=C1NCc1ccccc1C(F)(F)F] | C₂₀H₁₆F₃N₅O | 399.38 | >98% |
| 233 | 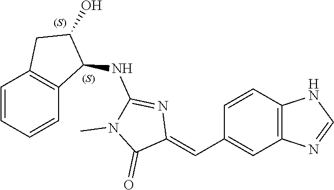 | C₂₁H₁₉N₅O₂ | 373.42 | 98% |
| 234 | 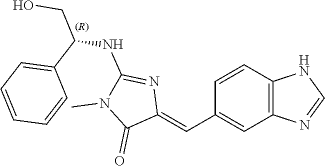 | C₂₀H₁₉N₅O₂ | 361.41 | >98% |
| 235 | 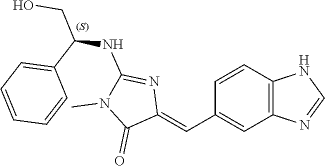 | C₂₀H₁₉N₅O₂ | 361.41 | >98% |
| 236 | 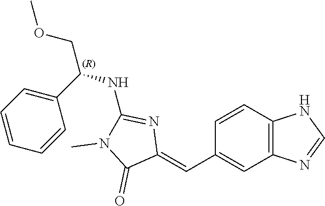 | C₂₁H₂₁N₅O₂ | 375.43 | >98% |
| 237 | 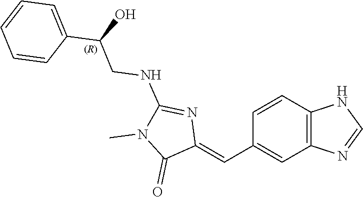 | C₂₀H₁₉N₅O₂ | 361.41 | 87% |
| 238 | 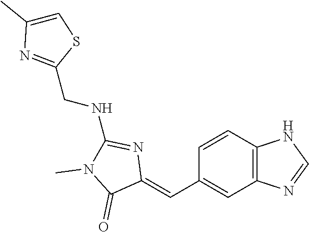 [SMILES: Cc1csc(CNC2=N/C(=C\c3ccc4[nH]cnc4c3)C(=O)N2C)n1] | C₁₇H₁₆N₆OS | 352.42 | >98% |
| 239 | 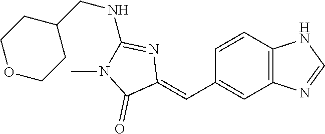 [SMILES: CN1C(=O)/C(=C/c2ccc3[nH]cnc3c2)N=C1NCC1CCOCC1] | C₁₈H₂₁N₅O₂ | 339.40 | >98% (¹H NMR) |
| 240 | 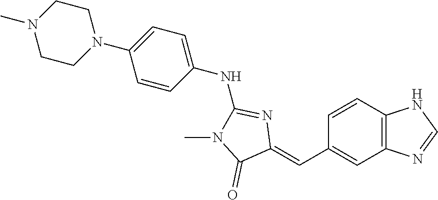 [SMILES: CN1CCN(c2ccc(NC3=N/C(=C\c4ccc5[nH]cnc5c4)C(=O)N3C)cc2)CC1] | C₂₃H₂₅N₇O | 415.50 | >98% (¹H NMR) |
| 241 | 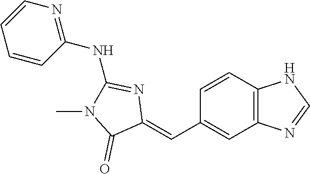 [SMILES: CN1C(=O)/C(=C/c2ccc3[nH]cnc3c2)N=C1Nc1ccccn1] | C₁₇H₁₄N₆O | 318.34 | >98% |
| 242 | 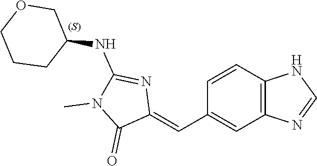 | C₁₇H₁₉N₅O₂ | 325.37 | 98% (¹H NMR) |
| 243 | 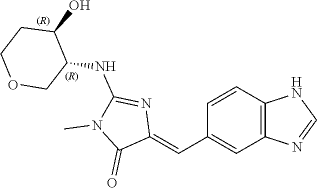 | C₁₇H₁₉N₅O₃ | 341.37 | 98% (¹H NMR) |
| 244 | 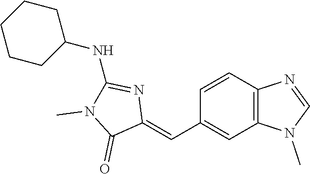 [SMILES: CN1C(=O)/C(=C/c2ccc3ncn(C)c3c2)N=C1NC1CCCCC1] | C₁₉H₂₃N₅O | 337.43 | >98% |
| 245 | 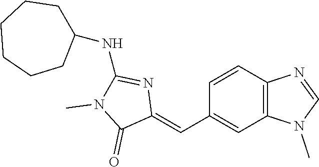 [SMILES: CN1C(=O)/C(=C/c2ccc3ncn(C)c3c2)N=C1NC1CCCCCC1] | C₂₀H₂₅N₅O | 351.45 | >98% |
| 246 | 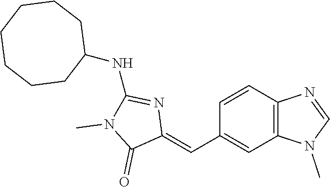 [SMILES: CN1C(=O)/C(=C/c2ccc3ncn(C)c3c2)N=C1NC1CCCCCCC1] | C₂₁H₂₇N₅O | 365.48 | >98% |
| 247 | 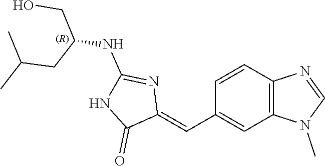 | C₁₉H₂₅N₅O₂ | 355.44 | >98% |
| 248 | 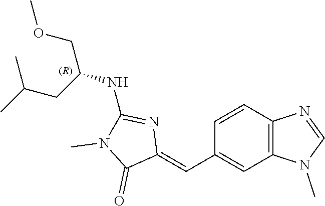 | C₂₀H₂₇N₅O₂ | 369.47 | >98% |
| 249 | 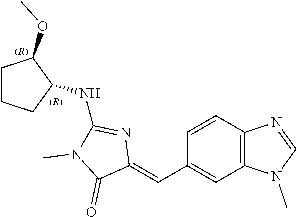 | C₁₉H₂₃N₅O₂ | 353.43 | >98% (¹H NMR) |
| 250 | 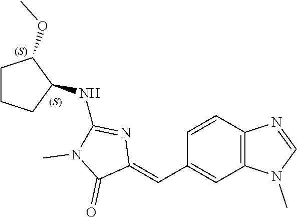 | C₁₉H₂₃N₅O₂ | 353.43 | >98% (¹H NMR) |
| 251 | 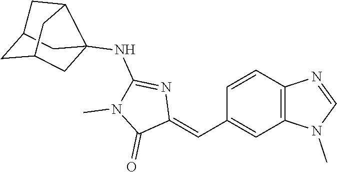 [SMILES: CN1C(=O)/C(=C/c2ccc3ncn(C)c3c2)N=C1NC12CC3CC(CC1C3)C2] | C₂₂H₂₅N₅O | 375.48 | >98% |
| 252 | 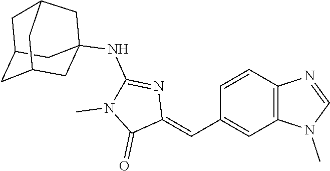 [SMILES: CN1C(=O)/C(=C/c2ccc3ncn(C)c3c2)N=C1NC12CC3CC(CC(C3)C1)C2] | C₂₃H₂₇N₅O | 389.50 | >98% |
| 253 | 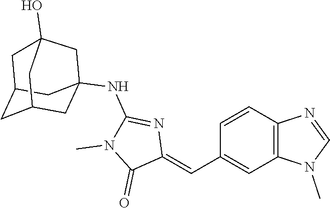 [SMILES: CN1C(=O)/C(=C/c2ccc3ncn(C)c3c2)N=C1NC12CC3CC(CC(O)(C3)C1)C2] | C₂₃H₂₇N₅O₂ | 405.50 | >98% |
| 254 | 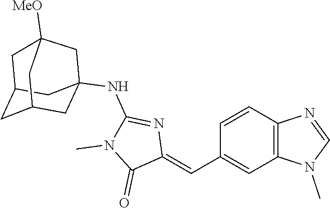 [SMILES: COC12CC3CC(CC(NC4=N/C(=C\c5ccc6ncn(C)c6c5)C(=O)N4C)(C3)C1)C2] | C₂₄H₂₉N₅O₂ | 419.53 | >98% |
| 255 | 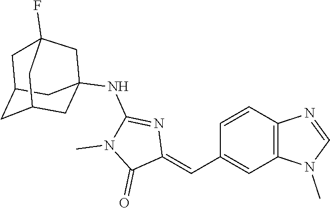 [SMILES: CN1C(=O)/C(=C/c2ccc3ncn(C)c3c2)N=C1NC12CC3CC(CC(F)(C3)C1)C2] | C₂₃H₂₆FN₅O | 407.49 | >98% |
| 256 | 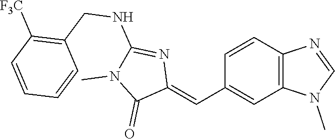 [SMILES: CN1C(=O)/C(=C/c2ccc3ncn(C)c3c2)N=C1NCc1ccccc1C(F)(F)F] | C₂₁H₁₈F₃N₅O | 413.40 | >98% |
| 257 | 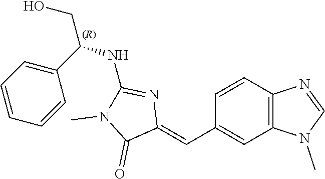 | C₂₁H₂₁N₅O₂ | 375.43 | 98% |
| 258 | 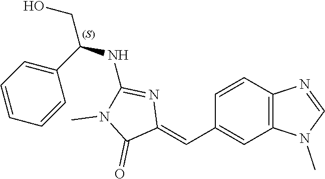 | C₂₁H₂₁N₅O₂ | 375.43 | 98% |
| 259 | 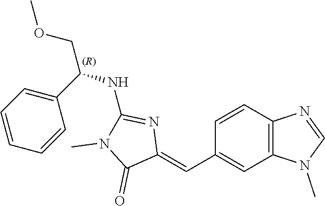 | C₂₂H₂₃N₅O₂ | 389.46 | >98% |
| 260 | 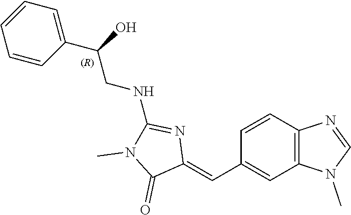 | C₂₁H₂₁N₅O₂ | 375.43 | 98% (¹H NMR) |
| 261 | 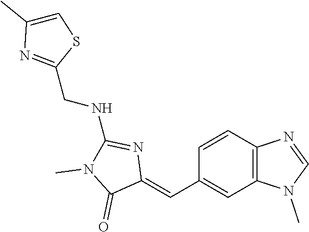 [SMILES: Cc1csc(CNC2=N/C(=C\c3ccc4ncn(C)c4c3)C(=O)N2C)n1] | C₁₈H₁₈N₆OS | 366.44 | >98% |
| 262 | 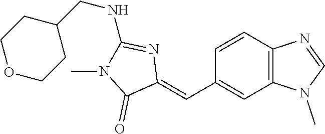 [SMILES: CN1C(=O)/C(=C/c2ccc3ncn(C)c3c2)N=C1NCC1CCOCC1] | C₁₉H₂₃N₅O₂ | 353.43 | >98% |
| 263 | 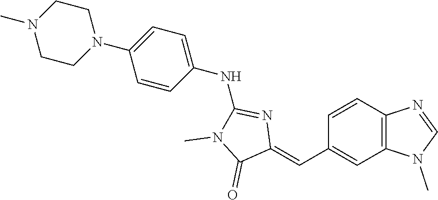 [SMILES: CN1CCN(c2ccc(NC3=N/C(=C\c4ccc5ncn(C)c5c4)C(=O)N3C)cc2)CC1] | C₂₄H₂₇N₇O | 429.53 | >98% (¹H NMR) |
| 264 | 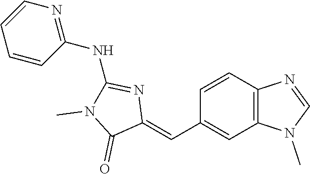 [SMILES: CN1C(=O)/C(=C/c2ccc3ncn(C)c3c2)N=C1Nc1ccccn1] | C₁₈H₁₆N₆0 | 332.37 | >98% |
| 265 | 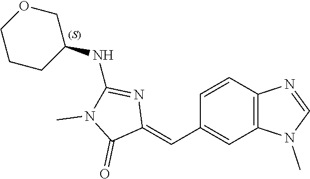 | C₁₈H₂₁N₅O₂ | 339.40 | >98% (¹H NMR) |
| 266 | 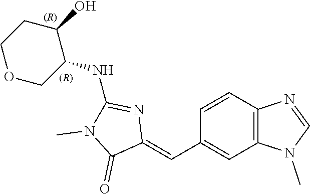 | C₁₈H₂₁N₅O₃ | 355.40 | >95% (¹H NMR) |
| 267 | 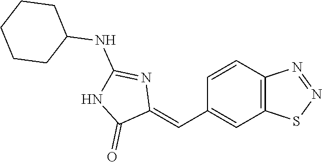 [SMILES: O=C1NC(NC2CCCCC2)=N/C1=C\c1ccc2nnsc2c1] | C₁₆H₁₇N₅OS | 327.41 | 96% (Z) + 2% (E) |
| 268 | 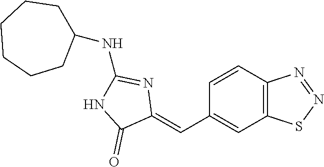 [SMILES: O=C1NC(NC2CCCCCC2)=N/C1=C\c1ccc2nnsc2c1] | C₁₇H₁₉N₅OS | 341.43 | 96% (Z) + 3% (E) |
| 269 | 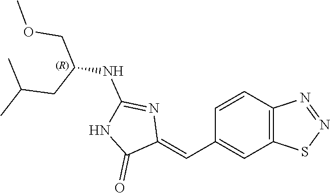 | C₁₇H₂₁N₅O₂S | 359.45 | 89% (Z) + 8% (E) |
| 270 | 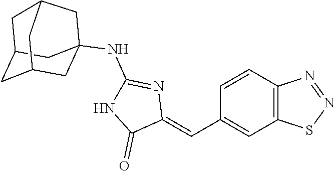 [SMILES: O=C1NC(NC23CC4CC(CC(C4)C2)C3)=N/C1=C\c1ccc2nnsc2c1] | C₂₀H₂₁N₅OS | 379.48 | 96% |
| 271 | 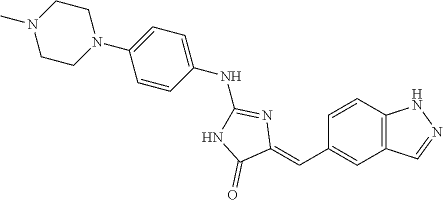 [SMILES: CN1CCN(c2ccc(NC3=N/C(=C\c4ccc5[nH]ncc5c4)C(=O)N3)cc2)CC1] | C₂₂H₂₃N₇O | 401.47 | 82% (Z) + 16% (E) |

Spectroscopic and analytical characterization of compounds (1) to (271)

| Cpd | |
|---|---|
| Nº | Description |
| 1 | Beige solid. ¹H NMR (400 MHz, DMSO-d₆, 343K) of major |
| | tautomer δ_{H} 10.39 (br s, 1H, NH, D₂O exchanged), 8.79 − 8.45 |
| | (m, 2H), 7.92 (d, J = 8.3 Hz, 1H), 7.71 (d, J = 8.3 Hz, 1H), 7.34 |
| | (br s, 1H, NH, D₂O exchanged), 6.41 (s, 1H), 3.73 (s, 1H), 1.94 |
| | (s, 2H), 1.83 − 1.66 (m, 2H), 1.68 − 1.55 (m, 1H), 1.48 − 1.28 (m, |
| | 4H), 1.28 − 1.11 (m, 1H). MS (ESI⁺): [M + H]⁺ 311.2. |
| 2 | Beige solid. ¹H NMR (400 MHz, DMSO-d₆, 343K) of major |
| | tautomer δ_{H} ¹H NMR 10.29 (br s, 1H, NH, D₂O exchanged), 8.63 |
| | (s, 2H), 7.92 (d, J = 8.3 Hz, 1H), 7.71 (d, J = 8.3 Hz, 1H), 7.33 |
| | (br s, 1H, NH, D₂O exchanged), 6.42 (s, 1H), 3.96 (s, 1H), 2.09 − |
| | 1.86 (m, 2H), 1.85 − 1.35 (m, 10H). MS (ESI⁺): [M + H]⁺ 325.2. |
| 3 | Beige solid. ¹H NMR (400 MHz, DMSO-d₆, 323K) of major |
| | tautomer δ_{H} 10.37 (br s, 1H, NH, D₂O exchanged), 8.67 (br s, 2H), |
| | 7.91 (br s, 1H), 7.72 (d, J = 8.2 Hz, 1H), 7.44 (br s, 1H, NH, D₂O |
| | exchanged), 6.42 (s, 1H), 4.02 (br s, 1H), 1.95 − 1.80 (m, 2H), |
| | 1.78 − 1.45 (m, 12H). MS (ESI⁺): [M + H]⁺ 339.3. |
| 4 | Yellow solid. ¹H NMR (400 MHz, DMSO-d₆, 323K) of major |
| | tautomer δ_{H} 10.35 (br s, 1H, NH, D₂O exchanged), 8.67 (s, 1H), |
| | 8.65 (s, 1H), 7.90 (br s, 1H), 7.72 (d, J = 8.2 Hz, 1H), 7.20 (br s, |
| | 1H, NH, D₂O exchanged), 6.42 (s, 1H), 4.77 (br s, 1H, OH, D₂O |
| | exchanged), 4.03 (br s, 1H), 3.55 − 3.45 (m, 2H), 1.78 − 1.59 (m, |
| | 1H), 1.56 − 1.36 (m, 2H), 1.04 − 0.90 (m, 6H). MS (ESI⁺): |
| | [M + H]⁺ 329.3. |
| 5 | Yellow solid. ¹H NMR (400 MHz, DMSO-d₆, 300K) of major |
| | tautomer δ_{H} 10.40 (br s, 1H, NH, D₂O exchanged), 8.67 (s, 1H), |
| | 8.65 (s, 1H), 7.92 (d, J = 8.3 Hz, 1H), 7.71 (d, J = 8.2 Hz, 1H), |
| | 7.31 (br s, 1H, NH, D₂O exchanged), 6.43 (s, 1H), 4.20 (br s, 1H), |
| | 3.52 − 3.36 (m, 2H), 3.32 (s, 3H), 1.77 − 1.63 (m, 1H), 1.59 − 1.46 |
| | (m, 1H), 1.46 − 1.32 (m, 1H), 1.02 − 0.88 (m, 6H). MS (ESI⁺): |
| | [M + H]⁺ 343.3. |
| 6 | Yellow solid. ¹H NMR (400 MHz, DMSO-d₆, 323K) of major |
| | tautomer δ_{H} 10.51 (br s, 1H, NH, D₂O exchanged), 8.68 (s, 1H), |
| | 8.63 (s, 1H), 7.93 (br s, 1H), 7.73 (d, J = 8.3 Hz, 1H), 7.64 (br s, |
| | 1H, NH, D₂O exchanged), 6.45 (s, 1H), 4.16 (br s, 1H), 3.84 − |
| | 3.75 (m, 1H), 3.36 (s, 3H), 2.15 − 2.02 (m, 1H), 1.98 − 1.85 (m, |
| | 1H), 1.80 − 1.50 (m, 4H). MS (ESI⁺): [M + H]⁺ 327.3. |
| 7 | Yellow solid. ¹H NMR (400 MHz, DMSO-d₆, 323K) of major |
| | tautomer δ_{H} 10.51 (br s, 1H, NH, D₂O exchanged), 8.68 (s, 1H), |
| | 8.63 (s, 1H), 7.93 (br s, 1H), 7.73 (d, J = 8.3 Hz, 1H), 7.64 (br s, |
| | 1H, NH, D₂O exchanged), 6.45 (s, 1H), 4.16 (br s, 1H), 3.84 − |
| | 3.75 (m, 1H), 3.36 (s, 3H), 2.15 − 2.02 (m, 1H), 1.98 − 1.85 (m, |
| | 1H), 1.80 − 1.50 (m, 4H). MS (ESI⁺): [M + H]⁺ 327.3. |
| 8 | Beige solid. ¹H NMR (400 MHz, DMSO-d₆, 323K) of major |
| | tautomer δ_{H} 10.35 (br s, 1H, NH, D₂O exchanged), 8.67 (s, 1H), |
| | 8.63 (br s, 1H), 7.89 (br s, 1H), 7.73 (d, J = 8.2 Hz, 1H), 7.46 (br |
| | s, 1H, NH, D₂O exchanged), 6.41 (s, 1H), 4.33 (d, J = 4.1 Hz, 1H, |
| | OH, D₂O exchanged), 3.96 (br s, 1H), 3.70 (br s, 1H), 2.05 − 1.73 |
| | (m, 4H), 1.67 − 1.40 (m, 6H). MS (ESI⁺): [M + H]⁺ 341.3. |
| 9 | Yellow solid. ¹H NMR (400 MHz, DMSO-d₆, 373K) of major |
| | tautomer δ_{H} 10.07 (br s, 1H, NH, D₂O exchanged), 8.80 − 8.38 |
| | (m, 2H), 7.90 (br s, 1H), 7.70 (d, J = 8.2 Hz, 1H), 7.11 (br s, 1H, |
| | NH, D₂O exchanged), 6.43 (s, 1H), 3.98 (br s, 1H), 3.45 − 3.33 |
| | (m, 1H), 3.26 (s, 3H), 2.09 − 1.89 (m, 3H), 1.89 − 1.75 (m, 1H), |
| | 1.73 − 1.48 (m, 6H). MS (ESI⁺): [M + H]⁺ 355.3. |
| 10 | Yellow solid. ¹H NMR (400 MHz, DMSO-d₆, 323K) of major |
| | tautomer δ_{H} 10.48 (br s, 1H, NH, D₂O exchanged), 8.67 (br s, 2H), |
| | 7.92 (br s, 1H), 7.71 (d, J = 8.2 Hz, 1H), 7.43 (br s, 1H, NH, D₂O |
| | exchanged), 6.43 (s, 1H), 3.93 (br s, 1H), 3.52 − 3.40 (m, 1H), |
| | 3.26 (s, 3H), 2.40 − 2.20 (m, 1H), 2.00 − 1.83 (m, 2H), 1.77 − 1.45 |
| | (m, 7H). MS (ESI⁺): [M + H]⁺ 355.2. |
| 11 | Beige solid. ¹H NMR (400 MHz, DMSO-d₆, 323K) of major |
| | tautomer δ_{H} 9.84 (br s, 1H, NH, D₂O exchanged), 8.75 (s, 1H), |
| | 8.67 (s, 1H), 7.87 (d, J = 8.4 Hz, 1H), 7.72 (d, J = 8.3 Hz, 1H), |
| | 6.88 (br s, 1H, NH, D₂O exchanged), 6.43 (s, 1H), 2.23 − 2.00 (m, |
| | 9H), 1.80 − 1.60 (m, 6H). MS (ESI⁺): [M + H]⁺ 363.3. |
| 12 | Beige solid. ¹H NMR (400 MHz, DMSO-d₆, 323K) of major |
| | tautomer δ_{H} 9.85 (br s, 1H, NH, D₂O exchanged), 8.72 (s, 1H), |
| | 8.67 (s, 1H), 7.87 (d, J = 8.5 Hz, 1H), 7.72 (d, J = 8.2 Hz, 1H), |
| | 6.96 (br s, 1H, NH, D₂O exchanged), 6.43 (s, 1H), 4.49 (s, 1H, |
| | OH, D₂O exchanged), 2.25 (br s, 2H), 2.15 − 1.87 (m, 6H), 1.72 |
| | 1.45 (m, 6H). MS (ESI⁺): [M + H]⁺ 379.2. |
| 13 | Beige solid. ¹H NMR (400 MHz, DMSO-d₆, 323K) of major |
| | tautomer δ_{H} 9.89 (br s, 1H, NH, D₂O exchanged), 8.75 (s, 1H), |
| | 8.67 (s, 1H), 7.88 (d, J = 8.4 Hz, 1H), 7.72 (d, J = 8.2 Hz, 1H), |
| | 7.05 (br s, 1H, NH, D₂O exchanged), 6.45 (s, 1H), 3.19 (s, 3H), |
| | 2.31 (br s, 2H), 2.21 (br s, 2H), 2.18 − 1.95 (m, 4H), 1.82 − 1.50 |
| | (m, 6H). MS (ESI⁺): [M + H]⁺ 393.3. |
| 14 | Beige solid. ¹H NMR (400 MHz, DMSO-d₆, 323K) of major |
| | tautomer δ_{H} 10.78 (br s, 1H, NH, D₂O exchanged), 8.67 (br s, 1H), |
| | 8.57 (br s, 1H), 8.04 (br s, 1H, NH, D₂O exchanged), 7.87 (br s, |
| | 1H), 7.80 − 7.60 (m, 4H), 7.55 − 7.42 (m,, 1H), 6.47(s, 1H), 4.82 |
| | (s, 2H). MS (ESI⁺): [M + H]⁺ 387.2. |
| 15 | Beige solid. ¹H NMR (400 MHz, DMSO-d₆, 323K) of major |
| | tautomer δ_{H} 10.69 (br s, 1H, NH, D₂O exchanged), 8.67 (s, 1H), |
| | 8.58 (br s, 1H), 7.96 (br s, 1H + NH, D₂O exchanged), 7.73 (d, J = |
| | 8.3 Hz, 1H), 7.35 − 7.15 (m, 4H), 6.50 (s, 1H), 5.45 (br s, 1H, |
| | OH, D₂O exchanged), 5.25 (br s, 1H), 4.45 (app q, J = 7.1 Hz, |
| | 1H), 3.28 − 3.16 (m, 1H), 2.81 (dd, J = 15.6, 7.5 Hz, 1H). MS |
| | (ESI⁺): [M + H]⁺ 361.2. |
| 16 | Beige solid. ¹H NMR (400 MHz, DMSO-d₆, 323K) of major |
| | tautomer δ_{H} 10.51 (br s, 1H, NH, D₂O exchanged), 8.68 (s, 1H), |
| | 8.57 (br s, 1H), 8.05 − 7.75 (br s, 1H, NH, D₂O exchanged), 7.91 |
| | (d, J = 8.4 Hz, 1H), 7.71 (d, J = 8.4 Hz, 1H), 7.50 − 7.42 (m, 2H), |
| | 7.40 − 7.32 (m, 2H), 7.30 − 7.22 (m, 1H), 6.42 (s, 1H), 5.16 − 4.94 |
| | (m, 1H + OH, D₂O exchanged), 3.84 − 3.67 (m, 2H). MS (ESI⁺): |
| | [M + H]⁺ 349.2. |
| 17 | Beige solid. ¹H NMR (400 MHz, DMSO-d₆, 323K) of major |
| | tautomer δ_{H} 10.51 (br s, 1H, NH, D₂O exchanged), 8.68 (s, 1H), |
| | 8.57 (br s, 1H), 8.05 − 7.75 (br s, 1H, NH, D₂O exchanged), 7.91 |
| | (d, J = 8.4 Hz, 1H), 7.71 (d, J = 8.4 Hz, 1H), 7.50 − 7.42 (m, 2H), |
| | 7.40 − 7.32 (m, 2H), 7.30 − 7.22 (m, 1H), 6.42 (s, 1H), 5.16 − 4.94 |
| | (m, 1H + OH, D₂O exchanged), 3.84 − 3.67 (m, 2H). MS (ESI⁺): |
| | [M + H]⁺ 349.2. |
| 18 | Yellow solid. ¹H NMR (400 MHz, DMSO-d₆, 373K) of major |
| | tautomer δ_{H} 10.18 (br s, 1H, NH, D₂O exchanged), 8.59 (s, 1H), |
| | 8.53 (br s, 1H), 7.89 (br s, 1H), 7.75 − 7.52 (m, 1H + NH, D₂O |
| | exchanged), 7.51 − 1.42 (m, 2H), 7.41 − 7.33 (m, 2H), 1.32 − 7.23 |
| | (m, 1H), 6.45 (s, 1H), 5.27 − 5.17 (m, 1H), 3.80 (dd, J = 10.2 and |
| | 7.0 Hz, 1H), 3.72 (dd, J = 10.2 and 5.2 Hz, 1H), 3.36 (s, 3H). MS |
| | (ESI⁺): [M + H]⁺ 363.2. |
| 19 | Beige solid. ¹H NMR (400 MHz, DMSO-d₆, 323K) of major |
| | tautomer δ_{H} 10.87 (br s, 1H, NH, D₂O exchanged), 8.69 (br s, 1H), |
| | 8.62 (br s, 1H), 8.20 (br s, 1H, NH, D₂O exchanged), 7.96 (d, J = |
| | 8.4 Hz, 1H), 7.73 (d, J = 8.2 Hz, 1H), 7.16 (s, 1H), 6.51 (s, 1H), |
| | 4.84 (s, 2H), 2.37 (s, 3H). MS (ESI⁺): [M + H]⁺ 340.2. |
| 20 | Beige solid. ¹H NMR (400 MHz, DMSO-d₆, 323K) of major |
| | tautomer δ_{H} 10.64 (br s, 1H, NH, D₂O exchanged), 8.67 (br s, 1H), |
| | 8.62 (br s, 1H), 7.94 (br s, 1H), 7.73 (d, J = 8.2 Hz, 1H), 7.55 (br |
| | s, 1H, NH, D₂O exchanged), 6.42 (s, 1H), 3.92 − 3.83 (m, 2H), |
| | 3.36 − 3.24 (m, 4H), 1.88 (br s, 1H), 1.70 − 1.57 (m, 2H), 1.40 − |
| | 1.16 (m, 2H). MS (ESI⁺): [M + H]⁺ 327.2. |
| 21 | Brown solid. ¹H NMR (400 MHz, DMSO-d₆, 323K) of major |
| | tautomer δ_{H} 10.54 (br s, 1H, NH, D₂O exchanged), 9.56 (br s, 1H, |
| | NH, D₂O exchanged), 8.71 (s, 1H), 8.61 (s, 1H), 8.03 (d, J = 8.3 |
| | Hz, 1H), 7.78 (d, J = 8.3 Hz, 1H), 7.63 (d, J = 8.4 Hz, 2H), 6.99 |
| | (d, J = 8.6 Hz, 2H), 6.59 (s, 1H), 3.17 (br s, 4H), 2.55 (br s, 4H), |
| | 2.29 (s, 3H). MS (ESI⁺): [M + H]⁺ 403.3. |
| 22 | Yellow solid. ¹H NMR (400 MHz, DMSO-d₆, 343K) of major |
| | tautomer δ_{H} 10.89 (br s, 2H, NH, D₂O exchanged), 8.71 (s, 1H), |
| | 8.56 (br s, 1H), 8.34 (d, J = 5.1 Hz, 1H), 8.02 (br s, 1H), 7.91 − |
| | 7.74 (m, 2H), 7.52 (br s, 1H), 7.19 − 6.97 (m, 1H), 6.75 (s, 1H). |
| | MS (ESI⁺): [M + H]⁺ 306.2. |
| 23 | Beige solid. ¹H NMR (400 MHz, DMSO-d₆, 323K) of major |
| | tautomer δ_{H} 10.48 (br s, 1H, NH, D₂O exchanged), 8.68 (s, 1H), |
| | 8.59 (s, 1H), 7.94 (s, 1H), 7.74 (d, J = 8.2 Hz, 1H), 7.43 (br s, 1H, |
| | NH, D₂O exchanged), 6.45 (s, 1H), 3.85 (br s, 1H), 3.78 (br d, J = |
| | 11.0 Hz, 1H), 3.51 (dd, J = 11.3, 7.8 Hz, 1H), 2.00 − 1.75 (m, |
| | 2H), 1.70 − 1.58 (m, 1H), 1.56 − 1.44 (m, 1H), 1.22 (s, 3H), 1.21 |
| | (s, 3H). MS (ESI⁺): [M + H]⁺ 341.3. |
| 24 | Orange solid. ¹H NMR (400 MHz, DMSO-d₆, 323K) of major |
| | tautomer δ_{H} 10.33 (br s, 1H, NH, D₂O exchanged), 8.68 (s, 1H), |
| | 8.59 (s, 1H), 7.94 (s, 1H), 7.74 (d, J = 8.2 Hz, 1H), 7.51 (br s, 1H, |
| | NH, D₂O exchanged), 6.46 (s, 1H), 4.05 − 3.81 (m, 2H), 3.79 − |
| | 3.63 (m, 1H), 3.52 − 3.25 (m, 2H), 2.10 − 1.93 (m, 1H), 1.85 − |
| | 1.50 (m, 3H). MS (ESI⁺): [M + H]⁺ 313.3. |
| 25 | Orange solid. ¹H NMR (400 MHz, DMSO-d₆, 323K) of major |
| | tautomer δ_{H} 10.32 (br s, 1H, NH, D₂O exchanged), 8.67 (s, 1H), |
| | 8.62 (s, 1H), 7.95 (d, J = 8.4 Hz, 1H), 7.73 (d, J = 8.2 Hz, 1H), |
| | 7.40 (br s, 1H, NH, D₂O exchanged), 6.44 (s, 1H), 4.12 (br s, 1H), |
| | 3.90 − 3.78 (m, 1H), 3.77 − 3.55 (m, 3H), 2.00 − 1.87 (m, 1H), |
| | 1.86 − 1.68 (m, 4H), 1.67 − 1.50 (m, 1H). MS (ESI⁺): [M + H]⁺ |
| 26 | Pale yellow solid. ¹H NMR (400 MHz, DMSO-d₆, 343K) of major |
| | tautomer δ_{H} 13.08 (br s, 1H, NH, D₂O exchanged), 10.51 (br s, |
| | 1H, NH, D₂O exchanged), 8.41 (br s, 1H), 8.13 (br s, 1H), 8.06 |
| | (s, 1H), 7.49 (d, J = 8.6 Hz, 1H), 7.31 (br s, 1H, NH, D₂O |
| | exchanged), 6.40 (s, 1H), 3.81 − 3.54 (m, 1H), 2.00 − 1.83 (m, |
| | 2H), 1.80 − 1.66 (m, 2H), 1.66 − 1.53 (m, 1H), 1.47 − 1.06 (m, |
| | 5H). MS (ESI⁺): [M + H]⁺ 310.2. |
| 27 | Pale yellow solid. ¹H NMR (400 MHz, DMSO-d₆, 343K) of major |
| | tautomer δ_{H} 12.91 (br s, 1H, NH, D₂O exchanged), 10.03 (br s, |
| | 1H, NH, D₂O exchanged), 8.42 (br s, 1H), 8.13 (br s, 1H), 8.03 |
| | (s, 1H), 7.49 (d, J = 8.6 Hz, 1H), 7.11 (br s, 1H, NH, D₂O |
| | exchanged), 6.42 (s, 1H), 4.03 − 3.84 (m, 1H), 2.12 − 1.91 (m, |
| | 2H), 1.82 − 1.34 (m, 10H). MS (ESI⁺): [M + H]⁺ 324.4. |
| 28 | Pale yellow solid. ¹H NMR (400 MHz, DMSO-d₆, 343K) of major |
| | tautomer δ_{H} 12.91 (br s, 1H, NH, D₂O exchanged), 10.03 (br s, |
| | 1H, NH, D₂O exchanged), 8.47 (br s, 1H), 8.12 (br s, 1H), 8.02 |
| | (s, 1H), 7.48 (d, J = 8.7 Hz, 1H), 7.09 (br s, 1H, NH, D₂O |
| | exchanged), 6.42 (s, 1H), 4.11 − 3.89 (m, 1H), 1.95 − 1.82 (m, |
| | 2H), 1.82 − 1.41 (m, 12H). MS (ESI⁺): [M + H]⁺ 338.4. |
| 29 | Pale yellow solid. ¹H NMR (400 MHz, DMSO-d₆, 343K) of major |
| | tautomer δ_{H} 12.89 (br s, 1H, NH, D₂O exchanged), 10.17 (br s, |
| | 1H, NH, D₂O exchanged), 8.44 (s, 1H), 8.13 (d, J = 8.8 Hz, 1H), |
| | 8.00 (s, 1H), 7.46 (d, J = 8.7 Hz, 1H), 6.82 (br s, 1H, NH, D₂O |
| | exchanged), 6.42 (s, 1H), 4.72 − 4.60 (m, 1H, OH, D₂O |
| | exchanged), 4.00 (br s, 1H), 3.62 − 3.43 (m, 2H), 1.81 − 1.60 (m, |
| | 1H), 1.57 − 1.38 (m, 2H), 1.10 − 0.81 (m, 6H). MS (ESI⁺): |
| | [M + H]⁺ 328.4. |
| 30 | Pale yellow solid. ¹H NMR (400 MHz, DMSO-d₆, 343K) of major |
| | tautomer δ_{H} 12.89 (br s, 1H, NH, D₂O exchanged), 10.20 (br s, |
| | 1H, NH, D₂O exchanged), 8.45 (br s, 1H), 8.12 (br s, 1H), 8.01 |
| | (s, 1H), 7.61 − 7.32 (m, 1H), 7.00 (br s, 1H, NH, D₂O exchanged), |
| | 6.43 (s, 1H), 4.15 (s, 1H), 3.55 − 3.38 (m, 2H), 3.33 (s, 3H), 1.86 − |
| | 1.62 (m, 1H), 1.61 − 1.36 (m, 2H), 1.12 − 0.82 (m, 6H). MS |
| | (ESI⁺): [M + H]⁺ 342.3. |
| 31 | Pale yellow solid. ¹H NMR (400 MHz, DMSO-d₆, 323K) of major |
| | tautomer δ_{H} 12.99 (br s, 1H, NH, D₂O exchanged), 9.72 (br s, 1H, |
| | NH, D₂O exchanged), 8.48 (s, 1H), 8.16 (d, J = 8.7 Hz, 1H), 7.99 |
| | (s, 1H), 7.48 (d, J = 8.8 Hz, 1H), 6.64 (br s, 1H, NH, D₂O |
| | exchanged), 6.42 (s, 1H), 2.32 − 1.99 (m, 9H), 1.88 − 1.59 (m, |
| | 6H). MS (ESI⁺): [M + H]⁺ 362.4. |
| 32 | Pale yellow solid. ¹H NMR (400 MHz, DMSO-d₆, 323K) of major |
| | tautomer δ_{H} 12.99 (br s, 1H, NH, D₂O exchanged), 9.73 (br s, 1H, |
| | NH, D₂O exchanged), 8.49 (s, 1H), 8.15 (d, J = 8.8 Hz, 1H), 8.00 |
| | (s, 1H), 7.48 (d, J = 8.9 Hz, 1H), 6.73 (br s, 1H, NH, D₂O |
| | exchanged), 6.43 (s, 1H), 4.49 (s, 1H, OH, D₂O exchanged), 2.30 − |
| | 2.16 (m, 2H), 2.14 − 1.88 (m, 6H), 1.78 − 1.43 (m, 6H). MS |
| | (ESI⁺): [M + H]⁺ 378.4. |
| 33 | Pale yellow solid. ¹H NMR (400 MHz, DMSO-d₆, 343K) of major |
| | tautomer δ_{H} 12.98 (br s, 1H, NH, D₂O exchanged), 10.42 (br s, |
| | 1H, NH, D₂O exchanged), 8.41 (s, 1H), 8.23 − 8.00 (m, 2H), 7.85 |
| | (br s, 1H, NH, D₂O exchanged), 7.63 − 7.44 (m, 3H), 7.43 − 7.33 |
| | (m, 2H), 7.32 − 7.23 (m, 1H), 6.43 (s, 1H), 5.19 (s, 1H), 3.84 |
| | 3.59 (m, 2H), 3.33 (s, 3H). MS (ESI⁺): [M + H]⁺ 362.4. |
| 34 | Yellow solid. ¹H NMR (400 MHz, DMSO-d₆, 373K) of major |
| | tautomer δ_{H} 10.24 (br s, 1H, NH, D₂O exchanged), 8.43 − 8.19 |
| | (m, 2H), 8.04 (d, J = 9.1 Hz, 1H), 7.51 (d, J = 9.0 Hz, 1H), 7.10 |
| | (br s, 1H, NH, D₂O exchanged), 6.37 (s, 1H), 4.15 (s, 3H), 3.71 |
| | (br s, 1H), 1.94 (br s, 2H), 1.84 − 1.68 (m, 2H), 1.66 − 1.52 (m, |
| | 1H), 1.47 − 1.13 (m, 5H). MS (ESI⁺): [M + H]⁺ 324.3. |
| 35 | Pale yellow solid. ¹H NMR (400 MHz, DMSO-d₆, 343K) of major |
| | tautomer δ_{H} 10.15 (br s, 1H, NH, D₂O exchanged), 8.32 (s, 1H), |
| | 8.28 (s, 1H), 8.16 − 7.96 (m, 1H), 7.67 − 7.45 (m, 1H), 7.09 (br s, |
| | 1H, NH, D₂O exchanged), 6.37 (s, 1H), 4.16 (s, 3H), 3.94 (s, 1H), |
| | 1.96 (q, J = 6.9, 5.0 Hz, 2H), 1.82 − 1.39 (m, 10H). MS (ESI⁺): |
| | [M + H]⁺ 338.4. |
| 36 | Pale yellow solid. ¹H NMR (400 MHz, DMSO-d₆, 343K) of major |
| | tautomer δ_{H} 10.11 (br s, 1H, NH, D₂O exchanged), 8.35 (s, 1H), |
| | 8.26 (s, 1H), 8.13 − 7.99 (m, 1H), 7.67 − 7.44 (m, 1H), 7.07 (br s, |
| | 1H, NH, D₂O exchanged), 6.37 (s, 1H), 4.16 (s, 3H), 4.07 − 3.91 |
| | (m, 1H), 1.96 − 1.81 (m, 2H), 1.81 − 1.42 (m, 12H). MS (ESI⁺): |
| | [M + H]⁺ 352.4. |
| 37 | Pale yellow solid. ¹H NMR (400 MHz, DMSO-d₆, 343K) of major |
| | tautomer δ_{H} 10.11 (br s, 1H, NH, D₂O exchanged), 8.58 − 8.17 |
| | (m, 2H), 8.03 (br s, 1H), 7.52 (d, J = 8.8 Hz, 1H), 6.87 (br s, 1H, |
| | NH, D₂O exchanged), 6.37 (s, 1H), 4.68 (br s, 1H, OH, D₂O |
| | exchanged), 4.16 (s, 3H), 3.98 (br s, 1H), 3.51 (d, J = 5.0 Hz, 2H), |
| | 1.82 − 1.63 (m, 1H), 1.59 − 1.37 (m, 2H), 1.12 − 0.79 (m, 6H). |
| | MS (ESI⁺): [M + H]⁺ 342.4. |
| 38 | Pale yellow solid. ¹H NMR (400 MHz, DMSO-d₆, 343K) of major |
| | tautomer δ_{H} 10.19 (br s, 1H, NH, D₂O exchanged), 8.33 (s, 1H), |
| | 8.25 (s, 1H), 8.04 (d, J = 9.1 Hz, 1H), 7.50 (d, J = 8.9 Hz, 1H), |
| | 6.97 (br s, 1H, NH, D₂O exchanged), 6.38 (s, 1H), 4.15 (s, 4H), |
| | 3.52 − 3.38 (m, 2H), 3.32 (s, 3H), 1.80 − 1.63 (m, 1H), 1.59 − 1.47 |
| | (m, 1H), 1.46 − 1.36 (m, 1H), 0.96 (d, J = 6.5 Hz, 6H). MS (ESI⁺): |
| | [M + H]⁺ 356.4. |
| 39 | Pale yellow solid. ¹H NMR (400 MHz, DMSO-d₆, 323K) of major |
| | tautomer δ_{H} 9.70 (br s, 1H, NH, D₂O exchanged), 8.38 (s, 1H), |
| | 8.28 (s, 1H), 8.04 (d, J = 9.1 Hz, 1H), 7.51 (d, J = 9.0 Hz, 1H), |
| | 6.63 (br s, 1H, NH, D₂O exchanged), 6.37 (s, 1H), 4.15 (s, 3H), |
| | 2.33 − 1.96 (m, 9H), 1.71 (d, J = 23.1 Hz, 6H). MS (ESI⁺): |
| | [M + H]⁺ 376.4. |
| 40 | Pale yellow solid. ¹H NMR (400 MHz, DMSO-d₆, 343K) of major |
| | tautomer δ_{H} 9.63 (br s, 1H, NH, D₂O exchanged), 8.37 (s, 1H), |
| | 8.24 (s, 1H), 8.04 (d, J = 9.1 Hz, 1H), 7.51 (d, J = 9.1 Hz, 1H), |
| | 6.58 (br s, 1H, NH, D₂O exchanged), 6.38 (s, 1H), 4.37 (s, 1H, |
| | OH, D₂O exchanged), 4.16 (s, 3H), 2.32 − 2.17 (m, 2H), 2.15 − |
| | 1.86 (m, 6H), 1.76 − 1.42 (m, 6H). MS (ESI⁺): [M + H]⁺ 392.4. |
| 41 | Yellow solid. ¹H NMR (400 MHz, DMSO-d₆, 300K) of major |
| | tautomer δ_{H} 9.96 (br s, 1H, NH, D₂O exchanged), 8.37 (s, 1H), |
| | 8.31 (s, 1H), 8.04 (d, J = 9.1 Hz, 1H), 7.52 (d, J = 9.1 Hz, 1H), |
| | 7.07 (br s, 1H, NH, D₂O exchanged), 6.39 (s, 1H), 4.15 (s, 3H), |
| | 2.46 − 2.27 (m, 4H), 2.27 − 1.97 (m, 4H), 1.96 − 1.77 (m, 4H), |
| | 1.67 − 1.46 (m, 2H). MS (ESI⁺): [M + H]⁺ 394.4. |
| 42 | Yellow solid. ¹H NMR (400 MHz, DMSO-d₆, 343K) of major |
| | tautomer δ_{H} 10.27 (br s, 1H, NH, D₂O exchanged), 8.31 − 8.24 |
| | (m, 2H), 8.00 (d, J = 9.0 Hz, 1H), 7.68 (br s, 1H, NH, D₂O |
| | exchanged), 7.56 − 7.45 (m, 3H), 7.45 − 7.34 (m, 2H), 7.34 − 7.23 |
| | (m, 1H), 6.39 (s, 1H), 5.27 − 5.11 (m, 1H), 4.16 (s, 3H), 3.83 − |
| | 3.61 (m, 2H), 3.34 (s, 3H). MS (ESI⁺): [M + H]⁺ 376.3. |
| 43 | Colorless solid. ¹H NMR (400 MHz, DMSO-d₆, 373K) of major |
| | tautomer δ_{H} 10.19 (br s, 1H, NH, D₂O exchanged), 8.37 (br s, 1H), |
| | 8.18 (br s, 1H), 8.01 (s, 1H), 7.58 (d, J = 8.7 Hz, 1H), 7.13 (br s, |
| | 1H, NH, D₂O exchanged), 6.42 (s, 1H), 4.04 (s, 3H), 3.72 (br s, |
| | 1H), 2.03 − 1.85 (m, 2H), 1.82 − 1.68 (m, 2H), 1.66 − 1.56 (m, |
| | 1H), 1.48 − 1.29 (m, 4H), 1.29 − 1.15 (m, 1H). MS (ESI⁺): |
| | [M + H]⁺ 324.3. |
| 44 | Offwhite solid. ¹H NMR (400 MHz, DMSO-d₆, 323K) of major |
| | tautomer δ_{H} 10.30 (br s, 1H, NH, D₂O exchanged), 8.40 (br s, 1H), |
| | 8.23 (d, J = 8.9 Hz, 1H), 8.01 (br s, 1H), 7.59 (d, J = 8.8 Hz, 1H), |
| | 7.26 (br s, 1H, NH, D₂O exchanged), 6.42 (br s, 1H), 4.04 (s, 3H), |
| | 3.93 (br s, 1H), 2.05 − 1.95 (m, 2H), 1.75 − 1.40 (m, 10H). MS |
| | (ESI⁺): [M + H]⁺ 338.3. |
| 45 | Offwhite solid. ¹H NMR (400 MHz, DMSO-d₆, 323K) of major |
| | tautomer δ_{H} 10.25 (br s, 1H, NH, D₂O exchanged), 8.43 (br s, 1H), |
| | 8.22 (d, J = 8.8 Hz, 1H), 8.03 (br s, 1H), 7.58 (d, J = 8.7 Hz, 1H), |
| | 7.25 (br s, 1H, NH, D₂O exchanged), 6.42 (br s, 1H), 4.04 (s, 3H), |
| | 3.99 (br s, 1H), 1.95 − 1.81 (m, 2H), 1.79 − 1.40 (m, 12H). MS |
| | (ESI⁺): [M + H]⁺ 352.3. |
| 46 | Beige solid. ¹H NMR (400 MHz, DMSO-d₆, 323K) of major |
| | tautomer δ_{H} 10.27 (br s, 1H, NH, D₂O exchanged), 8.42 (br s, 1H), |
| | 8.20 (d, J = 8.8 Hz, 1H), 7.98 (br s, 1H), 7.56 (d, J = 8.8 Hz, 1H), |
| | 6.99 (br s, 1H, NH, D₂O exchanged), 6.42 (br s, 1H), 4.76 (br s, |
| | 1H, OH, D₂O exchanged), 4.04 (s, 3H), 4.04 − 3.90 (m, 1H), 3.55 − |
| | 3.45 (br s, 2H), 1.80 − 1.65 (m, 1H), 1.58 − 1.35 (m, 2H), 1.05 − |
| | 0.85 (m, 6H). MS (ESI⁺): [M + H]⁺ 342.3. |
| 47 | Beige solid. ¹H NMR (400 MHz, DMSO-d₆, 323K) of major |
| | tautomer δ_{H} 10.33 (br s, 1H, NH, D₂O exchanged), 8.42 (br s, 1H), |
| | 8.21 (d, J = 8.8 Hz, 1H), 7.99 (br s, 1H), 7.57 (d, J = 8.8 Hz, 1H), |
| | 7.25 (br s, 1H, NH, D₂O exchanged), 6.43 (br s, 1H), 4.16 (br s, |
| | 1H), 4.04 (s, 3H), 3.50 − 3.35 (m, 2H), 3.32 (s, 3H), 1.80 − 1.61 |
| | (m, 1H), 1.58 − 1.45 (m, 1H), 1.45 − 1.32 (m, 1H), 1.02 − 0.85 |
| | (m, 6H). MS (ESI⁺): [M + H]⁺ 356.3. |
| 48 | Beige solid. ¹H NMR (400 MHz, DMSO-d₆, 323K) of major |
| | tautomer δ_{H} 10.49 (br s, 1H, NH, D₂O exchanged), 8.41 (br s, 1H), |
| | 8.21 (d, J = 8.9 Hz, 1H), 8.00 (br s, 1H), 7.58 (d, J = 8.8 Hz, 1H), |
| | 7.36 (br s, 1H, NH, D₂O exchanged), 6.47 (br s, 1H), 4.65 − 4.50 |
| | (m, 1H), 4.50 − 4.37 (m, 1H), 4.35 − 4.10 (m, 1H), 4.04 (s, 3H), |
| | 1.80 − 1.65 (m, 1H), 1.63 − 1.50 (m, 1H), 1.45 − 1.33 (m, 1H), |
| | 1.02 − 0.91 (m, 6H). MS (ESI⁺): [M + H]⁺ 344.2. |
| 49 | Beige solid. ¹H NMR (400 MHz, DMSO-d₆, 323K) of major |
| | tautomer δ_{H} 10.49 (br s, 1H, NH, D₂O exchanged), 8.41 (br s, 1H), |
| | 8.21 (d, J = 8.9 Hz, 1H), 8.00 (br s, 1H), 7.58 (d, J = 8.8 Hz, 1H), |
| | 7.36 (br s, 1H, NH, D₂O exchanged), 6.47 (br s, 1H), 4.65 − 4.50 |
| | (m, 1H), 4.50 − 4.37 (m, 1H), 4.35 − 4.10 (m, 1H), 4.04 (s, 3H), |
| | 1.80 − 1.65 (m, 1H), 1.63 − 1.50 (m, 1H), 1.45 − 1.33 (m, 1H), |
| | 1.02 − 0.91 (m, 6H). MS (ESI⁺): [M + H]⁺ 344.3. |
| 50 | Yellow solid. ¹H NMR (400 MHz, DMSO-d₆, 323K) of major |
| | tautomer δ_{H} 10.35 (br s, 1H, NH, D₂O exchanged), 8.44 (s, 1H), |
| | 8.20 (d, J = 8.8 Hz, 1H), 8.00 (s, 1H), 7.57 (d, J = 8.8 Hz, 1H), |
| | 7.41 (br s, 1H, NH, D₂O exchanged), 6.45 (br s, 1H), 4.14 (br s, |
| | 1H), 4.04 (s, 3H), 3.83 − 3.70 (m, 1H), 3.38 (s, 3H), 2.15 − 2.00 |
| | (m, 1H), 1.99 − 1.84 (m, 1H), 1.80 − 1.50 (m, 4H). MS (ESI⁺): |
| | [M + H]⁺ 340.3. |
| 51 | Yellow solid. ¹H NMR (400 MHz, DMSO-d₆, 323K) of major |
| | tautomer δ_{H} 10.35 (br s, 1H, NH, D₂O exchanged), 8.44 (s, 1H), |
| | 8.20 (d, J = 8.8 Hz, 1H), 8.00 (s, 1H), 7.57 (d, J = 8.8 Hz, 1H), |
| | 7.41 (br s, 1H, NH, D₂O exchanged), 6.45 (br s, 1H), 4.14 (br s, |
| | 1H), 4.04 (s, 3H), 3.83 − 3.70 (m, 1H), 3.38 (s, 3H), 2.15 − 2.00 |
| | (m, 1H), 1.99 − 1.84 (m, 1H), 1.80 − 1.50 (m, 4H). MS (ESI⁺): |
| | [M + H]⁺ 340.3. |
| 52 | Yellow solid. ¹H NMR (400 MHz, DMSO-d₆, 323K) of major |
| | tautomer δ_{H} 10.34 (br s, 1H, NH, D₂O exchanged), 8.41 (s, 1H), |
| | 8.22 (d, J = 8.8 Hz, 1H), 8.01 (s, 1H), 7.58 (d, J = 8.8 Hz, 1H), |
| | 7.25 (br s, 1H, NH, D₂O exchanged), 6.42 (s, 1H), 4.33 (d, J = 4.0 |
| | Hz, 1H, OH, D₂O exchanged), 4.04 (s, 3H), 3.93 (br s, 1H), 3.70 |
| | (br s, 1H), 2.07 − 1.72 (m, 4H), 1.66 − 1.40 (m, 6H). MS (ESI⁺): |
| | [M + H]⁺ 354.3. |
| 53 | Beige solid. ¹H NMR (400 MHz, DMSO-d₆, 323K) of major |
| | tautomer δ_{H} 10.35 (br s, 1H, NH, D₂O exchanged), 8.39 (s, 1H), |
| | 8.21 (d, J = 8.9 Hz, 1H), 8.00 (s, 1H), 7.58 (d, J = 8.8 Hz, 1H), |
| | 7.27 (br s, 1H, NH, D₂O exchanged), 6.42 (s, 1H), 4.03 (s, 3H), |
| | 3.92 (s, 1H), 3.39 − 3.34 (m, 1H), 3.24 (s, 3H), 2.06 − 1.87 (m, |
| | 3H), 1.85 − 1.72 (m, 1H), 1.71 − 1.43 (m, 6H). MS (ESI⁺): |
| | [M + H]⁺ 368.3. |
| 54 | Beige solid. ¹H NMR (400 MHz, DMSO-d₆, 323K) of major |
| | tautomer δ_{H} 10.38 (br s, 1H, NH, D₂O exchanged), 8.43 (br s, 1H), |
| | 8.23 (d, J = 8.9 Hz, 1H), 8.01 (s, 1H), 7.58 (d, J = 8.8 Hz, 1H), |
| | 7.24 (br s, 1H, NH, D₂O exchanged), 6.44 (s, 1H), 4.04 (s, 3H), |
| | 3.92 (br s, 1H), 3.55 − 3.36 (m, 1H), 3.26 (s, 3H), 2.40 − 2.20 (m, |
| | 1H), 2.00 − 1.85 (m, 2H), 1.77 − 1.40 (m, 7H). MS (ESI⁺): |
| | [M + H]⁺ 368.3. |
| 55 | Yellow solid. ¹H NMR (400 MHz, DMSO-d₆, 323K) of major |
| | tautomer δ_{H} 9.72 (br s, 1H, NH, D₂O exchanged), 8.38 (s, 1H), |
| | 8.30 (d, J = 8.8 Hz, 1H), 7.97 (s, 1H), 7.60 (d, J = 8.8 Hz, 1H), |
| | 6.65 (br s, 1H, NH, D₂O exchanged), 6.43 (s, 1H), 4.04 (s, 3H), |
| | 2.26 − 2.00 (m, 9H), 1.81 − 1.61 (m, 6H). MS (ESI⁺): [M + H]⁺ |
| | 376.3. |
| 56 | Pale yellow solid. ¹H NMR (400 MHz, DMSO-d₆, 323K) of |
| | major tautomer δ_{H} 9.73 (br s, 1H, NH, D₂O exchanged), 8.41 (br |
| | s, 1H), 8.28 (d, J = 9.0 Hz, 1H), 7.98 (s, 1H), 7.60 (d, J = 8.8 Hz, |
| | 1H), 6.73 (br s, 1H, NH, D₂O exchanged), 6.44 (s, 1H), 4.49 (s, |
| | 1H, OH, D₂O exchanged), 4.05 (s, 3H), 2.26 (br s, 2H), 2.15 |
| | 1.90 (m, 6H), 1.75 − 1.40 (m, 6H). MS (ESI⁺): [M + H]⁺ 392.2. |
| 57 | Yellow solid. ¹H NMR (400 MHz, DMSO-d₆, 323K) of major |
| | tautomer δ_{H} 9.75 (br s, 1H, NH, D₂O exchanged), 8.41 (br s, 1H), |
| | 8.27 (d, J = 8.8 Hz, 1H), 7.96 (s, 1H), 7.59 (d, J = 8.8 Hz, 1H), |
| | 6.83 (br s, 1H, NH, D₂O exchanged), 6.45 (s, 1H), 4.05 (s, 3H), |
| | 3.19 (s, 3H), 2.32 (br s, 2H), 2.22 − 1.90 (m, 6H), 1.80 − 1.45 (m, |
| | 6H). MS (ESI⁺): [M + H]⁺ 406.4. |
| 58 | Beige solid. ¹H NMR (400 MHz, DMSO-d₆, 323K) of major |
| | tautomer δ_{H} 9.84 (br s, 1H, NH, D₂O exchanged), 8.37 (br s, 1H), |
| | 8.27 (d, J = 8.9 Hz, 1H), 7.98 (s, 1H), 7. 61 (d, J = 8.8 Hz, 1H), |
| | 6.92 (br s, 1H, NH, D₂O exchanged), 6.47 (s, 1H), 4.05 (s, 3H), |
| | 2.45 − 2.25 (m, 4H), 2.20 − 2.10 (m, 2H), 2.10 − 2.00 (m, 2H), |
| | 2.00 − 1.80 (m, 4H), 1.70 − 1.50 (m, 2H). MS (ESI⁺): [M + H]⁺ |
| | 394.2. |
| 59 | Yellow solid. ¹H NMR (400 MHz, DMSO-d₆, 323K) of major |
| | tautomer δ_{H} 10.66 (br s, 1H, NH, D₂O exchanged), 8.39 (br s, 1H), |
| | 8.23 (d, J = 8.6 Hz, 1H), 7.95 (s, 1H), 7.85 (br s, 1H, NH, D₂O |
| | exchanged), 7.77 (d, J = 7.8 Hz, 1H), 7.74 − 7.62 (m, 2H), 7.56 − |
| | 7.42 (m, 2H), 6.48 (s, 1H), 4.81 (s, 2H), 4.03 (s, 3H). MS (ESI⁺): |
| | [M + H]⁺ 400.2. |
| 60 | Beige solid. ¹H NMR (400 MHz, DMSO-d₆, 323K) of major |
| | tautomer δ_{H} 10.53 (br s, 1H, NH, D₂O exchanged), 8.36 (br s, 1H), |
| | 8.21 (br s, 1H), 8.01 (s, 1H), 7.79 (br s, 1H, NH, D₂O exchanged), |
| | 7.58 (d, J = 8.7 Hz, 1H), 7.35 − 7.15 (m, 4H), 6.50 (s, 1H), 5.48 |
| | (br s, 1H, OH, D₂O exchanged), 5.21 (br s, 1H), 4.45 (app q, J = |
| | 7.1 Hz, 1H), 4.03 (s, 3H), 3.27 − 3.18 (m, 1H), 2.79 (dd, J = 15.6, |
| | 7.4 Hz, 1H). MS (ESI⁺): [M + H]⁺ 374.3. |
| 61 | Beige solid. ¹H NMR (400 MHz, DMSO-d₆, 323K) of major |
| | tautomer δ_{H} 10.39 (br s, 1H, NH, D₂O exchanged), 8.38 (br s, 1H), |
| | 8.13 (br s, 1H), 8.04 (s, 1H), 7.72 (br s, 1H, NH, D₂O exchanged), |
| | 7.58 (d, J = 8.6 Hz, 1H), 7.50 − 7.40 (m, 2H), 7.37 (t, J = 7.5 Hz, |
| | 2H), 7.30 − 7.20 (m, 1H), 6.43 (s, 1H), 5.02 (br s, 1H + OH, D₂O |
| | exchanged), 4.04 (s, 3H), 3.76 (d, J = 6.0 Hz, 2H). MS (ESI⁺): |
| | [M + H]⁺ 362.2. |
| 62 | Beige solid. ¹H NMR (400 MHz, DMSO-d₆, 323K) of major |
| | tautomer δ_{H} 10.39 (br s, 1H, NH, D₂O exchanged), 8.38 (br s, 1H), |
| | 8.13 (br s, 1H), 8.04 (s, 1H), 7.72 (br s, 1H, NH, D₂O exchanged), |
| | 7.58 (d, J = 8.6 Hz, 1H), 7.50 − 7.40 (m, 2H), 7.37 (t, J = 7.5 Hz, |
| | 2H), 7.30 − 7.20 (m, 1H), 6.43 (s, 1H), 5.02 (br s, 1H + OH, D₂O |
| | exchanged), 4.04 (s, 3H), 3.76 (d, J = 6.0 Hz, 2H). MS (ESI⁺): |
| | [M + H]⁺ 362.2. |
| 63 | Yellow solid. ¹H NMR (400 MHz, DMSO-d₆, 323K) of major |
| | tautomer δ_{H} 10.31 (br s, 1H, NH, D₂O exchanged), 8.39 (br s, 1H), |
| | 8.15 (d, J = 8.8 Hz, 1H), 8.04 (s, 1H), 7.87 (br s, 1H, NH, D₂O |
| | exchanged), 7.57 (d, J = 8.8 Hz, 1H), 7.53 − 7.42 (m, 2H), 7.38 |
| | (t, J = 7.5 Hz, 2H), 7.32 − 7.22 (m, 1H), 6.44 (s, 1H), 5.20 (br s, |
| | 1H), 4.04 (s, 3H), 3.80 − 3.71 (m, 1H), 3.69 − 3.60 (m, 1H), 3.34 |
| | (s, 3H). MS (ESI⁺): [M + H]⁺ 376.3. |
| 64 | Yellow solid. ¹H NMR (400 MHz, DMSO-d₆, 373K) of major |
| | tautomer δ_{H} 10.08 (br s, 1H, NH, D₂O exchanged), 8.40 (s, 1H), |
| | 8.18 (br s, 1H), 8.00 (s, 1H), 7.57 (d, J = 8.6 Hz, 1H), 7.51 − 7.42 |
| | (m, 2H), 7.38 (t, J = 7.6 Hz, 2H), 7.32 − 7.24 (m, 1H), 6.89 (br s, |
| | 1H, NH, D₂O exchanged), 6.48 (br s, 1H), 5.42 (br s, 1H, OH, |
| | D₂O exchanged), 4.92 (s, 1H), 4.05 (s, 3H), 3.71 (dd, J = 13.4, |
| | 4.1 Hz, 1H), 3.51 (dd, J= 13.4, 7.6 Hz, 1H). MS (ESI⁺): [M + H]⁺ |
| | 362.2. |
| 65 | Yellow foam. ¹H NMR (400 MHz, D₂O, 323K) of major tautomer |
| | 8H 7.89 (br s, 1H), 7.86 − 7.70 (m, 5H), 7.54 (br s, 1H), 7.34 − |
| | 7.26 (m, 2H), 6.51 (s, 1H), 5.48 − 5.40 (m, 1H), 3.97 (s, 3H), 3.88 − |
| | 3.72 (m, 2H), MS (ESI⁺): [M + H]⁺ 361.3 (+2HCl). |
| 66 | Yellow foam. ¹H NMR (400 MHz, D₂O, 323K) of major tautomer |
| | 8H 7.89 (br s, 1H), 7.86 − 7.70 (m, 5H), 7.54 (br s, 1H), 7.34 − |
| | 7.26 (m, 2H), 6.51 (s, 1H), 5.48 − 5.40 (m, 1H), 3.97 (s, 3H), 3.88 |
| | 3.72 (m, 2H), MS (ESI⁺): [M + H]⁺ 361.3 (+2HCl). |
| 67 | Yellow solid. ¹H NMR (400 MHz, DMSO-d₆, 323K) of major |
| | tautomer δ_{H} 10.74 (br s, 1H, NH, D₂O exchanged), 8.42 (br s, 1H), |
| | 8.22 (d, J = 8.8 Hz, 1H), 8.03 (br s, 1H + NH, D₂O exchanged), |
| | 8.02 (br s, 1H), 7.58 (d, J = 8.8 Hz, 1H), 7.16 (s, 1H), 6.52 (s, |
| | 1H), 4.84 (s, 2H), 4.04 (s, 3H), 2.37 (s, 3H). MS (ESI⁺): [M + H]⁺ |
| | 353.2. |
| 68 | Beige solid. ¹H NMR (400 MHz, DMSO-d₆, 323K) of major |
| | tautomer δ_{H} 10.53 (br s, 1H, NH, D₂O exchanged), 8.37 (br s, 1H), |
| | 8.19 (br s, 1H), 8.03 (s, 1H), 7.60 (d, J = 8.8 Hz, 1H), 7.40 (br s, |
| | 1H, NH, D₂O exchanged), 6.43 (s, 1H), 4.04 (s, 3H), 3.93 − 3.80 |
| | (m, 2H), 3.40 − 3.10 (m, 4H), 1.86 (br s, 1H), 1.71 − 1.59 (m, 2H), |
| | 1.34 − 1.19 (m, 2H). MS (ESI⁺): [M + H]⁺ 340.2. |
| 69 | Dark yellow solid. ¹H NMR (400 MHz, DMSO-d₆, 323K) of |
| | major tautomer δ_{H} 10.57 (br s, 1H, NH, D₂O exchanged), 9.56 (br |
| | s, 1H, NH, D₂O exchanged), 8.47 (s, 1H), 8.28 (d, J = 8.8 Hz, |
| | 1H), 8.14 (s, 1H), 7.76 − 7.55 (m, 3H), 7.02 (d, J = 8.7 Hz, 2H), |
| | 6.60 (s, 1H), 4.06 (s, 3H), 3.17 − 3.10 (m, 4H), 2.50 − 2.46 (m, |
| | 4H), 2.24 (s, 3H). MS (ESI⁺): [M + H]⁺ 416.2. |
| 70 | Yellow solid. ¹H NMR (400 MHz, DMSO-d₆, 373K) of major |
| | tautomer δ_{H} 10.62 (br s, 2H, NH, D₂O exchanged), 8.40 (br s, 1H), |
| | 8.35 (d, J = 5.1 Hz, 1H), 8.08 (s, 1H), 8.07 (br s, 1H), 7.83 (t, J = |
| | 7.9 Hz, 1H), 7.65 (d, J = 8.9 Hz, 1H), 7.54 (br s, 1H), 7.10 − 7.04 |
| | (m, 1H), 6.75 (s, 1H), 4.07 (s, 3H). MS (ESI⁺): [M + H]⁺ 319.2. |
| 71 | Beige solid. ¹H NMR (400 MHz, DMSO-d₆, 323K) of major |
| | tautomer δ_{H} 10.38 (br s, 1H, NH, D₂O exchanged), 8.36 (br s, 1H), |
| | 8.23 (d, J = 8.8 Hz, 1H), 8.03 (s, 1H), 7.60 (d, J = 8.8 Hz, 1H), |
| | 7.23 (br s, 1H, NH, D₂O exchanged), 6.45 (s, 1H), 4.04 (s, 3H), |
| | 3.83 (br s, 1H), 3.76 (dd, J= 11.4, 4.0 Hz, 1H), 3.51 (dd, J = 11.4, |
| | 7.8 Hz, 1H), 1.96 − 1.74 (m, 2H), 1.70 − 1.58 (m, 1H), 1.56 − 1.42 |
| | (m, 1H), 1.23 (s, 3H), 1.21 (s, 3H). MS (ESI⁺): [M + H]⁺ 354.3. |
| 72 | Beige solid. ¹H NMR (400 MHz, DMSO-d₆, 323K) of major |
| | tautomer δ_{H} 10.33 (br s, 1H, NH, D₂O exchanged), 8.36 (br s, 1H), |
| | 8.22 (br s, 1H), 8.04 (s, 1H), 7.61 (d, J = 8.7 Hz, 1H), 7.31 (br s, |
| | 1H, NH, D₂O exchanged), 6.46 (s, 1H), 4.04 (s, 3H), 3.97 − 3.82 |
| | (m, 2H), 3.79 − 3.65 (m, 1H), 3.52 − 3.25 (m, 2H), 2.08 − 1.90 |
| | (m, 1H), 1.80 − 1.50 (m, 3H). MS (ESI⁺): [M + H]⁺ 326.3. |
| 73 | Beige solid. ¹H NMR (400 MHz, DMSO-d₆, 323K) of major |
| | tautomer δ_{H} 10.37 (br s, 1H, NH, D₂O exchanged), 8.37 (br s, 1H), |
| | 8.23 (d, J = 8.8 Hz, 1H), 8.03 (s, 1H), 7.61 (d, J = 8.7 Hz, 1H), |
| | 7.23 (br s, 1H, NH, D₂O exchanged), 6.46 (s, 1H), 5.02 (br s, 1H, |
| | OH, D₂O exchanged), 4.04 (s, 3H), 4.04 − 3.95 (m, 1H), 3.90 − |
| | 3.80 (m, 1H), 3.75 − 3.58 (br s, 2H), 3.50 − 3.33 (m, 1H), 3.30 |
| | 3.15 (m, 1H), 2.00 − 1.87 (m, 1H), 1.62 − 1.45 (m, 1H). MS |
| | (ESI⁺): [M + H]⁺ 342.3. |
| 74 | Beige solid. ¹H NMR (400 MHz, DMSO-d₆, 323K) of major |
| | tautomer δ_{H} 10.21 (br s, 1H, NH, D₂O exchanged), 8.37 (br s, 1H), |
| | 8.22 (br s, 1H), 8.03 (s, 1H), 7.60 (d, J = 8.6 Hz, 1H), 7.20 (br s, |
| | 1H, NH, D₂O exchanged), 6.45 (s, 1H), 4.11 (br s, 1H), 4.04 (s, |
| | 3H), 3.90 − 3.55 (m, 4H). 2.00 − 1.50 (m, 6H). MS (ESI⁺): |
| | [M + H]⁺ 340.2. |
| 75 | Pale yellow solid. ¹H NMR (400 MHz, DMSO-d₆, 343K) of major |
| | tautomer δ_{H} 12.28 (br s, 1H, NH, D₂O exchanged), 10.21 (br s, |
| | 1H, NH, D₂O exchanged), 8.32 (br s, 1H), 8.16 (s, 1H), 7.77 (br |
| | s, 1H), 7.54 (d, J = 8.3 Hz, 1H), 7.08 (br s, 1H, NH, D₂O |
| | exchanged), 6.42 (s, 1H), 3.73 (br s, 1H), 2.09 − 1.83 (m, 2H), |
| | 1.80 − 1.49 (m, 3H), 1.47 − 1.12 (m, 5H). MS (ESI⁺): [M + H]⁺ |
| | 310.2. |
| 76 | Pale yellow solid. ¹H NMR (400 MHz, DMSO-d₆, 343K) of major |
| | tautomer δ_{H} 12.28 (br s, 1H, NH, D₂O exchanged), 10.13 (br s, |
| | 1H, NH, D₂O exchanged), 8.35 (br s, 1H), 8.16 (s, 1H), 7.81 (br |
| | s, 1H), 7.54 (d, J = 8.2 Hz, 1H), 7.07 (br s, 1H, NH, D₂O |
| | exchanged), 6.43 (s, 1H), 3.96 (br s, 1H), 2.15 − 1.81 (m, 2H), |
| | 1.77 − 1.33 (m, 10H). MS (ESI⁺): [M + H]⁺ 324.3. |
| 77 | Pale yellow solid. ¹H NMR (400 MHz, DMSO-d₆, 343K) of major |
| | tautomer δ_{H} 12.34 (br s, 1H, NH, D₂O exchanged), 10.24 (br s, |
| | 1H, NH, D₂O exchanged), 8.59 − 8.26 (m, 1H), 8.17 (s, 1H), 7.88 |
| | (d, J = 8.4 Hz, 1H), 7.70 − 7.45 (m, 1H), 7.19 (br s, 1H, NH, D₂O |
| | exchanged), 6.42 (s, 1H), 3.99 (br s, 1H), 2.03 − 1.34 (m, 14H). |
| | MS (ESI⁺): [M + H]⁺ 338.3. |
| 78 | Pale yellow solid. ¹H NMR (400 MHz, DMSO-d₆, 343K) of major |
| | tautomer δ_{H} 10.81 (br s, 2H, NH, D₂O exchanged), 8.37 − 8.07 |
| | (m, 2H), 7.76 (br s, 1H), 7.54 (d, J = 8.4 Hz, 1H), 6.90 (br s, 1H, |
| | NH, D₂O exchanged), 6.43 (s, 1H), 4.01 (s, 1H), 3.52 (d, J = 5.1 |
| | Hz, 2H), 1.80 − 1.64 (m, 1H), 1.57 − 1.38 (m, 2H), 0.96 (d, J = |
| | 6.6 Hz, 6H). MS (ESI⁺): [M + H]⁺ 328.3. |
| 79 | Pale yellow solid. ¹H NMR (400 MHz, DMSO-d₆, 343K) of major |
| | tautomer δ_{H} 12.28 (br s, 1H, NH, D₂O exchanged), 10.18 (br s, |
| | 1H, NH, D₂O exchanged), 8.58 − 8.26 (m, 1H), 8.14 (s, 1H), 7.88 |
| | (d, J = 8.3 Hz, 1H), 7.54 (s, 1H), 6.95 (br s, 1H, NH, D₂O |
| | exchanged), 6.44 (s, 1H), 4.18 (s, 1H), 3.51 − 3.39 (m, 2H), 3.33 |
| | (s, 3H), 1.72 (s, 1H), 1.59 − 1.35 (m, 2H), 1.01 − 0.85 (m, 6H). |
| | MS (ESI⁺): [M + H]⁺ 342.3. |
| 80 | Pale yellow solid. ¹H NMR (400 MHz, DMSO-d₆, 343K) of major |
| | tautomer δ_{H} 12.30 (br s, 1H, NH, D₂O exchanged), 10.33 (br s, |
| | 1H, NH, D₂O exchanged), 8.35 (br s, 1H), 8.15 (s, 1H), 7.98 |
| | 7.82 (m, 1H), 7.66 − 7.47 (m, 1H), 7.18 (br s, 1H, NH, D₂O |
| | exchanged), 6.47 (s, 1H), 4.66 − 4.51 (m, 1H), 4.51 − 4.38 (m, |
| | 1H), 4.35 − 4.17 (m, 1H), 1.82 − 1.65 (m, 1H), 1.65 − 1.52 (m, |
| | 1H), 1.50 − 1.36 (m, 1H), 1.03 0.87 (m, 6H). MS (ESI⁺): |
| | [M + H]⁺ 330.3. |
| 81 | Pale yellow solid. ¹H NMR (400 MHz, DMSO-d₆, 343K) of major |
| | tautomer δ_{H} 12.30 (br s, 1H, NH, D₂O exchanged), 10.33 (br s, |
| | 1H, NH, D₂O exchanged), 8.35 (br s, 1H), 8.15 (s, 1H), 7.98 |
| | 7.82 (m, 1H), 7.66 − 7.47 (m, 1H), 7.18 (br s, 1H, NH, D₂O |
| | exchanged), 6.47 (s, 1H), 4.66 − 4.51 (m, 1H), 4.51 − 4.38 (m, |
| | 1H), 4.35 − 4.17 (m, 1H), 1.82 − 1.65 (m, 1H), 1.65 − 1.52 (m, |
| | 1H), 1.50 − 1.36 (m, 1H), 1.03 − 0.87 (m, 6H). MS (ESI⁺): |
| | [M + H]⁺ 330.3. |
| 82 | Pale yellow solid. ¹H NMR (400 MHz, DMSO-d₆, 343K) of major |
| | tautomer δ_{H} 12.29 (br s, 1H, NH, D₂O exchanged), 10.22 (br s, |
| | 1H, NH, D₂O exchanged), 8.38 (br s, 1H), 8.15 (s, 1H), 7.98 |
| | 7.72 (m, 1H), 7.52 (br s, 1H), 7.24 (br s, 1H, NH, D₂O |
| | exchanged), 6.46 (s, 1H), 4.16 (s, 1H), 3.78 (s, 1H), 3.36 (s, 3H), |
| | 2.15 − 2.03 (m, 1H), 2.01 − 1.87 (m, 1H), 1.80 − 1.51 (m, 4H). |
| | MS (ESI⁺): [M + H]⁺ 326.3. |
| 83 | Pale yellow solid. ¹H NMR (400 MHz, DMSO-d₆, 343K) of major |
| | tautomer δ_{H} 12.28 (br s, 1H, NH, D₂O exchanged), 9.78 (br s, 1H, |
| | NH, D₂O exchanged), 8.35 (br s, 1H), 8.14 (br s, 1H), 7.93 − 7.88 |
| | (m, 1H), 7.56 − 7.45 (m, 1H), 7.02 (br s, 1H, NH, D₂O |
| | exchanged), 6.44 (s, 1H), 2.67 (s, 1H), 2.44 − 1.90 (m, 8H), 1.79 − |
| | 1.41 (m, 4H). MS (ESI⁺): [M + H]⁺ 348.4. |
| 84 | Pale yellow solid. ¹H NMR (400 MHz, DMSO-d₆, 323K) of major |
| | tautomer δ_{H} 12.40 (br s, 1H, NH, D₂O exchanged), 9.69 (br s, 1H, |
| | NH, D₂O exchanged), 8.40 (br s, 1H), 8.17 (s, 1H), 7.91 − 7.81 |
| | (m, 1H), 7.65 − 7.46 (m, 1H), 6.60 (br s, 1H, NH, D₂O |
| | exchanged), 6.42 (s, 1H), 2.37 − 1.94 (m, 9H), 1.92 − 1.52 (m, |
| | 6H). MS (ESI⁺): [M + H]⁺ 362.3. |
| 85 | Beige solid. ¹H NMR (400 MHz, DMSO-d₆, 323K) of major |
| | tautomer δ_{H} 12.38 (br s, 1H, NH, D₂O exchanged), 9.69 (br s, 1H, |
| | NH, D₂O exchanged), 8.67 − 8.09 (m, 2H), 7.90 (d, J = 8.3 Hz, |
| | 1H), 7.71 − 7.42 (m, 1H), 6.70 (br s, 1H, NH, D₂O exchanged), |
| | 6.43 (s, 1H), 4.46 (br s, 1H, OH, D₂O exchanged), 2.32 − 1.86 (m, |
| | 8H), 1.79 − 1.42 (m, 6H). MS (ESI⁺): [M + H]⁺ 378.3. |
| 86 | Beige solid. ¹H NMR (400 MHz, DMSO-d₆, 343K) of major |
| | tautomer δ_{H} 12.28 (br s, 1H, NH, D₂O exchanged), 9.66 (br s, 1H, |
| | NH, D₂O exchanged), 8.67 − 8.19 (m, 1H), 8.15 (s, 1H), 7.91 (br |
| | s, 1H), 7.68 − 7.41 (m, 1H), 6.64 (br s, 1H, NH, D₂O exchanged), |
| | 6.45 (s, 1H), 3.19 (s, 3H), 2.36 − 1.93 (m, 8H), 1.83 − 1.48 (m, |
| | 6H). MS (ESI⁺): [M + H]⁺ 392.4. |
| 87 | Pale yellow solid. ¹H NMR (400 MHz, DMSO-d₆, 343K) of major |
| | tautomer δ_{H} 12.31 (br s, 1H, NH, D₂O exchanged), 9.72 (br s, 1H, |
| | NH, D₂O exchanged), 8.44 (br s, 1H), 8.15 (s, 1H), 7.88 (d, J = |
| | 8.5 Hz, 1H), 7.52 (br s, 1H), 6.74 (br s, 1H, NH, D₂O exchanged), |
| | 6.47 (s, 1H), 2.44 − 2.23 (m, 4H), 2.23 − 1.77 (m, 8H), 1.71 − 1.49 |
| | (m, 2H). MS (ESI⁺): [M + H]⁺ 380.3. |
| 88 | Pale yellow solid. ¹H NMR (400 MHz, DMSO-d₆, 343K) of major |
| | tautomer δ_{H} 12.25 (br s, 1H, NH, D₂O exchanged), 10.47 (br s, |
| | 1H, NH, D₂O exchanged), 8.54 − 8.07 (m, 2H), 8.07 − 7.26 (m, |
| | 6H ^{+ 1H}, NH, D₂O exchanged), 6.49 (s, 1H), 4.82 (s, 2H). MS |
| | (ESI⁺): [M + H]⁺ 386.2. |
| 89 | Pale yellow solid. ¹H NMR (400 MHz, DMSO-d₆, 343K) of major |
| | tautomer δ_{H} 12.30 (br s, 1H, NH, D₂O exchanged), 10.42 (br s, |
| | 1H, NH, D₂O exchanged), 8.55 − 8.24 (m, 1H), 8.13 (s, 1H), 8.00 |
| | 7.76 (m, 1H), 7.74 − 7.43 (m, 1H + NH, D₂O exchanged), 7.41 − |
| | 7.08 (m, 4H), 6.51 (s, 1H), 5.41 (br s, 1H, OH, D₂O exchanged), |
| | 5.26 (br s, 1H), 4.44 (br s, 1H), 3.23 (dd, J = 15.7, 7.2 Hz, 1H), |
| | 2.80 (dd, J = 15.8, 7.3 Hz, 1H). MS (ESI⁺): [M + H]⁺ 360.3. |
| 90 | Pale yellow solid. ¹H NMR (400 MHz, DMSO-d₆, 343K) of major |
| | tautomer δ_{H} 12.30 (br s, 1H, NH, D₂O exchanged), 10.32 (br s, |
| | 1H, NH, D₂O exchanged), 8.35 (br s, 1H), 8.17 (s, 1H), 7.82 (br |
| | s, 1H), 7.72 − 7.05 (m, 6H ⁺ NH, D₂O exchanged), 6.44 (br s, |
| | 1H), 5.06 (br s, 1H), 4.94 (br s, 1H, OH, D₂O exchanged), 3.79 |
| | (br s, 2H). MS (ESI⁺): [M + H]⁺ 348.3. |
| 91 | Pale yellow solid. ¹H NMR (400 MHz, DMSO-d₆, 343K) of major |
| | tautomer δ_{H} 12.30 (br s, 1H, NH, D₂O exchanged), 10.32 (br s, |
| | 1H, NH, D₂O exchanged), 8.35 (br s, 1H), 8.17 (s, 1H), 7.82 (br |
| | s, 1H), 7.72 − 7.05 (m, 6H ⁺ NH, D₂O exchanged), 6.44 (br s, |
| | 1H), 5.06 (br s, 1H), 4.94 (br s, 1H, OH, D₂O exchanged), 3.79 |
| | (br s, 2H). MS (ESI⁺): [M + H]⁺ 348.3. |
| 92 | Pale yellow solid. ¹H NMR (400 MHz, DMSO-d₆, 373K) of major |
| | tautomer δ_{H} 12.30 (br s, 1H, NH, D₂O exchanged), 10.26 (br s, |
| | 1H, NH, D₂O exchanged), 8.36 (br s, 1H), 8.16 (s, 1H), 7.82 (br |
| | s, 1H), 7.68 (br s, 1H, NH, D₂O exchanged), 7.61 − 7.20 (m, 6H), |
| | 6.45 (s, 1H), 5.23 (br s, 1H), 3.85 − 3.64 (m, 2H), 3.34 (s, 3H). |
| | MS (ESI⁺): [M + H]⁺ 362.3. |
| 93 | Pale yellow solid. ¹H NMR (400 MHz, DMSO-d₆, 373K) of major |
| | tautomer δ_{H} 10.92 (br s, 2H, NH, D₂O exchanged), 8.24 (br s, 1H), |
| | 8.18 (s, 1H), 7.77 (br s, 1H), 7.56 (d, J = 8.4 Hz, 1H), 7.50 − 7.44 |
| | (m, 2H), 7.41 − 7.34 (m, 2H), 7.32 − 7.24 (m, 1H), 7.06 (br s, 1H, |
| | NH, D₂O exchanged), 6.46 (s, 1H), 5.61 (br s, 1H, OH, D₂O |
| | exchanged), 4.88 (dd, J = 7.9, 4.1 Hz, 1H), 3.69 (dd, J = 13.5, 4.2 |
| | Hz, 1H), 3.48 (dd, J = 13.5, 7.8 Hz, 1H). MS (ESI⁺): [M + H]⁺ |
| | 348.3. |
| 94 | Pale yellow solid. ¹H NMR (400 MHz, DMSO-d₆, 373K) of major |
| | tautomer δ_{H} 10.92 (br s, 2H, NH, D₂O exchanged), 8.24 (br s, 1H), |
| | 8.18 (s, 1H), 7.77 (br s, 1H), 7.56 (d, J = 8.4 Hz, 1H), 7.50 − 7.44 |
| | (m, 2H), 7.41 − 7.34 (m, 2H), 7.32 − 7.24 (m, 1H), 7.06 (br s, 1H, |
| | NH, D₂O exchanged), 6.46 (s, 1H), 5.61 (br s, 1H, OH, D₂O |
| | exchanged), 4.88 (dd, J = 7.9, 4.1 Hz, 1H), 3.69 (dd, J = 13.5, 4.2 |
| | Hz, 1H), 3.48 (dd, J = 13.5, 7.8 Hz, 1H). MS (ESI⁺): [M + H]⁺ |
| | 348.3. |
| 95 | Pale yellow solid. ¹H NMR (400 MHz, D₂O, 323K) of major |
| | tautomer δ_{H} 9.42 (s, 1H), 8.23 (s, 1H), 8.04 (d, J = 8.7 Hz, 1H), |
| | 7.92 (dd, J = 8.7, 1.5 Hz, 1H), 7.87 − 7.71 (m, 5H), 7.08 (s, 1H), |
| | 5.65 − 5.57 (m, 1H), 3.95 − 3.81 (m, 2H). MS (ESI⁺): [M + H]⁺ |
| | 347.2 (+2HCl). |
| 96 | Pale yellow solid. ¹H NMR (400 MHz, D₂O, 323K) of major |
| | tautomer δ_{H} 9.42 (s, 1H), 8.23 (s, 1H), 8.04 (d, J = 8.7 Hz, 1H), |
| | 7.92 (dd, J = 8.7, 1.5 Hz, 1H), 7.87 − 7.71 (m, 5H), 7.08 (s, 1H), |
| | 5.65 − 5.57 (m, 1H), 3.95 − 3.81 (m, 2H). MS (ESI⁺): [M + H]⁺ |
| | 347.2 (+2HCl). |
| 97 | Yellow solid. ¹H NMR (400 MHz, DMSO-d₆, 343K) of major |
| | tautomer δ_{H} 12.18 (br s, 1H, NH, D₂O exchanged), 10.09 (br s, |
| | 1H, NH, D₂O exchanged), 8.35 (br s, 1H), 8.16 (s, 1H), 7.83 (br |
| | s, 1H + NH, D₂O exchanged), 7.60 − 7.39 (m, 1H), 7.14 (s, 1H), |
| | 6.52 (s, 1H), 4.86 (s, 2H), 2.37 (s, 3H). MS (ESI⁺): [M + H]⁺ |
| | 339.2. |
| 98 | Pale yellow solid. ¹H NMR (400 MHz, DMSO-d₆, 343K) of major |
| | tautomer δ_{H} 12.29 (br s, 1H, NH, D₂O exchanged), 10.35 (br s, |
| | 1H, NH, D₂O exchanged), 8.62 − 8.27 (m, 1H), 8.15 (s, 1H), 7.84 |
| | (br s, 1H), 7.70 − 7.43 (m, 1H), 7.20 (br s, 1H, NH, D₂O |
| | exchanged), 6.43 (s, 1H), 4.00 − 3.79 (m, 2H), 3.52 − 3.18 (m, |
| | 4H), 1.88 (br s, 1H), 1.76 − 1.50 (m, 2H), 1.43 − 1.14 (m, 2H). |
| | MS (ESI⁺): [M + H]⁺ 326.3. |
| 99 | Yellow solid. ¹H NMR (400 MHz, DMSO-d₆, 343K) of major |
| | tautomer δ_{H} 12.38 (br s, 1H, NH, D₂O exchanged), 10.22 (br s, |
| | 1H, NH, D₂O exchanged), 9.23 (br s, 1H, NH, D₂O exchanged), |
| | 8.43 (br s, 1H), 8.20 (s, 1H), 8.07 − 7.77 (m, 1H), 7.74 − 7.36 (m, |
| | 3H), 6.99 (br s, 2H), 6.60 (s, 1H), 3.20 − 3.12 (m, 4H), 2.50 − 2.47 |
| | (m, 4H), 2.26 (s, 3H). MS (ESI⁺): [M + H]⁺ 402.4. |
| 100 | Yellow solid. ¹H NMR (400 MHz, DMSO-d₆, 343K) of major |
| | tautomer δ_{H} 12.41 (br s, 1H, NH, D₂O exchanged), 10.76 (br s, |
| | 2H, NH, D₂O exchanged), 8.75 − 8.12 (m, 3H), 8.09 − 7.73 (m, |
| | 2H), 7.69 − 7.33 (m, 2H), 7.20 − 6.96 (m, 1H), 6.76 (s, 1H). MS |
| | (ESI⁺): [M + H]⁺ 305.3. |
| 101 | Yellow solid. ¹H NMR (400 MHz, DMSO-d₆, 343K) of major |
| | tautomer δ_{H} 12.16 (br s, 1H, NH, D₂O exchanged), 10.24 (br s, |
| | 1H, NH, D₂O exchanged), 8.27 (br s, 1H), 8.17 (s, 1H), 7.76 (br |
| | s, 1H), 7.56 (d, J = 8.3 Hz, 1H), 7.17 (br s, 1H, NH, D₂O |
| | exchanged), 6.47 (s, 1H), 3.99 − 3.81 (m, 2H), 3.78 − 3.64 (m, |
| | 1H), 3.55 − 3.33 (m, 2H), 2.10 − 1.93 (m, 1H), 1.82 − 1.52 (m, |
| | 3H). MS (ESI⁺): [M + H]⁺ 312.3. |
| 102 | Pale yellow solid. ¹H NMR (400 MHz, DMSO-d₆, 343K) of major |
| | tautomer δ_{H} 12.37 (br s, 1H, NH, D₂O exchanged), 10.24 (br s, |
| | 1H, NH, D₂O exchanged), 8.66 − 8.24 (m, 1H), 8.15 (s, 1H), 7.84 |
| | (br s, 1H), 7.66 − 7.45 (m, 1H), 7.07 (br s, 1H, NH, D₂O |
| | exchanged), 6.46 (s, 1H), 4.97 (br s, 1H, OH, D₂O exchanged), |
| | 4.10 − 3.95 (m, 1H), 3.95 − 3.79 (m, 1H), 3.69 (br s, 2H), 3.56 − |
| | 3.35 (m, 1H), 3.32 − 3.18 (m, 1H), 2.04 − 1.87 (m, 1H), 1.68 − |
| | 1.44 (m, 1H). MS (ESI⁺): [M + H]⁺ 328.3. |
| 103 | Pale yellow solid. ¹H NMR (400 MHz, DMSO-d₆, 343K) of major |
| | tautomer δ_{H} 12.37 (br s, 1H, NH, D₂O exchanged), 10.24 (br s, |
| | 1H, NH, D₂O exchanged), 8.66 − 8.24 (m, 1H), 8.15 (s, 1H), 7.84 |
| | (br s, 1H), 7.66 − 7.45 (m, 1H), 7.07 (br s, 1H, NH, D₂O |
| | exchanged), 6.46 (s, 1H), 4.97 (br s, 1H, OH, D₂O exchanged), |
| | 4.10 − 3.95 (m, 1H), 3.95 − 3.79 (m, 1H), 3.69 (br s, 2H), 3.56 − |
| | 3.35 (m, 1H), 3.32 − 3.18 (m, 1H), 2.04 − 1.87 (m, 1H), 1.68 − |
| | 1.44 (m, 1H). MS (ESI⁺): [M + H]⁺ 328.3. |
| 104 | Pale yellow solid. ¹H NMR (400 MHz, DMSO-d₆, 343K) of major |
| | tautomer δ_{H} 12.31 (br s, 1H, NH, D₂O exchanged), 10.08 (br s, |
| | 1H, NH, D₂O exchanged), 8.37 (br s, 1H), 8.15 (s, 1H), 7.83 (br |
| | s, 1H), 7.53 (br s, 1H), 7.02 (br s, 1H, NH, D₂O exchanged), 6.45 |
| | (s, 1H), 4.14 (br s, 1H), 3.91 − 3.59 (m, 4H), 2.03 − 1.52 (m, 6H). |
| | MS (ESI⁺): [M + H]⁺ 326.3. |
| 105 | Pale yellow solid. ¹H NMR (400 MHz, DMSO-d₆, 343K) of major |
| | tautomer δ_{H} 10.23 (br s, 1H, NH, D₂O exchanged), 8.43 (br s, 1H), |
| | 8.14 (s, 1H), 7.79 (br s, 1H), 7.59 (d, J = 8.2 Hz, 1H), 7.13 (br s, |
| | 1H, NH, D₂O exchanged), 6.45 (s, 1H), 3.84 (s, 3H), 3.74 (br s, |
| | 1H), 1.99 (br s, 2H), 1.83 − 1.68 (m, 2H), 1.67 − 1.56 (m, 1H), |
| | 1.47 − 1.13 (m, 5H). MS (ESI⁺): [M + H]⁺ 324.2. |
| 106 | Pale yellow solid. ¹H NMR (400 MHz, DMSO-d₆, 343K) of major |
| | tautomer δ_{H} 10.15 (br s, 1H, NH, D₂O exchanged), 8.45 (s, 1H), |
| | 8.12 (s, 1H), 7.80 (d, J = 8.5 Hz, 1H), 7.57 (d, J = 8.4 Hz, 1H), |
| | 7.10 (br s, 1H, NH, D₂O exchanged), 6.44 (s, 1H), 3.97 (br s, 1H), |
| | 3.83 (s, 3H), 2.11 − 1.85 (m, 2H), 1.82 − 1.40 (m, 10H). MS |
| | (ESI ): [M + H]⁺ 338.3. |
| 107 | Pale yellow solid. ¹H NMR (400 MHz, DMSO-d₆, 343K) of major |
| | tautomer δ_{H} 10.12 (br s, 1H, NH, D₂O exchanged), 8.48 (s, 1H), |
| | 8.12 (s, 1H), 7.77 (d, J = 8.5 Hz, 1H), 7.57 (d, J = 8.4 Hz, 1H), |
| | 7.08 (br s, 1H, NH, D₂O exchanged), 6.44 (s, 1H), 4.07 (br s, 1H), |
| | 3.83 (s, 3H), 1.99 − 1.82 (m, 2H), 1.82 − 1.44 (m, 12H). MS |
| | (ESI⁺): [M + H]⁺ 352.3. |
| 108 | Yellow solid. ¹H NMR (400 MHz, DMSO-d₆, 343K) of major |
| | tautomer δ_{H} 10.05 (br s, 1H, NH, D₂O exchanged), 8.37 (s, 1H), |
| | 8.25 − 8.04 (m, 1H), 7.84 (d, J = 8.4 Hz, 1H), 7.56 (d, J = 8.3 Hz, |
| | 1H), 6.87 (br s, 1H, NH, D₂O exchanged), 6.45 (s, 1H), 4.85 − |
| | 4.64 (m, 1H, OH, D₂O exchanged), 4.15 − 3.95 (m, 1H), 3.82 (s, |
| | 3H), 3.53 (s, 2H), 1.81 − 1.63 (m, 1H), 1.59 − 1.41 (m, 2H), |
| | 1.03 − 0.85 (m, 6H). MS (ESI⁺): [M + H]⁺ 342.3. |
| 109 | Yellow solid. ¹H NMR (400 MHz, DMSO-d₆, 373K) of major |
| | tautomer δ_{H} 10.20 (br s, 1H, NH, D₂O exchanged), 8.38 (s, 1H), |
| | 8.12 (s, 1H), 7.83 (d, J = 8.5 Hz, 1H), 7.56 (d, J = 8.4 Hz, 1H), |
| | 6.99 (br s, 1H, NH, D₂O exchanged), 6.45 (s, 1H), 4.22 (s, 1H), |
| | 3.81 (s, 3H), 3.54 − 3.40 (m, 2H), 3.33 (s, 3H), 1.79 − 1.62 (m, |
| | 1H), 1.58 − 1.36 (m, 2H), 1.12 − 0.83 (m, 6H). MS (ESI⁺): |
| | [M + H]⁺ 356.4. |
| 110 | Yellow solid. ¹H NMR (400 MHz, DMSO-d₆, 373K) of major |
| | tautomer δ_{H} 10.04 (br s, 1H, NH, D₂O exchanged), 8.36 (s, 1H), |
| | 8.13 (s, 1H), 7.84 (d, J = 8.5 Hz, 1H), 7.57 (d, J = 8.3 Hz, 1H), |
| | 7.23 (br s, 1H, NH, D₂O exchanged), 6.49 (s, 1H), 4.63 − 4.52 (m, |
| | 1H), 4.51 − 4.41 (m, 1H), 4.30 (br s, 1H), 3.82 (s, 3H), 1.83 − 1.66 |
| | (m, 1H), 1.65 − 1.52 (m, 1H), 1.50 − 1.37 (m, 1H), 1.07 − 0.81 |
| | (m, 6H). MS (ESI⁺): [M + H]⁺ 344.3. |
| 111 | Yellow solid. ¹H NMR (400 MHz, DMSO-d₆, 373K) of major |
| | tautomer 8n 10.04 (br s, 1H, NH, D₂O exchanged), 8.36 (s, 1H), |
| | 8.13 (s, 1H), 7.84 (d, J = 8.5 Hz, 1H), 7.57 (d, J = 8.3 Hz, 1H), |
| | 7.23 (br s, 1H, NH, D₂O exchanged), 6.49 (s, 1H), 4.63 − 4.52 (m, |
| | 1H), 4.51 − 4.41 (m, 1H), 4.30 (br s, 1H), 3.82 (s, 3H), 1.83 − 1.66 |
| | (m, 1H), 1.65 − 1.52 (m, 1H), 1.50 − 1.37 (m, 1H), 1.07 − 0.81 |
| | (m, 6H). MS (ESI⁺): [M + H]⁺ 344.3. |
| 112 | Pale yellow solid. ¹H NMR (400 MHz, DMSO-d₆, 343K) of major |
| | tautomer δ_{H} 10.24 (br s, 1H, NH, D₂O exchanged), 8.33 (s, 1H), |
| | 8.13 (s, 1H), 7.90 (d, J = 8.5 Hz, 1H), 7.57 (d, J = 8.5 Hz, 1H), |
| | 7.27 (br s, 1H, NH, D₂O exchanged), 6.47 (s, 1H), 4.16 (br s, 1H), |
| | 3.93 − 3.74 (m, 4H), 3.36 (s, 3H), 2.15 − 2.04 (m, 1H), 1.99 − 1.87 |
| | (m, 1H), 1.83 − 1.48 (m, 4H). MS (ESI⁺): [M + H]⁺ 340.3. |
| 113 | Pale yellow solid. ¹H NMR (400 MHz, DMSO-d₆, 343K) of major |
| | tautomer δ_{H} 10.24 (br s, 1H, NH, D₂O exchanged), 8.33 (s, 1H), |
| | 8.13 (s, 1H), 7.90 (d, J = 8.5 Hz, 1H), 7.57 (d, J = 8.5 Hz, 1H), |
| | 7.27 (br s, 1H, NH, D₂O exchanged), 6.47 (s, 1H), 4.16 (br s, 1H), |
| | 3.93 − 3.74 (m, 4H), 3.36 (s, 3H), 2.15 − 2.04 (m, 1H), 1.99 − 1.87 |
| | (m, 1H), 1.83 − 1.48 (m, 4H). MS (ESI⁺): [M + H]⁺ 340.3. |
| 114 | Yellow solid. ¹H NMR (400 MHz, DMSO-d₆, 373K) of major |
| | tautomer δ_{H} 9.84 (br s, 1H, NH, D₂O exchanged), 8.50 (s, 1H), |
| | 8.12 (s, 1H), 7.68 (d, J = 8.6 Hz, 1H), 7.56 (d, J = 8.4 Hz, 1H), |
| | 7.10 (br s, 1H, NH, D₂O exchanged), 6.46 (s, 1H), 3.81 (s, 3H), |
| | 2.66 (d, J = 7.1 Hz, 1H), 2.35 (d, J = 10.3 Hz, 4H), 2.19 (t, J = 8.6 |
| | Hz, 2H), 2.03 (d, J = 10.1 Hz, 2H), 1.77 − 1.46 (m, 4H). MS |
| | (ESI⁺): [M + H]⁺ 362.4. |
| 115 | Yellow solid. ¹H NMR (400 MHz, DMSO-d₆, 323K) of major |
| | tautomer δ_{H} 9.71 (br s, 1H, NH, D₂O exchanged), 8.57 (s, 1H), |
| | 8.15 (s, 1H), 7.67 (d, J = 8.4 Hz, 1H), 7.57 (d, J = 8.4 Hz, 1H), |
| | 6.66 (br s, 1H, NH, D₂O exchanged), 6.44 (s, 1H), 3.82 (s, 3H), |
| | 2.29 − 2.02 (m, 9H), 1.81 − 1.61 (m, 6H). MS (ESI⁺): [M + H]⁺ |
| | 376.3. |
| 116 | Beige solid. ¹H NMR (400 MHz, DMSO-d₆, 300K) of major |
| | tautomer δ_{H} 9.85 (br s, 1H, NH, D₂O exchanged), 8.61 (s, 1H), |
| | 8.18 (s, 1H), 7.72 − 7.52 (m, 2H), 6.92 (s, 1H), 6.44 (s, 1H), 4.60 |
| | (s, 1H, OH, D₂O exchanged), 3.84 (s, 3H), 2.29 − 2.17 (m, 2H), |
| | 2.16 − 1.87 (m, 6H), 1.72 − 1.38 (m, 6H). MS (ESI⁺): [M + H]⁺ |
| | 392.3. |
| 117 | Pale yellow solid. ¹H NMR (400 MHz, DMSO-d₆, 323K) of major |
| | tautomer δ_{H} 9.72 (br s, 1H, NH, D₂O exchanged), 8.53 (s, 1H), |
| | 8.15 (s, 1H), 7.68 (d, J = 8.6 Hz, 1H), 7.57 (d, J = 8.3 Hz, 1H), |
| | 6.82 (br s, 1H, NH, D₂O exchanged), 6.46 (s, 1H), 3.84 (s, 3H), |
| | 3.21 (s, 3H), 2.40 − 1.91 (m, 8H), 1.81 − 1.43 (m, 6H). MS (ESI⁺): |
| | [M + H]⁺ 406.3. |
| 118 | Pale yellow solid. ¹H NMR (400 MHz, DMSO-d₆, 343K) of major |
| | tautomer δ_{H} 9.73 (br s, 1H, NH, D₂O exchanged), 8.52 (s, 1H), |
| | 8.14 (s, 1H), 7.67 (d, J = 8.5 Hz, 1H), 7.58 (d, J = 8.4 Hz, 1H), |
| | 6.80 (br s, 1H, NH, D₂O exchanged), 6.48 (s, 1H), 3.83 (s, 3H), |
| | 2.45 − 2.27 (m, 4H), 2.23 − 1.97 (m, 4H), 1.96 − 1.79 (m, 4H), |
| | 1.68 − 1.52 (m, 2H). MS (ESI⁺): [M + H]' 394.4. |
| 119 | Pale yellow solid. ¹H NMR (400 MHz, DMSO-d₆, 373K) of major |
| | tautomer δ_{H} 10.53 (br s, 1H, NH, D₂O exchanged), 8.32 (br s, 1H), |
| | 8.11 (br s, 1H), 7.98 − 7.30 (m, 6H + 1H, NH, D₂O exchanged), |
| | 6.50 (s, 1H), 4.84 (s, 2H), 3.74 (s, 3H). MS (ESI⁺): [M + H]⁺ |
| | 400.2. |
| 120 | Yellow solid. ¹H NMR (400 MHz, DMSO-d₆, 343K) of major |
| | tautomer δ_{H} 10.42 (br s, 1H, NH, D₂O exchanged), 8.32 (s, 1H), |
| | 8.11 (s, 1H), 7.96 − 7.84 (m, 1H), 7.67 (br s, 1H, NH, D₂O |
| | exchanged), 7.62 − 7.52 (m, 1H), 7.44 − 7.10 (m, 4H), 6.53 (s, |
| | 1H), 5.44 (br s, 1H, OH, D₂O exchanged), 5.29 − 5.14 (m, 1H), |
| | 4.53 − 4.36 (m, 1H), 3.79 (s, 3H), 3.25 (dd, J = 15.8, 7.2 Hz, 1H), |
| | 2.80 (dd, J = 15.7, 7.1 Hz, 1H). MS (ESI⁺): [M + H]⁺ 374.3. |
| 121 | Yellow solid. ¹H NMR (400 MHz, DMSO-d₆, 373K) of major |
| | tautomer δ_{H} 10.25 (br s, 1H, NH, D₂O exchanged), 8.27 (br s, 1H), |
| | 8.15 (s, 1H), 7.88 − 7.51 (m, 2H ^{+ 1H}, NH, D₂O exchanged), 7.50 − |
| | 7.20 (m, 5H), 6.45 (s, 1H), 5.06 (br s, 1H), 4.94 (br s, 1H, OH, |
| | D₂O exchanged), 3.85 (s, 3H), 3.80 − 3.71 (m, 2H). MS (ESI⁺): |
| | [M + H]⁺ 362.3. |
| 122 | Yellow solid. ¹H NMR (400 MHz, DMSO-d₆, 373K) of major |
| | tautomer δ_{H} 10.25 (br s, 1H, NH, D₂O exchanged), 8.27 (br s, 1H), |
| | 8.15 (s, 1H), 7.88 − 7.51 (m, 2H ^{+ 1H}, NH, D₂O exchanged), 7.50 |
| | - 7.20 (m, 5H), 6.45 (s, 1H), 5.06 (br s, 1H), 4.94 (br s, 1H, OH, |
| | D₂O exchanged), 3.85 (s, 3H), 3.80 − 3.71 (m, 2H). MS (ESI⁺): |
| | [M + H]⁺ 362.3. |
| 123 | Yellow solid. ¹H NMR (400 MHz, DMSO-d₆, 343K) of major |
| | tautomer δ_{H} 10.41 (brs, 1H, NH, D₂O exchanged), 8.35 (br s, 1H), |
| | 8.16 (s, 1H), 7.87 (br s, 1H, NH, D₂O exchanged), 7.78 (d, J = 8.3 |
| | Hz, 1H), 7.57 (d, J = 8.4 Hz, 1H), 7.49 (d, J = 7.6 Hz, 2H), 7.40 |
| | 7.32 (m, 2H), 7.32 − 7.23 (m, 1H), 6.45 (s, 1H), 5.23 (br s, 1H), |
| | 3.85 (s, 3H), 3.78 − 3.71 (m, 1H), 3.70 − 3.62 (m, 1H), 3.34 (s, |
| | 3H). MS (ESI⁺): [M + H]⁺ 376.3. |
| 124 | Pale Yellow solid. ¹H NMR (400 MHz, DMSO-d₆, 343K) of |
| | major tautomer δ_{H} 10.30 (br s, 1H, NH, D₂O exchanged), 8.40 − |
| | 8.31 (m, 1H), 8.15 (s, 1H), 7.93 − 7.86 (m, 1H), 7.70 − 7.55 (m, |
| | 1H), 7.46 (d, J = 7.5 Hz, 2H), 7.37 (t, J = 7.5 Hz, 2H), 7.28 (t, J = |
| | 7.2 Hz, 1H), 7.14 (br s, 1H, NH, D₂O exchanged), 6.49 (s, 1H), |
| | 5.62 (br s, 1H, OH, D₂O exchanged), 5.00 − 4.81 (m, 1H), 3.84 |
| | (s, 3H), 3.77 − 3.64 (m, 1H), 3.55 − 3.40 (m, 1H). MS (ESI⁺): |
| | [M + H]⁺ 362.3. |
| 125 | Yellow solid. ¹H NMR (400 MHz, DMSO-d₆, 343K) of major |
| | tautomer δ_{H} 10.30 (br s, 1H, NH, D₂O exchanged), 8.40 − 8.31 |
| | (m, 1H), 8.15 (s, 1H), 7.93 − 7.86 (m, 1H), 7.70 − 7.55 (m, 1H), |
| | 7.46 (d, J = 7.5 Hz, 2H), 7.37 (t, J = 7.5 Hz, 2H), 7.28 (t, J = 7.2 |
| | Hz, 1H), 7.14 (br s, 1H, NH, D₂O exch), 6.49 (s, 1H), 5.62 (br s, |
| | 1H, OH, D₂O exchanged), 5.00 − 4.81 (m, 1H), 3.84 (s, 3H), 3.77 − |
| | 3.64 (m, 1H), 3.55 − 3.40 (m, 1H). MS (ESI⁺): [M + H]⁺ 362.3. |
| 126 | Pale yellow solid. ¹H NMR (400 MHz, D₂O, 323K) of major |
| | tautomer δ_{H} 9.43 (s, 1H), 8.32 (s, 1H), 8.08 (s, 2H), 7.87 − 7.74 |
| | (m, 5H), 7.07 (s, 1H), 5.65 (dd, J = 8.6, 6.1 Hz, 1H), 4.36 (s, 3H), |
| | 3.97 − 3.79 (m, 2H). MS (ESI⁺): [M + H]⁺ 361.3 (+2HCl). |
| 127 | Pale yellow solid. ¹H NMR (400 MHz, D₂O, 323K) of major |
| | tautomer δ_{H} 9.43 (s, 1H), 8.32 (s, 1H), 8.08 (s, 2H), 7.87 − 7.74 |
| | (m, 5H), 7.07 (s, 1H), 5.65 (dd, J = 8.6, 6.1 Hz, 1H), 4.36 (s, 3H), |
| | 3.97 − 3.79 (m, 2H). MS (ESI⁺): [M + H]⁺ 361.2 (+2HCl). |
| 128 | Yellow solid. ¹H NMR (400 MHz, DMSO-d₆, 343K) of major |
| | tautomer δ_{H} 10.58 (br s, 1H, NH, D₂O exchanged), 8.52 − 8.35 |
| | (m, 1H), 8.14 (s, 1H), 7.93 (br s, 1H, NH, D₂O exchanged), 7.86 − |
| | 7.76 (m, 1H), 7.65 − 7.51 (m, 1H), 7.13 (s, 1H), 6.53 (s, 1H), |
| | 4.85 (s, 2H), 3.82 (s, 3H), 2.36 (s, 3H). MS (ESI⁺): [M + H]⁺ |
| | 353.3. |
| 129 | Yellow solid. ¹H NMR (400 MHz, DMSO-d₆, 343K) of major |
| | tautomer δ_{H} 10.40 (br s, 1H, NH, D₂O exchanged), 8.41 (br s, 1H), |
| | 8.13 (br s, 1H), 7.93 − 7.77 (m, 1H), 7.64 − 7.49 (m, 1H), 7.26 (br |
| | s, 1H, NH, D₂O exchanged), 6.45 (s, 1H), 4.02 − 3.71 (m, 5H), |
| | 3.43 − 3.24 (m, 4H), 1.93 (s, 1H), 1.74 − 1.56 (m, 2H), 1.39 − 1.19 |
| | (m, 2H). MS (ESI⁺): [M + H]⁺ 340.3. |
| 130 | Yellow solid. ¹H NMR (400 MHz, DMSO-d₆, 300K) of major |
| | tautomer δ_{H} 10.61 (br s, 1H, NH, D₂O exchanged), 9.58 (br s, 1H, |
| | NH, D₂O exchanged), 8.64 (s, 1H), 8.22 (s, 1H), 7.86 − 7.67 (m, |
| | 3H), 7.62 (d, J = 8.4 Hz, 1H), 6.98 (d, J = 8.7 Hz, 2H), 6.60 (s, |
| | 1H), 3.88 (s, 3H), 3.18 − 3.06 (m, 4H), 2.47 (d, J = 4.9 Hz, 4H), |
| | 2.24 (s, 3H). MS (ESI⁺): [M + H]⁺ 416.4. |
| 131 | Yellow solid. ¹H NMR (400 MHz, DMSO-d₆, 300K) of major |
| | tautomer δ_{H} 10.93 (br s, 2H, NH, D₂O exchanged), 8.63 − 8.14 |
| | (m, 3H), 7.98 (s, 1H), 7.90 − 7.56 (m, 3H), 7.16 − 7.01 (m, 1H), |
| | 6.77 (s, 1H), 3.89 (s, 3H). MS (ESI⁺): [M + H]⁺ 319.3. |
| 132 | Yellow solid. ¹H NMR (400 MHz, DMSO-d₆, 300K) δ_{H} 8.78 (s, |
| | 1H), 8.73 (s, 1H), 8.02 (dd, J = 8.4, 1.4 Hz, 1H), 7.80 (d, J = 8.3 |
| | Hz, 1H), 7.61 (d, J = 7.7 Hz, 1H, NH, D₂O exchanged), 6.59 (s, |
| | 1H), 3.97 (m, 1H), 3.14 (s, 3H), 2.15 − 2.00 (m, 2H), 1.95 − 1.80 |
| | (m, 2H), 1.78 − 1.66 (m, 1H), 1.55 − 1.35 (m, 4H), 1.32 − 1.15 |
| | (m, 1H). MS (ESI⁺): [M + H]⁺ 325.2. |
| 133 | Orange solid. ¹H NMR (400 MHz, DMSO-d₆, 300K) δ_{H} 8.77 (s, |
| | 1H), 8.73 (s, 1H), 7.95 (d, J = 8.4 Hz, 1H), 7.73 (d, J = 8.3 Hz, |
| | 1H), 7.59 (d, J = 7.3 Hz, 1H, NH, D₂O exchanged), 6.54 (s, 1H), |
| | 4.30 − 4.15 (m, 1H), 3.08 (s, 3H), 1.97 − 1.52 (m, 14H). MS |
| | (ESI⁺): [M + H]⁺ 353.3. |
| 134 | Yellow solid. ¹H NMR (400 MHz, DMSO-d₆, 300K) δ_{H} 8.72 (s, |
| | 1H), 8.70 (s, 1H), 8.96 (dd, J = 8.4, 1.4 Hz, 1H), 7.73 (d, J = 8.3 |
| | Hz, 1H), 7.58 (d, J = 8.4 Hz, 1H, NH, D₂O exchanged), 6.55 (s, |
| | 1H), 4.50 − 4.35 (m, 1H), 3.52 (dd, J = 9.8, 6.5 Hz, 1H), 3.44 (dd, |
| | J = 9.8, 5.5 Hz, 1H), 3.32 (s, 3H), 3.09 (s, 3H), 1.78 − 1.66 (m, |
| | 1H), 1.64 − 1.53 (m, 1H), 1.48 − 1.37 (m, 1H), 1.00 − 0.92 (m, |
| | 6H). MS (ESI⁺): [M + H]⁺ 357.2. |
| 135 | Yellow solid. ¹H NMR (400 MHz, DMSO-d₆, 300K) δ_{H} 8.73 (s, |
| | 1H), 8.71 (s, 1H), 7.99 (dd, J = 8.4, 1.4 Hz, 1H), 7.74 (d, J = 8.3 |
| | Hz, 1H), 7.62 (d, J = 6.4 Hz, 1H, NH, D₂O exchanged), 6.57 (s, |
| | 1H), 4.40 − 4.28 (m, 1H), 3.90 − 3.84 (m, 1H), 3.39 (s, 3H), 3.09 |
| | (s, 3H), 2.21 − 2.06 (m, 1H), 2.00 − 1.86 (m, 1H), 1.85 − 1.58 (m, |
| | 4H). MS (ESI⁺): [M + H]⁺ 341.2. |
| 136 | Yellow solid. ¹H NMR (400 MHz, DMSO-d₆, 300K) δ_{H} 8.73 (s, |
| | 1H), 8.71 (s, 1H), 7.99 (dd, J = 8.4, 1.4 Hz, 1H), 7.74 (d, J = 8.3 |
| | Hz, 1H), 7.62 (d, J = 6.4 Hz, 1H, NH, D₂O exchanged), 6.57 (s, |
| | 1H), 4.40 − 4.28 (m, 1H), 3.90 − 3.84 (m, 1H), 3.39 (s, 3H), 3.09 |
| | (s, 3H), 2.21 − 2.06 (m, 1H), 2.00 − 1.86 (m, 1H), 1.85 − 1.58 (m, |
| | 4H). MS (ESI⁺): [M + H]⁺ 341.2. |
| 137 | Yellow solid. ¹H NMR (400 MHz, DMSO-d₆, 300K) δ_{H} 8.75 (s, |
| | 1H), 8.72 (s, 1H), 7.93 (d, J = 8.2 Hz, 1H), 7.72 (d, J = 8.3 Hz, |
| | 1H), 7.51 (s, 1H, NH, D₂O exchanged), 6.56 (s, 1H), 3.11 (s, 3H), |
| | 2.80 (t, J = 6.6 Hz, 1H), 2.41 − 2.30 (m, 4H), 2.22 − 2.13 (m, 2H), |
| | 2.07 − 1.97 (m, 2H), 1.72 − 1.52 (m, 4H). MS (ESI⁺): [M + H]⁺ |
| | 363.2. |
| 138 | Yellow solid. ¹H NMR (400 MHz, DMSO-d₆, 300K) δ_{H} 8.84 (s, |
| | 1H), 8.72 (s, 1H), 7.96 − 7.66 (m, 2H), 6.90 (s, 1H, NH, D₂O |
| | exchanged), 6.55 (s, 1H), 3.08 (s, 3H), 2.28 (s, 6H), 2.16 (s, 3H), |
| | 1.75 (s, 6H). MS (ESI⁺): [M + H]⁺ 377.3. |
| 139 | Yellow solid. Isolated as an inseparable 71/29 mixture of (Z)/(E) |
| | isomers. ¹H NMR (400 MHz, DMSO-d₆, 300K) δ_{H} 8.83 & 8.80 |
| | (s, 1H), 8.74 & 8.73 (s, 1H), 7.93 & 7.89 (dd, J= 8.3, 1.5 Hz, 1H), |
| | 7.74 & 7.72 (d, J = 8.3 Hz, 1H), 6.97 & 6.84 (s, 1H, NH, D₂O |
| | exchanged), 6.56 & 6.52 (s, 1H), 4.62 & 4.56 (s, 1H, OH, D₂O |
| | exchanged), 3.08 (s, 3H), 2.30 − 2.00 (m, 8H), 1.75 − 1.45 (m, |
| | 6H). MS (ESI⁺): [M + H]⁺ 393.2. |
| 140 | Yellow solid. ¹H NMR (400 MHz, DMSO-d₆, 300K) δ_{H} 8.82 (s, |
| | 1H), 8.73 (s, 1H), 7.89 (dd, J = 8.3, 2.6 Hz, 1H), 7.73 (d, J = 8.3 |
| | Hz, 1H), 7.05 (s, 1H, NH, D₂O exchanged), 6.57 (s, 1H), 3.19 (s, |
| | 3H), 3.09 (s, 3H), 2.38 − 2.25 (m, 4H), 2.23 − 2.10 (m, 4H), 1.82 |
| | 1.53 (m, 6H). MS (ESI⁺): [M + H]⁺ 407.2. |
| 141 | Yellow solid. ¹H NMR (400 MHz, DMSO-d₆, 300K) δ_{H} 8.80 (s, |
| | 1H), 8.74 (s, 1H), 7.89 (dd, J = 8.3, 1.4 Hz, 1H), 7.75 (d, J = 8.3 |
| | Hz, 1H), 7.14 (s, 1H, NH, D₂O exchanged), 6.59 (s, 1H), 3.09 (s, |
| | 3H), 2.50 − 2.38 (m, 4H), 2.27 − 2.12 (m, 4H), 1.91 (br s, 4H), |
| | 1.61 (s, 2H). 1ºF NMR (376 MHz, DMSO-d₆, 300K) 8F −128.84. |
| | MS (ESI⁺): [M + H]⁺ 395.2. |
| 142 | Yellow solid. ¹H NMR (400 MHz, DMSO-d₆, 300K) δ_{H} 8.71 (s, |
| | 1H), 8.62 − 8.54 (m, 1H + NH, D₂O exchanged), 7.86 (dd, J = |
| | 8.5, 1.5 Hz, 1H), 7.81 − 7.64 (m, 4H), 7.49 (t, J = 7.6 Hz, 1H), |
| | 6.57 (s, 1H), 4.88 (d, J = 5.6 Hz, 2H), 3.18 (s, 3H). 19F NMR (376 |
| | MHz, DMSO-d₆, 300K) 8F −58.69. MS (ESI⁺): [M + H]⁺ 401.2. |
| 143 | Yellow solid. ¹H NMR (400 MHz, DMSO-d₆, 300K) δ_{H} 8.75 (s, |
| | 1H), 8.62 (s, 1H), 8.26 (d, J = 7.9 Hz, 1H, NH, D₂O exchanged), |
| | 7.98 (dd, J = 8.4, 1.4 Hz, 1H), 7.74 (d, J = 8.3 Hz, 1H), 7.57 − |
| | 7.50 (m, 2H), 7.43 − 7.36 (m, 2H), 7.32 − 7.25 (m, 1H), 6.55 (s, |
| | 1H), 5.57 − 5.38 (m, 1H), 3.84 (dd, J = 10.2, 8.7 Hz, 1H), 3.68 |
| | (dd, J = 10.2, 5.3 Hz, 1H), 3.36 (s, 3H), 3.16 (s, 3H). MS (ESI⁺): |
| | [M + H]' 377.2. |
| 144 | Yellow solid. ¹H NMR (400 MHz, DMSO-d₆, 300K) δ_{H} 8.80 − |
| | 8.74 (m, 1H + NH, D₂O exchanged), 8.70 (s, 1H), 8.00 (dd, J = |
| | 8.4, 1.5 Hz, 1H), 7.74 (d, J = 8.3 Hz, 1H), 7.19 (d, J = 1.2 Hz, |
| | 1H), 6.63 (s, 1H), 4.90 (s, 2H), 3.11 (s, 3H), 2.40 − 2.33 (m, 3H). |
| | MS (ESI⁺): [M + H]⁺ 354.2. |
| 145 | Yellow solid. ¹H NMR (400 MHz, DMSO-d₆, 300K) δ_{H} 13.09 (s, |
| | 1H, NH, D₂O exchanged), 8.44 (s, 1H), 8.21 (dd, J = 8.8, 1.5 Hz, |
| | 1H), 8.08 (s, 1H), 7.51 (d, J = 8.7 Hz, 1H), 7.35 (d, J = 7.8 Hz, |
| | 1H, NH, D₂O exchanged), 6.53 (s, 1H), 3.97 − 3.86 (m, 1H), 3.07 |
| | (s, 3H), 2.10 − 1.95 (m, 2H), 1.88 − 1.75 (m, 2H), 1.72 − 1.62 (m, |
| | 1H), 1.50 − 1.32 (m, 4H), 1.26 − 1.12 (m, 1H). MS (ESI⁺): |
| | [M + H]⁺ 324.2. |
| 146 | Yellow solid. ¹H NMR (400 MHz, DMSO-d₆, 300K) δ_{H} 13.09 (s, |
| | 1H, NH, D₂O exchanged), 8.47 (s, 1H), 8.23 (dd, J = 8.8, 1.5 Hz, |
| | 1H), 8.06 (s, 1H), 7.50 (d, J = 8.8 Hz, 1H), 7.37 (d, J = 7.7 Hz, |
| | 1H, NH, D₂O exchanged), 6.53 (s, 1H), 4.15 − 4.06 (m, 1H), 3.07 |
| | (s, 3H), 2.08 − 1.98 (m, 2H), 1.80 − 1.46 (m, 10H). MS (ESI⁺): |
| | [M + H]⁺ 338.3. |
| 147 | Yellow solid. ¹H NMR (400 MHz, DMSO-d₆, 300K) δ_{H} 13.09 (s, |
| | 1H, NH, D₂O exchanged), 8.55 (s, 1H), 8.20 (dd, J = 8.8, 1.5 Hz, |
| | 1H), 8.02 (s, 1H), 7.49 (d, J = 8.8 Hz, 1H), 7.40 (d, J = 7.8 Hz, |
| | 1H, NH, D₂O exchanged), 6.53 (s, 1H), 4.26 − 4.17 (m, 1H), 3.07 |
| | (s, 3H), 1.95 − 1.55 (m, 14H). MS (ESI⁺): [M + H]⁺ 352.3. |
| 148 | Yellow solid. ¹H NMR (400 MHz, DMSO-d₆, 300K) δ_{H} 13.07 (s, |
| | 1H, NH, D₂O exchanged), 8.47 (s, 1H), 8.21 (dd, J = 8.8, 1.4 Hz, |
| | 1H), 8.03 (s, 1H), 7.48 (d, J = 8.8 Hz, 1H), 7.22 (d, J = 8.5 Hz, |
| | 1H, NH, D₂O exchanged), 6.52 (s, 1H), 4.82 (t, J = 5.8 Hz, 1H, |
| | OH, D₂O exchanged), 4.28 − 4.16 (m, 1H), 3.60 − 3.48 (m, 2H), |
| | 3.09 (s, 3H), 1.80 − 1.65 (m, 1H), 1.56 (ddd, J = 14.1, 9.3, 5.2 Hz, |
| | 1H), 1.45 (ddd, J = 13.6, 8.8, 4.9 Hz, 1H), 0.99 (d, J = 4.3 Hz, |
| | 3H), 0.97 (d, J = 4.4 Hz, 3H). MS (ESI⁺): [M + H]⁺ 342.2. |
| 149 | Yellow solid. ¹H NMR (400 MHz, DMSO-d₆, 300K) δ_{H} 13.09 (s, |
| | 1H, NH, D₂O exchanged), 8.47 (s, 1H), 8.21 (d, J = 8.8 Hz, 1H), |
| | 8.03 (s, 1H), 7.49 (d, J = 8.8 Hz, 1H), 7.39 (d, J = 8.5 Hz, 1H, |
| | NH, D₂O exchanged), 6.54 (s, 1H), 4.45 − 4.36 (m, 1H), 3.51 (dd, |
| | J = 9.8, 6.4 Hz, 1H), 3.44 (dd, J = 9.8, 4.6 Hz, 1H), 3.33 (s, 3H), |
| | 3.08 (s, 3H), 1.77 − 1.67 (m, 1H), 1.61 − 1.53 (m, 1H), 1.45 − 1.37 |
| | (m, 1H), 1.00 − 0.93 (m, 6H). MS (ESI⁺): [M + H]⁺ 356.3. |
| 150 | Yellow solid. ¹H NMR (400 MHz, DMSO-d₆, 300K) δ_{H} 13.08 (s, |
| | 1H, NH, D₂O exchanged), 8.51 (s, 1H), 8.20 (dd, J = 8.8, 1.5 Hz, |
| | 1H), 8.05 (s, 1H), 7.49 (d, J = 8.7 Hz, 1H), 7.41 (d, J = 7.2 Hz, |
| | 1H, NH, D₂O exchanged), 6.57 (s, 1H), 4.37 − 4.29 (m, 1H), 3.88 |
| | 3.82 (m, 1H), 3.32 (s, 3H), 3.08 (s, 3H), 2.22 − 2.05 (m, 1H), |
| | 2.00 − 1.95 (m, 1H), 1.95 − 1.55 (m, 4H). MS (ESI⁺): [M + H]⁺ |
| | 340.2. |
| 151 | Yellow solid. ¹H NMR (400 MHz, DMSO-d₆, 300K) δ_{H} 13.08 (s, |
| | 1H, NH, D₂O exchanged), 8.51 (s, 1H), 8.20 (dd, J = 8.8, 1.5 Hz, |
| | 1H), 8.05 (s, 1H), 7.49 (d, J = 8.7 Hz, 1H), 7.41 (d, J = 7.2 Hz, |
| | 1H, NH, D₂O exchanged), 6.57 (s, 1H), 4.37 − 4.29 (m, 1H), 3.88 |
| | 3.82 (m, 1H), 3.32 (s, 3H), 3.08 (s, 3H), 2.22 − 2.05 (m, 1H), |
| | 2.00 − 1.95 (m, 1H), 1.95 − 1.55 (m, 4H). MS (ESI⁺): [M + H]⁺ |
| | 340.2. |
| 152 | Orange solid. ¹H NMR (400 MHz, DMSO-d₆, 300K) δ_{H} 13.09 (s, |
| | 1H, NH, D₂O exchanged), 8.53 (s, 1H), 8.17 (d, J = 8.9 Hz, 1H), |
| | 8.01 (s, 1H), 7.49 (d, J = 8.8 Hz, 1H), 7.32 (s, 1H, NH, D₂O |
| | exchanged), 6.55 (s, 1H), 3.10 (s, 3H), 2.82 − 2.74 (m, 1H), 2.42 |
| | 2.30 (m, 4H), 2.25 − 2.15 (m, 2H), 2.05 − 1.95 (m, 2H), 1.74 − |
| | 1.54 (m, 4H). MS (ESI⁺): [M + H]⁺ 362.3. |
| 153 | Yellow solid. ¹H NMR (400 MHz, DMSO-d₆, 300K) δ_{H} 13.10 (s, |
| | 1H, NH, D₂O exchanged), 8.54 (s, 1H), 8.20 (d, J = 8.8 Hz, 1H), |
| | 8.00 (s, 1H), 7.49 (d, J = 8.8 Hz, 1H), 6.71 (s, 1H, NH, D₂O |
| | exchanged), 6.55 (s, 1H), 3.07 (s, 3H), 2.32 − 2.24 (m, 6H), 2.16 |
| | (s, 3H), 1.75 (s, 6H). MS (ESI⁺): [M + H]⁺ 376.2. |
| 154 | Yellow solid. ¹H NMR (400 MHz, DMSO-d₆, 300K) δ_{H} 13.10 (s, |
| | 1H, NH, D₂O exchanged), 8.45 (s, 1H), 8.19 (d, J = 8.9 Hz, 1H), |
| | 8.01 (s, 1H), 7.49 (d, J = 8.8 Hz, 1H), 6.78 (s, 1H, NH, D₂O |
| | exchanged), 6.55 (s, 1H), 4.63 (s, 1H, OH, D₂O exchanged), 3.07 |
| | (s, 3H), 2.32 − 2.06 (m, 8H), 1.73 − 1.52 (m, 6H). MS (ESI⁺): |
| | [M + H]⁺ 392.1. |
| 155 | Yellow solid. ¹H NMR (400 MHz, DMSO-d₆, 300K) δ_{H} 13.10 (s, |
| | 1H, NH, D₂O exchanged), 8.55 (s, 1H), 8.20 (d, J = 8.8 Hz, 1H), |
| | 7.99 (s, 1H), 7.48 (d, J = 8.7 Hz, 1H), 6.86 (s, 1H, NH, D₂O |
| | exchanged), 6.56 (s, 1H), 3.19 (s, 3H), 3.07 (s, 3H), 2.38 − 2.26 |
| | (m, 4H), 2.24 − 2.09 (m, 4H), 1.84 − 1.54 (m, 6H). MS (ESI⁺): |
| | [M + H]⁺ 406.2. |
| 156 | Yellow solid. ¹H NMR (400 MHz, DMSO-d₆, 300K) δ_{H} 13.11 (s, |
| | 1H, NH, D₂O exchanged), 8.52 (s, 1H), 8.19 (d, J = 8.9 Hz, 1H), |
| | 8.01 (s, 1H), 7.50 (d, J = 8.8 Hz, 1H), 6.95 (s, 1H, NH, D₂O |
| | exchanged), 6.58 (s, 1H), 3.08 (s, 3H), 2.48 − 2.35 (m, 4H), 2.27 |
| | 2.12 (m, 4H), 1.91 (s, 4H), 1.68 − 1.56 (m, 2H). 1ºF NMR (376 |
| | MHz, DMSO-d₆, 300K) 8F −128.72. MS (ESI⁺): [M + H]⁺ 394.3. |
| 157 | Yellow solid. ¹H NMR (400 MHz, DMSO-d₆, 300K) δ_{H} 13.06 (s, |
| | 1H, NH, D₂O exchanged), 8.44 (s, 1H), 8.40 (m, 1H, NH, D₂O |
| | exchanged), 8.08 (dd, J = 8.8, 1.4 Hz, 1H), 7.97 (s, 1H), 7.83 − |
| | 7.65 (m, 3H), 7.49 (t, J = 7.6 Hz, 1H), 7.42 (d, J = 8.7 Hz, 1H), |
| | 6.57 (s, 1H), 4.87 (s, 2H), 3.17 (s, 3H). 19F NMR (376 MHz, |
| | DMSO-d₆, 300K) 8F −58.56. MS (ESI⁺): [M + H]⁺ 400.1. |
| 158 | Yellow solid. ¹H NMR (400 MHz, DMSO-d₆, 300K) δ_{H} 13.06 (s, |
| | 1H), 8.36 (s, 1H), 8.19 (d, J = 8.8 Hz, 1H), 8.05 (s, 1H), 7.98 (d, |
| | J = 8.4 Hz, 1H, NH, D₂O exchanged), 7.48 (d, J = 8.8 Hz, 1H), |
| | 7.32 − 7.15 (m, 4H), 6.60 (s, 1H), 5.54 (d, J = 5.2 Hz, 1H, OH, |
| | D₂O exchanged), 5.45 (t, J = 7.7 Hz, 1H), 4.60 − 4.48 (m, 1H), |
| | 3.25 (dd, J = 15.6, 7.4 Hz, 1H), 3.13 (s, 3H), 2.83 (dd, J = 15.6, |
| | 7.8 Hz, 1H). MS (ESI⁺): [M + H]⁺ 374.2. |
| 159 | Yellow solid. ¹H NMR (400 MHz, DMSO-d₆, 300K) oH 13.08 (s, |
| | 1H, NH, D₂O exchanged), 8.45 (s, 1H), 8.18 − 8.08 (m, 2H), 7.94 |
| | (d, J = 7.9 Hz, 1H, NH, D₂O exchanged), 7.57 − 7.46 (m, 3H), |
| | 7.38 (t, J = 7.6 Hz, 2H), 7.26 (t, J = 7.4 Hz, 1H), 6.52 (s, 1H), |
| | 5.28 − 5.16 (m, 1H), 5.06 (t, J= 5.8 Hz, 1H, OH, D₂O exchanged), |
| | 3.90 − 3.80 (m, 1H), 3.79 − 3.70 (m, 1H), 3.16 (s, 3H). MS (ESI⁺): |
| | [M + H]⁺ 362.2. |
| 160 | Yellow solid. ¹H NMR (400 MHz, DMSO-d₆, 300K) δ_{H} 13.08 (s, |
| | 1H, NH, D₂O exchanged), 8.45 (s, 1H), 8.18 − 8.08 (m, 2H), 7.94 |
| | (d, J = 7.9 Hz, 1H, NH, D₂O exchanged), 7.57 − 7.46 (m, 3H), |
| | 7.38 (t, J = 7.6 Hz, 2H), 7.26 (t, J = 7.4 Hz, 1H), 6.52 (s, 1H), |
| | 5.28 − 5.16 (m, 1H), 5.06 (t, J= 5.8 Hz, 1H, OH, D₂O exchanged), |
| | 3.90 − 3.80 (m, 1H), 3.79 − 3.70 (m, 1H), 3.16 (s, 3H). MS (ESI⁺): |
| | [M + H]⁺ 362.2. |
| 161 | Yellow solid. ¹H NMR (400 MHz, DMSO-d₆, 300K) δ_{H} 13.09 (s, |
| | 1H, NH, D₂O exchanged), 8.45 (s, 1H), 8.14 (dd, J = 8.8, 1.5 Hz, |
| | 1H), 8.12 − 8.09 (m, 1H), 8.06 (d, J = 7.9 Hz, 1H, NH, D₂O |
| | exchanged), 7.60 − 7.47 (m, 3H), 7.43 − 7.34 (m, 2H), 7.30 − 7.23 |
| | (m, 1H), 6.53 (s, 1H), 5.46 − 5.36 (m, 1H), 3.88 − 3.78 (m, 1H), |
| | 3.66 (dd, J = 10.1, 5.3 Hz, 1H), 3.32 (s, 3H), 3.14 (s, 3H). MS |
| | (ESI⁺): [M + H]⁺ 376.1. |
| 162 | Yellow solid. ¹H NMR (400 MHz, DMSO-d₆, 300K) δ_{H} 13.12 (s, |
| | 1H, NH, D₂O exchanged), 8.56 (s, 1H), 8.25 (d, J = 8.9 Hz, 1H), |
| | 8.07 (s, 1H), 7.96 (t, J = 5.6 Hz, 1H, NH, D₂O exchanged), 7.58 − |
| | 7.48 (m, 3H), 7.44 (t, J = 7.5 Hz, 2H), 7.36 − 7.28 (m, 1H), 6.59 |
| | (s, 1H), 5.70 (d, J = 4.3 Hz, 1H, OH, D₂O exchanged), 5.16 − 5.06 |
| | (m, 1H), 3.80 − 3.66 (m, 1H), 3.43 − 3.32 (m, 1H), 3.10 (s, 3H). |
| | MS (ESI⁺): [M + H]⁺ 362.2. |
| 163 | Yellow solid. ¹H NMR (400 MHz, DMSO-d₆, 300K) δ_{H} 7.74 (s, |
| | 1H), 7.65 − 7.50 (m, 5H), 7.38 (s, 1H), 7.25 − 7.14 (m, 2H), 6.29 |
| | (s, 1H), 5.24 (dd, J = 8.6, 6.1 Hz, 1H), 3.62 (dd, J = 13.4, 8.7 Hz, |
| | 1H), 3.52 (dd, J = 13.4, 6.0 Hz, 1H), 2.97 (s, 3H). MS (ESI⁺): |
| | [M + H]⁺ 361.2 (+2HCl). |
| 164 | Yellow solid. ¹H NMR (400 MHz, DMSO-d₆, 300K) δ_{H} 13.09 (s, |
| | 1H, NH, D₂O exchanged), 8.54 (t, J = 5.8 Hz, 1H, NH, D₂O |
| | exchanged), 8.49 (s, 1H), 8.22 (dd, J = 8.8, 1.5 Hz, 1H), 8.07 (s, |
| | 1H), 7.50 (d, J = 8.8 Hz, 1H), 7.20 − 7.18 (m, 1H), 6.63 (s, 1H), |
| | 4.90 (d, J = 5.6 Hz, 2H), 3.10 (s, 3H), 2.37 (s, 3H). MS (ESI⁺): |
| | [M + H]⁺ 353.1. |
| 165 | Yellow solid. ¹H NMR (400 MHz, DMSO-d₆, 300K) δ_{H} 13.08 (s, |
| | 1H, NH, D₂O exchanged), 8.46 (s, 1H), 8.23 (d, J = 8.8 Hz, 1H), |
| | 8.07 (s, 1H), 7.63 (t, J = 5.8 Hz, 1H, NH, D₂O exchanged), 7.51 |
| | (d, J = 8.7 Hz, 1H), 6.54 (s, 1H), 3.95 − 3.85 (m, 2H), 3.0 − 3.25 |
| | (m, 4H), 3.08 (s, 3H), 2.06 − 1.92 (m, 1H), 1.76 − 1.65 (m, 2H), |
| | 1.38 − 1.22 (m, 2H). MS (ESI⁺): [M + H]⁺ 340.1. |
| 166 | Yellow solid. ¹H NMR (400 MHz, DMSO-d₆, 300K) δ_{H} 13.15 (s, |
| | 1H, NH, D₂O exchanged), 9.21 (s, 1H, NH, D₂O exchanged), |
| | 8.53 (s, 1H), 8.21 (d, J = 8.8 Hz, 1H), 8.17 (s, 1H), 7.84 (d, J = |
| | 8.9 Hz, 2H), 7.56 (d, J = 8.8 Hz, 1H), 7.06 (d, J = 8.8 Hz, 2H), |
| | 6.71 (s, 1H), 3.23 (s, 3H), 3.20 − 3.13 (m, 4H), 2.52 − 2.45 (m, |
| | 4H), 2.25 (s, 3H). MS (ESI⁺): [M + H]⁺ 416.1. |
| 167 | Yellow solid. ¹H NMR (400 MHz, CF₃COOD, 300K) δ_{H} 9.01 (s, |
| | 1H), 8.60 − 8.52 (m, 2H), 8.47 (s, 1H), 8.25 (d, J = 8.9 Hz, 1H), |
| | 8.16 (d, J = 9.1 Hz, 1H), 7.97 − 7.90 (m, 1H), 7.71 (t, J = 6.8 Hz, |
| | 1H), 7.53 (s, 1H), 3.65 (s, 3H). MS (ESI⁺): [M + H]⁺ 319.1. |
| 168 | Yellow solid. ¹H NMR (400 MHz, DMSO-d₆, 300K) δ_{H} 13.10 (s, |
| | 1H, NH, D₂O exchanged), 8.38 (s, 1H), 8.25 (d, J = 8.8 Hz, 1H), |
| | 8.09 (s, 1H), 7.53 (d, J = 8.8 Hz, 1H), 7.33 (d, J = 7.6 Hz, 1H, |
| | NH, D₂O exchanged), 6.58 (s, 1H), 4.08 (br s, 1H), 4.03 − 3.94 |
| | (m, 1H), 3.87 − 3.76 (m, 1H), 3.45 − 3.22 (m, 2H), 3.08 (s, 3H), |
| | 2.15 − 2.00 (m, 1H), 1.85 − 1.60 (m, 3H). MS (ESI⁺): [M + H]⁺ |
| | 326.2. |
| 169 | Yellow solid. ¹H NMR (400 MHz, DMSO-d₆, 300K) δ_{H} 13.10 (s, |
| | 1H, NH, D₂O exchanged), 8.37 (s, 1H), 8.25 (d, J = 8.9 Hz, 1H), |
| | 8.09 (s, 1H), 7.54 (d, J = 8.8 Hz, 1H), 7.34 (d, J = 7.8 Hz, 1H, |
| | NH, D₂O exchanged), 6.57 (s, 1H), 5.10 (d, J = 4.7 Hz, 1H, OH, |
| | D₂O exchanged), 4.03 − 3.84 (m, 3H), 3.82 − 3.72 (m, 1H), 3.38 |
| | (t, J = 11.6 Hz, 1H), 3.20 (t, J = 10.4 Hz, 1H), 3.10 (s, 3H), 2.02 − |
| | 1.90 (m, 1H), 1.65 − 1.51 (m, 1H). MS (ESI⁺): [M + H]⁺ 342.2. |
| 170 | Yellow solid. ¹H NMR (400 MHz, DMSO-d₆, 300K) δ_{H} 8.49 |
| | 8.33 (m, 2H), 8.11 (dd, J = 9.1, 1.5 Hz, 1H), 7.54 (d, J = 9.0 Hz, |
| | 1H), 7.33 (d, J = 7.7 Hz, 1H, NH, D₂O exchanged), 6.48 (s, 1H), |
| | 4.15 (s, 3H), 3.90 (m, 1H), 3.06 (s, 3H), 2.10 − 1.95 (m, 2H), 1.90 |
| | 1.72 (m, 2H), 1.73 − 1.60 (m, 1H), 1.50 − 1.30 (m, 4H), 1.28 − |
| | 1.10 (m, 1H). MS (ESI⁺): [M + H]⁺ 338.3. |
| 171 | Yellow solid. ¹H NMR (400 MHz, DMSO-d₆, 300K) δ_{H} 8.38 (s, |
| | 1H), 8.35 (s, 1H), 8.11 (dd, J = 9.0, 1.5 Hz, 1H), 7.53 (d, J = 9.0 |
| | Hz, 1H), 7.36 (d, J = 7.6 Hz, 1H, NH, D₂O exchanged), 6.48 (s, |
| | 1H), 4.15 (s, 3H), 4.13 − 4.02 (m, 1H), 3.06 (s, 3H), 2.10 − 1.95 |
| | (m, 2H), 1.82 − 1.45 (m, 10H). MS (ESI⁺): [M + H]⁺ 352.3. |
| 172 | Yellow solid. ¹H NMR (400 MHz, DMSO-d₆, 300K) δ_{H} 8.43 (s, |
| | 1H), 8.31 (s, 1H), 8.10 (dd, J = 9.1, 1.5 Hz, 1H), 7.51 (d, J = 9.0 |
| | Hz, 1H), 7.37 (d, J = 7.6 Hz, 1H, NH, D₂O exchanged), 6.48 (s, |
| | 1H), 4.26 − 4.17 (m, 1H), 4.16 (s, 3H), 3.06 (s, 3H), 2.00 − 1.50 |
| | (m, 14H). MS (ESI⁺): [M + H]⁺ 366.3. |
| 173 | Yellow solid. ¹H NMR (400 MHz, DMSO-d₆, 300K) δ_{H} 8.36 (s, |
| | 1H), 8.33 (s, 1H), 8.12 (d, J = 9.0 Hz, 1H), 7.51 (d, J = 9.0 Hz, |
| | 1H), 7.24 (d, J = 8.5 Hz, 1H, NH, D₂O exchanged), 6.47 (s, 1H), |
| | 4.83 (t, J = 5.8 Hz, 1H, OH, D₂O exchanged), 4.26 − 4.17 (m, |
| | 1H), 4.15 (s, 3H), 3.60 − 3.48 (m, 2H), 3.08 (s, 3H), 1.78 − 1.66 |
| | (m, 1H), 1.56 (ddd, J = 14.0, 9.5, 5.2 Hz, 1H), 1.44 (ddd, J = 13.7, |
| | 8.8, 4.7 Hz, 1H), 1.01 − 0.94 (m, 6H). MS (ESI⁺): [M + H]⁺ 356.2. |
| 174 | Yellow solid. ¹H NMR (400 MHz, DMSO-d₆, 300K) δ_{H} 8.37 (s, |
| | 1H), 8.33 (s, 1H), 8.11 (dd, J = 9.1, 1.6 Hz, 1H), 7.52 (d, J = 9.1 |
| | Hz, 1H), 7.36 (d, J = 8.5 Hz, 1H, NH, D₂O exchanged), 6.49 (s, |
| | 1H), 4.47 − 4.32 (m, 1H), 4.15 (s, 3H), 3.51 (dd, J = 9.8, 6.3 Hz, |
| | 1H), 3.43 (dd, J = 9.8, 5.6 Hz, 1H), 3.32 (s, 3H), 3.07 (s, 3H), 1.80 |
| | 1.66 (m, 1H), 1.62 − 1.52 (m, 1H), 1.46 − 1.36 (m, 1H), 1.02 − |
| | 0.91 (m, 6H). MS (ESI⁺): [M + H]⁺ 370.3. |
| 175 | Yellow solid. ¹H NMR (400 MHz, DMSO-d₆, 300K) δ_{H} 8.40 (s, |
| | 1H), 8.34 (s, 1H), 8.12 (dd, J = 9.1, 1.5 Hz, 1H), 7.52 (d, J = 9.0 |
| | Hz, 1H), 7.41 (d, J = 7.2 Hz, 1H, NH, D₂O exchanged), 6.52 (s, |
| | 1H), 4.39 − 4.24 (m, 1H), 4.15 (s, 3H), 3.88 − 3.80 (m, 1H), 3.41 |
| | (s, 3H), 3.07 (s, 3H), 2.20 − 2.06 (m, 1H), 1.98 − 1.86 (m, 1H), |
| | 1.84 − 1.56 (m, 4H). MS (ESI⁺): [M + H]⁺ 354.2. |
| 176 | Yellow solid. ¹H NMR (400 MHz, DMSO-d₆, 300K) δ_{H} 8.40 (s, |
| | 1H), 8.34 (s, 1H), 8.12 (dd, J = 9.1, 1.5 Hz, 1H), 7.52 (d, J = 9.0 |
| | Hz, 1H), 7.41 (d, J = 7.2 Hz, 1H, NH, D₂O exchanged), 6.52 (s, |
| | 1H), 4.39 − 4.24 (m, 1H), 4.15 (s, 3H), 3.88 − 3.80 (m, 1H), 3.41 |
| | (s, 3H), 3.07 (s, 3H), 2.20 − 2.06 (m, 1H), 1.98 − 1.86 (m, 1H), |
| | 1.84 − 1.56 (m, 4H). MS (ESI⁺): [M + H]⁺ 354.2. |
| 177 | Pale yellow. ¹H NMR (400 MHz, DMSO-d₆, 300K) δ_{H} 8.47 (s, |
| | 1H), 8.32 (s, 1H), 8.04 (d, J = 9.1 Hz, 1H), 7.50 (d, J = 9.0 Hz, |
| | 1H), 7.32 (s, 1H, NH, D₂O exchanged), 6.50 (s, 1H), 4.15 (s, 3H), |
| | 3.09 (s, 3H), 2.78 (t, J = 6.7 Hz, 1H), 2.42 − 2.30 (m, 4H), 2.24 − |
| | 2.13 (m, 2H), 2.06 − 1.94 (m, 2H), 1.73 − 1.53 (m, 4H). MS |
| | (ESI⁺): [M + H]⁺ 376.3. |
| 178 | Yellow solid. ¹H NMR (400 MHz, DMSO-d₆, 300K) δ_{H} 8.44 (s, |
| | 1H), 8.31 (s, 1H), 8.09 (d, J = 9.1 Hz, 1H), 7.53 (d, J = 9.0 Hz, |
| | 1H), 6.69 (s, 1H, NH, D₂O exchanged), 6.49 (s, 1H), 4.16 (s, 3H), |
| | 3.07 (s, 3H), 2.27 (s, 6H), 2.16 (s, 3H), 1.75 (s, 6H). MS (ESI⁺): |
| | [M + H]⁺ 390.1. |
| 179 | Pale yellow solid. ¹H NMR (400 MHz, DMSO-d₆, 300K) δ_{H} 8.43 |
| | (s, 1H), 8.30 (s, 1H), 8.09 (d, J = 9.1 Hz, 1H), 7.53 (d, J = 9.0 Hz, |
| | 1H), 6.76 (s, 1H, NH, D₂O exchanged), 6.50 (s, 1H), 4.62 (s, 1H, |
| | OH, D₂O exchanged), 4.17 (s, 3H), 3.07 (s, 3H), 2.32 − 2.05 (m, |
| | 8H), 1.75 − 1.50 (m, 6H). MS (ESI⁺): [M + H]⁺ 406.2. |
| 180 | Pale yellow solid. ¹H NMR (400 MHz, DMSO-d₆, 300K) δ_{H} 8.42 |
| | (s, 1H), 8.29 (s, 1H), 8.10 (d, J = 9.1 Hz, 1H), 7.51 (d, J = 9.0 Hz, |
| | 1H), 6.84 (s, 1H, NH, D₂O exchanged), 6.51 (s, 1H), 4.16 (s, 3H), |
| | 3.19 (s, 3H), 3.07 (s, 3H), 2.38 − 2.24 (m, 4H), 2.16 (s, 4H), 1.82 − |
| | 1.54 (m, 6H). MS (ESI⁺): [M + H]⁺ 420.3. |
| 181 | Pale yellow solid. ¹H NMR (400 MHz, DMSO-d₆, 300K) δ_{H} 8.42 |
| | (s, 1H), 8.31 (s, 1H), 8.08 (d, J = 9.1 Hz, 1H), 7.53 (d, J = 9.1 Hz, |
| | 1H), 6.93 (s, 1H, NH, D₂O exchanged), 6.53 (s, 1H), 4.16 (s, 3H), |
| | 3.07 (s, 3H), 2.48 − 2.37 (m, 4H), 2.26 − 2.10 (m, 4H), 1.94 − 1.86 |
| | (m, 4H), 1.61 (s, 2H). ¹⁹F NMR (376 MHz, DMSO-d₆, 300K) |
| | δ_{F} −128.67. MS (ESI⁺): [M + H]⁺ 408.2. |
| 182 | Yellow solid. ¹H NMR (400 MHz, DMSO-d₆, 300K) δ_{H} 8.37 (t, |
| | J = 5.7 Hz, 1H, NH, D₂O exchanged), 8.32 (s, 1H), 8.25 (s, 1H), |
| | 7.99 (dd, J = 9.1, 1.5 Hz, 1H), 7.82 − 7.65 (m, 3H), 7.54 − 7.40 |
| | (m, 2H), 6.52 (s, 1H), 4.87 (d, J = 4.5 Hz, 2H), 4.15 (s, 3H), 3.17 |
| | (s, 3H). ¹⁹F NMR (376 MHz, DMSO-d₆, 300K) δ_{F} −58.61. MS |
| | (ESI⁺): [M + H]⁺ 414.2. |
| 183 | Yellow solid. ¹H NMR (400 MHz, DMSO-d₆, 300K) δ_{H} 8.34 (s, |
| | 1H), 8.28 (s, 1H), 8.13 (d, J = 9.2 Hz, 1H), 7.99 (d, J = 8.3 Hz, |
| | 1H, NH D₂O exchanged), 7.51 (d, J = 9.1 Hz, 1H), 7.32 − 7.17 |
| | (m, 4H), 6.56 (s, 1H), 5.53 (d, J = 5.2 Hz, 1H, OH, D₂O |
| | exchanged), 5.44 (t, J = 7.6 Hz, 1H), 4.59 − 4.49 (m, 1H), 4.13 (s, |
| | 3H), 3.25 (dd, J = 15.6, 7.4 Hz, 1H), 3.13 (s, 3H), 2.84 (dd, J = |
| | 15.6, 7.9 Hz, 1H). MS (ESI⁺): [M + H]⁺ 388.3. |
| 184 | Yellow solid. ¹H NMR (400 MHz, DMSO-d₆, 300K) δ_{H} 8.39 (s, |
| | 1H), 8.34 (s, 1H), 8.05 (dd, J = 9.1, 1.5 Hz, 1H), 7.93 (d, J = 7.8 |
| | Hz, 1H, NH, D₂O exchanged), 7.57 − 7.48 (m, 3H), 7.42 − 7.34 |
| | (m, 2H), 7.29 − 7.22 (m, 1H), 6.47 (s, 1H), 5.25 − 5.16 (m, 1H), |
| | 5.02 (t, J = 5.8 Hz, 1H, OH, D₂O exchanged), 4.16 (s, 3H), 3.89 − |
| | 3.79 (m, 1H), 3.78 − 3.69 (m, 1H), 3.16 (s, 3H). MS (ESI⁺): |
| | [M + H]⁺ 376.2. |
| 185 | Yellow solid. ¹H NMR (400 MHz, DMSO-d₆, 300K) δ_{H} 8.39 (s, |
| | 1H), 8.34 (s, 1H), 8.05 (dd, J = 9.1, 1.5 Hz, 1H), 7.93 (d, J = 7.8 |
| | Hz, 1H, NH, D₂O exchanged), 7.57 − 7.48 (m, 3H), 7.42 − 7.34 |
| | (m, 2H), 7.29 − 7.22 (m, 1H), 6.47 (s, 1H), 5.25 − 5.16 (m, 1H), |
| | 5.02 (t, J = 5.8 Hz, 1H, OH, D₂O exchanged), 4.16 (s, 3H), 3.89 |
| | 3.79 (m, 1H), 3.78 − 3.69 (m, 1H), 3.16 (s, 3H). MS (ESI⁺): |
| | [M + H]⁺ 376.2. |
| 186 | Yellow solid. ¹H NMR (400 MHz, DMSO-d₆, 300K) 8H. 8.38 (s, |
| | 1H), 8.34 (s, 1H), 8.05 (d, J = 8.6 Hz, 1H + NH, D₂O exchanged), |
| | 7.53 (d, J = 8.2 Hz, 3H), 7.38 (t, J = 7.5 Hz, 2H), 7.31 − 7.21 (m, |
| | 1H), 6.48 (s, 1H), 5.46 − 5.30 (m, 1H), 4.16 (s, 3H), 3.88 − 3.74 |
| | (m, 1H), 3.65 (dd, J= 10.2, 5.3 Hz, 1H), 3.34 (s, 3H), 3.13 (s, 3H) |
| | MS (ESI⁺): [M + H]⁺ 390.1. |
| 187 | Yellow solid. ¹H NMR (400 MHz, DMSO-d₆, 300K) δ_{H} 8.39 (s, |
| | 1H), 8.34 (s, 1H), 8.20 (d, J = 9.1 Hz, 1H), 7.93 (t, J = 5.7 Hz, |
| | 1H, NH, D₂O exchanged), 7.57 (d, J = 9.1 Hz, 1H), 7.53 − 7.40 |
| | (m, 4H), 7.36 − 7.27 (m, 1H), 6.53 (s, 1H), 5.68 (d, J = 4.3 Hz, |
| | 1H, OH, D₂O exchanged), 5.14 − 5.02 (m, 1H), 4.18 (s, 3H), 3.78 − |
| | 3.64 (m, 1H), 3.43 − 3.30 (m, 1H), 3.08 (s, 3H). MS (ESI⁺): |
| | [M + H]⁺ 376.2. |
| 188 | Yellow solid. ¹H NMR (400 MHz, D₂O, 300K) δ_{H} 8.12 (s, 1H), |
| | 8.02 (s, 1H), 7.71 (dd, J = 9.1, 1.6 Hz, 1H), 7.53 (d, J = 9.0 Hz, |
| | 1H), 7.50 − 7.36 (m, 6H), 6.73 (s, 1H), 4.12 (s, 3H), 3.92 − 3.75 |
| | (m, 2H), 2.99 (s, 3H). MS (ESI⁺): [M + H]⁺ 375.2 (+2HCl). |
| 189 | Yellow solid. ¹H NMR (400 MHz, DMSO-d₆, 300K) δ_{H} 8.54 (br |
| | s, 1H, NH, D₂O exchanged), 8.38 (s, 1H), 8.36 (s, 1H), 8.15 (d, J = |
| | 9.1 Hz, 1H), 7.53 (d, J = 9.0 Hz, 1H), 7.19 (s, 1H), 6.58 (s, 1H), |
| | 4.89 (s, 2H), 4.15 (s, 3H), 3.10 (s, 3H), 2.37 (s, 3H). MS (ESI⁺): |
| | [M + H] 367.1. |
| 190 | Yellow solid. ¹H NMR (400 MHz, DMSO-d₆, 300K) δ_{H} 8.38 |
| | 8.34 (m, 2H), 8.13 (dd, J = 9.1, 1.6 Hz, 1H), 7.63 (t, J = 5.7 Hz, |
| | 1H, NH, D₂O exchanged), 7.54 (d, J = 9.1 Hz, 1H), 6.49 (s, 1H), |
| | 4.16 (s, 3H), 3.94 − 3.86 (m, 2H), 3.38 (t, J = 6.3 Hz, 2H), 3.34 − |
| | 3.26 (m, 2H), 3.08 (s, 3H), 2.04 − 1.91 (m, 1H), 1.76 − 1.65 (m, |
| | 2H), 1.37 − 1.23 (m, 2H). MS (ESI⁺): [M + H]⁺ 354.2. |
| 191 | Yellow solid. ¹H NMR (400 MHz, DMSO-d₆, 300K) δ_{H} 9.20 (s, |
| | 1H, NH, D₂O exchanged), 8.43 (s, 1H), 8.41 (s, 1H), 8.13 (d, J = |
| | 9.2 Hz, 1H), 7.88 − 7.81 (m, 2H), 7.59 (d, J = 9.0 Hz, 1H), 7.09 − |
| | 7.01 (m, 2H), 6.66 (s, 1H), 4.17 (s, 3H), 3.23 (s, 3H), 3.20 − 3.13 |
| | (m, 4H), 2.51 − 2.45 (m, 4H), 2.25 (s, 3H). MS (ESI⁺): [M + H]⁺ |
| | 430.1. |
| 192 | Orange solid. ¹H NMR (400 MHz, CDCl₃, 300K) δ_{H} 12.41 (br s, |
| | 1H, NH, D₂O exchanged), 8.37 (ddd, J = 5.1, 2.0, 0.8 Hz, 1H), |
| | 8.00 (s, 1H), 7.85 (s, 1H), 7.80 (d, J = 9.0 Hz, 1H), 7.70 (td, J = |
| | 7.3, 2.0 Hz, 1H), 7.50 (dd, J = 9.0, 1.6 Hz, 1H), 7.22 (d, J = 8.2 |
| | Hz, 1H), 7.00 (ddd, J = 7.3, 5.0, 1.1 Hz, 1H), 6.79 (s, 1H), 4.28 |
| | (s, 3H), 3.34 (s, 3H). MS (ESI⁺): [M + H]⁺ 333.2. |
| 193 | Yellow solid. ¹H NMR (400 MHz, DMSO-d₆, 300K) δ_{H} 8.38 (s, |
| | 1H), 8.30 (s, 1H), 8.16 (dd, J = 9.2, 1.5 Hz, 1H), 7.56 (d, J = 9.1 |
| | Hz, 1H), 7.33 (d, J = 7.6 Hz, 1H, NH, D₂O exchanged), 6.53 (s, |
| | 1H), 4.15 (s, 3H), 4.12 − 3.96 (m, 2H), 3.87 − 3.77 (m, 1H), 3.40 |
| | 3.22 (m, 2H), 3.07 (s, 3H), 2.13 − 2.00 (m, 1H), 1.82 − 1.62 (m, |
| | 3H). MS (ESI⁺): [M + H]⁺ 340.2. |
| 194 | Yellow solid. ¹H NMR (400 MHz, DMSO-d₆, 300K) δ_{H} 8.38 (s, |
| | 1H), 8.29 (s, 1H), 8.16 (dd, J = 9.1, 1.6 Hz, 1H), 7.57 (d, J = 9.1 |
| | Hz, 1H), 7.35 (d, J = 7.6 Hz, 1H, NH, D₂O echanged), 6.52 (s, |
| | 1H), 5.10 (d, J = 4.7 Hz, 1H, OH, D₂O exchanged), 4.15 (s, 3H), |
| | 3.99 (dd, J = 11.0, 4.7 Hz, 1H), 3.94 − 3.82 (m, 2H), 3.82 − 3.72 |
| | (m, 1H), 3.38 (td, J = 11.7, 2.2 Hz, 1H), 3.19 (t, J = 10.5 Hz, 1H), |
| | 3.09 (s, 3H), 2.02 − 1.90 (m, 1H), 1.65 − 1.50 (m, 1H). MS (ESI⁺): |
| | [M + H]⁺ 356.2. |
| 195 | Orange solid. ¹H NMR (400 MHz, DMSO-d₆, 300K) δ_{H} 8.41 (s, |
| | 1H), 8.30 (d, J = 8.9 Hz, 1H), 8.06 (s, 1H), 7.63 (d, J = 8.8 Hz, |
| | 1H), 7.37 (d, J = 7.7 Hz, 1H, NH, D₂O exchanged), 6.54 (s, 1H), |
| | 4.04 (s, 3H), 3.92 (s, 1H), 3.07 (s, 3H), 2.04 (br s, 2H), 1.81 (br s, |
| | 2H), 1.73 − 1.65 (m, 1H), 1.50 − 1.31 (m, 4H), 1.28 − 1.12 (m, |
| | 1H). MS (ESI ): [M + H]⁺ 338.2. |
| 196 | Orange solid. ¹H NMR (400 MHz, DMSO-d₆, 300K) δ_{H} 8.41 (s, |
| | 1H), 8.32 (d, J = 8.9 Hz, 1H), 8.03 (s, 1H), 7.62 (d, J = 8.8 Hz, |
| | 1H), 7.39 (d, J = 7.6 Hz, 1H, NH, D₂O exchanged), 6.53 (s, 1H), |
| | 4.16 − 4.06 (m, 1H), 4.04 (s, 3H), 3.06 (s, 3H), 2.10 − 1.94 (m, |
| | 2H), 1.85 − 1.43 (m, 10H). MS (ESI⁺): [M + H]⁺ 352.2. |
| 197 | Orange solid. ¹H NMR (400 MHz, DMSO-d₆, 300K) δ_{H} 8.48 (s, |
| | 1H), 8.29 (d, J = 8.8 Hz, 1H), 7.99 (s, 1H), 7.60 (d, J = 8.9 Hz, |
| | 1H), 7.40 (d, J = 7.6 Hz, 1H, NH, D₂O exchanged), 6.53 (s, 1H), |
| | 4.27 − 4.15 (m, 1H), 4.04 (s, 3H), 3.06 (s, 3H), 2.00 − 1.41 (m, |
| | 14H). MS (ESI⁺): [M + H]⁺ 366.2. |
| 198 | Yellow solid. ¹H NMR (400 MHz, DMSO-d₆, 300K) δ_{H} 8.43 (s, |
| | 1H), 8.28 (dd, J = 8.9, 1.5 Hz, 1H), 8.01 (d, J = 1.3 Hz, 1H), 7.60 |
| | (d, J = 8.9 Hz, 1H), 7.25 (d, J = 8.5 Hz, 1H, NH, D₂O exchanged), |
| | 6.53 (s, 1H), 4.82 (t, J = 5.8 Hz, 1H, OH, D₂O exchanged), 4.23 |
| | (m, 1H), 4.04 (s, 3H), 3.60 − 3.48 (m, 2H), 3.09 (s, 3H), 1.80 − |
| | 1.65 (m, 1H), 1.56 (ddd, J = 14.0, 9.4, 5.2 Hz, 1H), 1.45 (ddd, J = |
| | 13.7, 8.8, 4.9 Hz, 1H), 0.99 (d, J = 5.2 Hz, 3H). 0.97 (d, J = 5.4 |
| | Hz, 3H). MS (ESI⁺): [M + H]⁺ 356.2. |
| 199 | Yellow solid. ¹H NMR (400 MHz, DMSO-d₆, 300K) δ_{H} 8.41 (s, |
| | 1H), 8.29 (d, J = 8.8 Hz, 1H), 8.00 (s, 1H), 7.60 (d, J = 8.9 Hz, |
| | 1H), 7.39 (d, J = 8.5 Hz, 1H, NH, D₂O exchanged), 6.54 (s, 1H), |
| | 4.48 − 4.34 (m, 1H), 4.03 (s, 3H), 3.51 (dd, J = 9.8, 6.4 Hz, 1H), |
| | 3.43 (dd, J = 9.8, 5.6 Hz, 1H), 3.32 (s, 3H), 3.07 (s, 3H), 1.78 − |
| | 1.66 (m, 1H), 1.63 − 1.52 (m, 1H), 1.46 − 1.34 (m, 1H), 1.01 − |
| | 0.89 (m, 6H). MS (ESI⁺): [M + H]⁺ 370.2. |
| 200 | Beige solid. ¹H NMR (400 MHz, DMSO-d₆, 300K) δ_{H} 8.48 (s, |
| | 1H), 8.27 (d, J = 8.4 Hz, 1H), 8.02 (s, 1H), 7.60 (d, J = 8.8 Hz, |
| | 1H), 7.43 (d, J = 7.2 Hz, 1H, NH, D₂O exchanged), 6.57 (s, 1H), |
| | 4.40 − 4.27 (m, 1H), 4.04 (s, 3H), 3.90 − 3.82 (m, 1H), 3.42 (s, |
| | 3H), 3.08 (s, 3H), 2.20 − 2.05 (m, 1H), 2.00 − 1.86 (m, 1H), 1.85 − |
| | 1.56 (m, 4H). MS (ESI⁺): [M + H]⁺ 354.3. |
| 201 | Beige solid. ¹H NMR (400 MHz, DMSO-d₆, 300K) δ_{H} 8.48 (s, |
| | 1H), 8.27 (d, J = 8.4 Hz, 1H), 8.02 (s, 1H), 7.60 (d, J = 8.8 Hz, |
| | 1H), 7.43 (d, J = 7.2 Hz, 1H, NH, D₂O exchanged), 6.57 (s, 1H), |
| | 4.40 − 4.27 (m, 1H), 4.04 (s, 3H), 3.90 − 3.82 (m, 1H), 3.42 (s, |
| | 3H), 3.08 (s, 3H), 2.20 − 2.05 (m, 1H), 2.00 − 1.86 (m, 1H), 1.85 − |
| | 1.56 (m, 4H). MS (ESI⁺): [M + H]⁺ 354.3. |
| 202 | Orange solid. ¹H NMR (400 MHz, DMSO-d₆, 300K) δ_{H} 8.45 (s, |
| | 1H), 8.29 (d, J = 8.8 Hz, 1H), 7.99 (s, 1H), 7.59 (d, J = 8.8 Hz, |
| | 1H), 7.34 (s, 1H, NH, D₂O exchanged), 6.56 (s, 1H), 4.04 (s, 3H), |
| | 3.10 (s, 3H), 2.79 (t, J = 6.6 Hz, 1H), 2.42 − 2.30 (m, 4H), 2.26 − |
| | 2.14 (m, 2H), 2.08 − 1.96 (m, 2H), 1.74 − 1.54 (m, 4H). MS |
| | (ESI⁺): [M + H]⁺ 376.2. |
| 203 | Pale Yellow solid. ¹H NMR (400 MHz, DMSO-d₆, 300K) δ_{H} 8.42 |
| | (s, 1H), 8.36 (d, J = 8.9 Hz, 1H), 7.98 (s, 1H), 7.63 (d, J = 8.8 Hz, |
| | 1H), 6.71 (s, 1H, NH, D₂O exchanged), 6.55 (s, 1H), 4.05 (s, 3H), |
| | 3.08 (s, 3H), 2.28 (s, 6H), 2.16 (s, 3H), 1.75 (s, 6H). MS (ESI⁺): |
| | [M + H]⁺ 390.1. |
| 204 | Pale Yellow solid. ¹H NMR (400 MHz, DMSO-d₆, 300K) δ_{H} 8.45 |
| | (s, 1H), 8.33 (d, J = 8.9 Hz, 1H), 7.99 (s, 1H), 7.62 (d, J = 8.8 Hz, |
| | 1H), 6.78 (s, 1H, NH, D₂O exchanged), 6.56 (s, 1H), 4.63 (s, 1H, |
| | OH, D₂O exchanged), 4.05 (s, 3H), 3.07 (s, 3H), 2.32 − 2.06 (m, |
| | 8H), 1.74 − 1.50 (m, 6H). MS (ESI⁺): [M + H]⁺ 406.1. |
| 205 | Beige solid. ¹H NMR (400 MHz, DMSO-d₆, 300K) δ_{H} 8.45 (s, |
| | 1H), 8.33 (d, J = 8.9 Hz, 1H), 7.97 (s, 1H), 7.62 (d, J = 8.9 Hz, |
| | 1H), 6.89 (s, 1H, NH, D₂O exchanged), 6.57 (s, 1H), 4.05 (s, 3H), |
| | 3.19 (s, 3H), 3.07 (s, 3H), 2.33 (s, 2H), 2.28 (s, 2H), 2.17 (s, 4H), |
| | 1.85 − 1.50 (m, 6H). MS (ESI⁺): [M + H]⁺ 420.3. |
| 206 | Orange solid. ¹H NMR (400 MHz, DMSO-d₆, 300K) δ_{H} 8.41 (s, |
| | 1H), 8.35 (d, J = 8.9 Hz, 1H), 7.99 (s, 1H), 7.65 (d, J = 8.9 Hz, |
| | 1H), 6.96 (s, 1H, NH, D₂O exchanged), 6.59 (s, 1H), 4.06 (s, 3H), |
| | 3.08 (s, 3H), 2.49 − 2.37 (m, 4H), 2.26 − 2.10 (m, 4H), 1.91 (s, |
| | 4H), 1.62 (s, 2H). 19F NMR (376 MHz, DMSO-d₆, 300K) |
| | δ_{F} −128.73. MS (ESI⁺): [M + H]⁺ 408.2. |
| 207 | Yellow solid. ¹H NMR (400 MHz, DMSO-d₆, 300K) δ_{H} 8.45 − |
| | 8.37 (m, 1H + NH, D₂O exchanged), 8.13 (dd, J = 9.0, 1.5 Hz, |
| | 1H), 7.94 (s, 1H), 7.85 − 7.65 (m, 3H), 7.56 − 7.45 (m, 2H), 6.57 |
| | (s, 1H), 4.87 (d, J = 5.2 Hz, 2H), 4.02 (s, 3H), 3.17 (s, 3H). 19F |
| | NMR (376 MHz, DMSO-d₆, 300K) δ_{F} −58.59. MS (ESI⁺): |
| | [M + H]⁺ 414.2. |
| 208 | Yellow solid. ¹H NMR (400 MHz, DMSO-d₆, 300K) δ_{H} 8.38 (s, |
| | 1H), 8.26 (dd, J = 8.9, 1.5 Hz, 1H), 8.06 − 7.98 (m, 1H + NH, |
| | D₂O exchanged), 7.59 (d, J = 8.9 Hz, 1H), 7.35 − 7.18 (m, 4H), |
| | 6.61 (s, 1H), 5.54 (d, J = 5.2 Hz, 1H, OH, D₂O exchanged), 5.45 |
| | (t, J = 4.6 Hz, 1H), 4.62 − 4.49 (m, 1H), 4.01 (s, 3H), 3.25 (dd, J = |
| | 15.6, 7.5 Hz, 1H), 3.14 (s, 3H), 2.85 (dd, J = 15.5, 7.8 Hz, 1H). |
| | MS (ESI⁺): [M + H]⁺ 388.2. |
| 209 | Yellow solid. ¹H NMR (400 MHz, DMSO-d₆, 300K) δ_{H} 8.43 (s, |
| | 1H), 8.19 (dd, J = 8.9, 1.4 Hz, 1H), 8.08 (s, 1H), 7.97 (d, J = 7.8 |
| | Hz, 1H, NH, D₂O exchanged), 7.61 (d, J = 8.8 Hz, 1H), 7.55 − |
| | 7.50 (m, 2H), 7.42 − 7.34 (m, 2H), 7.29 − 7.22 (m, 1H), 6.53 (s, |
| | 1H), 5.28 − 5.16 (m, 1H), 5.08 (br s, 1H, OH, D₂O exchanged), |
| | 4.05 (s, 3H), 3.90 − 3.69 (m, 2H), 3.16 (s, 3H). MS (ESI⁺): |
| | [M + H]⁺ 376.2. |
| 210 | Yellow solid. ¹H NMR (400 MHz, DMSO-d₆, 300K) δ_{H} 8.43 (s, |
| | 1H), 8.19 (dd, J = 8.9, 1.4 Hz, 1H), 8.08 (s, 1H), 7.97 (d, J = 7.8 |
| | Hz, 1H, NH, D₂O exchanged), 7.61 (d, J = 8.8 Hz, 1H), 7.55 − |
| | 7.50 (m, 2H), 7.42 − 7.34 (m, 2H), 7.29 − 7.22 (m, 1H), 6.53 (s, |
| | 1H), 5.28 − 5.16 (m, 1H), 5.08 (br s, 1H, OH, D₂O exchanged), |
| | 4.05 (s, 3H), 3.90 − 3.69 (m, 2H), 3.16 (s, 3H). MS (ESI⁺): |
| | [M + H]⁺ 376.2. |
| 211 | Yellow solid. ¹H NMR (400 MHz, DMSO-d₆, 300K) δ_{H} 8.43 (s, |
| | 1H), 8.20 (d, J = 8.9 Hz, 1H), 8.14 − 8.02 (m, 1H + NH, D₂O |
| | exchanged), 7.61 (d, J = 8.8 Hz, 1H), 7.54 (d, J = 7.6 Hz, 2H), |
| | 7.38 (t, J = 7.5 Hz, 2H), 7.29 − 7.23 (m, 1H), 6.54 (s, 1H), 5.46 |
| | 5.34 (m, 1H), 4.04 (s, 3H), 3.86 − 3.79 (m, 1H), 3.65 (dd, J= 10.0, |
| | 5.3 Hz, 1H), 3.35 (s, 3H), 3.14 (s, 3H). MS (ESI⁺): [M + H]⁺ |
| | 390.1. |
| 212 | Yellow solid. ¹H NMR (400 MHz, DMSO-d₆, 300K) δ_{H} 8.52 (s, |
| | 1H), 8.30 (dd, J = 8.9, 1.5 Hz, 1H), 8.04 (s, 1H), 7.97 (t, J = 5.7 |
| | Hz, 1H, NH, D₂O exchanged), 7.64 (d, J = 8.8 Hz, 1H), 7.53 − |
| | 7.48 (m, 2H), 7.48 − 7.41 (m, 2H), 7.35 − 7.28 (m, 1H), 6.59 (s, |
| | 1H), 5.69 (d, J = 4.2 Hz, 1H, OH, D₂O exchanged), 5.13 − 5.05 |
| | (m, 1H), 4.06 (s, 3H), 3.78 − 3.67 (m, 1H), 3.42 − 3.31 (m, 1H), |
| | 3.09 (s, 3H). MS (ESI⁺): [M + H]⁺ 376.2. |
| 213 | Yellow solid. ¹H NMR (400 MHz, D₂O, 300K) δ_{H} 7.68 − 7.50 (m, |
| | 6H), 7.37 (s, 1H), 7.29 (d, J = 9.2 Hz, 1H), 7.12 (d, J = 8.8 Hz, |
| | 1H), 6.21 (s, 1H), 5.27 (dd, J = 8.8, 5.9 Hz, 1H), 3.81 (s, 3H), 3.63 |
| | (dd, J = 13.4, 8.8 Hz, 1H), 3.54 (dd, J = 13.4, 6.0 Hz, 1H), 3.03 |
| | (s, 3H). MS (ESI⁺): [M + H]⁺ 375.3 (+2HCl). |
| 214 | Yellow solid. ¹H NMR (400 MHz, DMSO-d₆, 300K) δ_{H} 8.57 (t, J = |
| | 5.9 Hz, 1H, NH, D₂O exchanged), 8.48 (s, 1H), 8.28 (dd, J = |
| | 8.9, 1.6 Hz, 1H), 8.05 (s, 1H), 7.61 (d, J = 8.8 Hz, 1H), 7.21 − |
| | 7.19 (m, 1H), 6.64 (s, 1H), 4.90 (d, J = 5.9 Hz, 2H), 4.04 (s, 3H), |
| | 3.10 (s, 3H), 2.37 (d, J = 1.0 Hz, 3H). MS (ESI⁺): [M + H]⁺ 367.1. |
| 215 | Yellow solid. ¹H NMR (400 MHz, DMSO-d₆, 300K) δ_{H} 8.42 (s, |
| | 1H), 8.30 (dd, J = 9.0, 1.6 Hz, 1H), 8.05 (d, J = 0.9 Hz, 1H), 7.70 − |
| | 7.60 (m, 1H + NH, D₂O exchanged), 6.55 (s, 1H), 4.04 (s, 3H), |
| | 3.94 − 3.86 (m, 2H), 3.38 (t, J = 6.3 Hz, 2H), 3.35 − 3.27 (m, 2H), |
| | 3.08 (s, 3H), 2.07 − 1.93 (m, 1H), 1.76 − 1.66 (m, 2H), 1.39 − 1.23 |
| | (m, 2H). MS (ESI⁺): [M + H]⁺ 354.2. |
| 216 | Yellow solid. ¹H NMR (400 MHz, DMSO-d₆, 300K) δ_{H} 9.22 (s, |
| | 1H, NH, D₂O exchanged), 8.49 (s, 1H), 8.31 (d, J = 8.9 Hz, 1H), |
| | 8.14 (s, 1H), 7.85 (d, J = 9.0 Hz, 2H), 7.67 (d, J = 8.9 Hz, 1H), |
| | 7.06 (d, J = 9.1 Hz, 2H), 6.72 (s, 1H), 4.06 (s, 3H), 3.23 (s, 3H), |
| | 3.20 − 3.14 (m, 4H), 2.51 − 2.47 (m, 4H), 2.25 (s, 3H). MS (ESI⁺): |
| | [M + H]⁺ 430.1. |
| 217 | Yellow solid. ¹H NMR (400 MHz, CDCl₃, 300K) δ_{H} 12.45 (br s, |
| | 1H, NH, D₂O exchanged), 8.38 (dd, J = 5.0, 2.0 Hz, 1H), 8.07 (s, |
| | 1H), 7.94 (s, 1H), 7.70 (td, J = 8.2, 2.0 Hz, 1H), 7.58 (dd, J = 8.7, |
| | 1.6 Hz, 1H), 7.51 (d, J = 8.8 Hz, 1H), 7.23 (d, J = 8.2 Hz, 1H), |
| | 7.00 (ddd, J = 7.2, 5.0, 1.1 Hz, 1H), 6.82 (s, 1H), 4.15 (s, 3H), |
| | 3.34 (s, 3H). MS (ESI⁺): [M + H]⁺ 333.1. |
| 218 | Beige solid. ¹H NMR (400 MHz, DMSO-d₆, 300K) δ_{H} 8.37 (s, |
| | 1H), 8.31 (d, J = 8.9 Hz, 1H), 8.07 (s, 1H), 7.65 (d, J = 8.9 Hz, |
| | 1H), 7.36 (d, J = 7.4 Hz, 1H, NH, D₂O exchanged), 6.58 (s, 1H), |
| | 4.15 − 3.95 (m, 5H), 3.87 − 3.77 (m, 1H), 3.42 − 3.22 (m, 2H), |
| | 3.08 (s, 3H), 2.14 − 2.00 (m, 1H), 1.84 − 1.58 (m, 3H). MS (ESI⁺): |
| | [M + H]⁺ 340.2. |
| 219 | Yellow solid. ¹H NMR (400 MHz, DMSO-d₆, 300K) δ_{H} 8.36 (s, |
| | 1H), 8.32 (dd, J = 8.8, 1.4 Hz, 1H), 8.07 (d, J = 0.9 Hz, 1H), 7.66 |
| | (d, J= 8.8 Hz, 1H), 7.38 (d, J = 7.8 Hz, 1H, NH, D₂O exchanged), |
| | 6.58 (s, 1H), 5.10 (d, J = 4.7 Hz, 1H, OH, D₂O exchanged), 4.04 |
| | (s, 3H), 3.99 (dd, J = 10.9, 4.8 Hz, 1H), 3.95 − 3.84 (m, 2H), 3.83 |
| | 3.72 (m, 1H), 3.38 (td, J = 11.7; 2.3 Hz, 1H), 3.20 (t, J = 10.4 |
| | Hz, 1H), 3.10 (s, 3H), 2.02 − 1.92 (m, 1H), 1.65 − 1.51 (m, 1H). |
| | MS (ESI⁺): [M + H]⁺ 356.2. |
| 220 | Yellow solid. ¹H NMR (400 MHz, DMSO-d₆, 300K) δ_{H} 12.54 (br |
| | s, 1H, NH, D₂O exchanged), 8.47 (s, 1H), 8.23 (s, 1H), 7.85 (d, J = |
| | 8.4 Hz, 1H), 7.54 (d, J = 8.4 Hz, 1H), 7.34 (d, J = 7.8 Hz, 1H, |
| | NH, D₂O exchanged), 6.53 (s, 1H), 3.93 (br s, 1H), 3.07 (s, 3H), |
| | 2.10 − 2.95 (m, 2H), 1.90 − 1.75 (m, 2H), 1.72 − 1.64 (m, 1H), |
| | 1.50 − 1.34 (m, 4H), 1.30 − 1.10 (m, 1H). MS (ESI⁺): [M + H]⁺ |
| | 324.2. |
| 221 | Yellow solid. ¹H NMR (400 MHz, DMSO-d₆, 300K, mixture of |
| | tautomers) δ_{H} 12.52 & 12.44 (br s, 1H, NH, D₂O exchanged), 8.52 |
| | & 8.42 (s, 1H), 8.22 & 8.20 (s, 1H), 7.92 & 7.82 (d, J = 8.5 Hz, |
| | 1H), 7.58 & 7.48 (d, J = 8.4 Hz, 1H), 7.37 & 7.33 (d, J = 7.6 Hz, |
| | 1H, NH, D₂O exchanged), 6.53 (s, 1H), 4.20 − 4.07 (m, 1H), 3.07 |
| | (s, 3H), 2.12 − 1.95 (m, 2H), 1.82 − 1.45 (m, 10H). MS (ESI⁺): |
| | [M + H]⁺ 338.2. |
| 222 | Yellow solid. ¹H NMR (400 MHz, DMSO-d₆, 300K, mixture of |
| | tautomers) δ_{H} 12.49 & 12.43 (br s, 1H, NH, D₂O exchanged), 8.53 |
| | & 8.33 (s, 1H), 8.22 & 8.20 (s, 1H), 8.00 − 7.88 (m, 1H), 7.58 & |
| | 7.47 (d, J = 8.5 Hz, 1H), 7.40 − 7.27 (m, 1H, NH, D₂O |
| | exchanged), 6.54 (s, 1H), 4.27 − 4.14 (m, 1H), 3.07 (s, 3H), 2.00 − |
| | 1.45 (m, 14H). MS (ESI⁺): [M + H]⁺ 352.2. |
| 223 | Yellow solid. ¹H NMR (400 MHz, DMSO-d₆, 300K, mixture of |
| | tautomers) δ_{H} 12.51 & 12.43 (br s, 1H, NH, D₂O exchanged), 8.49 |
| | & 8.34 (s, 1H), 8.20 (s, 1H), 7.93 & 7.88 (d, J = 8.4 Hz, 1H), 7.58 |
| | & 7.46 (d, J = 8.4 Hz, 1H), 7.23 & 7.19 (d, J = 7.1 Hz, 1H, NH, |
| | D₂O exchanged), 6.53 (s, 1H), 4.83 (t, J = 5.7 Hz, 1H, OH, D₂O |
| | exchanged), 4.25 (br s, 1H), 3.60 − 3.48 (m, 2H), 3.09 (s, 3H), |
| | 1.80 − 1.65 (m, 1H), 1.64 − 1.37 (m, 2H), 1.01 − 0.92 (m, 6H). |
| | MS (ESI⁺): [M + H]⁺ 342.2. |
| 224 | Yellow solid. ¹H NMR (400 MHz, DMSO-d₆, 300K, mixture of |
| | tautomers) δ_{H} 12.52 & 12.43 (br s, 1H, NH, D₂O exchanged), 8.51 |
| | & 8.37 (s, 1H), 8.20 (s, 1H), 7.92 & 7.86 (d, J = 8.5 Hz, 1H), 7.58 |
| | & 7.46 (d, J = 8.4 Hz, 1H), 7.41 − 7.28 (m, 1H, NH, D₂O |
| | exchanged), 6.54 (s, 1H), 4.44 (br s, 1H), 3.51 (dd, J = 9.7, 6.3 |
| | Hz, 1H), 3.43 (dd, J = 9.7, 5.8 Hz, 1H), 3.33 (s, 3H), 3.08 (s, |
| | 3H), 1.80 − 1.66 (m, 1H), 1.64 − 1.54 (m, 1H), 1.48 − 1.38 (m, |
| | 1H), 1.01 − 0.92 (m, 6H). MS (ESI⁺): [M + H]⁺ 356.2. |
| 225 | Beige solid. ¹H NMR (400 MHz, DMSO-d₆, 300K, mixture of |
| | tautomers) δ_{H} 12.51 & 12.44 (br s, 1H, NH, D₂O exchanged), 8.53 |
| | & 8.39 (s, 1H), 8.21 (s, 1H), 7.94 & 7.86 (d, J = 8.4 Hz, 1H), 7.64 − |
| | 7.36 (m, 1H + NH, D₂O exchanged), 6.57 (s, 1H), 4.42 − 4.27 |
| | (m, 1H), 3.92 − 3.77 (m, 1H), 3.40 & 3.38 (s, 3H), 3.08 (s, 3H), |
| | 2.22 − 2.06 (m, 1H), 2.00 − 1.86 (m, 1H), 1.84 − 1.56 (m, 4H). |
| | MS (ESI⁺): [M + H]⁺ 340.2. |
| 226 | Beige solid. ¹H NMR (400 MHz, DMSO-d₆, 300K, mixture of |
| | tautomers) δ_{H} 12.51 & 12.44 (br s, 1H, NH, D₂O exchanged), 8.53 |
| | & 8.39 (s, 1H), 8.21 (s, 1H), 7.94 & 7.86 (d, J = 8.4 Hz, 1H), 7.64 − |
| | 7.36 (m, 1H + NH, D₂O exchanged), 6.57 (s, 1H), 4.42 − 4.27 |
| | (m, 1H), 3.92 − 3.77 (m, 1H), 3.40 & 3.38 (s, 3H), 3.08 (s, 3H), |
| | 2.22 − 2.06 (m, 1H), 2.00 − 1.86 (m, 1H), 1.84 − 1.56 (m, 4H). |
| | MS (ESI⁺): [M + H]⁺ 340.2. |
| 227 | Pale yellow solid. ¹H NMR (400 MHz, DMSO-d6, 300K, mixture |
| | of tautomers) δ_{H} 12.48 & 12.44 (br s, 1H, NH, D₂O exchanged), |
| | 8.50 & 8.30 (s, 1H), 8.20 (s, 1H), 7.93 (s, 1H), 7.56 & 7.45 (s, |
| | 1H), 7.33 & 7.28 (s, 1H, NH, D₂O exchanged), 6.55 (s, 1H), 3.09 |
| | (s, 3H), 2.76 (br s, 1H), 2.34 (br s, 4H), 2.28 − 1.98 (m, 4H), 1.74 − |
| | 1.52 (m, 4H). MS (ESI⁺): [M + H]+ 362.3. |
| 228 | Pale yellow solid. ¹H NMR (400 MHz, DMSO-d₆, 300K, mixture |
| | of tautomers) δ_{H} 12.52 & 12.43 (br s, 1H, NH, D₂O exchanged), |
| | 8.24 (s, 1H), 8.23 & 8.20 (s, 1H), 7.96 & 7.87 (d, J = 8.5 Hz, 1H), |
| | 7.59 & 7.47 (d, J = 8.5 Hz, 1H), 6.66 & 6.64 (s, 1H, NH, D₂O |
| | exchanged), 6.55 (s, 1H), 3.07 (s, 3H), 2.32 − 2.22 (m, 6H), 2.15 |
| | (s, 3H), 1.84 − 1.66 (s, 6H). MS (ESI⁺): [M + H]⁺ 376.2. |
| 229 | Pale yellow solid. ¹H NMR (400 MHz, DMSO-d₆, 300K, mixture |
| | of tautomers) on 12.43 & 12.37 (br s, 1H, NH, D₂O exchanged), |
| | 8.48 & 8.16 (s, 1H), 8.17 & 8.14 (s, 1H), 7.91 & 7.87 (d, J = 8.5 |
| | Hz, 1H), 7.52 & 7.39 (d, J = 8.5 Hz, 1H), 6.68 & 6.67 (s, 1H, NH, |
| | D₂O exchanged), 6.49 (s, 1H), 4.54 & 4.52 (s, 1H, OH, D₂O |
| | exchanged), 3.00 (s, 3H), 2.25 − 2.11 (m, 4H), 2.07 − 1.98 (m, |
| | 4H), 1.67 − 1.41 (s, 6H). MS (ESI⁺): [M + H]⁺ 392.3. |
| 230 | Orange solid. ¹H NMR (400 MHz, DMSO-d₆, 300K, mixture of |
| | tautomers) δ_{H} 12.51 & 12.42 (s, 1H, NH, D₂O exchanged), 8.63 |
| | & 8.20 (s, 1H), 8.23 & 8.18 (s, 1H), 8.04 & 7.88 (d, J = 8.5 Hz, |
| | 1H), 7.58 & 7.46 (d, J = 8.5 Hz, 1H), 6.82 & 6.80 (s, 1H, NH, |
| | D₂O exchanged), 6.56 (s, 1H), 4.19 & 4.17 (s, 3H), 3.07 (s, 3H), |
| | 2.38 − 2.10 (m, 8H), 1.86 − 1.51 (m, 6H). MS (ESI⁺): [M + H]⁺ |
| | 406.3. |
| 231 | Orange solid. ¹H NMR (400 MHz, DMSO-d₆, 300K, mixture of |
| | tautomers) δ_{H} 12.55 & 12.44 (br s, 1H, NH, D₂O exchanged), 8.61 |
| | & 8.22 (s, 1H), 8.24 & 8.21 (s, 1H), 7.96 & 7.86 (d, J = 8.6 Hz, |
| | 1H), 7.60 & 7.48 (d, J = 8.5 Hz, 1H), 6.91 & 6.89 (s, 1H, NH, |
| | D₂O exchanged), 6.58 (s, 1H), 3.08 (s, 3H), 2.50 − 2.36 (m, 4H), |
| | 2.30 − 2.12 (m, 4H), 2.00 − 1.82 (m, 4H), 1.72 − 1.54 (m, 2H). 19F |
| | NMR (376 MHz, DMSO-d₆, 300K, mixture of tautomers) 8F − |
| | 128.73. MS (ESI⁺): [M + H]⁺ 394.3. |
| 232 | Yellow solid. ¹H NMR (400 MHz, DMSO-d₆, 300K, mixture of |
| | tautomers) δ_{H} 12.43 & 12.41 (br s, 1H, NH, D₂O exchanged), 8.39 − |
| | 8.16 (m, 2H ⁺ NH, D₂O exchanged), 7.98 − 7.87 (m, 1H), 7.81 − |
| | 7.65 (m, 3H), 7.57 − 7.37 (m, 2H), 6.60 & 6.59 (s, 1H), 4.89 (br |
| | s, 2H), 3.17 & 3.16 (s, 3H). 19F NMR (376 MHz, DMSO-d₆, |
| | 300K, mixture of tautomers) δ_{H} −58.51 & −58.68. MS (ESI⁺): |
| | [M + H]⁺ 400.1. |
| 233 | Yellow solid. ¹H NMR (400 MHz, DMSO-d₆, 300K, mixture of |
| | tautomers) δ_{H} 12.49 & 12.44 (s, 1H, NH, D₂O exchanged), 8.46 |
| | & 8.41 (s, 1H), 8.18 (s, 1H), 8.05 − 7.78 (m, 1H + NH, D₂O |
| | exchanged), 7.58 & 7.47 (d, J = 8.5 Hz, 1H), 7.35 − 7.15 (m, 4H), |
| | 6.62 (s, 1H), 5.62 − 5.43 (m, 1H + OH, D₂O exchanged), 4.60 − |
| | 4.48 (m, 1H), 3.25 (dd, J= 15.5, 7.5 Hz, 1H), 3.14 (s, 3H), 2.92 |
| | 2.76 (m, 1H). MS (ESI⁺): [M + H]⁺ 374.2. |
| 234 | Yellow solid. ¹H NMR (400 MHz, DMSO-d₆, 300K, mixture of |
| | tautomers) δ_{H} 12.56 & 12.45 (s, 1H, NH, D₂O exchanged), 8.47 |
| | & 8.46 (s, 1H), 8.24 & 8.22 (s, 1H), 8.00 − 7.72 (m, 1H + NH, |
| | D₂O exchanged), 7.62 − 7.45 (m, 3H), 7.41 − 7.37 (m, 2H), 7.28 − |
| | 7.21 (m, 1H), 6.54 & 6.53 (s, 1H), 5.32 − 5.20 (m, 1H), 5.11 |
| | 5.02 (m, 1H, OH, D₂O exchanged), 3.92 − 3.81 (m, 1H), 3.80 |
| | 3.70 (m, 1H), 3.16 (s, 3H). MS (ESI⁺): [M + H]⁺ 362.2. |
| 235 | Yellow solid. ¹H NMR (400 MHz, DMSO-d₆, 300K, mixture of |
| | tautomers) δ_{H} 12.56 & 12.45 (s, 1H, NH, D₂O exchanged), 8.47 |
| | & 8.46 (s, 1H), 8.24 & 8.22 (s, 1H), 8.00 − 7.72 (m, 1H + NH, |
| | D₂O exchanged), 7.62 − 7.45 (m, 3H), 7.41 − 7.37 (m, 2H), 7.28 |
| | 7.21 (m, 1H), 6.54 & 6.53 (s, 1H), 5.32 − 5.20 (m, 1H), 5.11 − |
| | 5.02 (m, 1H, OH, D₂O exchanged), 3.92 − 3.81 (m, 1H), 3.80 − |
| | 3.70 (m, 1H), 3.16 (s, 3H). MS (ESI⁺): [M + H]⁺ 362.2. |
| 236 | Yellow solid. ¹H NMR (400 MHz, DMSO-d₆, 300K, mixture of |
| | tautomers) δ_{H} 12.55 & 12.45 (br s, 1H, NH, D₂O exchanged), 8.52 − |
| | 8.40 (m, 1H), 8.28 − 8.18 (m, 1H), 8.15 − 7.70 (m, 1H + NH, |
| | D₂O exchanged), 7.65 − 7.43 (m, 3H), 7.40 − 7.34 (m, 2H), 7.31 − |
| | 7.24 (m, 1H), 6.55 (s, 1H), 5.45 (s, 1H), 3.84 (dd, J = 10.0, 8.5 |
| | Hz, 1H), 3.68 (dd, J = 10.0, 5.5 Hz, 1H), 3.36 (s, 3H), 3.14 (s, |
| | 3H). MS (ESI⁺): [M + H]⁺ 376.2. |
| 237 | Yellow solid. ¹H NMR (400 MHz, DMSO-d₆, 300K, mixture of |
| | tautomers) δ_{H} 12.53 & 12.46 (br s, 1H, NH, D₂O exchanged), 8.61 |
| | & 8.26 (s, 1H), 8.23 (s, 1H), 8.05 − 7.75 (m, 1H + NH, D₂O |
| | exchanged), 7.70 − 7.25 (m, 6H), 6.59 (s, 1H), 5.69 (d, J = 4.2 Hz, |
| | 1H, OH, D₂O exchanged), 5.15 − 4.95 (m, 1H), 3.88 − 3.65 (m, |
| | 1H), 3.50 − 3.25 (m, 1H), 3.09 (s, 3H). MS (ESI⁺): [M + H]⁺ 362.3. |
| 238 | Yellow solid. ¹H NMR (400 MHz, DMSO-d₆, 300K, mixture of |
| | tautomers) δ_{H} 12.52 & 12.46 (br s, 1H, NH, D₂O exchanged), 8.65 − |
| | 8.40 (m, 1H + NH, D₂O exchanged), 8.22 & 8.21 (s, 1H), 8.00 |
| | & 7.86 (d, J = 8.4 Hz, 1H), 7.59 & 7.48 (d, J = 8.4 Hz, 1H), 7.19 |
| | (s, 1H), 6.64 (s, 1H), 4.98 − 4.86 (m, 2H), 3.11 (s, 3H), 2.37 (s, |
| | 3H). MS (ESI⁺): [M + H]⁺ 353.1. |
| 239 | Yellow solid. ¹H NMR (400 MHz, DMSO-d₆, 300K, mixture of |
| | tautomers) δ_{H} 12.53 & 12.45 (br s, 1H, NH, D₂O exchanged), 8.51 |
| | 8.46 (s, 1H), 8.23 & 8.20 (s, 1H), 7.94 & 7.80 (d, J = 8.4 Hz, |
| | 1H), 7.70 − 7.45 (m, 1H + NH, D₂O exchanged), 6.55 (s, 1H), |
| | 3.94 − 3.80 (m, 2H), 3.43 − 3.36 (m, 2H), 3.35 − 3.26 (m, 2H), |
| | 3.08 (s, 3H), 2.05 − 1.89 (m, 1H), 1.77 − 1.65 (m, 2H), 1.38 − 1.21 |
| | (m, 2H). MS (ESI⁺): [M + H]⁺ 340.2. |
| 240 | Yellow solid. ¹H NMR (400 MHz, DMSO-d₆, 300K, mixture of |
| | tautomers) δ_{H} 12.55 & 12.44 (br s, 1H, NH, D₂O exchanged), 9.14 |
| | & 9.11 (s, 1H, NH, D₂O exchanged), 8.42 & 8.40 (s, 1H), 8.21 & |
| | 8.16 (s, 1H), 8.00 − 7.68 (m, 3H), 7.57 & 7.46 (d, J = 8.4 Hz, 1H), |
| | 7.00 & 6.94 (d, J = 8.5 Hz, 2H), 6.64 (s, 1H), 3.16 (s, 3H), 3.08 |
| | (br s, 4H), 2.42 (br s, 4H), 2.17 (s, 3H). MS (ESI⁺): [M + H]⁺ |
| | 416.2. |
| 241 | Yellow solid. ¹H NMR (400 MHz, CF₃COOD, 300K) δ_{H} 9.34 (s, |
| | 1H), 8.61 − 8.49 (m, 2H), 8.28 (s, 1H), 8.13 (d, J = 8.7 Hz, 1H), |
| | 8.03 (d, J = 8.8 Hz, 1H), 7.93 (d, J = 8.6 Hz, 1H), 7.71 (t, J = 6.8 |
| | Hz, 1H), 7.52 (s, 1H), 3.63 (s, 3H). MS (ESI⁺): [M + H]⁺ 319.1. |
| 242 | Yellow solid. ¹H NMR (400 MHz, DMSO-d₆, 300K, mixture of |
| | tautomers) on 12.58 & 12.48 (s, 1H, NH, D₂O exchanged), 8.49 |
| | & 8.39 (s, 1H), 8.22 (s, 1H), 7.93 & 7.82 (d, J = 8.5 Hz, 1H), 7.61 |
| | & 7.51 (d, J = 8.5 Hz, 1H), 7.41 − 7.28 (m, 1H, NH, D₂O |
| | exchanged), 6.58 (s, 1H), 4.20 − 3.96 (m, 2H), 3.89 − 3.78 (m, |
| | 1H), 3.42 − 3.22 (m, 2H), 3.08 (s, 3H), 2.14 − 2.02 (m, 1H), 1.82 − |
| | 1.60 (m, 3H). MS (ESI⁺): [M + H]⁺ 326.2. |
| 243 | Beige solid. ¹H NMR (400 MHz, DMSO-d₆, 300K, mixture of |
| | tautomers) δ_{H} 12.60 & 12.47 (s, 1H, NH, D₂O exchanged), 8.49 |
| | & 8.37 (s, 1H), 8.21 (s, 1H), 7.92 & 7.82 (d, J = 8.4 Hz, 1H), 7.61 |
| | & 7.51 (d, J = 8.4 Hz, 1H), 7.38 − 7.30 (m, 1H, NH, D₂O |
| | exchanged), 6.57 (s, 1H), 5.14 − 5.08 (m, 1H, OH, D₂O |
| | exchanged), 4.05 − 3.85 (m, 3H), 3.82 − 3.70 (m, 1H), 3.38 (t, J = |
| | 11.8 Hz, 1H), 3.18 (t, J = 10.4 Hz, 1H), 3.10 (s, 3H), 2.05 − 1.93 |
| | (m, 1H), 1.67 − 1.50 (m, 1H). MS (ESI⁺): [M + H]⁺ 342.2. |
| 244 | Yellow solid. ¹H NMR (400 MHz, DMSO-d₆, 300K) δ_{H} 8.61 (s, |
| | 1H), 8.19 (s, 1H), 7.76 (dd, J = 8.5, 1.5 Hz, 1H), 7.59 (d, J = 8.4 |
| | Hz, 1H), 7.43 (d, J = 7.4 Hz, 1H, NH, D₂O exchanged), 6.55 (s, |
| | 1H), 4.00 − 3.88 (m, 1H), 3.84 (s, 3H), 3.08 (s, 3H), 2.16 − 2.03 |
| | (m, 2H), 1.87 − 1.75 (m, 2H), 1.72 − 1.62 (m, 1H)), 1.50 − 1.30 |
| | (m, 4H), 1.27 − 1.10 (m, 1H). MS (ESI⁺): [M + H]⁺ 338.3. |
| 245 | Yellow solid. ¹H NMR (400 MHz, DMSO-d₆, 300K) δ_{H} 8.60 (s, |
| | 1H), 8.19 (s, 1H), 7.73 (dd, J = 8.4, 1.4 Hz, 1H), 7.58 (d, J = 8.4 |
| | Hz, 1H), 7.46 (d, J = 7.6 Hz, 1H, NH, D₂O exchanged), 6.55 (s, |
| | 1H), 4.16 − 4.04 (m, 1H), 3.84 (s, 3H), 3.07 (s, 3H), 2.12 − 2.00 |
| | (m, 2H), 1.80 − 1.48 (m, 10H). MS (ESI⁺): [M + H]⁺ 352.3. |
| 246 | Orange solid. ¹H NMR (400 MHz, DMSO-d₆, 300K) δ_{H} 8.63 (s, |
| | 1H), 8.19 (s, 1H), 7.76 (dd, J = 8.5, 1.5 Hz, 1H), 7.58 (d, J = 8.4 |
| | Hz, 1H), 7.46 (d, J = 7.7 Hz, 1H, NH, D₂O exchanged), 6.55 (s, |
| | 1H), 4.33 − 4.19 (m, 1H), 3.83 (s, 3H), 3.08 (s, 3H), 1.97 − 1.50 |
| | (m, 14H). MS (ESI⁺): [M + H]⁺ 366.3. |
| 247 | Yellow solid. ¹H NMR (400 MHz, DMSO-d₆, 300K) δ_{H} 8.47 (s, |
| | 1H), 8.19 (s, 1H), 7.85 (d, J = 8.5 Hz, 1H), 7.58 (d, J = 8.5 Hz, |
| | 1H), 7.31 (d, J = 8.6 Hz, 1H, NH, D₂O exchanged), 6.54 (s, 1H), |
| | 4.84 (t, J = 5.7 Hz, 1H, OH, D₂O exchanged), 4.31 − 4.20 (m, |
| | 1H), 3.82 (s, 3H), 3.60 − 3.48 (m, 2H), 3.09 (s, 3H), 1.77 − 1.67 |
| | (m, 1H), 1.61 − 1.51 (m, 1H), 1.51 − 1.41 (m, 1H), 1.01 (d, J = |
| | 6.5 Hz, 3H), 0.96 (d, J = 6.6 Hz, 3H). MS (ESI⁺): [M + H]⁺ 356.3. |
| 248 | Yellow solid. ¹H NMR (400 MHz, DMSO-d₆, 300K) δ_{H} 8.48 (s, |
| | 1H), 8.19 (s, 1H), 7.83 (d, J = 8.5 Hz, 1H), 7.58 (d, J = 8.7 Hz, |
| | 1H), 7.44 (d, J = 8.6 Hz, 1H, NH, D₂O exchanged), 6.56 (s, 1H), |
| | 4.51 − 4.40 (m, 1H), 3.81 (s, 3H), 3.52 (dd, J = 9.9, 6.5 Hz, 1H), |
| | 3.44 (dd, J = 9.9, 4.8 Hz, 1H), 3.32 (s, 3H), 3.09 (s, 3H), 1.78 |
| | 1.66 (m, 1H), 1.63 − 1.53 (m, 1H), 1.47 − 1.38 (m, 1H), 0.99 (d, |
| | J = 6.5 Hz, 3H), 0.95 (d, J = 6.6 Hz, 3H). MS (ESI⁺): [M + H]⁺ |
| | 370.3. |
| 249 | Beige solid. ¹H NMR (400 MHz, DMSO-d₆, 300K) δ_{H} 8.40 (s, |
| | 1H), 8.20 (s, 1H), 7.94 (dd, J = 8.5, 1.5 Hz, 1H), 7.59 (d, J = 8.5 |
| | Hz, 1H), 7.49 (d, J = 7.3 Hz, 1H, NH, D₂O exchanged), 6.59 (s, |
| | 1H), 4.40 − 4.27 (m, 1H), 3.89 − 3.83 (m, 1H), 3.82 (s, 3H), 3.37 |
| | (s, 3H), 3.08 (s, 3H), 2.22 − 2.07 (m, 1H), 2.00 − 1.86 (m, 1H), |
| | 1.84 − 1.56 (m, 4H). MS (ESI⁺): [M + H]⁺ 354.3. |
| 250 | Beige solid. ¹H NMR (400 MHz, DMSO-d₆, 300K) δ_{H} 8.40 (s, |
| | 1H), 8.20 (s, 1H), 7.94 (dd, J = 8.5, 1.5 Hz, 1H), 7.59 (d, J = 8.5 |
| | Hz, 1H), 7.49 (d, J = 7.3 Hz, 1H, NH, D₂O exchanged), 6.59 (s, |
| | 1H), 4.40 − 4.27 (m, 1H), 3.89 − 3.83 (m, 1H), 3.82 (s, 3H), 3.37 |
| | (s, 3H), 3.08 (s, 3H), 2.22 − 2.07 (m, 1H), 2.00 − 1.86 (m, 1H), |
| | 1.84 − 1.56 (m, 4H). MS (ESI⁺): [M + H]⁺ 354.3. |
| 251 | Yellow solid. ¹H NMR (400 MHz, DMSO-d₆, 300K) δ_{H} 8.54 (s, |
| | 1H), 8.19 (s, 1H), 7.71 (d, J = 8.4 Hz, 1H), 7.57 (d, J = 8.4 Hz, |
| | 1H), 7.37 (s, 1H, NH, D₂O exchanged), 6.56 (s, 1H), 3.82 (s, 3H), |
| | 3.10 (s, 3H), 2.79 − 2.69 (m, 1H), 2.46 − 2.29 (m, 4H), 2.26 − 2.00 |
| | (m, 4H), 1.74 − 1.52 (m, 4H). MS (ESI⁺): [M + H]⁺ 376.2. |
| 252 | Pale yellow solid. ¹H NMR (400 MHz, DMSO-d₆, 300K) δ_{H} 8.55 |
| | (s, 1H), 8.19 (s, 1H), 7.73 (d, J = 8.4 Hz, 1H), 7.58 (d, J = 8.4 Hz, |
| | 1H), 6.68 (s, 1H, NH, D₂O exchanged), 6.56 (s, 1H), 3.83 (s, 3H), |
| | 3.08 (s, 3H), 2.29 (s, 6H), 2.14 (s, 3H), 1.73 (s, 6H). MS (ESI⁺): |
| | [M + H]⁺ 390.2. |
| 253 | Yellow solid. ¹H NMR (400 MHz, DMSO-d₆, 300K) δ_{H} 8.58 (s, |
| | 1H), 8.20 (s, 1H), 7.69 (d, J = 8.5 Hz, 1H), 7.59 (d, J = 8.4 Hz, |
| | 1H), 6.76 (s, 1H, NH, D₂O exchanged), 6.57 (s, 1H), 4.61 (s, 1H, |
| | OH, D₂O exchanged), 3.86 (s, 3H), 3.09 (s, 3H), 2.32 − 2.10 (m, |
| | 8H), 1.73 − 1.48 (m, 6H). MS (ESI⁺): [M + H]⁺ 406.2. |
| 254 | Yellow solid. ¹H NMR (400 MHz, DMSO-d₆, 300K) δ_{H} 8.51 (s, |
| | 1H), 8.20 (s, 1H), 7.76 (d, J = 8.5 Hz, 1H), 7.59 (d, J = 8.4 Hz, |
| | 1H), 6.83 (s, 1H, NH, D₂O exchanged), 6.58 (s, 1H), 3.85 (s, 3H), |
| | 3.17 (s, 3H), 3.09 (s, 3H), 2.38 − 2.24 (m, 4H), 2.18 (br s, 4H), |
| | 1.72 (s, 4H), 1.66 − 1.51 (m, 2H). MS (ESI⁺): [M + H]⁺ 420.3. |
| 255 | Yellow solid. ¹H NMR (400 MHz, DMSO-d₆, 300K) δ_{H} 8.51 (s, |
| | 1H), 8.20 (s, 1H), 7.73 (d, J = 8.5 Hz, 1H), 7.59 (d, J = 8.4 Hz, |
| | 1H), 6.92 (s, 1H, NH, D₂O exchanged), 6.60 (s, 1H), 3.84 (s, 3H), |
| | 3.09 (s, 3H), 2.55 − 2.30 (m, 4H), 2.26 − 2.10 (m, 4H), 1.89 (s, |
| | 4H), 1.64 − 1.52 (m, 2H). 19F NMR (376 MHz, DMSO-d₆) δ_{F} − |
| | 129.02. MS (ESI⁺): [M + H]⁺ 408.2. |
| 256 | Yellow solid. ¹H NMR (400 MHz, DMSO-d₆, 300K) δ_{H} 8.30 (t, J = |
| | 5.9 Hz, 1H, NH, D₂O exchanged), 8.25 (s, 1H), 8.09 (s, 1H), |
| | 7.74 − 7.65 (m, 3H), 7.62 (t, J = 7.7 Hz, 1H), 7.48 − 7.38 (m, 2H), |
| | 6.52 (s, 1H), 4.84 (d, J = 5.6 Hz, 2H), 3.64 (s, 3H), 3.11 (s, 3H). |
| | ¹⁹F NMR (376 MHz, DMSO-d₆) δ_{F} −58.93. MS (ESI⁺): [M + H]⁺ |
| | 414.2. |
| 257 | Yellow solid. ¹H NMR (400 MHz, DMSO-d₆, 300K) δ_{H} 8.45 (s, |
| | 1H), 8.21 (s, 1H), 8.01 (d, J = 7.8 Hz, 1H, NH, D₂O exchanged), |
| | 7.77 (dd, J = 8.5, 1.5 Hz, 1H), 7.59 (d, J = 8.4 Hz, 1H), 7.55 − |
| | 7.50 (m, 2H), 7.40 − 7.34 (m, 2H), 7.29 − 7.22 (m, 1H), 6.55 (s, |
| | 1H), 5.28 − 5.19 (m, 1H), 5.07 (t, J = 5.8 Hz, 1H, OH, D₂O |
| | exchanged), 3.88 (s, 3H), 3.87 − 3.80 (m, 1H), 3.78 − 3.68 (m, |
| | 1H), 3.18 (s, 3H). MS (ESI⁺): [M + H]⁺ 376.2. |
| 258 | Yellow solid. ¹H NMR (400 MHz, DMSO-d₆, 300K) δ_{H} 8.45 (s, |
| | 1H), 8.21 (s, 1H), 8.01 (d, J = 7.8 Hz, 1H, NH, D₂O exchanged), |
| | 7.77 (dd, J = 8.5, 1.5 Hz, 1H), 7.59 (d, J = 8.4 Hz, 1H), 7.55 − |
| | 7.50 (m, 2H), 7.40 − 7.34 (m, 2H), 7.29 − 7.22 (m, 1H), 6.55 (s, |
| | 1H), 5.28 − 5.19 (m, 1H), 5.07 (t, J = 5.8 Hz, 1H, OH, D₂O |
| | exchanged), 3.88 (s, 3H), 3.87 − 3.80 (m, 1H), 3.78 − 3.68 (m, |
| | 1H), 3.18 (s, 3H). MS (ESI⁺): [M + H]⁺ 376.2. |
| 259 | Yellow solid. ¹H NMR (400 MHz, DMSO-d₆, 300K) δ_{H} 8.46 (s, |
| | 1H), 8.21 (s, 1H), 8.14 (d, J = 7.8 Hz, 1H, NH, D₂O exchanged), |
| | 7.76 (dd, J = 8.4, 1.5 Hz, 1H), 7.59 (d, J = 8.4 Hz, 1H), 7.56 − |
| | 7.52 (m, 2H), 7.42 − 7.35 (m, 2H), 7.30 − 7.25 (m, 1H), 6.55 (s, |
| | 1H), 5.47 − 5.37 (m, 1H), 3.88 (s, 3H), 3.82 (dd, J = 10.2, 8.8 Hz, |
| | 1H), 3.64 (dd, J = 10.2, 5.2 Hz, 1H), 3.35 (s, 3H), 3.16 (s, 3H). |
| | MS (ESI⁺): [M + H]⁺ 390.2. |
| 260 | Yellow solid. ¹H NMR (400 MHz, DMSO-d₆, 300K) δ_{H} 8.45 (s, |
| | 1H), 8.20 (s, 1H), 8.05 − 7.98 (m, 1H, NH, D₂O exchanged), 7.94 |
| | (dd, J= 8.5, 1.5 Hz, 1H), 7.62 (d, J = 8.4 Hz, 1H), 7.51 − 7.45 (m, |
| | 2H), 7.43 − 7.37 (m, 2H), 7.33 − 7.26 (m, 1H), 6.60 (s, 1H), 5.75 |
| | (d, J = 4.2 Hz, 1H, OH, D₂O exchanged), 5.10 − 5.03 (m, 1H), |
| | 3.84 (s, 3H), 3.81 − 3.72 (m, 1H), 3.45 − 3.34 (m, 1H), 3.10 (s, |
| | 3H). MS (ESI⁺): [M + H]⁺ 376.2. |
| 261 | Yellow solid. ¹H NMR (400 MHz, DMSO-d₆, 300K) δ_{H} 8.63 (br |
| | s, 1H, NH, D₂O exchanged), 8.57 (s, 1H), 8.20 (s, 1H), 7.84 (d, J = |
| | 8.5 Hz, 1H), 7.59 (d, J = 8.4 Hz, 1H), 7.18 (s, 1H), 6.65 (s, 1H), |
| | 4.90 (d, J = 5.9 Hz, 2H), 3.83 (s, 3H), 3.11 (s, 3H), 2.36 (s, 3H). |
| | MS (ESI⁺): [M + H]⁺ 367.2. |
| 262 | Yellow solid. ¹H NMR (400 MHz, DMSO-d₆, 300K) δ_{H} 8.57 (s, |
| | 1H), 8.19 (s, 1H), 7.85 − 7.70 (m, 1H + NH, D₂O exchanged), |
| | 7.59 (d, J = 8.4Hz, 1H), 6.56 (s, 1H), 3.93 − 3.85 (m, 2H), 3.83 |
| | (s, 3H), 3.38 (t, J = 6.3 Hz, 2H), 3.35 − 3.26 (m, 2H), 3.08 (s, 3H), |
| | 2.13 − 1.97 (m, 1H), 1.76 − 1.64 (m, 2H), 1.38 − 1.22 (m, 2H). |
| | MS (ESI⁺): [M + H]⁺ 354.2. |
| 263 | Yellow solid. ¹H NMR (400 MHz, DMSO-d₆, 300K) δ_{H} 9.30 (s, |
| | 1H, NH, D₂O exchanged), 8.71 (s, 1H), 8.23 (s, 1H), 7.88 − 7.80 |
| | (m, 2H), 7.74 (d, J = 8.4 Hz, 1H), 7.62 (d, J = 8.4 Hz, 1H), 7.05 − |
| | 6.97 (m, 2H), 6.71 (s, 1H), 3.87 (s, 3H), 3.23 (s, 3H), 3.18 − |
| | 3.10 (m, 4H), 2.52 − 2.44 (m, 4H), 2.24 (s, 3H). MS (ESI⁺): |
| | [M + H]⁺ 430.2. |
| 264 | Yellow solid. ¹H NMR (400 MHz, CDCl₃, 300K) δ_{H} 12.41 (br s, |
| | 1H, NH, D₂O exchanged), 8.33 (dd, J = 5.0, 2.0 Hz, 1H), 7.97 (s, |
| | 1H), 7.90 (d, J = 8.4 Hz, 1H), 7.72 − 7.64 (m, 1H), 7.57 (s, 1H), |
| | 7.51 (dd, J = 8.4, 1.7 Hz, 1H), 7.23 (d, J = 8.1 Hz, 1H), 7.00 − |
| | 6.94 (m, 1H), 6.49 (s, 1H), 3.95 (s, 3H), 3.34 (s, 3H). MS (ESI⁺): |
| | [M + H]⁺ 333.2. |
| 265 | Yellow solid. ¹H NMR (400 MHz, DMSO-d₆, 300K) δ_{H} 8.42 (s, |
| | 1H), 8.20 (s, 1H), 7.94 (d, J = 8.4 Hz, 1H), 7.61 (d, J = 8.4 Hz, |
| | 1H), 7.41 (d, J = 7.2 Hz, 1H, NH, D₂O exchanged), 6.60 (s, 1H), |
| | 4.14 − 3.98 (m, 2H), 3.86 − 3.77 (m, 4H), 3.42 − 3.26 (m, 2H), |
| | 3.08 (s, 3H), 2.17 − 2.05 (m, 1H), 1.82 − 1.56 (m, 3H). MS (ESI⁺): |
| | [M + H]⁺ 340.2. |
| 266 | Beige solid. ¹H NMR (400 MHz, DMSO-d₆, 300K) δ_{H} 8.36 (s, |
| | 1H), 8.20 (s, 1H), 7.99 (dd, J = 8.6, 1.5 Hz, 1H), 7.62 (d, J = 8.5 |
| | Hz, 1H), 7.45 (d, J = 6.9 Hz, 1H, NH, exchanged), 6.59 (s, 1H), |
| | 5.12 (d, J = 4.5 Hz, 1H, OH, D₂O exchanged), 4.03 (dd, J = 10.8, |
| | 4.4 Hz, 1H), 3.95 − 3.75 (m, 6H), 3.39 (td, J = 11.6, 2.2 Hz, 1H), |
| | 3.23 (t, J = 10.3 Hz, 1H), 3.10 (s, 3H), 2.03 − 1.92 (m, 1H), 1.64 − |
| | 1.48 (m, 1H). MS (ESI⁺): [M + H]⁺ 356.3. |
| 267 | Pale yellow solid. ¹H NMR (400 MHz, DMSO-d₆, 343K) of major |
| | tautomer δ_{H} 10.59 (br. s, 1H, NH, D₂O exchanged), 8.91 (s, 1H), |
| | 8.58 (d, J = 8.8 Hz, 1H), 8.45 (d, J = 8.8 Hz, 1H), 7.63 (br. s, 1H, |
| | NH, D₂O exchanged), 6.41 (s, 1H), 3.76 (br. s, 1H), 1.94 (br. s, |
| | 2H), 1.81 − 1.72 (m, 2H), 1.67 − 1.56 (m, 1H), 1.48 − 1.31 (m, |
| | 4H), 1.30 − 1.16 (m, 1H). MS (ESI⁺): [M + H]⁺ 328.3. |
| 268 | Pale yellow solid. ¹H NMR (400 MHz, DMSO-d₆, 373K) of major |
| | tautomer δ_{H} 10.49 (br. s, 1H, NH, D₂O exchanged), 8.94 (s, 1H), |
| | 8.58 (d, J = 8.8 Hz, 1H), 8.45 (d, J = 8.8 Hz, 1H), 7.62 (br. s, 1H, |
| | NH, D₂O exchanged), 6.42 (s, 1H), 3.98 (br. s, 1H), 2.07 − 1.88 |
| | (m, 2H), 1.77 − 1.42 (m, 10H). MS (ESI⁺): [M + H]⁺ 342.3. |
| 269 | Yellow solid. ¹H NMR (400 MHz, DMSO-d₆, 343K) δ_{H} 10.48 (br. |
| | s, 1H, NH, D₂O exchanged), 8.96 (s, 1H), 8.57 (d, J = 8.8 Hz, |
| | 1H), 8.43 (d, J = 8.8 Hz, 1H), 7.48 (br. s, 1H, NH, D₂O |
| | exchanged), 6.44 (s, 1H), 4.21 (br. s, 1H), 3.54 − 3.39 (m, 2H), |
| | 3.33 (s, 3H), 1.78 − 1.61 (m, 1H), 1.61 − 1.35 (m, 2H), 1.05 − 0.80 |
| | (m, 6H). MS (ESI⁺): [M + H]⁺ 360.3. |
| 270 | Beige solid. ¹H NMR (400 MHz, DMSO-d₆, 343K) of major |
| | tautomer δ_{H} 9.89 (br. s, 1H, NH, D₂O exchanged), 8.98 (s, 1H), |
| | 8.60 (d, J = 8.8 Hz, 1H), 8.52 − 8.39 (m, 1H), 7.02 (br. s, 1H, NH, |
| | D₂O exchanged), 6.45 (s, 1H), 2.17 (d, J = 18.6 Hz, 9H), 1.75 (s, |
| | 6H). MS (ESI⁺): [M + H]⁺ 380.3. |
| 271 | Yellow solid. ¹H NMR (400 MHz, DMSO-d₆, 300K) of major |
| | tautomer δ_{H} 13.13 (br. s, 1H, NH, D₂O exchanged), 10.54 (br. s, |
| | 1H, NH, D₂O exchanged), 9.53 (br. s, 1H, NH, D₂O exchanged), |
| | 8.50 (s, 1H), 8.25 − 8.10 (m, 2H), 7.68 (d, J = 8.5 Hz, 2H), 7.55 |
| | (d, J = 8.7 Hz, 1H), 7.02 (d, J = 8.7 Hz, 2H), 6.59 (s, 1H), 3.18 − |
| | 3.08 (m, 4H), 2.49 − 2.40 (m, 4H), 2.24 (s, 3H). MS (ESI⁺): |
| | [M + H]⁺ 402.3. |

### Pathologies

The compounds of formula (I) may be useful in the treatment and/or in the prevention of a disease selected from cognitive deficits and neuroinflammation associated with Down syndrome (Trisomy 21), Alzheimer's disease and related diseases, dementia; tauopathies; and other neurodegenerative diseases (Parkinson's disease; Pick disease, including Niemann-Pick Type C Disease); CDKL5 Deficiency Disorder; McDermid syndrome; autism; type 1 and type 2 diabetes; abnormal folate and methionine metabolism; tendinopathy and osteoarthritis, in particular knee osteoarthritis; Duchenne muscular dystrophy; cancers, such as brain cancer, including glioblastoma, leukemia, including megakaryoblastic leukemia and acute lymphoblastic leukemia, head and neck squamous cell carcinoma, pancreatic cancer, including pancreatic ductal adenocarcinoma, prostate cancer, gastrointestinal cancer, breast cancer, such as Triple-negative breast cancer (TNBC), tissue cancer, including liposarcoma, Hedgehog/GLI-dependent cancer, liver cancer, including Hepatocellular carcinoma and viral infections, such as caused by Human immunodeficiency virus type 1 (HIV-1), Human cytomegalovirus (HCMV), Influenza A, Herpes virus, rhesus macaque cytomegalovirus, varicella-zoster virus, herpes simplex virus (HSV), Hepatitis C virus, Chikungunya virus, Dengue virus, Influenza virus and Severe acute respiratory syndrome (SARS) coronavirus, Cytomegalovirus and Human papillomavirus; neuroinflammation; anemia; infections caused by unicellular parasites, such as malaria, Leishmaniasis, Chagas and sleeping sickness (Trypanosoma sp.), cattle diseases due to unicellular pathogens and for regulating body temperature.

According to a particular embodiment, the compounds of formula (I) of the present invention may be useful in the treatment and/or in the prevention of a disease selected from cognitive deficits and neuroinflammation associated with Down syndrome (Trisomy 21), Alzheimer's disease and related diseases, dementia or tauopathies; other neurodegenerative diseases (Parkinson's disease; Pick disease, including Niemann-Pick Type C Disease); CDKL5 Deficiency Disorder; type 1 and type 2 diabetes; abnormal folate and methionine metabolism; tendinopathy and osteoarthritis, in particular knee osteoarthritis; Duchenne muscular dystrophy; cancers, such as brain cancer, including glioblastoma, leukemia, including megakaryoblastic leukemia and acute lymphoblastic leukemia, head and neck squamous cell carcinoma, pancreatic cancer, including pancreatic ductal adenocarcinoma, prostate cancer, gastrointestinal cancer and breast cancer, such as Triple-negative breast cancer (TNBC) and viral infections, such as caused by Human immunodeficiency virus type 1 (HIV-1), Human cytomegalovirus (HCMV), Influenza A, Herpes virus, rhesus macaque cytomegalovirus, varicella-zoster virus and herpes simplex virus (HSV) and for regulating body temperature. Said diseases are more particularly associated with the abnormalities in DYRK1A and/or CLK1 dosage.

Still according to this particular embodiment, the compounds of formula (I) of the present invention may be useful in the treatment and/or prevention of a disease selected from Down syndrome, Alzheimer's disease, dementia, tauopathies, Parkinson's disease, Niemann-Pick Type C Disease, CDKL5 Deficiency Disorder and Phelan-McDermid syndrome and their associated cognitive and motor conditions, more particularly due to high expression and activity of DYRK1A.

Still according to this particular embodiment, the compounds of formula (I) of the present invention may be useful in the treatment and/or prevention of a disease selected from Down syndrome, Alzheimer's disease and related Tauopathies, Parkinson's disease, the cognitive/motor disorders associated therewith, or one or more symptoms of such diseases. As a typical symptom of such diseases is a decline in learning and memory and social interactions.

Still according to this particular embodiment, the compounds of formula (I) of the present invention may be useful in combatting cognitive decline associated with Down syndrome (Trisomy 21), in learning and memory, in particular associated with the cognitive or neurodegenerative disorders as mentioned above.

Still according to this particular embodiment, the compounds of formula (I) of the present invention may be useful in the treatment and/or prevention of type 1 and type 2 diabetes.

The compounds of formula (I) of the present invention may be useful in the treatment and/or prevention of type 1 and type 2 diabetes either by direct treatment of the diabetic patient or by treating isolated/cultured pancreatic islets or β-cells in vitro or ex vivo prior to transplantation into diabetic patients.

Herein is further provided a method of treatment of type 1 and type 2 diabetes in a patient in need thereof comprising a step of administering a compound of formula (I) as defined above.

Herein is further provided a method of treatment of type 1 and type 2 diabetes in a patient in need thereof comprising a step of treating isolated or cultured pancreatic islets or β-cells in vitro or ex vivo prior to transplantation into said patient with a compound of formula (I) as defined above.

Still according to this particular embodiment, the compounds of formula (I) of the present invention may be useful in the treatment and/or prevention of viral infections, in particular caused by such as caused by Human immunodeficiency virus type 1 (HIV-1), Human cytomegalovirus (HCMV), Influenza A, Herpes virus, rhesus macaque cytomegalovirus, varicella-zoster virus and herpes simplex virus (HSV), and in particular by Herpes, Coronaviruses, Cytomegaloviruses and Influenzas. These infections may be associated with high expression and activity of DYRK1A and/or CLK1, and optionally additionally with dual inhibitors of CLKs/DYRKS.

Acute respiratory disease has recently been caused by a new coronavirus (SARS-CoV-2, previously known as 2019-nCoV), also known here as coronavirus 2019 (COVID-19), which belongs to Coronaviridae. The compounds of formula (I) according to the present invention can also treat said infection caused by SARS-CoV-2 virus.

Still according to this particular embodiment, the compounds of formula (I) of the present invention may be useful in the treatment and/or prevention of cancers, such as brain cancer, including glioblastoma, leukemia, including megakaryoblastic leukemia and acute lymphoblastic leukemia, head and neck squamous cell carcinoma, pancreatic cancer, including pancreatic ductal adenocarcinoma, prostate cancer, gastrointestinal cancer and breast cancer, such as Triple-negative breast cancer (TNBC). These cancers may be associated with high expression and activity of DYRK1A and/or CLK1, and optionally additionally with dual inhibitors of CLKs/DYRKS.

Still according to this particular embodiment, the compounds of formula (I) of the present invention may be useful in the treatment and/or prevention of tendinopathy and osteoarthritis. Tendinopathy and osteoarthritis may be associated with high expression and activity of DYRK1A and/or CLK2.

Still according to this particular embodiment, the compounds of formula (I) of the present invention may be useful in the treatment and/or prevention of infections caused by unicellular parasites, such as such as malaria, Leishmaniasis, Chagas and sleeping sickness (Trypanosoma sp.), and cattle diseases due to unicellular pathogens Said parasitic infections may be associated with expression and activity of DYRKs/CLKs.

Still according to this particular embodiment, the compounds of formula (I) of the present invention may be useful in the regulation of the body temperature. Said body temperature regulation may be associated with expression and activity of CLKs.

According to another particular embodiment, the compounds of formula (I) of the present invention may be useful in the treatment and/or in the prevention of a disease selected from Phelan-McDermid syndrome; autism; further viral infections, such as caused by Hepatitis C virus, Chikungunya virus, Dengue virus, Influenza virus and Severe acute respiratory syndrome (SARS) coronavirus, Cytomegalovirus and Human papillomavirus; further cancers, such as tissue cancer, including liposarcoma, Hedgehog/GLI-dependent cancer, liver cancer, including Hepatocellular carcinoma, neuroinflammation, anemia, infections caused by unicellular parasites, such as such as malaria, Leishmaniasis, Chagas and sleeping sickness (Trypanosoma sp.), and cattle diseases due to unicellular pathogens. Said diseases are more particularly associated with the abnormalities in other DYRKs (DYR1B, 2, 3, 4) and the closely related further cdc2-like kinases (CLKs) (CLK 2, 3, 4).

The following examples are provided as illustrations and in no way limit the scope of this invention.

The following examples illustrate in detail the preparation of some compounds according to the invention. The structures of the products obtained have been confirmed by NMR analyses and mass spectroscopy.

### Example 1: S-alkylation of 2-thiohydantoins

### Example 1.1: Synthesis of 2-methylsulfanyl-1,4-dihydroimidazol-5-one (1.1)

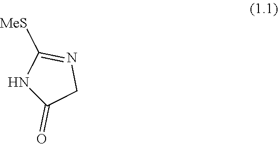

MeI (51.4 mL, 0.827 mol, 4 eq) was slowly added dropwise to a stirred suspension of 2-thiohydantoin (24 g, 206.6 mmol, 1 eq), DIPEA (72 mL, 413.2 mmol, 2 eq) and DMAP (10.096 g, 82.64 mmol, 0.4 eq) in DCM (413 mL) maintained at 0° C. The resulting mixture was stirred 6 h at 0° C. A precipitate gradually appeared. Upon completion (TLC), the precipitate was filtered on a fritted glass funnel. The resulting solid was adsorbed on silica and purified by FC on silica gel (elution: cyclohexane/AcOEt/DCM 70/30/3 to 0/60/40/). The volume of the collected fractions was reduced to approximately an eighth of its initial volume, until yellow crystals started to appear. The mixture was stirred 30 min at 0° C. and the solid was collected by filtration on a fritted glass funnel to yield 2-methylsulfanyl-1,4-dihydroimidazol-5-one (15.368 g, 118.1 mmol, 57%) in analytically pure form. Pale yellow solid. ¹H NMR (400 MHz, DMSO-d₆, 300K) of major tautomer δ_{H} 11.24 (br s, 1H, NH, D₂O exchanged), 4.01 (s, 2H), 2.47 (s, 3H). MS (ESI⁺): [M+H]⁺ 131.0.

### Example 1.2: Synthesis of 1-methyl-2-methylsulfanyl-4H-imidazol-5-one (1.2)

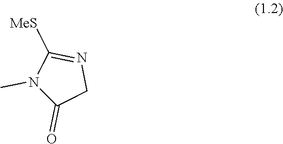

MeO₃BF₄ (6.818 g, 46.1 mmol, 1.5 eq) was added portionwise to a stirred solution of 3-methyl-2-thiohydantoin (4 g, 30.73 mmol, 1 eq) in DCM. The reaction medium was stirred at room temperature for 12 h. Upon completion (TLC), the resulting mixture was quenched with sat. Na₂CO_{3(aq)}. The aqueous layer was extracted three times with DCM. The combined organic layers were dried over MgSO₄, filtered, concentrated in vacuo, adsorbed on silica, and purified by FC on silica gel (elution: cyclohexane/AcOEt/DCM: 92/5/3 to 50/47/3) to give the desired isothiourea (3.797 g, 26.33 mmol, 85%) in analytically pure form. ¹H NMR (400 MHz, DMSO-d₆, 300K) δ_{H} 4.11 (s, 2H), 2.93 (s, 3H), 2.51 (s, 3H). ¹³C NMR (101 MHz, DMSO-d₆, 300K) δ_{C} 179.6, 162.6, 58.3, 25.9, 11.8. MS (ESI⁺): [M+H]⁺ 145.0.

### Example 2: General Protocol 1—Addition of aliphatic and aromatic amines on 2-alkylsulfanyl-1,4-dihydroimidazol-5-ones—ROUTE 1

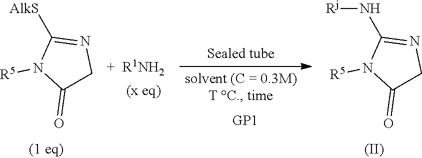

In the above scheme, R¹ and R⁵ are as defined above and Alk is a (C₁-C₅)alkyl.
GP1: the appropriate amine (x eq) was added to a stirred solution of the appropriate 2-alkylsulfanyl-1,4-dihydroimidazol-5-one) (1 eq) in dry THF (C=0.3 M/isothiourea) in a sealed tube or sealed round flask (heating block). The mixture was thoroughly purged with vacuum/argon cycles and heated at the appropriate temperature for the indicated time (see details below). Upon completion (followed by consumption of the isothiourea on TLC), the mixture was brought back to room temperature.
   GP1-A: direct precipitation of the desired product: The reaction medium was stirred 1 h at 0° C. The precipitated solid was filtered off on a fritted glass funnel. High purity may be achieved after filtration by washing, reprecipitation, trituration, or recrystallization.
   GP1-B: the product failed to precipitate: the reaction mixture was poured on Et₂O maintained at 0° C. The precipitated solid was filtered off on a fritted glass funnel. High purity may be achieved after filtration by washing, reprecipitation, trituration, or recrystallization.
   GP1-C: the product failed to precipitate: the reaction mixture was concentrated in vacuo, adsorbed on silica, and purified by FC. High purity may be achieved after purification by washing, reprecipitation, trituration, or recrystallization.
(a) May require activation with AcOH, depending on the amine (see details below).

### Example 2.1: Synthesis of 2-(cyclohexylamino)-1,4-dihydroimidazol-5-one

(2.1)

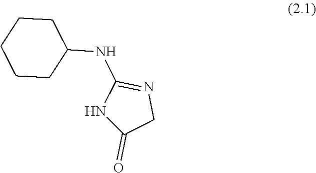 [SMILES: O=C1CN=C(NC2CCCCC2)N1]

Compound (2.1) was synthesized according to GP1-A: reaction carried out in THF, with intermediate (1.1) (8.91 mmol) and 4 eq of cyclohexylamine, at 120° C. (heating block), for 12 h. The product directly precipitated in the reaction medium: it was isolated after filtration, washing with cold THF, then pentane. Off-white solid, 35% (571 mg). ¹H NMR (400 MHz, DMSO-d₆, 300K) of major tautomer δ_{H} 7.41 (br s, 1H, NH, D₂O exchanged), 6.95 (br s, 1H, NH, D₂O exchanged), 3.61 (s, 2H), 3.39-3.23 (m, 1H), 1.88-1.75 (m, 2H), 1.74-1.62 (m, 2H), 1.61-1.51 (m, 1H), 1.33-1.03 (m, 5H). MS (ESI⁺): [M+H]⁺ 182.0.

### Example 2.2: Synthesis of 2-(cycloheptylamino)-1,4-dihydroimidazol-5-one (2.2)

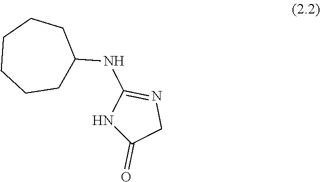 [SMILES: O=C1CN=C(NC2CCCCCC2)N1]

Compound (2.2) was synthesized according to GP1-A: reaction carried out in THF, with intermediate (1.1) (11.52 mmol) and 4 eq of cycloheptylamine, at 120° C. (sealed tube, heating bock), for 12 h. The product directly precipitated in the reaction medium: it was isolated after filtration, washing with cold THF, then pentane. Off-white solid, 64% (1.429 g). ¹H NMR (400 MHz, DMSO-d₆, 300K) of major tautomer δ_{H} 7.42 (br s, 1H, NH, D₂O exchanged), 6.88 (br s, 1H, NH, D₂O exchanged), 3.86-3.68 (m, 1H), 3.59 (s, 2H), 1.89-1.76 (m, 2H), 1.70-1.32 (m, 10H). MS (ESI⁺): [M+H]⁺ 196.0.

### Example 2.3: Synthesis of 2-(cyclooctylamino)-1,4-dihydroimidazol-5-one (2.3)

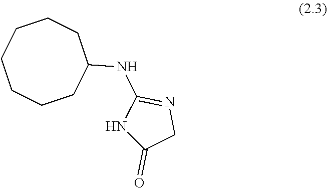 [SMILES: O=C1CN=C(NC2CCCCCCC2)N1]

Compound (2.3) was synthesized according to GP1-A: reaction carried out in THF, with intermediate (1.1) (11.52 mmol) and 4 eq of cyclooctylamine, at 120° C. (sealed tube, heating bock), for 12 h. The product directly precipitated in the reaction medium: it was isolated after filtration, washing with cold THF, then pentane. Off-white solid, 72% (1.739 g). ¹H NMR (400 MHz, DMSO-d₆, 300K) of major tautomer δ_{H} 7.43 (br s, 1H, NH, D₂O exchanged), 6.86 (br s, 1H, NH, D₂O exchanged), 3.89-3.74 (m, 1H), 3.59 (s, 2H), 1.90-1.27 (m, 14H). MS (ESI⁺): [M+H]⁺ 210.0.

### Example 2.4: Synthesis of 2-[[(1R)-1-(hydroxymethyl)-3-methyl-butyl]amino]-1,4-dihydroimidazol-5-one (2.4)

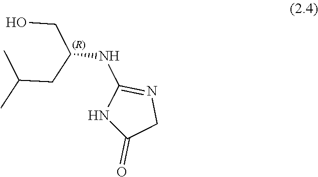

Compound (2.4) was synthesized according to GP1-A: reaction carried out in THF with intermediate (1.1) (16.90 mmol), 2.5 eq of (D)-leucinol, at 125° C., for 4 h. The product directly precipitated in the reaction medium: it was isolated after filtration, washing with cold THF, then pentane. Light beige solid, 52% (1.750 g). ¹H NMR (400 MHz, DMSO-d₆, 343K) of major tautomer δ_{H} 7.11 (br s, 2H, NH, D₂O exchanged), 4.61 (s, 1H, OH, D₂O exchanged), 3.91-3.70 (m, 1H), 3.61 (s, 2H), 3.46-3.28 (m, 2H), 1.70-1.56 (m, 1H), 1.37 (t, J=7.0 Hz, 2H), 0.89 (t, J=7.1 Hz, 6H). MS (ESI⁺): [M+H]⁺ 200.1.

### Example 2.5: Synthesis of 2-[[(1R)-1-(methoxymethyl)-3-methyl-butyl]amino]-1,4-dihydroimidazol-5-one (2.5)

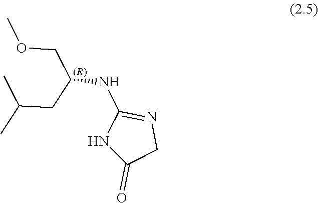

Compound (2.5) was synthesized according to GP1-B: reaction carried out in THF, with intermediate (1.1) (11.52 mmol), 2 eq of (2R)-1-methoxy-4-methyl-pentan-2-amine and 4 eq of AcOH. Light beige solid, 39% (948 mg). ¹H NMR (400 MHz, DMSO-d₆, 343K) of major tautomer δ_{H} 7.20 (br s, 1H, NH, D₂O exchanged), 6.98 (br s, 1H, NH, D₂O exchanged), 4.09-3.75 (m, 1H), 3.61 (s, 2H), 3.39-3.29 (m, 2H), 3.27 (s, 3H), 1.71-1.56 (m, 1H), 1.47-1.27 (m, 2H), 0.89 (t, J=6.7 Hz, 6H). MS (ESI⁺): [M+H]⁺ 214.3.

### Example 2.6: Preparation of 2-[[(1R)-1-(fluoromethyl)-3-methyl-butyl]amino]-1,4-dihydroimidazol-5-one (2.6)

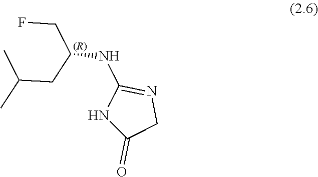

Compound (2.6) was synthesized according to GP1-B: reaction carried out in THF, with intermediate (1.1) (4.088 mmol), 1.6 eq of (2R)-1-fluoro-4-methyl-pentan-2-amine and 4 eq of AcOH, at 120° C. (heating block), for 4 h. Beige solid, 32% (267 mg). ¹H NMR (400 MHz, DMSO-d₆, 343K) of major tautomer δ_{H} 7.39 (br s, 1H, NH, D₂O exchanged), 7.12 (br s, 1H, NH, D₂O exchanged), 4.47-4.38 (m, 1H), 4.36-4.26 (m, 1H), 4.11-3.89 (m, 1H), 3.63 (s, 2H), 1.73-1.59 (m, 1H), 1.52-1.42 (m, 1H), 1.40-1.29 (m, 1H), 0.97-0.86 (m, 6H). MS (ESI⁺): [M+H]⁺ 202.1.

### Example 2.7: Synthesis of 2-[[(1S)-1-(fluoromethyl)-3-methyl-butyl]amino]-1,4-dihydroimidazol-5-one (2.7)

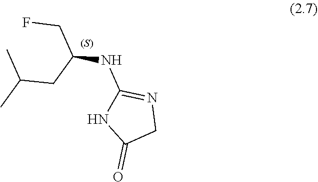

Compound (2.7) was synthesized according to GP1-B: reaction carried out in THF, with intermediate (1.1) (4.088 mmol), 1.6 eq of (2S)-1-fluoro-4-methyl-pentan-2-amine and 4 eq of AcOH, at 120° C. (heating block), for 4 h. Beige solid, 44% (366 mg). ¹H NMR (400 MHz, DMSO-d₆, 343K) of major tautomer δ_{H} 7.39 (br s, 1H, NH, D₂O exchanged), 7.12 (br s, 1H, NH, D₂O exchanged), 4.47-4.38 (m, 1H), 4.36-4.26 (m, 1H), 4.11-3.89 (m, 1H), 3.63 (s, 2H), 1.73-1.59 (m, 1H), 1.52-1.42 (m, 1H), 1.40-1.29 (m, 1H), 0.97-0.86 (m, 6H). MS (ESI⁺): [M+H]⁺ 202.1.

### Example 2.8: Synthesis of 2-[[(1R,2R)-2-methoxycyclopentyl]amino]-1,4-dihydroimidazol-5-one (2.8)

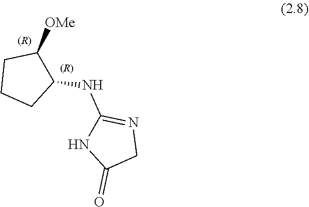

Compound (2.8) was synthesized according to GP1-A: reaction carried out in THF with intermediate (1.1) (868 μmol), 3 eq of (1R,2R)-2-methoxycyclopentanamine, at 115° C., for 12 h. The product directly precipitated in the reaction medium: it was isolated after filtration, washing with cold THF, then pentane. Colorless solid, 74% (127 mg). ¹H NMR (400 MHz, DMSO-d₆, 343K) of major tautomer δ_{H} 7.57 (br s, 1H, NH, D₂O exchanged), 7.13 (br s, 1H, NH, D₂O exchanged), 3.99-3.79 (m, 1H), 3.69-3.63 (m, 1H), 3.61 (s, 2H), 3.25 (s, 3H), 2.05-1.83 (m, 2H), 1.73-1.40 (m, 4H). MS (ESI⁺): [M+H]⁺ 198.1.

### Example 2.9: Synthesis of 2-[[(1S,2S)-2-methoxycyclopentyl]amino]-1,4-dihydroimidazol-5-one (2.9)

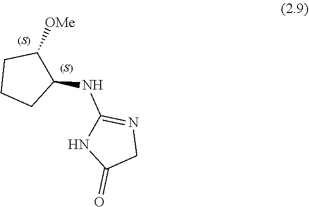

Compound (2.9) was synthesized according to GP1-A: reaction carried out in THF with intermediate (1.1) (2.89 mmol), 3 eq of (1SR,2S)-2-methoxycyclopentanamine, at 115° C., for 12 h. The product directly precipitated in the reaction medium: it was isolated after filtration, washing with cold THF, then pentane. Colorless solid, 71% (405 mg). ¹H NMR (400 MHz, DMSO-d₆, 343K) of major tautomer δ_{H} 7.57 (br s, 1H, NH, D₂O exchanged), 7.13 (br s, 1H, NH, D₂O exchanged), 3.99-3.79 (m, 1H), 3.69-3.63 (m, 1H), 3.61 (s, 2H), 3.25 (s, 3H), 2.05-1.83 (m, 2H), 1.73-1.40 (m, 4H). MS (ESI⁺): [M+H]⁺ 198.1.

### Example 2.10: Synthesis of 2-(3-noradamantylamino)-1,4-dihydroimidazol-5-one (2.10)

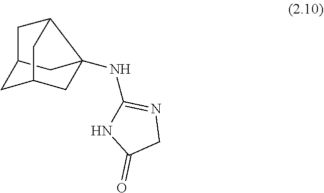 [SMILES: O=C1CN=C(NC23CC4CC(CC2C4)C3)N1]

Compound (2.10) was synthesized according to GP1-C: reaction carried out in dioxane, with intermediate (1.1) (3.55 mmol), 3 eq of 3-noradamantanamine and 4 eq of AcOH, at 130° C. (heating block), for 12 h. Purification by FC (elution: DCM/MeOH (7N NH₃): 99/1 to 9/1). The final product required a trituration in DCM at 0° C. Light beige solid, 65% (502 mg). ¹H NMR (400 MHz, DMSO-d₆, 343K) δ_{H} 7.47 (br s, 1H, NH, D₂O exchanged), 6.64 (br s, 1H, NH, D₂O exchanged), 3.56 (s, 2H), 2.41 (t, J=6.8 Hz, 1H), 2.29-2.20 (m, 2H), 2.10-1.90 (m, 6H), 1.64-1.45 (m, 4H). MS (ESI⁺): [M+H]⁺ 220.2.

### Example 2.11: Synthesis of 2-(1-adamantylamino)-1,4-dihydroimidazol-5-one (2.11)

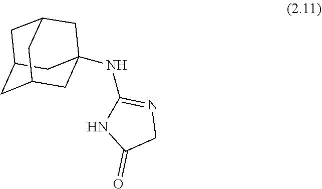 [SMILES: O=C1CN=C(NC23CC4CC(CC(C4)C2)C3)N1]

Compound (2.11) was synthesized according to GP1-C: reaction carried out in dioxane, with intermediate (1.1) (3.073 mmol), 3 eq of adamantan-1-amine and 4 eq of AcOH, at 150° C. (heating block), for 16 h. Purification by FC (elution: DCM/MeOH (7N NH₃): 99/1 to 9/1). The final product required a trituration in DCM at 0° C. Beige solid, 35% (254 mg). ¹H NMR (400 MHz, DMSO-d₆, 300K) δ_{H} 7.18 (br s, 1H, NH, D₂O exchanged), 6.73 (br s, 1H, NH, D₂O exchanged), 3.52 (s, 2H), 2.03 (s, 3H), 1.96 (s, 6H), 1.62 (s, 6H). MS (ESI⁺): [M+H]⁺ 234.2.

### Example 2.12: Synthesis of 2-[(3-hydroxy-1-adamantyl)amino]-1,4-dihydroimidazol-5-one (2.12)

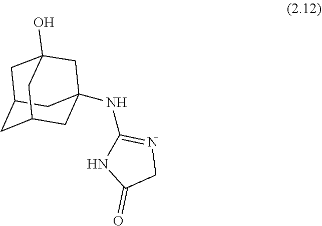 [SMILES: O=C1CN=C(NC23CC4CC(CC(O)(C4)C2)C3)N1]

Compound (2.11) was synthesized according to GP1-C: reaction carried out in dioxane, with intermediate (1.1) (9.22 mmol), 3 eq of 3-aminoadamantan-1-ol and 4 eq of AcOH, at 145° C. (heating block), for 12 h. Purification by FC (elution: DCM/MeOH (7N NH₃): 99/1 to 85/15). The final product required a trituration in refluxing DCM. Beige solid, 58% (1.336 g). ¹H NMR (400 MHz, DMSO-d₆, 300K) δ_{H} 7.25 (br s, 1H, NH, D₂O exchanged), 6.75 (br s, 1H, NH, D₂O exchanged), 4.53 (s, 1H, OH, D₂O exchanged), 3.53 (s, 2H), 2.15 (br s, 2H), 1.90-1.78 (m, 6H), 1.60-1.35 (m, 6H). MS (ESI⁺): [M+H]⁺ 250.3.

### Example 2.13: Synthesis of 2-[(3-methoxy-1-adamantyl)amino]-1,4-dihydroimidazol-5-one (2.13)

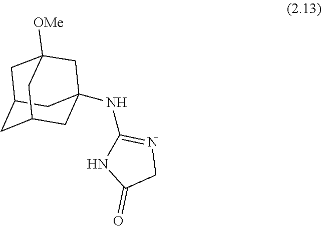 [SMILES: COC12CC3CC(CC(NC4=NCC(=O)N4)(C3)C1)C2]

Compound (2.13) was synthesized according to GP1-C: reaction carried out in dioxane, with intermediate (1.1) (768 μmol), 3 eq of 3-methoxyadamantan-1-amine and 4 eq of AcOH, at 150° C. (heating block), for 16 h. Purification by FC (elution: DCM/MeOH (7N NH₃): 99/1 to 9/1). Brown solid, 50% (102 mg). ¹H NMR (400 MHz, DMSO-d₆, 300K) δ_{H} 7.30 (br s, 1H, NH, D₂O exchanged), 6.77 (br s, 1H, NH, D₂O exchanged), 3.53 (s, 2H), 3.12 (s, 3H), 2.21 (br s, 2H), 1.95-1.80 (m, 6H), 1.67-1.56 (m, 4H), 1.55-1.40 (m, 2H). MS (ESI⁺): [M+H]⁺ 264.3.

### Example 2.14: Synthesis of 2-[(3-fluoro-1-adamantyl)amino]-1,4-dihydroimidazol-5-one (2.14)

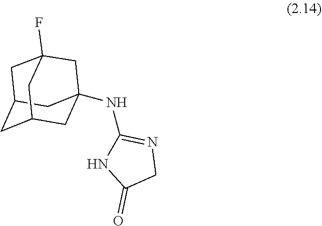 [SMILES: O=C1CN=C(NC23CC4CC(CC(F)(C4)C2)C3)N1]

Compound (2.14) was synthesized according to GP1-C: reaction carried out in dioxane, with intermediate (1.1) (3.073 mmol), 3 eq of 3-fluoro-adamantan-1-amine and 4 eq of AcOH, at 130° C. (heating block), for 12 h. Purification by FC (elution: DCM/MeOH (7N NH₃): 99/1 to 85/15). The final product required a trituration in Et₂O at 0° C. Beige solid, 44% (343 mg). ¹H NMR (400 MHz, DMSO-d₆, 300K) δ_{H} 7.41 (br s, 1H, NH, D₂O exchanged), 6.83 (br s, 1H, NH, D₂O exchanged), 3.54 (s, 2H), 2.35-2.24 (m, 2H), 2.14 (d, J=5.9 Hz, 2H), 2.01-1.72 (m, 8H), 1.56-1.43 (m, 2H). MS (ESI⁺): [M+H]⁺ 252.3.

### Example 2.15: Synthesis of 2-[[(1R)-2-methoxy-1-phenyl-ethyl]amino]-1,4-dihydroimidazol-5-one (2.15)

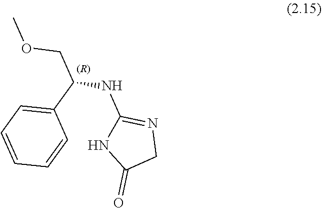

Compound (2.15) was synthesized according to GP1-A: reaction carried out in THF, with intermediate (1.1) (3.073 mmol), 3 eq of (1R)-2-methoxy-1-phenyl-ethanamine and 4 eq of AcOH, at 115° C. (sealed tube, heating bock), for 12 h. The product directly precipitated in the reaction medium: it was isolated after filtration, washing with cold THF, then pentane. Beige solid, 58% (414 mg). ¹H NMR (400 MHz, DMSO-d₆, 343K) 7.95 (bs, 1H, NH, D₂O exchanged), 7.60-7.22 (m, 5H), 7.17 (bs, 1H, NH, D₂O exchanged), 5.09-4.85 (m, 1H), 3.67-3.63 (m, 1H), 3.62 (s, 2H), 3.56 (dd, J=10.2, 5.2 Hz, 1H), 3.29 (s, 3H). MS (ESI⁺): [M+H]⁺ 234.2.

### Example 2.16: Synthesis of tert-butyl N-[(2R)-2-[(5-oxo-1,4-dihydroimidazol-2-yl)amino]-2-phenyl-ethyl]carbamate (2.16)

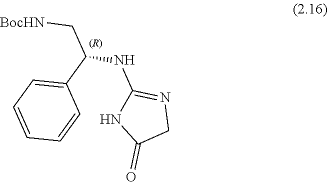

Compound (2.16) was synthesized according to GP1-A: reaction carried out in THF, with intermediate (1.1) (10.18 mmol), 1.5 eq of tert-butyl N-[(2R)-2-amino-2-phenyl-ethyl]carbamate and 2.3 eq of AcOH, at 115° C. (sealed tube, heating bock), for 6 h. The product directly precipitated in the reaction medium: it was isolated after filtration, washing with cold THF, then pentane. Colorless solid, 40% (1.517 g). ¹H NMR (400 MHz, DMSO-d₆, 343K) 7.83 (br s, 1H, NH, D₂O exchanged), 7.56-7.05 (m, 5H+NH, D₂O exchanged), 6.63 (br s, 1H, NH, D₂O exchanged), 4.91 (br s, 1H), 3.59 (s, 2H), 3.29 (t, J=6.5 Hz, 2H), 1.36 (s, 9H). MS (ESI⁺): [M+H]⁺ 319.2.

### Example 2.17: Synthesis of tert-butyl N-[(2S)-2-[(5-oxo-1,4-dihydroimidazol-2-yl)amino]-2-phenyl-ethyl]carbamate (2.17)

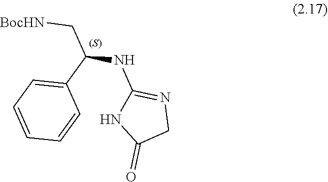

Compound (2.17) was synthesized according to GP1-A: reaction carried out in THF, with intermediate (1.1) (10.92 mmol), 1.5 eq of tert-butyl N-[(2S)-2-amino-2-phenyl-ethyl]carbamate and 2.3 eq of AcOH, at 115° C. (heating bock), for 6 h. The product directly precipitated in the reaction medium: it was isolated after filtration, washing with cold THF, then pentane. Colorless solid, 38% (1.306 g). ¹H NMR (400 MHz, DMSO-d₆, 343K) 7.83 (br s, 1H, NH, D₂O exchanged), 7.56-7.05 (m, 5H+NH, D₂O exchanged), 6.63 (br s, 1H, NH, D₂O exchanged), 4.91 (br s, 1H), 3.59 (s, 2H), 3.29 (t, J=6.5 Hz, 2H), 1.36 (s, 9H). MS (ESI⁺): [M+H]⁺ 319.3.

### Example 2.18: Synthesis of 2-[(4-methylthiazol-2-yl)methylamino]-1,4-dihydroimidazol-5-one (2.18)

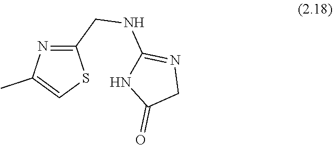

Compound (2.18) was synthesized according to GP1-A: reaction carried out in THF, with intermediate (1.1) (3.073 mmol), 2 eq of (4-methylthiazol-2-yl)methanamine and 3 eq of AcOH, at 110° C. (sealed tube, heating block), for 3 h. The product directly precipitated in the reaction medium: it was isolated after filtration, washing with cold THF, then pentane. Beige solid, 35% (248 mg). ¹H NMR (400 MHz, DMSO-d₆, 300K) δ_{H} 8.21 (br s, 1H, NH, D₂O exchanged), 7.59 (br s, 1H, NH, D₂O exchanged), 7.17 (s, 1H), 4.65 (s, 2H), 3.69 (s, 2H), 2.33 (s, 3H). MS (ESI⁺): [M+H]⁺ 211.1.

### Example 2.19: Synthesis of 2-(tetrahydropyran-4-ylmethylamino)-1,4-dihydroimidazol-5-one (2.19)

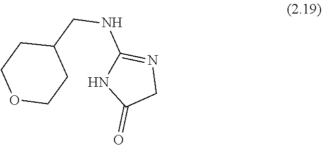 [SMILES: O=C1CN=C(NCC2CCOCC2)N1]

Compound (2.19) was synthesized according to GP1-A: reaction carried out in THF, with intermediate (1.1) (3.073 mmol) and 3 eq of tetrahydropyran-4-ylmethanamine, at 110° C. (sealed tube, heating block), for 16 h. The product directly precipitated in the reaction medium: it was isolated after filtration, washing with cold THF, then pentane. Beige solid, 70% (424 mg). ¹H NMR (400 MHz, DMSO-d₆, 343K) δ_{H} 7.47 (br s, 1H, NH, D₂O exchanged), 7.22 (br s, 1H, NH, D₂O exchanged), 3.90-3.80 (m, 2H), 3.61 (s, 2H), 3.28 (td, J=11.6, 2.0 Hz, 2H), 3.11-3.05 (m, 2H), 1.80-1.73 (m, 1H), 1.64-1.52 (m, 2H), 1.19 (qd, J=11.8, 4.4 Hz, 2H). MS (ESI): [M+H]⁺ 198.1.

### Example 2.20: Synthesis of 2-[4-(4-methylpiperazin-1-yl)anilino]-1,4-dihydroimidazol-5-one (2.20)

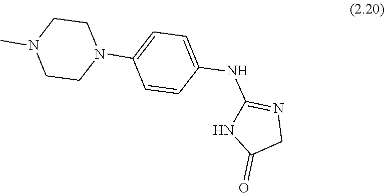 [SMILES: CN1CCN(c2ccc(NC3=NCC(=O)N3)cc2)CC1]

Compound (2.20) was synthesized according to GP1-C: reaction carried out in dioxane, with intermediate (1.1) (4.61 mmol), 2 eq of 4-(4-methylpiperazin-1-yl)aniline and 3 eq of AcOH, at 130° C. (heating block), for 3 h. Purification by FC (elution: DCM/MeOH (7N NH₃): 99/1 to 85/15). The final product required two successive triturations in EtOH at 0° C. Beige solid, 59% (749 mg). ¹H NMR (400 MHz, DMSO-d₆, 300K) δ_{H} 9.66 (br s, 1H, NH, D₂O exchanged), 7.43 (br s, 1H, NH, D₂O exchanged), 7.33-7.19 (m, 2H), 6.91 (d, J=8.9 Hz, 2H), 3.67 (s, 2H), 3.13-3.03 (m, 4H), 2.47-2.41 (m, 4H), 2.21 (s, 3H). MS (ESI⁺): [M+H]⁺ 274.2.

### Example 2.21: Synthesis of 2-(2-pyridylamino)-1,4-dihydroimidazol-5-one

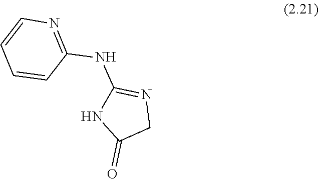 [SMILES: O=C1CN=C(Nc2ccccn2)N1]

Compound (2.21) was synthesized according to GP1-C: reaction carried out in dioxane, with intermediate (1.1) (6.15 mmol), 2.5 eq of 2-aminopyridine and 3 eq of AcOH, at 130° C. (heating block), for 12 h. Purification by FC (elution: DCM/MeOH (7N NH₃): 99/1 to 85/15). The final product required a trituration in DCM at room temperature. Light beige solid, 35% (384 mg). ¹H NMR (400 MHz, DMSO-d₆, 300K) δ_{H} 10.99 (bs, 1H, NH, D₂O exchanged), 9.20 (bs, 1H, NH, D₂O exchanged), 8.30-8.22 (m, 1H), 7.79-7.73 (m, 1H), 7.15 (d, J=8.3 Hz, 1H), 7.05 (dd, J=7.3, 5.0 Hz, 1H), 3.91 (s, 2H). MS (ESI⁺): [M+H]⁺ 177.2.

### Example 2.22: Synthesis of (f)-2-(oxepan-3-ylamino)-1,4-dihydroimidazol-5-one (2.22)

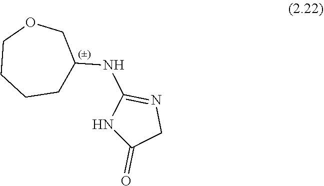 [SMILES: O=C1CN=C(NC2CCCCOC2)N1]

Compound 2.22 was synthesized according to GP1-A: reaction carried out in THF, with intermediate (1.1) (851 mol), 2.5 eq of oxepan-3-amine, at 110° C. (heating block), for 12h The product directly precipitated in the reaction medium: it was isolated after filtration, washing with cold THF, then pentane. Light beige solid, 73% (122 mg). ¹H NMR (400 MHz, DMSO-d₆, 343K) δ_{H} 7.33 (br s, 1H, NH, D₂O exchanged), 7.05 (br s, 1H, NH, D₂O exchanged), 3.96-3.79 (m, 1H), 3.76-3.62 (m, 3H), 3.61 (s, 2H), 3.56-3.46 (m, 1H), 1.91-1.77 (m, 1H), 1.77-1.60 (m, 4H), 1.59-1.46 (m, 1H). MS (ESI⁺): [M+H]⁺ 198.1.

### Example 2.23: Synthesis of 2-(cyclohexylamino)-1-methyl-4H-imidazol-5-one (2.23)

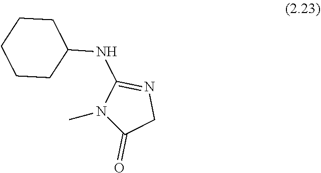

Compound (2.23) was synthesized according to GP1-C: reaction carried out in dioxane, with intermediate (1.2) (1.04 mmol), 3 eq of cyclohexylamine and 5 eq of AcOH, at 150° C. (heating block), for 3 h. Purification by FC (elution: DCM/MeOH: 99/1 to 9/1). Brown solid, 44% (89 mg). ¹H NMR (400 MHz, DMSO-d₆, 300K) δ_{H} 6.64 (br s, 1H, NH, D₂O exchanged), 3.81 (s, 2H), 3.48-3.37 (m, 1H), 2.88 (s, 3H), 1.87-1.55 (m, 4H), 1.34-1.07 (m, 6H). MS (ESI⁺): [M+H]⁺ 196.3.

### Example 2.24: Synthesis of 2-(cyclooctylamino)-1-methyl-4H-imidazol-5-one (2.24)

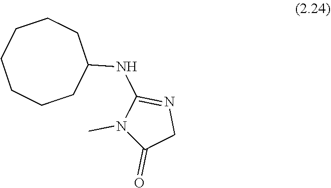 [SMILES: CN1C(=O)CN=C1NC1CCCCCCC1]

Compound (2.24) was synthesized according to GP1-C: reaction carried out in dioxane, with intermediate (1.2) (1.04 mmol), 3 eq of cyclooctylamine and 5 eq of AcOH, at 150° C. (heating block), for 3 h. Purification by FC (elution: DCM/MeOH: 99/1 to 9/1). Brown oil, 41% (105 mg). ¹H NMR (400 MHz, DMSO-d₆, 300K) δ_{H} 6.71 (br s, 1H, NH, D₂O exchanged), 3.84 (s, 2H), 3.70 (br s, 1H), 2.89 (s, 3H), 1.78-1.41 (m, 14H). MS (ESI⁺): [M+H]⁺ 224.3.

### Example 2.25: Synthesis of 2-[[(1R)-1-(methoxymethyl)-3-methyl-butyl]amino]-1-methyl-4H-imidazol-5-one (2.25)

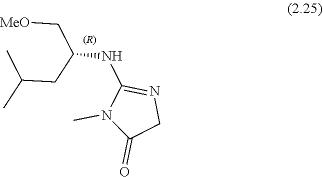

Compound (2.25) was synthesized according to GP1-C: reaction carried out in dioxane, with intermediate (1.2) (1.04 mmol), 3 eq of (2R)-1-methoxy-4-methyl-pentan-2-amine and 3 eq of AcOH, at 150° C. (heating block), for 3 h. Purification by FC (elution: DCM/MeOH: 99/1 to 9/1). Brown oil, 41% (97 mg). ¹H NMR (400 MHz, DMSO-d₆, 300K) δ_{H} n.d. MS (ESI⁺) [M+H]⁺ 228.3.

### Example 2.26: Synthesis of 2-[[(1R,2R)-2-methoxycyclopentyl]amino]-1-methyl-4H-imidazol-5-one (2.26)

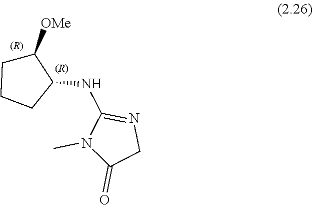

Compound (2.26) was synthesized according to GP1-C: reaction carried out in dioxane, with intermediate (1.04 mmol), 3 eq of (1R,2R)-2-methoxycyclopentanamine and 5 eq of AcOH, at 130° C. (heating block), for 3 h. Purification by FC (elution: DCM/MeOH: 99/1 to 9/1). Brown oil, 47% (130 mg). ¹H NMR (400 MHz, DMSO-d₆, 300K) δ_{H} 7.07 (br s, 1H, NH, D₂O exchanged), 3.83 (s, 2H), 3.76 (br s, 1H), 3.60 (br s, 1H), 3.22 (s, 3H), 2.86 (s, 3H), 1.98-1.82 (m, 2H), 1.69-1.47 (m, 3H), 1.43-1.33 (m, 1H). MS (ESI⁺): [M+H]⁺ 212.3.

### Example 2.27: Synthesis of 2-[[(1S,2S)-2-methoxycyclopentyl]amino]-1-methyl-4H-imidazol-5-one (2.27)

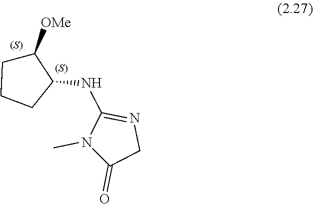

Compound (2.26) was synthesized according to GP1-C: reaction carried out in dioxane, with intermediate (1.04 mmol), 3 eq of (1S,2S)-2-methoxycyclopentanamine and 5 eq of AcOH, at 130° C. (heating block), for 3 h. Purification by FC (elution: DCM/MeOH: 99/1 to 9/1). Brown oil, 33% (96 mg). ¹H NMR (400 MHz, DMSO-d₆, 300K) δ_{H} 7.07 (br s, 1H, NH, D₂O exchanged), 3.83 (s, 2H), 3.76 (br s, 1H), 3.60 (br s, 1H), 3.22 (s, 3H), 2.86 (s, 3H), 1.98-1.82 (m, 2H), 1.69-1.47 (m, 3H), 1.43-1.33 (m, 1H). MS (ESI⁺): [M+H]⁺ 212.3.

### Example 2.28: Synthesis of 2-(3-noradamantylamino)-1-methyl-4H-imidazol-5-one (2.28)

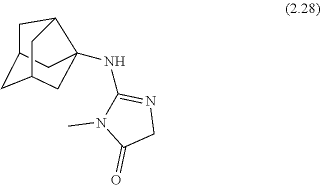 [SMILES: CN1C(=O)CN=C1NC12CC3CC(CC1C3)C2]

Compound (2.28) was synthesized according to GP1-C: reaction carried out in dioxane, with intermediate (1.04 mmol), 3 eq of 3-noradamantanamine and 5 eq of AcOH, at 150° C. (heating block), for 6 h. Purification by FC (elution: DCM/MeOH: 99/1 to 9/1). Beige solid, 37% (100 mg). ¹H NMR (400 MHz, DMSO-d₆, 300K) δ_{H} ¹H NMR (400 MHz, DMSO-d₆, 300K) δ 6.33 (s, 1H, NH, D₂O exchanged), 3.84 (s, 2H), 2.92 (s, 3H), 2.26-2.09 (m, 3H), 2.01-1.84 (m, 6H), 1.61-1.43 (m, 4H). MS (ESI⁺): [M+H]⁺ 234.3.

### Example 2.29: Synthesis of 2-(1-adamantylamino)-1-methyl-4H-imidazol-5-one (2.29)

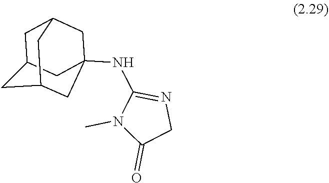 [SMILES: CN1C(=O)CN=C1NC12CC3CC(CC(C3)C1)C2]

Compound (2.29) was synthesized according to GP1-C: reaction carried out in dioxane, with intermediate (1.2) (1.04 mmol), 3 eq of adamantan-1-amine and 5 eq of AcOH, at 150° C. (heating block), for 24 h. Purification by FC (elution: DCM/MeOH: 99/1 to 9/1). Brown solid, 7% (19 mg). ¹H NMR (400 MHz, DMSO-d₆, 300K) δ_{H} 5.58 (br s, 1H, NH, D₂O exchanged), 3.83 (s, 2H), 2.90 (s, 3H), 2.11-1.97 (m, 8H), 1.71-1.59 (m, 7H). MS (ESI): [M+H]⁺ 248.3.

### Example 2.30: Synthesis of 2-[(3-hydroxy-1-adamantyl)amino]-1-methyl-4H-imidazol-5-one (2.30)

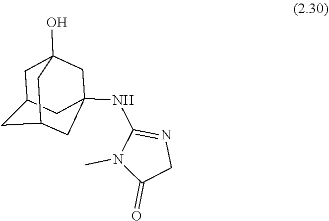 [SMILES: CN1C(=O)CN=C1NC12CC3CC(CC(O)(C3)C1)C2]

Compound (2.30) was synthesized according to GP1-C: reaction carried out in dioxane, with intermediate (1.2) (1.04 mmol), 3 eq of 3-hydroxyadamantan-1-amine and 5 eq of AcOH, at 150° C. (heating block), for 24 h. Purification by FC (elution: DCM/MeOH: 99/1 to 9/1). Beige solid, 11% (52 mg). ¹H NMR (400 MHz, DMSO-d₆, 300K) δ_{H} 5.76 (br s, 1H, NH, D₂O exchanged), 4.46 (s, 1H, OH, D₂O exchanged), 3.85 (s, 2H), 2.90 (s, 3H), 2.15 (s, 2H), 2.03-1.91 (m, 6H), 1.60-1.39 (m, 6H). MS (ESI⁺): [M+H]⁺ 264.2.

### Example 2.31: Synthesis of 2-[(3-methoxy-1-adamantyl)amino]-1-methyl-4H-imidazol-5-one (2.31)

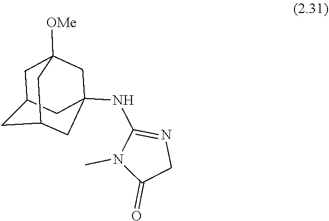 [SMILES: COC12CC3CC(CC(NC4=NCC(=O)N4C)(C3)C1)C2]

Compound (2.31) was synthesized according to GP1-C: reaction carried out in dioxane, with intermediate (1.2) (1.04 mmol), 3 eq of 3-methoxyadamantan-1-amine and 5 eq of AcOH, at 150° C. (heating block), for 24 h. Purification by FC (elution: DCM/MeOH: 99/1 to 9/1). Beige solid, 10% (36 mg). ¹H NMR (400 MHz, DMSO-d₆, 300K) δ_{H} 5.75 (br s, 1H, NH, D₂O exchanged), 3.84 (s, 2H), 3.11 (s, 3H), 2.89 (br s, 3H), 2.20 (br s, 2H), 2.09-1.91 (m, 6H), 1.68-1.54 (m, 4H), 1.54-1.41 (m, 2H). MS (ESI): [M+H]⁺ 278.3.

### Example 2.32: Synthesis of 2-[(3-fluoro-1-adamantyl)amino]-1-methyl-4H-imidazol-5-one (2.32)

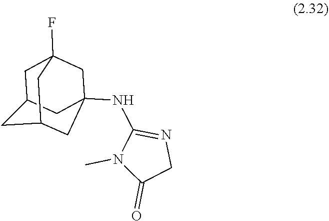 [SMILES: CN1C(=O)CN=C1NC12CC3CC(CC(F)(C3)C1)C2]

Compound (2.32) was synthesized according to GP1-C: reaction carried out in dioxane, with intermediate (1.2) (1.04 mmol), 3 eq of 3-fluoroadamantan-1-amine and 5 eq of AcOH, at 150° C. (heating block), for 24 h. Purification by FC (elution: DCM/MeOH: 99/1 to 9/1). Beige solid, 14% (64 mg). ¹H NMR (400 MHz, DMSO-d₆, 300K) δ_{H} n.d. MS (ESI⁺): [M+H]⁺ 266.2.

### Example 2.33: Synthesis of 1-methyl-2-[[2-(trifluoromethyl)phenyl]methylamino]-4H-imidazol-5-one (2.33)

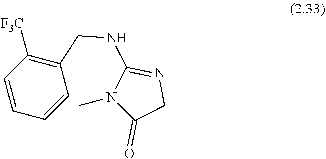 [SMILES: CN1C(=O)CN=C1NCc1ccccc1C(F)(F)F]

Compound (2.33) was synthesized according to GP1-C: reaction carried out in dioxane, with intermediate (1.2) (1.04 mmol), 3 eq of [2-(trifluoromethyl)phenyl]methanamine and 5 eq of AcOH, at 150° C. (heating block), for 1.5 h. Purification by FC (elution: DCM/MeOH: 99/1 to 9/1). Brown oil, 33% (131 mg). ¹H NMR (400 MHz, DMSO-d₆, 300K) δ_{H} 7.87-7.76 (m, 1H), 7.72-7.61 (m, 2H), 7.47-7.38 (m, 1H), 7.25 (br s, 1H, NH, D₂O exchanged), 4.49 (s, 2H), 3.86 (s, 2H), 2.96 (s, 3H). MS (ESI⁺): [M+H]⁺ 272.2.

### Example 2.34: Synthesis of 2-[[(1R)-2-methoxy-1-phenyl-ethyl]amino]-1-methyl-4H-imidazol-5-one (2.34)

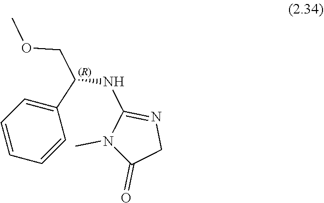

Compound (2.34) was synthesized according to GP1-C: reaction carried out in dioxane, with intermediate (1.2) (1.04 mmol), 3 eq of 3 eq of (1R)-2-methoxy-1-phenyl-ethanamine and 3 eq of AcOH, at 150° C. (heating block), for 1.5 h. Purification by FC (elution: DCM/MeOH: 99/1 to 9/1). Beige oil, 45% (195 mg). ¹H NMR (400 MHz, DMSO-d₆, 300K) δ_{H} 7.40-7.36 (m, 2H), 7.29 (t, J=7.4 Hz, 2H), 7.23-7.17 (m, 1H), 6.96 (br s, 1H, NH, D₂O exchanged), 4.60 (br s, 1H), 3.88-3.70 (m, 2H), 3.46 (br s, 2H), 3.24 (s, 3H), 2.92 (br s, 3H). MS (ESI⁺): [M+H]⁺ 248.2.

### Example 2.35: Synthesis of 1-methyl-2-[(4-methylthiazol-2-yl)methylamino]-4H-imidazol-5-one (2.35)

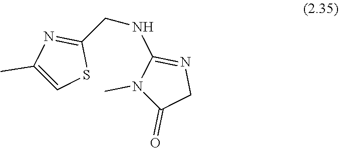

Compound (2.35) was synthesized according to GP1-C: reaction carried out in dioxane, with intermediate (1.2) (1.04 mmol), 3 eq of (4-methylthiazol-2-yl)methanamine and 5 eq of AcOH, at 150° C. (heating block), for 1.5 h. Purification by FC (elution: DCM/MeOH: 99/1 to 9/1). Beige solid, 39% (130 mg). ¹H NMR (400 MHz, DMSO-d₆, 300K) δ_{H} 7.32 (bs, 1H, NH, D₂O exchanged), 7.06 (s, 1H), 4.46 (s, 2H), 3.89 (s, 2H), 2.92 (s, 3H), 2.32 (s, 3H). MS (ESI⁺): [M+H]⁺ 225.2.

### Example 3: General Protocol 2: Synthesis of 4- and 5-substituted N-alkyl-2-nitro-aniline

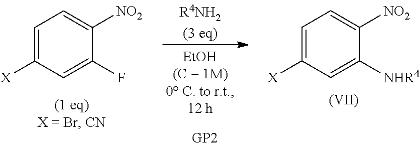

In the above scheme, X is either a cyano group or a halogen atom, in particular a bromine atom and R⁴ is a (C₁-C₃)alkyl.

GP2: The appropriate amine (3 eq) was added dropwise to a stirred solution of the suitably substituted o-fluoro-nitro-benzene (1 eq) in EtOH (C=1 M) maintained at 0° C. The resulting mixture was stirred 1 h at 0° C., brought back to room temperature and stirred another 12 h. Upon completion (TLC), the mixture was partially concentrated in vacuo and poured onto water. The precipitated solid was filtered off on a Buchner funnel and thoroughly dried in vacuo. The resulting bright red/orange solid was reprecipitated from DCM/pentane at 0° C. to yield the desired product in analytically pure form.

### Example 3.1: Synthesis of 5-bromo-N-methyl-2-nitro-aniline (3.1)

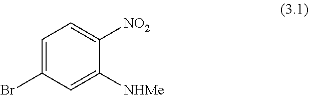 [SMILES: CNc1cc(Br)ccc1[N+](=O)[O-]]

Compound (3.1) was synthesized according to GP2: reaction carried out with 4-bromo-2-fluoro-1-nitro-benzene (68.18 mmol) with MeNH₂ (2 M solution in MeOH or 33% w/w in EtOH, 3 eq). Bright orange solid, 87% (13.763 g). ¹H NMR (400 MHz, DMSO-d₆, 300K) δ_{H} 8.30-8.20 (m, 1H, NH D₂O exchanged), 7.98 (d, J=9.1 Hz, 1H), 7.16 (s, 1H), 6.82 (d, J=9.1 Hz, 1H), 2.94 (d, J=5.0 Hz, 3H). ¹³C NMR (101 MHz, DMSO-d₆, 300K) δ_{C} 146.3, 130.9, 130.1, 127.9, 117.7, 116.4, 29.8. MS (ESI⁺): [M+H]⁺ 232.8.

### Example 3.2: Synthesis of 3-(methylamino)-4-nitro-benzonitrile (3.2)

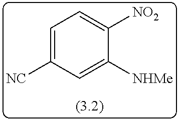 [SMILES: CNc1cc(C#N)ccc1[N+](=O)[O-]]

Compound (3.2) was synthesized according to GP2: reaction carried out with 3-fluoro-4-nitro-benzonitrile (150.5 mmol) and MeNH₂ (2 M solution in MeOH or 33% w/w in EtOH, 3 eq). Bright orange solid, 97% (25.917 g). ¹H NMR (400 MHz, DMSO-d₆, 300K) S_{H} 8.25 (d, J=5.3 Hz, 1H, NH, D₂O exchanged), 8.18 (d, J=8.7 Hz, 1H), 7.51 (s, 1H), 7.01 (dd, J=8.8, 1.7 Hz, 1H), 2.97 (d, J=5.0 Hz, 3H). ¹³C NMR (101 MHz, DMSO-d₆, 300K) δ_{C} 145.2, 132.9, 127.4, 119.4, 118.1, 117.7, 116.4, 29.9. MS (ESI⁺): [M+H]⁺ 178.0.

### Example 4: General Protocol 3—Synthesis of substituted N1- and N2-alkyl-benzene-1,2-diamine

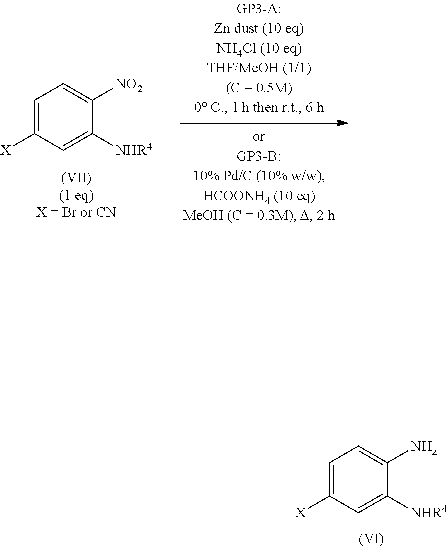

In the above scheme, X is either a cyano group or a halogen atom, in particular a bromine atom and R⁴ is a (C₁-C₃)alkyl.

GP3-A (X=Br): NH₄Cl (10 eq) was carefully added portionwise over 30 min to a stirred solution of the appropriate bromo-N-alkyl-2-nitro-aniline (1 eq) and zinc dust (10 eq) in a THF/MeOH mixture (1/1) (C=0.5 M) maintained at 0° C. When the addition ended, the mixture was stirred another hour at 0° C. (until the orange color fully disappeared) and gradually brought back to room temperature. The reaction media was stirred another 6 h at room temperature. Upon completion (TLC), the mixture was filtered off on a pad of celite. The celite was rinsed with MeOH. The filtrate was concentrated in vacuo, adsorbed on silica and purified by FC on silica gel (elution: cyclohexane/AcOEt: 8/2 to 1/1) to yield the desired bromo-N-alkyl-benzene-1,2-diamine in analytically pure form.

### Example 4.1: Synthesis of 4-bromo-N2-methyl-benzene-1,2-diamine (4.1)

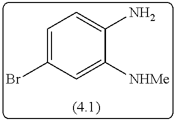

Compound (4.1) was synthesized according to GP3-A: reaction carried out with intermediate (3.1) (17.31 mmol). Brown oil, 81% (2.83 g). ¹H NMR (400 MHz, DMSO-d₆, 300K) δ_{H} 6.52 (dd, J=8.1, 2.2 Hz, 1H), 6.44 (d, J=8.1 Hz, 1H), 6.40 (d, J=2.2 Hz, 1H), 4.88 (bs, 1H), 4.61 (bs, 2H), 2.68 (d, J=3.8 Hz, 3H). ¹³C NMR (101 MHz, DMSO-d_{b}, 300K) δ_{C} 138.8, 134.4, 118.4, 114.6, 110.9, 108.8, 29.9. MS (ESI⁺): [M+H]⁺ 201.1.

GP3-B (X=CN): 10% Pd/C (10% w/w) was carefully added portionwise to a stirred solution of the appropriate cyano-substituted N-alkyl-2-nitro-aniline (1 eq) and ammonium formiate (10 eq) in MeOH (C=0.3 M). The resulting mixture was refluxed 6 h. Upon completion (TLC), the mixture was filtered off on pad of celite. The celite was rinsed with MeOH. The filtrate was concentrated in vacuo, the resulting crude was partitioned between AcOEt and sat. NaHCO_{3(aq)}. The aqueous layer was extracted three times with AcOEt. The combined organic layers were washed with water and brine, dried over MgSO₄, filtered, and concentrated in vacuo. The resulting solid was reprecipitated from DCM/pentane at 0° C. to yield the desired product in analytically pure form.

### Example 4.2: Synthesis of 4-amino-3-(methylamino)benzonitrile (4.2)

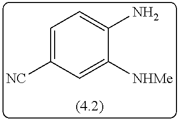 [SMILES: CNc1cc(C#N)ccc1N]

Compound (4.2) was synthesized according to GP3-B: reaction carried out with intermediate (3.2) (146.2 mmol). Beige solid, 87% (18.764 g). ¹H NMR (400 MHz, DMSO-d₆, 300K) δ_{H} 6.85 (dd, J=8.0, 1.8 Hz, 1H), 6.61-6.52 (m, 2H), 5.48 (bs, 2H, NH, D₂O exchanged), 5.01 (q, J=5.0 Hz, 1H, NH, D₂O exchanged), 2.72 (d, J=4.9 Hz, 3H). ¹³C NMR (101 MHz, DMSO-d₆, 300K) 8_{C} 140.4, 136.4, 122.2, 121.4, 112.2, 110.4, 97.3, 29.9. MS (ESI⁺): [M+H]⁺ 148.1.

### Example 5: General protocol 4: cyclization of brominated 2-aminophenols and N2-methylbenzene-1,2-diamines with triethyl orthoformate

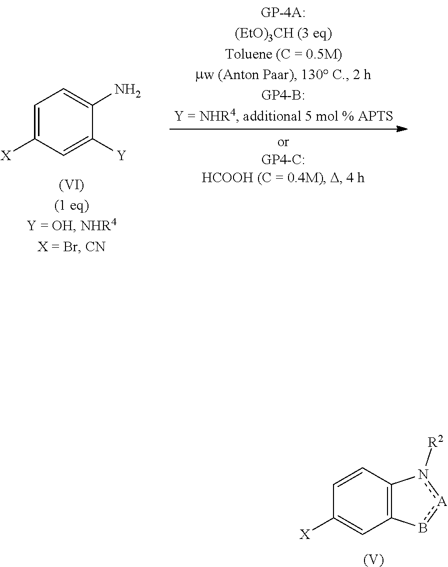

In the above scheme, Y is either a —OH group or a —NHR⁴, X is either a cyano group or a halogen atom, in particular a bromine atom and R⁴ is a (C₁-C₃)alkyl.

GP4-A (Y=OH and X=Br): a solution of the appropriate 2-amino-bromo-phenol (1 eq) and (EtO)₃CH (2 eq) in toluene (C=0.5 M) was irradiated at 130° C. for 2 h. Upon completion (TLC), the mixture was cooled down room temperature, directly adsorbed on silica, and purified by FC on silica gel using the appropriate gradient of solvents (elution cyclohexane/AcOEt: 1/0 to 6/4) to yield the desired bromo-1,3-benzoxazole in analytically pure form.

### Example 5.1: Synthesis of 6-bromo-1,3-benzoxazole (5.1)

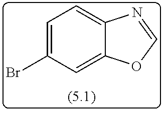

Compound (5.1) was synthesized according to GP4-A: reaction carried out with 2-amino-5-bromo-phenol (21.27 mmol). Light beige solid, 89% (3.745 g). ¹H NMR (400 MHz, DMSO-d₆, 300K) δ_{H} 8.79 (s, 1H), 8.12 (s, 1H), 7.77 (d, J=8.4 Hz, 1H), 7.58 (d, J=8.5 Hz, 1H). ¹³C NMR (101 MHz, DMSO-d₆, 300K) δ_{C} 154.8, 150.0, 139.1, 127.8, 121.5, 117.7, 114.5. MS (ESI⁺): [M+H]⁺ 199.7.

GP4-B (Y=NHR⁴ and X=Br): a solution of the appropriate 4-bromo-N-methyl-benzene-1,2-diamine (1 eq), (EtO)₃CH (2 eq), and APTS·H₂O (5 mol %) in toluene (C=0.5 M) was irradiated at 130° C. for 2 h. Upon completion (TLC), the mixture was partitioned between AcOEt and sat. NaHCO_{3(aq)}. The aqueous layer was extracted three times with AcOEt. The combined organic layers were washed with brine and water, dried over MgSO₄, filtered, and concentrated in vacuo. The resulting brown solid was dissolved in DCM at 0° C. and precipitated from pentane. The precipitated light beige solid was filtered off on a Buchner funnel, thoroughly rinsed with pentane, and dried in vacuo to yield the desired bromo-1-methyl-benzimidazole in analytically pure form.

### Example 5.2: Synthesis of 6-bromo-1-methyl-benzimidazole (5.2)

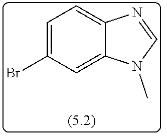

Compound (5.2) was synthesized according to GP4-B: reaction carried out with intermediate (4.1) (10.94 mmol). Light beige solid, 79% (1.815 g). ¹H NMR (400 MHz, DMSO-d₆, 300K) & 8.21 (s, 1H), 7.86 (s, 1H), 7.60 (d, J=8.5 Hz, 1H), 7.33 (d, J=8.4 Hz, 1H), 3.83 (s, 3H). ¹³C NMR (101 MHz, DMSO-d₆, 300K) δ_{C} 145.6, 142.4, 135.8, 124.3, 121.0, 114.7, 113.3, 30.8. MS (ESI⁺): [M+H]⁺ 212.8.

GP4-C(Y=NHR⁴ and X=CN): a solution of cyano-substituted N2-methylbenzene-1,2-diamine (1 eq) was refluxed in HCOOH (C=0.4 M) for 4 h. Upon completion (TLC), the mixture was brought back to room temperature, and concentrated in vacuo. The resulting crude was carefully treated with sat. NaHCO_{3(aq)} to neutral pH. The precipitated solid was filtered on a Büchner funnel. The solid was solubilized in DCM and washed with water, sat. NaHCO_{3(aq)}, dried over MgSO₄, filtered and concentrated in vacuo to yield the desired solid in analytically pure form. Higher purity may be achieved by reprecipitation from DCM/pentane at 0° C.

### Example 5.3: Synthesis of 3-methylbenzimidazole-5-carbonitrile (5.3)

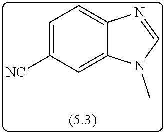

Compound (5.3) was synthesized according to GP4-C: reaction carried out with intermediate (4.2) (127.5 mmol). Beige solid, 94% (18.829 g). ¹H NMR (400 MHz, DMSO-d_{b}, 300K) δ_{H} 8.46 (s, 1H), 8.24 (s, 1H), 7.82 (d, J=8.4 Hz, 1H), 7.59 (dd, J=8.3, 1.6 Hz, 1H), 3.90 (s, 3H). ¹³C NMR (101 MHz, DMSO-d₆, 300K) δ_{C} 148.1, 146.2, 134.3, 124.8, 120.4, 119.9, 115.9, 104.0, 31.1. MS (ESI⁺): [M+H]⁺ 158.1.

### Example 6: General protocol 5—Palladium-catalyzed vinylation of heteroarylbromides

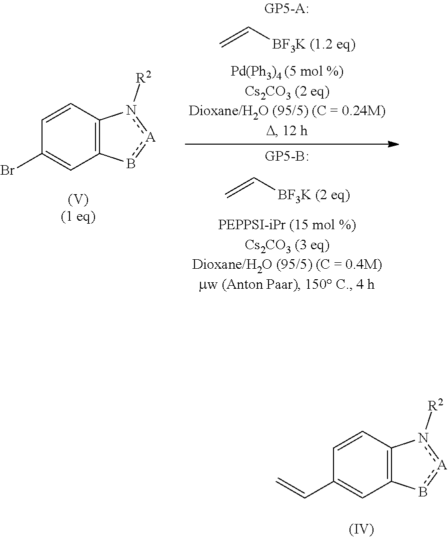

In the above scheme, A, B and R² are as defined above. GP5-A: a mixture of the appropriate heteroarylbromide (1 eq), potassium vinyltrifluoroborate (1.2 eq), Cs₂CO₃ (2 eq), and Pd(PPh₃)₄ (5 mol %) in dioxane/H₂O (95/5) (C=0.4 M) was thoroughly purged with vacuum/argon cycles. The mixture was brought to reflux and heated for 12 h. Upon completion (¹H NMR), the mixture was partitioned between H₂O and AcOEt. The aqueous layer was extracted three times with AcOEt. The combined organic layers were dried over MgSO₄, filtered, concentrated in vacuo, adsorbed on silica, and purified by FC on silica gel using the appropriate gradient of solvents (see details below for each compound) to yield the desired vinylheteroaryl in analytically pure form.

### Example 6.1: Synthesis of 6-vinyl-1,3-benzoxazole (6.1)

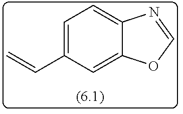

Compound (6.1) was synthesized according to GP5-A: reaction carried out with intermediate (5.1) (40.70 mmol). Purification by FC: elution: cyclohexane/AcOEt: 99/1 to 8/2. Brown oil, 74% (4.345 g). ¹H NMR (400 MHz, DMSO-d₆, 300K) δ_{H} 8.73 (s, 1H), 7.90 (s, 1H), 7.75 (d, J=8.3 Hz, 1H), 7.53 (d, J=8.2 Hz, 2H), 6.86 (dd, J=17.7, 11.0 Hz, 1H), 5.94 (d, J=17.6 Hz, 1H), 5.33 (d, J=10.9 Hz, 1H). ¹³C NMR (101 MHz, DMSO-d₆, 300K) δ_{C} 154.7, 149.9, 139.5, 136.2, 135.3, 123.2, 119.9, 115.0, 108.4. MS (ESI⁺): [M+H]⁺ 145.9. (N.B.: (6.1) has nearly identical Rf as starting material (5.1) in cyclohexane/AcOEt: 8/2).

GP5-B: In a sealed tube thoroughly purged with vacuum/argon cycles, a mixture of the appropriate heteroarylbromide (1 eq), potassium vinyltrifluoroborate (2 eq), Cs₂CO₃ (3 eq), and PEPPSI-iPr (15 mol %) in dioxane/H₂O (95/5) (C=0.4 M) was irradiated for 4 h at the adequate temperature (see details below for each compound). Upon completion (¹H NMR), the mixture was cooled down room temperature, directly adsorbed on silica, and purified by FC on silica gel using the appropriate gradient of solvents (see details below for each compound) to yield the desired vinylheteroaryl in analytically pure form.

### Example 6.2: Synthesis of 1-methyl-6-vinyl-benzimidazole (6.2)

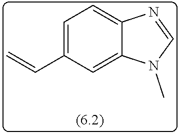

Compound (6.2) was synthesized according to GP5-B: reaction carried out with intermediate (5.2) (4.26 mmol). Purification by FC: elution: DCM/MeOH: 99/1 to 94/6. Brown oil, 82% (551 mg). ¹H NMR (400 MHz, CDCl₃, 300K) S₁H 7.87 (s, 1H), 7.74 (d, J=8.3 Hz, 1H), 7.49-7.33 (m, 2H), 6.86 (dd, J=17.5, 10.9 Hz, 1H), 5.79 (d, J=17.5 Hz, 1H), 5.26 (d, J=10.9 Hz, 1H), 3.85 (s, 3H). ¹³C NMR (101 MHz, CDCl₃) S_{C} 144.2, 143.8, 137.3, 135.0, 133.2, 120.8, 120.2, 113.2, 107.3, 31.1. MS (ESI⁺): [M+H]⁺ 159.1.

### Example 6.3: Synthesis of 6-vinyl-1,2,3-benzothiadiazole (6.3)

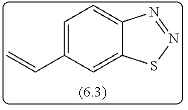

Compound (6.3) was synthesized according to GP5-B: reaction carried out with 6-bromo-1,2,3-benzothiadiazole (15.38 mmol). Purification by FC: elution: cyclohexane/AcOEt: 98/2 to 8/2. Brown oil, 59% (1.466 g). ¹H NMR (400 MHz, DMSO-d₆, 300K) δ_{H} 8.66 (d, J=8.7 Hz, 1H), 8.45 (s, 1H), 7.93 (d, J=8.7 Hz, 1H), 6.97 (dd, J=17.6, 10.9 Hz, 1H), 6.13 (d, J=17.6 Hz, 1H), 5.55 (d, J=10.9 Hz, 1H). ¹³C NMR (101 MHz, DMSO-d₆, 300K) δ_{C} 157.4, 141.4, 138.3, 135.5, 125.5, 123.3, 118.5, 117.8. MS (ESI⁺): [M+H]⁺ 163.1.

### Example 7: General Protocol 6—Synthesis of Heteroarylcarbaldehydes from Vinylheteroaryls—Osmium-Catalyzed Lemieux-Johnson Oxidation

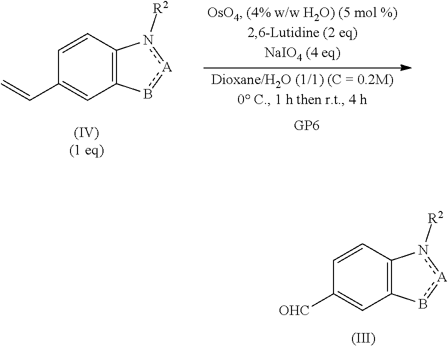

In the above scheme, A, B and R² are as defined above.

GP6: NaIO₄ (4 eq) was added portionwise to a stirred solution of the appropriate vinylheteroaryl (1 eq), 2,6-lutidine (2 eq), OSO₄ (4% w/w H₂O sol., 5 mol %), in dioxane/H₂O (1/1) (C=0.2 M) maintained at 0° C. The reaction mixture was vigorously stirred at 0° C. for 1 h, brought back to room temperature and stirred another 4 h. Upon completion (TLC), the mixture was partitioned between AcOEt and H₂O. The aqueous layer was extracted with AcOEt (×3). The combined organic layers were washed with sat. NH₄Cl_{(aq)}, sat. NaHCO_{3(aq)}, dried over MgSO₄, filtered, and concentrated in vacuo (trace amounts of 2,6-lutidine were removed by co-evaporation with toluene). The solid residue was adsorbed on silica and purified by FC on silica gel using the appropriate gradient of solvents (see details below for each compound).

### Example 7.1: Synthesis of 1,3-benzoxazole-6-carbaldehyde (7.1)

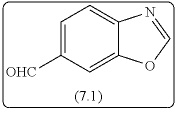 [SMILES: O=Cc1ccc2ncoc2c1]

Compound (7.1) was synthesized according to GP6: reaction carried out with intermediate (6.1) (20.66 mmol). Purification by FC: elution: cyclohexane/AcOEt: 99/1 to 7/3. Light beige solid, 87% (2.654 g). ¹H NMR (400 MHz, DMSO-d₆, 300K) δ_{H} 10.12 (s, 1H), 9.01 (s, 1H), 8.34 (s, 1H), 8.16-7.91 (m, 2H). ¹³C NMR (101 MHz, DMSO-d_{b}, 300K) δ_{C} 192.1, 157.5, 149.5, 144.6, 134.0, 126.0, 120.7, 112.6. MS (ESI⁺): [M+H]⁺ 147.9.

### Example 7.2: Synthesis of 3-methylbenzimidazole-5-carbaldehyde (7.2)

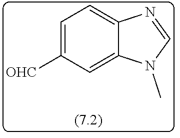

Compound (7.2) was synthesized according to GP6: reaction carried out with intermediate (6.2) (3.48 mmol). Purification by FC: elution: DCM/MeOH: 99/1 to 94/6. Colorless solid, 54% (300 mg). ¹H NMR (400 MHz, CDCl₃, 300K) δ_{H} 10.12 (s, 1H), 8.39-8.22 (m, 1H), 8.02 (s, 1H), 7.95 (d, J=8.4 Hz, 1H), 7.87 (d, J=8.3 Hz, 1H), 3.98 (s, 3H). ¹³C NMR (101 MHz, CDCl₃, 300K) δ 192.1, 148.5, 147.0, 134.9, 132.1, 124.7, 120.9, 111.4, 31.5. MS (ESI⁺): [M+H]⁺ 161.1.

### Example 7.3: Synthesis of 1,2,3-benzothiadiazole-6-carbaldehyde (7.3)

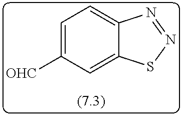 [SMILES: O=Cc1ccc2nnsc2c1]

Compound (7.3) was synthesized according to GP6: reaction carried out with intermediate (6.3) (9.04 mmol). Purification by FC: elution: cyclohexane/AcOEt: 95/5 to 80/20. Colorless solid, 41% (609 mg). ¹H NMR (400 MHz, DMSO-d₆, 300K) δ_{H} 10.27 (s, 1H), 9.03 (s, 1H), 8.89 (d, J=8.6 Hz, 1H), 8.20 (dd, J=8.6, 1.5 Hz, 1H). ¹³C NMR (101 MHz, DMSO-d₆, 300K) δ_{C} 192.9, 159.4, 141.1, 135.5, 126.5, 124.1, 124.1. MS (ESI⁺): [M+H]⁺ 165.0.

### Example 8: General Protocol 7—Synthesis of Heteroarylcarbaldehydes from Heteroarylcarbonitriles—Adam's Catalyst-Mediated Oxidation

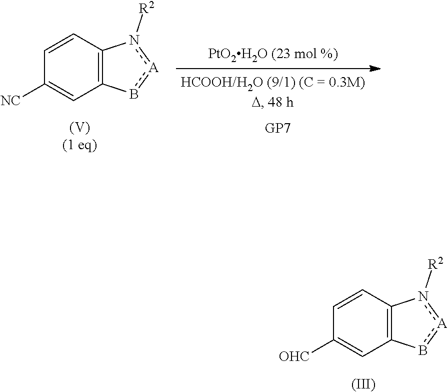

In the above scheme, A, B and R² are as defined above.

GP7: Adam's catalyst (23 mol %) was carefully added portionwise to a stirred solution of the appropriate carbonitrile (1 eq) in a HCOOH/H₂O solution (9/1) (C=0.3 M). The resulting mixture was refluxed for 48 h. Upon completion (TLC), the mixture was brought back to room temperature and filtered off on pad of celite. The celite was rinsed with HCOOH. The filtrate was concentrated in vacuo and partitioned between DCM and sat. NaHCO_{3(aq)}. The aqueous layer was extracted three times with DCM. The combined organic layers were washed with sat. NaHCO_{3(aq)}, water, dried over MgSO₄, filtered, concentrated in vacuo, adsorbed on silica, and purified by FC on silica gel (see details below).

### Example 8.1: Synthesis of 3-methylbenzimidazole-5-carbaldehyde (7.2)

Compound (7.2) was synthesized according to GP7 from intermediate (5.3) (30.32 mmol). Purification by FC: elution: DCM/MeOH: 99/1 to 94/6. Colorless solid, 61% (2.986 g). The final product required a reprecipitation from DCM/pentane at 0° C. Spectroscopic and analytical data were identical to those described above in example 7.2.

### Example 9: General Protocol 8—Knoevenagel Condensation Between Thiohydantoin and Heteroarylcarbalhydes (ROUTE 2)

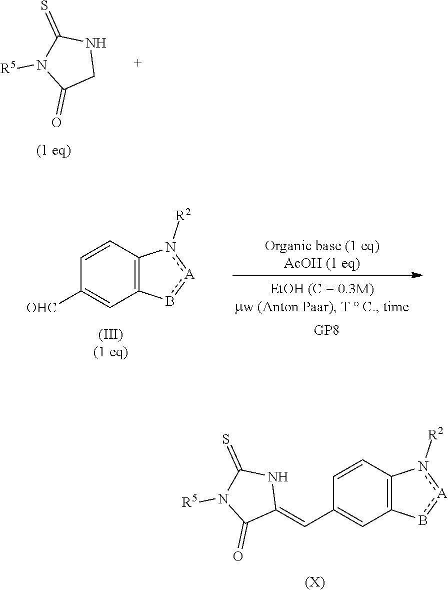

In the above scheme, A, B, R² and R⁵ are as defined above.

GP8: a stirred solution of thiohydantoin (1 eq), the appropriate heteroarylcarbaldehyde (1 eq), organic base (1 eq) and AcOH (1 eq) in EtOH (C=0.3 M) was heated in a sealed tube in a microwave oven (Anton Paar) for the indicated time at the adequate temperature (see details below for each compound). Upon completion (followed by consumption of the heteroarylcarbaldehyde on TLC), the reaction media was cooled down and added onto stirred water. The precipitated solid was stirred for 30 min and filtered off on a fritted glass funnel, thoroughly dried, and could be used in the next step without further purification. Higher purity may be achieved by trituration.

### Example 9.1: Synthesis of (5Z)-5-(1,3-benzoxazol-6-ylmethylene)-2-thioxo-imidazolidin-4-one (9.1)

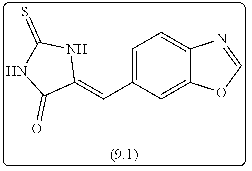 [SMILES: O=C1NC(=S)N/C1=C\c1ccc2ncoc2c1]

Compound 9.1 was synthesized according to GP8: reaction carried out with 2-thiohydantoin (2.72 mmol), intermediate (7.1), AcOH, and ethanolamine as the organic base. Reaction temperature: 80° C., time: 15 min. The final product required a trituration in EtOH. Yellow solid, 72% (483 mg). ¹H NMR (400 MHz, DMSO-d₆, 300K) δ_{H} 12.33 (bs, 2H, NH, D₂O exchanged), 8.84 (s, 1H), 8.25 (s, 1H), 7.82 (d, J=8.3 Hz, 1H), 7.75 (d, J=8.4 Hz, 1H), 6.64 (s, 1H). ¹³C NMR (101 MHz, DMSO-d₆, 300K) δ_{C} 179.4, 165.8, 155.7, 149.8, 140.3, 130.2, 128.0, 127.7, 120.2, 112.2, 111.1. MS (ESI⁺): [M+H]⁺ 245.8.

### Example 9.2: Synthesis of (5Z)-5-(1H-indazol-5-ylmethylene)-2-thioxo-imidazolidin-4-one (9.2)

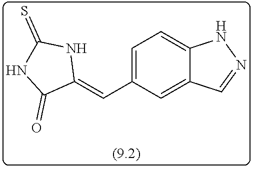 [SMILES: O=C1NC(=S)N/C1=C\c1ccc2[nH]ncc2c1]

Compound (9.2) was synthesized according to GP8: reaction carried out with 2-thiohydantoin (4.42 mmol), indazole-5-carbaldehyde, AcOH, and piperidine as the organic base. Reaction temperature: 110° C., time: 60 min. The final product required a trituration in EtOH. Yellow solid, 89% (742 mg). ¹H NMR (400 MHz, DMSO-d₆, 300K) δ_{H} 13.24 (bs, 1H, NH, D₂O exchanged), 12.33 (bs, 1H, NH, D₂O exchanged), 12.19 (bs, 1H, NH, D₂O exchanged), 8.27 (s, 1H), 8.14 (s, 1H), 7.70 (d, J=8.7 Hz, 1H), 7.55 (d, J=8.7 Hz, 1H), 6.64 (s, 1H). ¹³C NMR (101 MHz, DMSO-d, 300K) δ_{C} 179.2, 166.3, 140.1, 135.0, 129.0, 126.8, 125.2, 123.8, 113.6, 111.0. MS (ESI⁺): [M+H]T 244.9.

### Example 9.3: Synthesis of (5Z)-5-[(2-methylindazol-5-yl)methylene]-2-thioxo-imidazolidin-4-one (9.3)

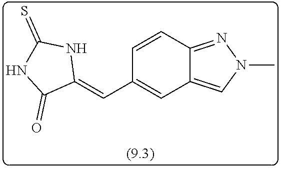 [SMILES: Cn1cc2cc(/C=C3\NC(=S)NC3=O)ccc2n1]

Compound (9.3) was synthesized according to GP8: reaction carried out with 2-thiohydantoin (2.12 mmol), 2-methylindazole-5-carbaldehyde, AcOH, with piperidine as the organic base, Reaction temperature: 110° C., time: 60 min. The final product required a trituration in EtOH. Yellow solid, 89% (485 mg). ¹H NMR (400 MHz, DMSO-d₆, 300K) δ_{H} 12.31 (bs, 1H, NH, D₂O exchanged), 12.16 (bs, 1H, NH, D₂O exchanged), 8.44 (s, 1H), 8.22 (s, 1H), 7.62-7.54 (m, 2H), 6.58 (s, 1H), 4.19 (s, 3H). ¹³C NMR (101 MHz, DMSO-d₆, 300K) δ_{C} 178.6, 165.8, 147.7, 127.5, 126.3, 126.1, 125.2, 123.9, 121.9, 117.0, 113.3, 39.9. MS (ESI⁺): [M+H]⁺ 259.1.

### Example 9.4: Synthesis of (5Z)-5-[(1-methylindazol-5-yl)methylene]-2-thioxo-imidazolidin-4-one (9.4)

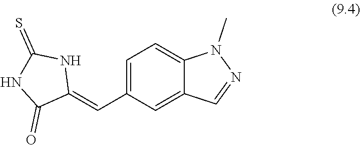 [SMILES: Cn1ncc2cc(/C=C3\NC(=S)NC3=O)ccc21]

Compound (9.4) was synthesized according to GP8: reaction carried out with 2-thiohydantoin (1.72 mmol), 1-methylindazole-5-carbaldehyde, AcOH, and piperidine as the organic base. Reaction temperature: 110° C., time: 60 min. The final product required a trituration in EtOH. Yellow solid, 92% (408 mg). ¹H NMR (400 MHz, DMSO-d₆, 300K) δ_{H}12.33 (bs, 1H, NH, D₂O exchanged), 12.20 (bs, 1H, NH, D₂O exchanged), 8.24 (s, 1H), 8.11 (d, J=0.9 Hz, 1H), 7.75 (dd, J=8.9, 1.4 Hz, 1H), 7.67 (d, J=8.8 Hz, 1H), 6.64 (s, 1H), 4.06 (s, 3H). ¹³C NMR (101 MHz, DMSO-d₆, 300K) δ_{C} 178.8, 165.8, 139.4, 133.5, 128.4, 126.4, 124.8, 123.9, 123.6, 112.9, 110.1, 35.5. MS (ESI⁺): [M+H]⁺ 244.9.

### Example 9.5: Synthesis of (5Z)-5-(1H-benzimidazol-5-ylmethylene)-2-thioxo-imidazolidin-4-one (9.5)

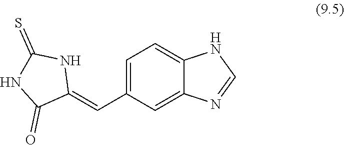 [SMILES: O=C1NC(=S)N/C1=C\c1ccc2[nH]cnc2c1]

Compound (9.5) was synthesized according to GP8: reaction carried out with 2-thiohydantoin (4.2 mmol), 1H-benzimidazole-5-carbaldehyde, AcOH, and piperidine as the organic base. Reaction temperature: 110° C., time: 60 min. The final product required a trituration in EtOH. Yellow solid, 95% (975 mg). ¹H NMR (400 MHz, DMSO-d₆, 343K) of major tautomer δ_{H} 12.42 (bs, 1H, NH, D₂O exchanged), 12.03 (bs, 2H, NH, D₂O exchanged), 8.25 (s, 1H), 8.03 (br s, 1H), 7.69-7.51 (m, 2H), 6.64 (s, 1H). MS (ESI⁺): [M+H]⁺ 245.8.

### Example 9.6: Synthesis of (5Z)-5-[(3-methylbenzimidazol-5-yl)methylene]-2-thioxo-imidazolidin-4-one (9.6)

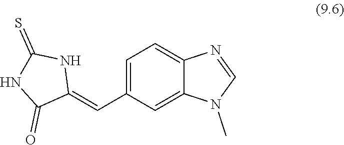 [SMILES: Cn1cnc2ccc(/C=C3\NC(=S)NC3=O)cc21]

Compound (9.6) was synthesized according to GP8: reaction carried out with 2-thiohydantoin (4.37 mmol), intermediate (7.2), AcOH, and piperidine as the organic base. Reaction temperature: 110° C., time: 60 min. The final product required a trituration in EtOH. Yellow solid, 76% (858 mg). ¹H NMR (400 MHz, DMSO-d₆, 300K) δ_{H} 12.38 (bs, 1H, NH, D₂O exchanged), 12.24 (bs, 1H, NH, D₂O exchanged), 8.29 (s, 1H), 8.02 (s, 1H), 7.66 (d, J=8.4 Hz, 1H), 7.52 (d, J=8.2 Hz, 1H), 6.65 (s, 1H), 3.92 (s, 3H). ¹³C NMR (101 MHz, DMSO-d₆, 300K) δ_{C} 178.8, 165.8, 146.6, 144.3, 135.1, 126.5, 126.4, 125.4, 119.6, 113.2, 111.6, 31.2. MS (ESI⁺): [M+H]⁺ 259.1.

### Example 9.7: Synthesis of (5Z)-5-(1,3-benzoxazol-6-ylmethylene)-3-methyl-2-thioxo-imidazolidin-4-one (9.7)

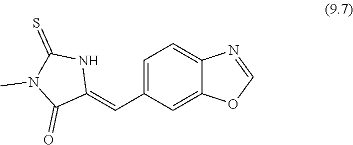 [SMILES: CN1C(=O)/C(=C/c2ccc3ncoc3c2)NC1=S]

Compound (9.7) was synthesized according to GP8: reaction carried out with 3-methyl-2-thiohydantoin (13.83 mmol), intermediate (7.1), AcOH, and ethanolamine as the organic base. Reaction temperature: 80° C., time: 20 min. The final product required a trituration in EtOH. Yellow solid, 80% (2.90 g). ¹H NMR (400 MHz, DMSO-d₆, 300K) δ_{H} 12.46 (bs, 1H, NH, D₂O exchanged), 8.83 (s, 1H), 8.25 (s, 1H), 7.82 (d, J=8.3 Hz, 1H), 7.78-7.73 (m, 1H), 6.76 (s, 1H), 3.21 (s, 3H). ¹³C NMR (101 MHz, DMSO-d₆, 300K) Se 179.2, 164.1, 155.7, 149.8, 140.5, 130.0, 127.7, 126.5, 120.2, 112.3, 112.2, 27.3. MS (ESI⁺): [M+H]⁺ 260.1.

### Example 9.8: Synthesis of (5Z)-5-(1H-indazol-5-ylmethylene)-3-methyl-2-thioxo-imidazolidin-4-one (9.8)

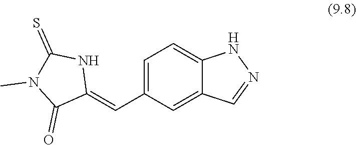 [SMILES: CN1C(=O)/C(=C/c2ccc3[nH]ncc3c2)NC1=S]

Compound (9.8) was synthesized according to GP8: reaction carried out with 3-methyl-2-thiohydantoin (13.67 mmol), indazole-5-carbaldehyde, AcOH, and piperidine as the organic base. Reaction temperature: 110° C., time: 60 min. The final product required a trituration in EtOH. Yellow solid, 92% (3.25 g). ¹H NMR (400 MHz, DMSO-d₆, 300K) δ_{H} 13.25 (bs, 1H, NH, D₂O exchanged), 12.38 (bs, 1H, NH, D₂O exchanged), 8.30 (s, 1H), 8.15 (s, 1H), 7.72 (dd, J=8.8, 1.7 Hz, 1H), 7.56 (d, J=8.7 Hz, 1H), 6.78 (s, 1H), 3.21 (s, 3H). ¹³C NMR (101 MHz, DMSO-d₆, 300K) δ_{C} 178.7, 164.1, 139.7, 134.6, 128.6, 124.9, 124.7, 123.6, 123.4, 114.3, 110.5, 27.2. MS (ESI⁺): [M+H]⁺ 259.1.

### Example 9.9: Synthesis of (5Z)-3-methyl-5-[(2-methylindazol-5-yl)methylene]-2-thioxo-imidazolidin-4-one (9.9)

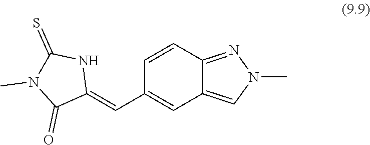

Compound (9.9) was synthesized according to GP8: reaction carried out with 3-methyl-2-thiohydantoin (8.07 mmol), 2-methylindazole-5-carbaldehyde, AcOH, and piperidine as the organic base. Reaction temperature: 110° C., time: 60 min. The final product required a trituration in EtOH. Yellow solid, 92% (2.01 g). ¹H NMR (400 MHz, DMSO-d₆) δ_{H} ¹H NMR (400 MHz, DMSO-d₆, 300K) δ 12.35 (bs, 1H, NH, D₂O exchanged), 8.45 (s, 1H), 8.25 (s, 1H), 7.63-7.57 (m, 2H), 6.72 (s, 1H), 4.19 (s, 3H), 3.21 (s, 3H). ¹³C NMR (101 MHz, DMSO-d₆, 300K) δ_{C} 178.5, 164.2, 147.8, 127.6, 126.2, 125.2, 124.8, 124.2, 121.9, 117.1, 114.5, 40.1, 27.2. MS (ESI⁺): [M+H]⁺ 273.1.

### Example 9.10: Synthesis of (5Z)-3-methyl-5-[(2-methylindazol-5-yl)methylene]-2-thioxo-imidazolidin-4-one (9.10)

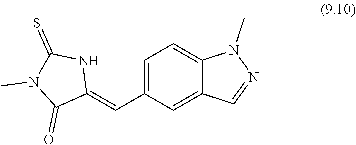 [SMILES: CN1C(=O)/C(=C/c2ccc3c(cnn3C)c2)NC1=S]

Compound (9.9) was synthesized according to GP8: reaction carried out with 3-methyl-2-thiohydantoin (14.90 mmol), 1-methylindazole-5-carbaldehyde, AcOH, and piperidine as the organic base. Reaction temperature: 110° C., time: 60 min. The final product required a trituration in EtOH. Yellow solid, 96% (3.90 g). ¹H NMR (400 MHz, DMSO-d₆, 300K) δ_{H} 12.39 (bs, 1H, NH, D₂O exchanged), 8.27 (s, 1H), 8.12 (d, J=0.9 Hz, 1H), 7.78 (dd, J=8.8, 1.7 Hz, 1H), 7.67 (d, J=8.9 Hz, 1H), 6.78 (s, 1H), 4.07 (s, 3H), 3.21 (s, 3H). ¹³C NMR (101 MHz, DMSO-d₆, 300K) δ_{C} 178.7, 164.1, 139.4, 133.5, 128.5, 124.9, 124.8, 123.9, 123.8, 114.1, 110.1, 35.5, 27.2. MS (ESI⁺): [M+H]⁺ 273.1.

### Example 9.11: Synthesis of (5Z)-5-(1H-benzimidazol-5-ylmethylene)-3-methyl-2-thioxo-imidazolidin-4-one (9.11)

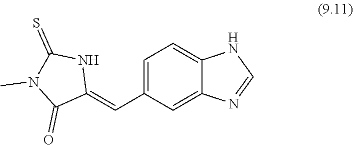 [SMILES: CN1C(=O)/C(=C/c2ccc3[nH]cnc3c2)NC1=S]

Compound (9.11) was synthesized according to GP8: reaction carried out with 3-methyl-2-thiohydantoin (8.07 mmol), 1H-benzimidazole-5-carbaldehyde, AcOH, and piperidine as the organic base. Reaction temperature: 110° C., time: 60 min. The final product required a trituration in EtOH. Yellow solid, 90% (1.88 g). ¹H NMR (400 MHz, DMSO-d₆, 300K) of major tautomer δ_{H} 12.63 (bs, 1H, NH, D₂O exchanged), 12.42 (bs, 1H, NH, D₂O exchanged), 8.32 (s, 1H), 8.08 (s, 1H), 7.62 (s, 2H), 6.80 (s, 1H), 3.21 (s, 3H). MS (ESI⁺) [M+H]⁺ 259.1.

### Example 9.12: Synthesis of (5Z)-5-(1H-benzimidazol-5-ylmethylene)-3-methyl-2-thioxo-imidazolidin-4-one (9.11)

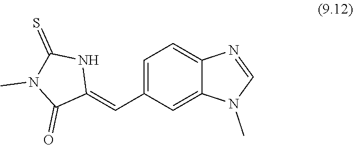 [SMILES: CN1C(=O)/C(=C/c2ccc3ncn(C)c3c2)NC1=S]

Compound (9.12) was synthesized according to GP8: reaction carried out with 3-methyl-2-thiohydantoin (8.53 mmol), intermediate (7.2), AcOH, and piperidine as the organic base. Reaction temperature: 110° C., time: 60 min. The final product required a trituration in EtOH. Yellow solid, 95% (2.21 g). ¹H NMR (400 MHz, CF₃COOD, 300K) δ_{H} 9.03 (s, 1H), 8.03 (d, J=1.5 Hz, 1H), 7.89 (d, J=8.7 Hz, 1H), 7.82 (dd, J=8.8, 1.5 Hz, 1H), 7.10 (s, 1H), 4.22 (s, 3H), 3.40 (s, 3H). ¹³C NMR (101 MHz, CF₃COOD, 300K) δ_{C} 182.5, 168.1, 143.2, 134.6, 134.1, 133.2, 132.7, 129.4, 117.9, 117.4, 114.8, 35.0, 29.1. MS (ESI⁺): [M+H]⁺ 273.1.

### Example 10: General protocol 9—S-Alkylation of (5Z)-5-heteroarylmethylene-2-thioxo-imidazolidin-4-ones (ROUTE 2)

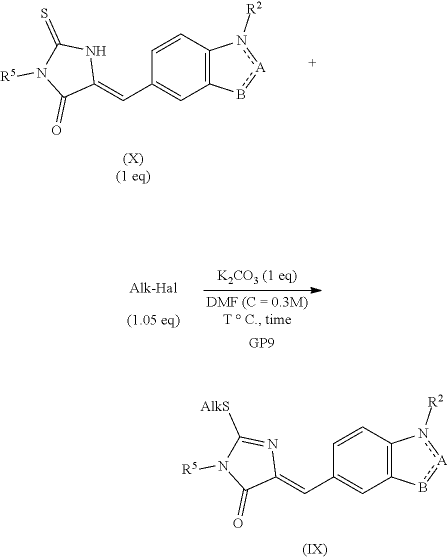

In the above scheme, A, B, R² and R⁵ are as defined above and Alk is a (C₁-C₅)alkyl.

GP9: The appropriate alkyliodide (1.05 eq) was added dropwise to a stirred solution of the adequate 5-heteroaryl-2-thioxo-imidazolidin-4-one (1 eq) and K₂CO₃ (1 eq) in DMF (C=0.3 M) at the appropriate temperature (see details below). The resulting mixture was stirred at the appropriate temperature, for the indicated time. Upon completion (TLC), the mixture was poured into water. The precipitated solid was stirred for 30 min and filtered off on a fritted glass funnel, thoroughly dried, and could be used in the next step without further purification. Trace impurities resulting from N3-alkylation may be removed by trituration or FC.

### Example 10.1: Synthesis of (4Z)-4-(1,3-benzoxazol-6-ylmethylene)-2-ethylsulfanyl-1H-imidazol-5-one (10.1)

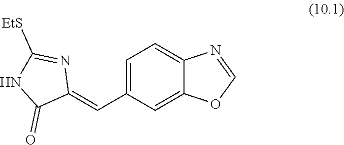

Compound (10.1) was synthesized according to GP9: reaction carried out with intermediate (9.1) (1.97 mmol) and EtI, at r.t. for 12 h. Yellow solid, 76% (411 mg). ¹H NMR (400 MHz, DMSO-cd₆, 300K) δ_{H} 11.85 (bs, 1H, NH, D₂O exchanged), 8.82 (s, 1H), 8.69 (s, 1H), 8.16 (d, J=8.4 Hz, 1H), 7.83 (d, J=8.3 Hz, 1H), 6.90 (s, 1H), 3.33-3.27 (m, 2H), 1.45 (t, J=7.3 Hz, 3H). ¹³C NMR (101 MHz, DMSO-d₆, 300K) δ_{C} 170.4, 165.0, 155.7, 149.6, 140.6, 139.4, 132.3, 128.6, 120.1, 120.0, 113.1, 24.4, 14.5. MS (ESI⁺): [M+H]⁺ 273.9.

### Example 10.2: Synthesis of (4Z)-4-(1,3-benzoxazol-6-ylmethylene)-2-methylsulfanyl-1H-imidazol-5-one (10.2)

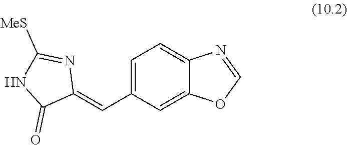 [SMILES: CSC1=N/C(=C\c2ccc3ncoc3c2)C(=O)N1]

Compound (10.2) was synthesized according to GP9: reaction carried out with intermediate (9.1) (11.89 mmol) and MeI, at 0° C. for 6 h, then at r.t for 12 h. The final product required a trituration in DCM. Yellow solid, 91% (2.805 g). ¹H NMR (400 MHz, DMSO-d₆, 300K) δ 11.88 (bs, 1H, NH, D₂O exchanged), 8.82 (s, 1H), 8.69 (s, 1H), 8.19 (d, J=8.4 Hz, 1H), 7.83 (d, J=8.3 Hz, 1H), 6.91 (s, 1H), 2.71 (s, 3H). ¹³C NMR (101 MHz, DMSO-d_{b}, 300K) δ_{C} 170.5, 165.7, 155.7, 149.6, 140.6, 139.4, 132.3, 128.6, 120.2, 120.0, 113.3, 12.3. MS (ESI⁺): [M+H]⁺ 260.2.

### Example 10.3: Synthesis of (4Z)-2-ethylsulfanyl-4-(1H-indazol-5-ylmethylene)-1H-imidazol-5-one (10.3)

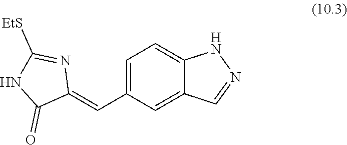

Compound (10.3) was synthesized according to GP9: reaction carried out with intermediate (9.2) (2.05 mmol) and EtI, at r.t. for 12 h. Yellow solid, 86% (481 mg). ¹H NMR (400 MHz, DMSO-d₆, 300K) δ_{H} 13.24 (bs, 1H, NH, D₂O exchanged), 11.71 (bs, 1H, NH, D₂O exchanged), 8.52 (s, 1H), 8.36 (d, J=8.9 Hz, 1H), 8.16 (s, 1H), 7.58 (d, J=8.8 Hz, 1H), 6.88 (s, 1H), 3.58-3.03 (m, 2H), 1.44 (t, J=7.3 Hz, 3H). ¹³C NMR (101 MHz, DMSO-d₆, 300K) δ_{C} 170.6, 162.8, 139.9, 137.8, 134.8, 129.0, 127.1, 125.2, 123.3, 122.2, 110.4, 24.2, 14.6. MS (ESI⁺): [M+H]⁺ 272.9.

### Example 10.4: Synthesis of (4Z)-2-ethylsulfanyl-4-[(2-methylindazol-5-yl)methylene]-1H-imidazol-5-one (10.4)

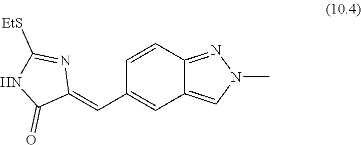

Compound (10.4) was synthesized according to GP9: reaction carried out with intermediate (9.3) (1.87 mmol) and EtI, at r.t. for 12 h. Yellow solid, 88% (471 mg). 1H NMR (400 MHz, DMSO-d₆, 300K) δ_{H} 11.70 (bs, 1H, NH, D₂O exchanged), 8.46 (s, 1H), 8.40 (s, 1H), 8.31 (d, J=9.1 Hz, 1H), 7.60 (d, J=9.1 Hz, 1H), 6.83 (s, 1H), 4.17 (s, 3H), 3.32-3.26 (m, 2H), 1.44 (t, J=7.3 Hz, 3H). ¹³C NMR (101 MHz, DMSO-d₆, 300K) δ_{C} 170.6, 162.6, 148.0, 137.7, 127.8, 127.7, 126.4, 126.0, 122.4, 121.9, 116.9, 39.9, 24.2, 14.6. MS (ESI⁺): [M+H]⁺ 287.9.

### Example 10.5: (4Z)-2-ethylsulfanyl-4-[(1-methylindazol-5-yl)methylene]-1H-imidazol-5-one (10.5)

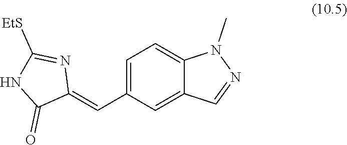

Compound (10.5) was synthesized according to GP9: reaction carried out with intermediate (9.4) (1.58 mmol) and EtI, at r.t. for 12 h. Yellow solid, 76% (344 mg). ¹H NMR (400 MHz, DMSO-d₆, 300K) δ_{H} 11.73 (bs, 1H, NH, D₂O exchanged), 8.50 (s, 1H), 8.41 (d, J=8.9 Hz, 1H), 8.14 (s, 1H), 7.69 (d, J=9.0 Hz, 1H), 6.89 (s, 1H), 4.05 (s, 3H), 3.31 (q, J=7.3 Hz, 2H), 1.45 (t, J=7.3 Hz, 3H). ¹³C NMR (101 MHz, DMSO-d₆, 300K) δ_{C} 170.6, 163.0, 139.5, 137.9, 133.7, 128.9, 127.1, 125.3, 123.8, 122.0, 110.0, 35.5, 24.3, 14.6. MS (ESI⁺): [M+H]y 287.9.

### Example 10.6: (4Z)-4-[(1-methylindazol-5-yl)methylene]-2-methylsulfanyl-1H-imidazol-5-one (10.6)

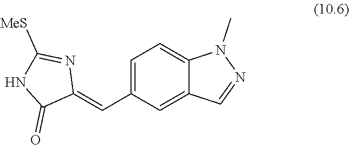 [SMILES: CSC1=N/C(=C\c2ccc3c(cnn3C)c2)C(=O)N1]

Compound (10.6) was synthesized according to GP9: reaction carried out with intermediate (9.4) (12.00 mmol) and MeI, at 0° C. for 6 h, then at r.t for 12 h. The final product required a trituration in DCM. Yellow solid, 94% (3.060 g). ¹H NMR (400 MHz, DMSO-d₆, 300K) δ_{H} 11.75 (bs, 1H, NH, D₂O exchanged), 8.53 (s, 1H), 8.39 (d, J=8.9 Hz, 1H), 8.14 (s, 1H), 7.69 (d, J=8.9 Hz, 1H), 6.89 (s, 1H), 4.06 (s, 3H), 2.70 (s, 3H). ¹³C NMR (101 MHz, DMSO-d₆, 300K) δ_{C} 170.7, 163.7, 139.5, 137.9, 133.7, 129.1, 127.1, 125.3, 123.8, 122.0, 109.9, 35.4, 12.2. MS (ESI⁺): [M+H]⁺ 273.2.

### Example 10.7: Synthesis of (4Z)-4-(1H-Benzimidazol-5-ylmethylene)-2-ethylsulfanyl-1H-imidazol-5-one (10.7)

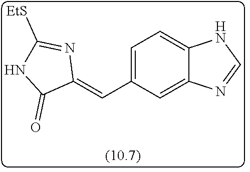

Compound (10.7) was synthesized according to GP9: reaction carried out with intermediate (9.5) (1.64 mmol) and EtI, at r.t. for 12 h. Yellow solid, 61% (274 mg). ¹H NMR (400 MHz, DMSO-d₆, 300K) δ_{H} 12.85-12.51 (br m, 1H, NH, D₂O exchanged), 11.71 (br s, 1H, NH, D₂O exchanged), 8.52 (s, 1H), 8.29 (s, 1H), 8.17-7.90 (m, 1H), 7.74-7.48 (m, 1H), 6.89 (s, 1H), 3.38-3.29 (m, 2H), 1.45 (t, J=7.3 Hz, 3H). MS (ESI⁺): [M+H]⁺ 272.9.

### Example 10.8: Synthesis of (4Z)-4-(1H-Benzimidazol-5-ylmethylene)-2-methylsulfanyl-1H-imidazol-5-one (10.8)

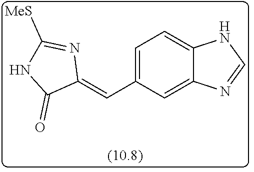 [SMILES: CSC1=N/C(=C\c2ccc3[nH]cnc3c2)C(=O)N1]

Compound (10.8) was synthesized according to GP9: reaction carried out with intermediate (9.5) (16.04 mmol) and MeI, at 0° C. for 6 h, then at r.t for 12 h. The final product required a trituration in DCM. Yellow solid, 92% (3.827 g). ¹H NMR (400 MHz, DMSO-d_{b}, 300K) δ_{H} 12.64 (br s, 1H, NH, D₂O exchanged), 11.75 (br s, 1H, NH, D₂O exchanged), 8.53 (s, 1H), 8.29 (s, 1H), 8.05 (br s, 1H), 7.72-7.53 (m, 1H), 6.89 (s, 1H), 2.71 (s, 3H). MS (ESI⁺): [M+H]⁺ 259.1.

### Example 10.9: Synthesis of (4Z)-4-[(3-methylbenzimidazol-5-yl)methylene]-2-methylsulfanyl-1H-imidazol-5-one (10.9)

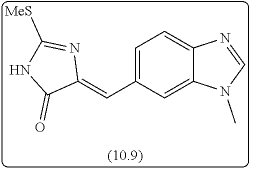 [SMILES: CSC1=N/C(=C\c2ccc3ncn(C)c3c2)C(=O)N1]

Compound (10.9) was synthesized according to GP9: reaction carried out with intermediate (9.6) (12.99 mmol) and MeI, at 0° C. for 6 h, then at r.t for 12 h. The final product required a trituration in DCM. Yellow solid, 96% (3.395 g). ¹H NMR (400 MHz, DMSO-d_{b}, 300K) δ_{H} 11.79 (br s, 1H, NH, D₂O exchanged), 8.52 (s, 1H), 8.28 (s, 1H), 8.07 (d, J=8.5 Hz, 1H), 7.67 (d, J=8.5 Hz, 1H), 6.90 (s, 1H), 3.86 (s, 3H), 2.71 (s, 3H). MS (ESI⁺): [M+H]⁺ 273.1.

### Example 10.10: Synthesis of (5Z)-5-(1,3-benzoxazol-6-ylmethylene)-3-methyl-2-methylsulfanyl-imidazol-4-one (10.10)

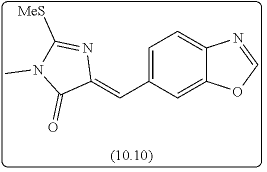 [SMILES: CSC1=N/C(=C\c2ccc3ncoc3c2)C(=O)N1C]

Compound (10.10) was synthesized according to GP9: reaction carried out with intermediate (9.7) (11.18 mmol) and MeI, at 0° C. for 30 min, then at r.t. for 12 h. Yellow solid, 96% (2.92 g). ¹H NMR (400 MHz, CDCl₃, 300K) δ_{H} 8.66 (s, 1H), 8.15 (s, 1H), 7.98 (dd, J=8.4, 1.5 Hz, 1H), 7.78 (d, J=8.3 Hz, 1H), 7.04 (s, 1H), 3.19 (s, 3H), 2.78 (s, 3H). ¹³C NMR (101 MHz, CDCl₃, 300K) δ_{C} 170.0, 166.3, 154.1, 150.5, 141.3, 138.9, 132.7, 129.3, 123.1, 120.5, 114.0, 26.7, 13.2. MS (ESI⁺): [M+H]⁺ 274.1.

### Example 10.11: Synthesis of (5Z)-5-(1H-indazol-5-ylmethylene)-3-methyl-2-methylsulfanyl-imidazol-4-one (10.11)

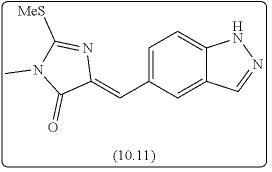 [SMILES: CSC1=N/C(=C\c2ccc3[nH]ncc3c2)C(=O)N1C]

Compound (10.11) was synthesized according to GP9: reaction carried out with intermediate (9.8) (12.27 mmol) and MeI, at 0° C. for 30 min, then at r.t. for 12 h. Yellow solid, 99% (3.30 g). ¹H NMR (400 MHz, DMSO-d₆, 300K) δ_{H} 13.25 (br s, 1H, NH, D₂O exchanged), 8.57 (s, 1H), 8.40 (dd, J=8.9, 1.5 Hz, 1H), 8.17 (s, 1H), 7.59 (d, J=8.8 Hz, 1H), 7.01 (s, 1H), 3.09 (s, 3H), 2.76 (s, 3H). ¹³C NMR (101 MHz, DMSO-d₆, 300K) δ_{C} 169.0, 164.6, 139.9, 136.7, 134.8, 129.3, 127.0, 125.6, 123.5, 123.3, 110.4, 26.3, 12.5. MS (ESI⁺): [M+H]⁺ 273.1.

### Example 10.12: Synthesis of (5Z)-3-methyl-5-[(2-methylindazol-5-yl)methylene]-2-methylsulfanyl-imidazol-4-one (10.12)

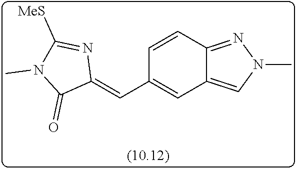

Compound (10.12) was synthesized according to GP9: reaction carried out with intermediate (9.9) (7.12 mmol) and MeI, at 0° C. for 30 min, then at r.t. for 12 h. Yellow solid, 95% (1.93 g). ¹H NMR (400 MHz, DMSO-d₆, 300K) δ_{H} 8.47 (s, 1H), 8.44 (s, 1H), 8.35 (d, J=1.7 Hz, 1H), 7.61 (d, J=9.1 Hz, 1H), 6.96 (s, 1H), 4.17 (s, 3H), 3.09 (s, 3H), 2.75 (s, 3H). ¹³C NMR (101 MHz, DMSO-d₆, 300K) δ_{C} 169.0, 164.3, 148.0, 136.7, 127.9, 127.6, 126.5, 126.4, 123.7, 121.9, 116.9, 40.1, 26.3, 12.5. MS (ESI⁺): [M+H]T 287.1.

### Example 10.13: Synthesis of (5Z)-3-methyl-5-[(1-methylindazol-5-yl)methylene]-2-methylsulfanyl-imidazol-4-one (10.13)

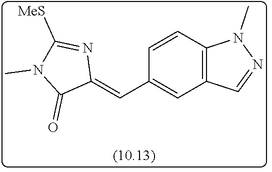 [SMILES: CSC1=N/C(=C\c2ccc3c(cnn3C)c2)C(=O)N1C]

Compound (10.13) was synthesized according to GP9: reaction carried out with intermediate (9.10) (14.14 mmol) and MeI, at 0° C. for 30 min, then at r.t. for 12 h. Yellow solid, 100% (4.05 g). ¹H NMR (400 MHz, TFA-d, 300K) h 8.77 (s, 1H), 8.30 (s, 1H), 7.97 (dd, J=9.2, 1.6 Hz, 1H), 7.78 (d, J=9.1 Hz, 1H), 7.67 (s, 1H), 4.31 (s, 3H), 3.40 (s, 3H), 2.97 (s, 3H). ¹³C NMR (101 MHz, CF₃COOD, 300K) δ_{C} 177.0, 164.0, 142.6, 136.8, 133.4, 130.6, 129.7, 129.5, 127.5, 122.9, 114.0, 36.8, 29.4, 15.0. MS (ESI⁺): [M+H]⁺ 287.1.

### Example 10.14: Synthesis of (5Z)-5-(1H-benzimidazol-5-ylmethylene)-3-methyl-2-methylsulfanyl-imidazol-4-one (10.14)

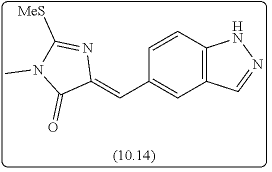 [SMILES: CSC1=N/C(=C\c2ccc3[nH]ncc3c2)C(=O)N1C]

Compound (10.14) was synthesized according to GP9: reaction carried out with intermediate (9.11) (7.24 mmol) and MeI, at 0° C. for 30 min, then at r.t. for 12 h. Yellow solid, 81% (1.59 g). ¹H NMR (400 MHz, DMSO-d₆, 300K) δ_{H} 12.65 (br s, 1H, NH, D₂O exchanged), 8.56 (s, 1H), 8.30 (s, 1H), 8.09 (d, J=8.4 Hz, 1H), 7.62 (d, J=8.4 Hz, 1H), 7.03 (s, 1H), 3.10 (s, 3H), 2.77 (s, 3H). MS (ESI⁺): [M+H]⁺ 273.1.

### Example 10.15: Synthesis of (5Z)-3-methyl-5-[(3-methylbenzimidazol-5-yl)methylene]-2-methylsulfanyl-imidazol-4-one (10.15)

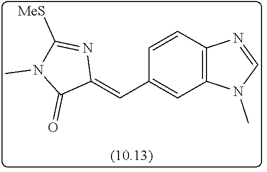 [SMILES: CSC1=N/C(=C\c2ccc3ncn(C)c3c2)C(=O)N1C]

Compound (10.15) was synthesized according to GP9: reaction carried out with intermediate (9.11) (7.93 mmol) and MeI, at 0° C. for 30 min, then at r.t. for 12 h. Yellow solid, 95% (2.15 g). ¹H NMR (400 MHz, DMSO-d₆, 300K) δ_{H} 8.55 (s, 1H), 8.29 (s, 1H), 8.09 (dd, J=8.5, 1.5 Hz, 1H), 7.67 (d, J=8.5 Hz, 1H), 7.02 (s, 1H), 3.86 (s, 3H), 3.10 (s, 3H), 2.77 (s, 3H). ¹³C NMR (101 MHz, DMSO-d₆, 300K) δ_{C} 168.9, 165.0, 146.6, 144.6, 137.1, 134.9, 128.6, 125.8, 123.3, 119.3, 113.9, 30.6, 26.3, 12.5. MS (ESI⁺): [M+H]⁺ 287.1.

### Example 11: Synthesis of compounds of formula (I) according to general protocols 10 (GP10), 11 (GP11) and 12 (GP12)

General protocol 10—Knoevenagel condensation between N2-functionalized 2-amino-1,4-dihydroimidazol-5-ones and heteroarylcarbalhydes (ROUTE 1)

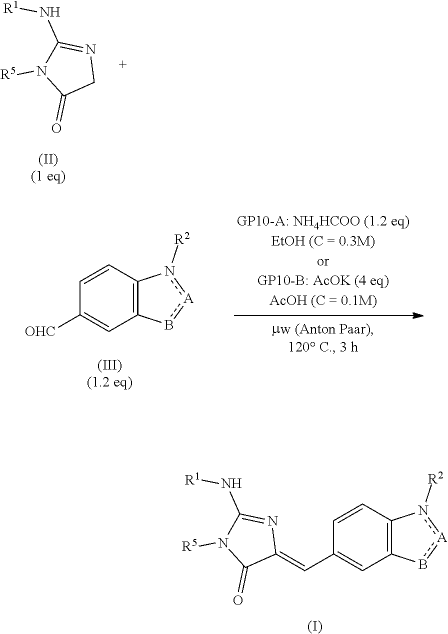

In the above scheme, A, B, R¹, R² and R⁵ are as defined above. GP10-A: a stirred solution of the appropriate N2-functionalized 2-amino-1,4-dihydroimidazol-5-one (1 eq), heteroarylcarboxaldehyde (1.2 eq) and NH₄HCOO (1.2 eq) in EtOH (C=0.3 M) was heated in a sealed tube in a microwave oven (Anton Paar) at 120° C. for 3 h. Upon completion (followed by consumption of the heteroarylcarboxaldehyde on TLC), the mixture was brought back to room temperature, adsorbed on silica and purified by FC (see details below). After FC, higher purity may be achieved by reprecipitation, trituration, or recrystallization (see details below).

GP10-B: a stirred solution of the appropriate N2-functionalized 2-amino-1,4-dihydroimidazol-5-one (1 eq), heteroarylcarboxaldehyde (1.2 eq) and AcOK (4 eq) in AcOH (C=0.1 M) was heated in a sealed tube in a microwave oven (Anton Paar) at 120° C. for 3 h.

Upon completion (followed by consumption of the heteroarylcarboxaldehyde on TLC), the mixture was brought back to room temperature, slowly added on sat. Na₂CO₃(aq). The precipitated solid was filtered off on a fritted-glass funnel, adsorbed on silica and purified by FC (see details below). After FC, higher purity may be achieved by reprecipitation, trituration, or recrystallization (see details below).

### General Protocol 11—Addition of amines on (Z)-heteroarylmethylene-2-alkylsulfanyl-1H-imidazol-5-ones (ROUTE 2)

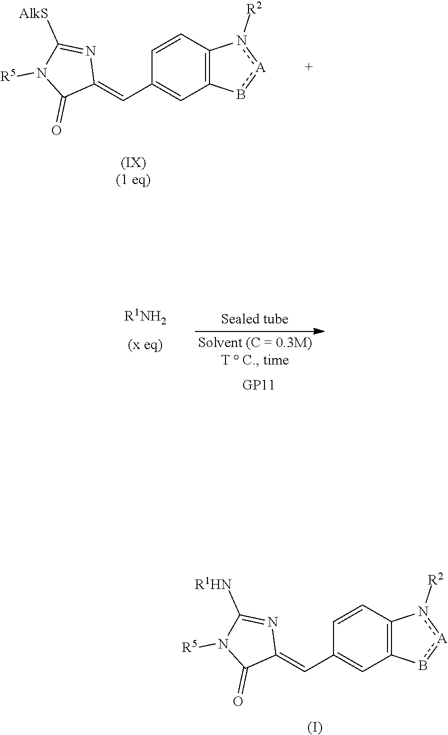

In the above scheme, A, B, R¹, R² and R⁵ are as defined above and Alk is a (C₁-C₅)alkyl.

GP11: A stirred solution of the appropriate amine (x eq), (4Z)-4-heteroaryl-2-alkylsulfanyl-1H-imidazol-5-one^{(a)} (1 eq) in the appropriate solvent (C=0.3 M) was heated in a sealed tube (heating block). Upon completion (followed by consumption of the isothiourea on TLC), the mixture was brought back to room temperature.
GP11-A: direct precipitation of the desired product: The reaction medium was stirred 1 h at 0° C. The precipitated solid was filtered off on a fritted-glass funnel. High purity may be achieved after filtration by washing, reprecipitation, trituration, or recrystallization (see Table 3 for details).
GP11-B: the product failed to precipitate: the reaction mixture was concentrated in vacuo, adsorbed on silica, and purified by FC. High purity may be achieved after filtration by reprecipitation, trituration, or recrystallization.
GP11-C: the product failed to precipitate: the reaction mixture was concentrated in vacuo. The resulting crude was triturated in EtOH (at r.t. or reflux), filtered off on a fritted-glass funnel.
(a) May require activation with AcOH or TEA·HCl depending on the amine (see details below).

### General protocol 12—Addition of amines on (4Z)-4-(1,3-benzoxazol-6-ylmethylene)-2-alkylsulfanyl-1H-imidazol-5-ones (ROUTE 2′)

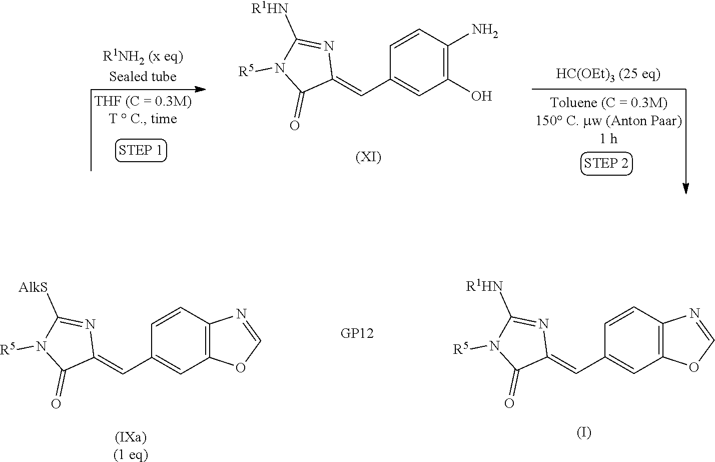

In the above scheme, R¹ and R⁵ are as defined above and Alk is a (C₁-C₅)alkyl.

GP12—Step 1: a stirred solution of the appropriate amine (x eq), (4Z)-4-(1,3-benzoxazol-6-ylmethylene)-2-alkylsulfanyl-1H-imidazol-5-one (1 eq) in THF (C=0.3 M) was heated in a sealed tube (heating block). Upon completion (followed by consumption of the isothiourea on TLC), the mixture was brought back to room temperature. The precipitated red/brown solid was isolated by filtration, washed with ice-cold THF or dioxane and dried.

GP12—Step 2: a stirred solution of the previously isolated solid (1 eq) and HC(OEt)₃ (25 eq) in toluene (C=0.3 M) was heated in a sealed tube in a microwave oven (Anton Paar) at 150° C. for 1 h. Upon completion, the mixture was directly adsorbed on silica and purified by FC (see details below). Higher purity may be achieved by reprecipitation, trituration, or recrystallization (see details below).

### Selected Examples from Benzoxazole Sub-Series

### Example 11.1: Synthesis of (4Z)-4-(1,3-benzoxazol-6-ylmethylene)-2-(cyclohexylamino)-1H-imidazol-5-one (1)

Reaction was carried out according to GP12—step 1, in THF, on a 915 μmol scale of intermediate (10.1), with 12 eq of cyclohexylamine, at 110° C. (sealed tube, heating block), for 12 h. The isolated aminophenol intermediate was cyclized according to GP12—step 2. Purification by FC (elution: DCM/MeOH: 99/1 to 9/1). The final product required a reprecipitation from DCM/pentane at 0° C. Isolated yield: 34% (2 steps).

### Example 11.2: Synthesis of (4Z)-4-(1,3-benzoxazol-6-ylmethylene)-2-(cycloheptylamino)-1H-imidazol-5-one (2)

Reaction was carried out according to GP12—step 1, in THF, on a 695 μmol scale of intermediate (10.1), with 12 eq of cycloheptylamine, at 110° C. (sealed tube, heating block), for 12 h. The isolated aminophenol intermediate was cyclized according to GP12—step 2. Purification by FC (elution: DCM/MeOH: 99/1 to 9/1). The final product required a reprecipitation from DCM/pentane at 0° C. Isolated yield: 38% (2 steps).

### Example 11.3: Synthesis of (4Z)-4-(1,3-benzoxazol-6-ylmethylene)-2-[[(1R)-1-(methoxymethyl)-3-methyl-butyl]amino]-1H-imidazol-5-one (5)

Reaction was carried out according to GP12—step 1, in THF, on a 352 μmol scale of intermediate (10.2), with 5 eq of (2R)-1-methoxy-4-methyl-pentan-2-amine, at 120° C. (sealed tube, heating block), for 72 h. The isolated aminophenol intermediate was cyclized according to GP12—step 2. Purification by FC (elution: DCM/MeOH: 99/1 to 9/1). Isolated yield: 39% (2 steps).

### Example 11.4: Synthesis of (4Z)-4-(1,3-benzoxazol-6-ylmethylene)-2-[[(1R,2R)-2-methoxycyclopentyl]amino]-1H-imidazol-5-one (6)

Reaction was carried out according to GP12—step 1, in THF, on a 386 μmol scale of intermediate (10.2), with 5 eq of (1R,2R)-2-methoxycyclopentan-1-amine, at 120° C. (sealed tube, heating block), for 28 h. The isolated aminophenol intermediate was cyclized according to GP12—step 2. Purification by PTLC (elution: DCM/MeOH: 97/3). Isolated yield: 39% (2 steps).

### Example 11.5: Synthesis of (4Z)-2-(1-adamantylamino)-4-(1,3-benzoxazol-6-ylmethylene)-1H-imidazol-5-one (11)

Reaction carried out according to GP10-A, on a 429 μmol scale of intermediate (2.11), with aldehyde (7.1). Purification by FC (elution: DCM/MeOH: 99/1 to 93/7). Isolated yield: 19%.

preparing a drug for combatting a disease selected from type 1 and type 2 diabetes, viral infections, in particular as mentioned above, tendinopathy and osteoarthritis, cancers, in particular as mentioned above, infections caused by unicellular parasites, such malaria, Leishmaniasis, Chagas and sleeping sickness (Trypanosoma sp.), cattle diseases due to unicellular pathogens, and to regulate body temperature.

According to a particular embodiment, the treatment is continuous or non-continuous.

A “continuous treatment” means a long-term treatment which can be implemented with various administration frequencies, such as once or twice every day, every three days, once a week, or once every two weeks or once every month or by transdermal patch delivery.

According to one embodiment, the compound of formula (I), or any of its pharmaceutically acceptable salts, is administered at a dose varying from 0.1 to 1000 mg. in particular varying from 0.5 to 500 mg, or for example varying from 5 to 100 mg.

Another object of the invention relates to a therapeutic method for the treatment and/or for the prevention of a disease selected from cognitive deficits and neuroinflammation associated with Down syndrome (Trisomy 21), Alzheimer's disease and related diseases, dementia and/or tauopathies as well as other neurodegenerative diseases such as Parkinson's disease and Pick disease, in particular Niemann-Pick Type C Disease; CDKL5 Deficiency Disorder; McDermid syndrome; autism; type 1 and type 2 diabetes; abnormal folate and methionine metabolism; tendinopathy and osteoarthritis, in particular knee osteoarthritis; Duchenne muscular dystrophy; cancers, such as brain cancer, including glioblastoma, leukemia, including megakaryoblastic leukemia, head and neck squamous cell carcinoma, pancreatic cancer, including pancreatic ductal adenocarcinoma, prostate cancer, gastrointestinal cancer, breast cancer, such as Triple-negative breast cancer (TNBC), tissue cancer, including liposarcoma, Hedgehog/GLI-dependent cancer, liver cancer, including Hepatocellular carcinoma and viral infections, such as infections caused by Human immunodeficiency virus type 1 (HIV-1), Human cytomegalovirus (HCMV), Influenza A, Herpes virus, rhesus macaque cytomegalovirus, varicella-zoster virus, herpes simplex virus (HSV), Hepatitis C virus, Chikungunya virus, Dengue virus, Influenza virus and Severe acute respiratory syndrome (SARS) coronavirus, Cytomegalovirus and Human papillomavirus; neuroinflammation; anemia: infections caused by unicellular parasites, such malaria, Leishmaniasis, Chagas and sleeping sickness (Trypanosoma sp.), cattle diseases due to unicellular pathogens, and for the regulation of the body temperature, in a patient in need thereof, comprising at least a step of administering a therapeutically effective amount of a compound of formula (I) or of compounds (1) to (271), as defined above. or one of their acceptable salts.

In a specific embodiment, the invention provides a use of a compound of formula (I) according to the invention or a pharmaceutically acceptable salt thereof or a pharmaceutically active derivative thereof or a method according to the invention wherein the compound of formula (I) is to be administered in combination with a co-agent useful in anyone of the hereabove mentioned diseases.

The compounds can be administered through any mode of administration such as, for example, intramuscular, intravenous, intranasal or oral route, transdermal patch, etc.

The inventive composition can further include one or more additives such as diluents, excipients, stabilizers and preservatives. Such additives are well known to those skilled in the art and are described notably in “Ullmann's Encyclopedia of Industrial Chemistry, 6^{th} Ed.” (various editors, 1989-1998, Marcel Dekker) and in “Pharmaceutical Dosage Forms and Drug Delivery Systems” (ANSEL et al, 1994, WILLIAMS & WILKINS).

The aforementioned excipients are selected according to the dosage form and the desired mode of administration.

Compositions of this invention may be administered in any manner, including, but not limited to, orally, parenterally, sublingually, transdermally, vaginally, rectally, transmucosally, topically, intranasally via inhalation, via buccal or intranasal administration, or combinations thereof. Parenteral administration includes, but is not limited to, intravenous, intra-arterial, intra-peritoneal, subcutaneous, intramuscular, intra-thecal, and intra-articular. The compositions of this invention may also be administered in the form of an implant, which allows slow release of the compositions as well as a slow controlled i.v. infusion.

For example, a compound of formula (I) can be present in any pharmaceutical form which is suitable for enteral or parenteral administration, in association with appropriate excipients, for example in the form of plain or coated tablets, hard gelatine, soft shell capsules and other capsules, suppositories, or drinkable, such as suspensions, syrups, or injectable solutions or suspensions.

In a particular embodiment, a compound of formula (I) according to the invention is administered orally.

Oral route of administration is in particular preferred in the prophylaxis or treatment aspect of the invention.

1. A compound of formula (I) 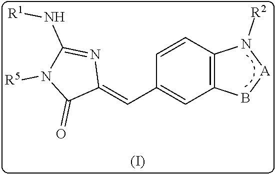
wherein:
A is selected from the group consisting of ═CH—, —N═ and —NR³—, B is selected from the group consisting of —O—, ═CH—, ═N—, —NH—, —S— and
—NR⁴—,
at least one of A and B is ═CH— or —S—,
A and B can not both represent a ═CH—,
R² is selected from the group consisting of a hydrogen atom and a (C₁-C₃)alkyl group or does not exist when the nitrogen atom to which it is attached is —N═,
R³ and R⁴ are independently selected from (C₁-C₃)alkyl groups,
R⁵ represents a hydrogen atom, a (C₁-C₄)alkyl group or a (C₃-C₆)cycloalkyl group, and
when A represents ═CH— and B represents ═N— or —NH—, then R² respectively represents a hydrogen atom or does not exist, and wherein R¹ represents:
(i) a (C₄-C₆)alkyl group substituted by a group selected from a hydroxy group, a halogen and a (C₁-C₃)alkoxy group,
(ii) a (C₃-C₈)cycloalkyl group, optionally substituted by a group selected from a hydroxy group and a (C₁-C₃) alkoxy group,
(iii) a bridged (C₆-C₁₀)cycloalkyl group, optionally substituted by a group selected from a (C₁-C₄)alkyl group, a (C₁-C₄)alkoxy group, a halogen atom and a hydroxy group,
(iv) a fused phenyl group, selected from phenyl groups fused with a (C₅-C₆)cycloalkyl, which (C₅-C₆)cycloalkyl is optionally substituted by a hydroxy group,
(v) a phenyl group, substituted by a (C₄-C₇)heterocycloalkyl group, said (C₄-C₇)heterocycloalkyl group being itself optionally substituted by a (C₁-C₄)alkyl group, or
(vi) a R′-L- group, wherein L is either a single bond or a (C₁-C₃)alkanediyl group, optionally substituted by a group selected from a hydroxy group and a (C₁-C₃)alkoxy group, and
R′ represents:
(vi.1) a (C₃-C₈)heterocycloalkyl group, optionally substituted by one or two groups selected from a hydroxy group, and a (C₁-C₄)alkyl group, or
(vi.2) a heteroaryl group selected from an oxazolyl, a isoxazolyl, a pyridinyl, a pyrimidinyl, a pyridazinyl, a triazinyl, a pyrazinyl, an oxadiazolyl, a furanyl, a pyrazolyl, a thiazolyl, an isothiazolyl, a thiadiazolyl, an imidazolyl and a triazolyl, optionally substituted by a (C₁-C₄)alkyl group, or
(vii) a R″-L- group wherein L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group,
R^{b} and R^{c} independently represent a (C₁-C₆)alkyl group or a hydrogen atom,
or any of its pharmaceutically acceptable salt.

2. A compound of formula (I) according to claim 1, wherein R¹ represents:
(i) a (C₅-C₆)alkyl group substituted by a group selected from a hydroxy group, a halogen and a (C₁-C₃)alkoxy group,
(ii) a (C₅-C₈)cycloalkyl group, optionally substituted by a group selected from a hydroxy group and a (C₁-C₃) alkoxy group,
(iii) a bridged (C₉-C₁₀)cycloalkyl group, optionally substituted by a group selected from a (C₁-C₄)alkyl group, a (C₁-C₄)alkoxy group, a halogen atom and a hydroxy group,
(iv) a phenyl group fused with a cyclopentyl, which cyclopentyl is optionally substituted by a hydroxy group,
(v) a phenyl group, substituted by a (C₄-C₇)heterocycloalkyl group, said (C₄-C₇)heterocycloalkyl group being itself optionally substituted by a (C₁-C₄) alkyl group, or
(vi) a R′-L- group wherein
L is either a single bond or a (C₁-C₃)alkanediyl group, optionally substituted by a group selected from a hydroxy group and a (C₁-C₃)alkoxy group, and
R′ represents:
(vi.1) a (C₆-C₇)heterocycloalkyl group, optionally substituted by one or two groups selected from a hydroxy group and a (C₁-C₄)alkyl group, or
(vi.2) a heteroaryl group selected from a pyridinyl, an imidazolyl, a pyrazinyl, a furanyl, a thiazolyl, a pyrazolyl, a thiadiazolyl, a pyridazinyl and a pyrimidinyl, optionally substituted by a (C₁-C₄)alkyl group,
(vi) a R″-L- group wherein
L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group, and
R^{b} and R^{c} independently represent a (C₁-C₆)alkyl group or a hydrogen atom,
or any of its pharmaceutically acceptable salt.

3. A compound of formula (I) according to claim 1, wherein R¹ represents:
(i) a (C₅-C₆)alkyl group substituted by a group selected from a hydroxy group, a fluorine atom and a methoxy group,
(ii) a (C₅-C₈)cycloalkyl group in, optionally substituted by a group selected from a hydroxy group and a methoxy group,
(iii) a bridged (C₉-C₁₀)cycloalkyl group, optionally substituted by a group selected from a methoxy group, a fluorine atom and a hydroxy group,
(iv) a phenyl group fused with a cyclopentyl, which cyclopentyl is substituted by a hydroxy group,
(v) a phenyl group, substituted by a N-methylpiperazinyl group, or
(vi) a R′-L- group wherein L is either a single bond or a (C₁-C₃)alkanediyl group, optionally substituted by a group chosen from a hydroxy group and a methoxy group,
and R′ is selected from the group consisting of:
(vi.1) a (C₆-C₇)heterocycloalkyl group, optionally substituted by one or two groups selected from a hydroxy group and a methyl group,
(vi.2) a heteroaryl group selected from a pyridinyl, an imidazolyl, a pyrazinyl, a furanyl, a thiazolyl, a pyrazolyl, a thiadiazolyl, a pyridazinyl and a pyrimidinyl, optionally substituted by a methyl group, or
(vii) a R″-L- group wherein L is a (C₁-C₃)alkanediyl group, optionally substituted by a group selected from the group consisting of a —NH₂ group, a methoxy group, a hydroxy group, a —COOR^{a} group and a halogen atom, and
R″ is a phenyl group, optionally substituted by a trifluoromethyl group,
or any of its pharmaceutically acceptable salts.

4. A compound of formula (I) according to claim 1, wherein L is selected from a group consisting of a —CH₂— group, a —CH(CH₂OH)— group, a —CH(CH₂OCH₃)— group, a —CH(OH)—CH₂— group and a —CH(CH₂NH₂)— group, or any of its pharmaceutically acceptable salts.

5. A compound of formula (I) according to claim 1, wherein:
(vi.1) when R′ is a (C₅-C₈)heterocycloalkyl group, L is a —CH₂— group,
(vi.2) when R′ is a phenyl, L is selected from the group consisting of a —CH₂— group, a —CH(CH₂OH)— group, a —CH(CH₂OCH₃)— group, a —CH(OH)—CH₂— group and a —CH(CH₂NH₂)— group,
(vi.3) when R′ is a heteroaryl group, L is a —CH₂— group,
or any of its pharmaceutically acceptable salts.

6. A compound of formula (I) according to claim 1 chosen from the following compounds 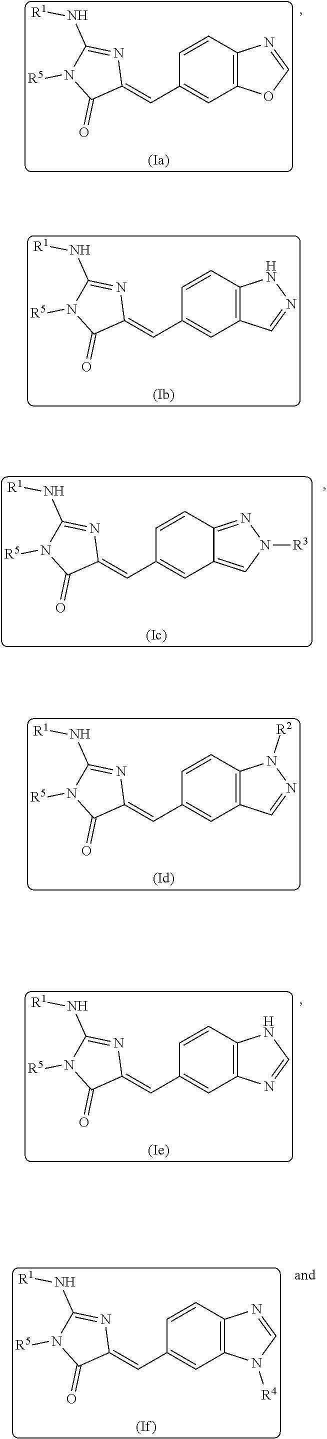 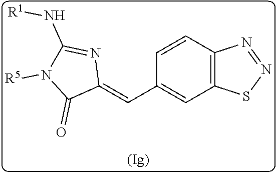
or any of its pharmaceutically acceptable salts.

7. A compound of formula (I) according to claim 6, wherein when the compound of formula (I) is chosen from the subformulae (Ib), (Id), (Ie) and (If), R¹ represents:
(i) a (C₄-C₆)alkyl group substituted by a group selected from a hydroxy group, a halogen and a (C₁-C₃)alkoxy group,
(ii) a (C₃-C₈)cycloalkyl group, optionally substituted by a group selected from a hydroxy group and a (C₁-C₃) alkoxy group,
(iii) a bridged (C₆-C₁₀)cycloalkyl group, optionally substituted by a group selected from a (C₁-C₄)alkyl group, a (C₁-C₄)alkoxy group, a halogen atom and a hydroxy group, or
(iv) a R′-L- group, wherein L is either a single bond or a (C₁-C₃)alkanediyl group, optionally substituted by a group selected from a hydroxy group and a (C₁-C₃)alkoxy group, and R′ represents:
(iv.1) a (C₃-C₈)heterocycloalkyl group, optionally substituted by one or two groups selected from a hydroxy group, and a (C₁-C₄)alkyl group, or
(iv.2) a heteroaryl group selected from a pyridinyl, an imidazolyl, a pyrazinyl, a furanyl, a thiazolyl, a pyrazolyl, a thiadiazolyl, a pyridazinyl and a pyrimidinyl, optionally substituted by a (C₁-C₄)alkyl group, or
(v) a R″-L- group wherein L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group,
R^{b} and R^{c} independently represent a (C₁-C₆)alkyl group or a hydrogen atom,
or any of its pharmaceutically acceptable salt.

8. A compound of formula (I) according to claim 1, wherein R¹ represents:
(i) a (C₄-C₆)alkyl group substituted by a group selected from a hydroxy group, a halogen and a (C₁-C₃)alkoxy group,
(ii) a (C₃-C₈)cycloalkyl group, optionally substituted by a group selected from a hydroxy group and a (C₁-C₃) alkoxy group,
(iii) a bridged (C₆-C₁₀)cycloalkyl group, optionally substituted by a group selected from a (C₁-C₄)alkyl group, a (C₁-C₄)alkoxy group, a halogen atom and a hydroxy group, or
(iv) a R′-L- group, wherein L is either a single bond or a (C₁-C₃)alkanediyl group, optionally substituted by a group selected from a hydroxy group and a (C₁-C₃)alkoxy group, and
R′ represents:
(iv.1) a (C₃-C₈)heterocycloalkyl group, optionally substituted by one or two groups selected from a hydroxy group, and a (C₁-C₄)alkyl group, or
(iv.2) a heteroaryl group selected from a pyridinyl, an imidazolyl, a pyrazinyl, a furanyl, a thiazolyl, a pyrazolyl, a thiadiazolyl, a pyridazinyl and a pyrimidinyl, optionally substituted by a (C₁-C₄)alkyl group, or
(v) a R″-L- group wherein L is a (C₁-C₃)alkanediyl group, optionally substituted by a group selected from a —NR^{b}R^{c} group, a (C₁-C₃)alkoxy group and a hydroxy group, and
R″ is a phenyl group, optionally substituted by a fluoro(C₁-C₄)alkyl group,
R^{b} and R^{c} independently represent a (C₁-C₆)alkyl group or a hydrogen atom,
or any pharmaceutically acceptable salt thereof.

9. A compound of formula (I) according to claim 1, wherein R⁵ represents a hydrogen atom or a (C₁-C₄)alkyl group.

10. A compound of formula (I) according to claim 1 selected from:
(1) (4Z)-4-(1,3-Benzoxazol-6-ylmethylene)-2-(cyclohexylamino)-1H-imidazol-5-one,
(2) (4Z)-4-(1,3-Benzoxazol-6-ylmethylene)-2-(cycloheptylamino)-1H-imidazol-5-one,
(3) (4Z)-4-(1,3-Benzoxazol-6-ylmethylene)-2-(cyclooctylamino)-1H-imidazol-5-one,
(4) (4Z)-4-(1,3-Benzoxazol-6-ylmethylene)-2-[[(1R)-1-(hydroxymethyl)-3-methyl-butyl]amino]-1H-imidazol-5-one,
(5) (4Z)-4-(1,3-Benzoxazol-6-ylmethylene)-2-[[(1R)-1-(methoxymethyl)-3-methyl-butyl]amino]-1H-imidazol-5-one,
(6) (4Z)-4-(1,3-Benzoxazol-6-ylmethylene)-2-[[(1R,2R)-2-methoxycyclopentyl]amino]-1H-imidazol-5-one,
(7) (4Z)-4-(1,3-Benzoxazol-6-ylmethylene)-2-[[(1S,2S)-2-methoxycyclopentyl]amino]-1H-imidazol-5-one,
(8) (±)-(4Z)-4-(1,3-Benzoxazol-6-ylmethylene)-2-[[trans-4-hydroxycycloheptyl]amino]-1H-imidazol-5-one,
(9) (±)-(4Z)-4-(1,3-Benzoxazol-6-ylmethylene)-2-[[trans-4-methoxycycloheptyl]amino]-1H-imidazol-5-one,
(10) (±)-(4Z)-4-(1,3-Benzoxazol-6-ylmethylene)-2-[[cis-3-methoxycycloheptyl]amino]-1H-imidazol-5-one,
(11) (4Z)-2-(1-Adamantylamino)-4-(1,3-benzoxazol-6-ylmethylene)-1H-imidazol-5-one,
(12) (4Z)-4-(1,3-Benzoxazol-6-ylmethylene)-2-[(3-hydroxy-1-adamantyl)amino]-1H-imidazol-5-one,
(13) (4Z)-4-(1,3-Benzoxazol-6-ylmethylene)-2-[(3-methoxy-1-adamantyl)amino]-1H-imidazol-5-one,
(14) (4Z)-4-(1,3-Benzoxazol-6-ylmethylene)-2-[[2-(trifluoromethyl)phenyl]methylamino]-1H-imidazol-5-one,
(15) (4Z)-4-(1,3-Benzoxazol-6-ylmethylene)-2-[[(1S,2S)-2-hydroxyindan-1-yl]amino]-1H-imidazol-5-one,
(16) (4Z)-4-(1,3-Benzoxazol-6-ylmethylene)-2-[[(1R)-2-hydroxy-1-phenyl-ethyl]amino]-1H-imidazol-5-one,
(17) (4Z)-4-(1,3-Benzoxazol-6-ylmethylene)-2-[[(1S)-2-hydroxy-1-phenyl-ethyl]amino]-1H-imidazol-5-one,
(18) (4Z)-4-(1,3-Benzoxazol-6-ylmethylene)-2-[[(1R)-2-methoxy-1-phenyl-ethyl]amino]-1H-imidazol-5-one,
(19) (4Z)-4-(1,3-Benzoxazol-6-ylmethylene)-2-[(4-methylthiazol-2-yl)methylamino]-1H-imidazol-5-one,
(20) (4Z)-4-(1,3-Benzoxazol-6-ylmethylene)-2-(tetrahydropyran-4-ylmethylamino)-1H-imidazol-5-one,
(21) (4Z)-4-(1,3-Benzoxazol-6-ylmethylene)-2-[4-(4-methylpiperazin-1-yl)anilino]-1H-imidazol-5-one,
(22) (4Z)-4-(1,3-Benzoxazol-6-ylmethylene)-2-(2-pyridylamino)-1H-imidazol-5-one,
(23) (±)-(4Z)-4-(1,3-Benzoxazol-6-ylmethylene)-2-[(6,6-dimethyltetrahydropyran-3-yl)amino]-1H-imidazol-5-one,
(24) (4Z)-4-(1,3-Benzoxazol-6-ylmethylene)-2-[[(3S)-tetrahydropyran-3-yl]amino]-1H-imidazol-5-one,
(25) (±)-(4Z)-4-(1,3-Benzoxazol-6-ylmethylene)-2-(oxepan-3-ylamino)-1H-imidazol-5-one,
(26) (4Z)-2-(Cyclohexylamino)-4-(1H-indazol-5-ylmethylene)-1H-imidazol-5-one,
(27) 4Z)-2-(Cycloheptylamino)-4-(1H-indazol-5-ylmethylene)-1H-imidazol-5-one,
(28) 4Z)-2-(Cyclooctylamino)-4-(1H-indazol-5-ylmethylene)-1H-imidazol-5-one,
(29) (4Z)-2-[[(1R)-1-(Hydroxymethyl)-3-methyl-butyl]amino]-4-(1H-indazol-5-ylmethylene)-1H-imidazol-5-one,
(30) (4Z)-4-(1H-Indazol-5-ylmethylene)-2-[[(1R)-1-(methoxymethyl)-3-methyl-butyl]amino]-1H-imidazol-5-one,
(31) (4Z)-2-(1-Adamantylamino)-4-(1H-indazol-5-ylmethylene)-1H-imidazol-5-one,
(32) (4Z)-2-[(3-Hydroxy-1-adamantyl)amino]-4-(1H-indazol-5-ylmethylene)-1H-imidazol-5-one,
(33) (4Z)-4-(1H-Indazol-5-ylmethylene)-2-[[(1R)-2-methoxy-1-phenyl-ethyl]amino]-1H-imidazol-5-one,
(34) (4Z)-2-(Cyclohexylamino)-4-[(2-methylindazol-5-yl)methylene]-1H-imidazol-5-one,
(35) (4Z)-2-(Cycloheptylamino)-4-[(2-methylindazol-5-yl)methylene]-1H-imidazol-5-one,
(36) (4Z)-2-(Cyclooctylamino)-4-[(2-methylindazol-5-yl)methylene]-1H-imidazol-5-one,
(37) (4Z)-2-[[(1R)-1-(Hydroxymethyl)-3-methyl-butyl]amino]-4-[(2-methylindazol-5-yl)methylene]-1H-imidazol-5-one,
(38) (4Z)-2-[[(1R)-1-(Methoxymethyl)-3-methyl-butyl]amino]-4-[(2-methylindazol-5-yl)methylene]-1H-imidazol-5-one,
(39) (4Z)-2-(1-Adamantylamino)-4-[(2-methylindazol-5-yl)methylene]-1H-imidazol-5-one,
(40) (4Z)-2-[(3-Hydroxy-1-adamantyl)amino]-4-[(2-methylindazol-5-yl)methylene]-1H-imidazol-5-one,
(41) (4Z)-2-[(3-Fluoro-1-adamantyl)amino]-4-[(2-methylindazol-5-yl)methylene]-1H-imidazol-5-one,
(42) (4Z)-2-[[(1R)-2-Methoxy-1-phenyl-ethyl]amino]-4-[(2-methylindazol-5-yl)methylene]-1H-imidazol-5-one,
(43) (4Z)-2-(Cyclohexylamino)-4-[(1-methylindazol-5-yl)methylene]-1H-imidazol-5-one,
(44) (4Z)-2-(Cycloheptylamino)-4-[(1-methylindazol-5-yl)methylene]-1H-imidazol-5-one,
(45) (4Z)-2-(Cyclooctylamino)-4-[(1-methylindazol-5-yl)methylene]-1H-imidazol-5-one,
(46) (4Z)-2-[[(1R)-1-(Hydroxymethyl)-3-methyl-butyl]amino]-4-[(1-methylindazol-5-yl)methylene]-1H-imidazol-5-one,
(47) (4Z)-2-[[(1R)-1-(Methoxymethyl)-3-methyl-butyl]amino]-4-[(1-methylindazol-5-yl)methylene]-1H-imidazol-5-one,
(48) (4Z)-2-[[(1R)-1-(Fluoromethyl)-3-methyl-butyl]amino]-4-[(1-methylindazol-5-yl)methylene]-1H-imidazol-5-one,
(49) (4Z)-2-[[(1S)-1-(Fluoromethyl)-3-methyl-butyl]amino]-4-[(1-methylindazol-5-yl)methylene]-1H-imidazol-5-one,
(50) (4Z)-2-[[(1R,2R)-2-Methoxycyclopentyl]amino]-4-[(1-methylindazol-5-yl)methylene]-1H-imidazol-5-one,
(51) (4Z)-2-[[(1S,2S)-2-Methoxycyclopentyl]amino]-4-[(1-methylindazol-5-yl)methylene]-1H-imidazol-5-one,
(52) (±)-(4Z)-2-[[trans-4-Hydroxycycloheptyl]amino]-4-[(1-methylindazol-5-yl)methylene]-1H-imidazol-5-one,
(53) (±)-(4Z)-2-[[trans-4-Methoxycycloheptyl]amino]-4-[(1-methylindazol-5-yl)methylene]-1H-imidazol-5-one,
(54) (±)-(4Z)-2-[[cis-3-Methoxycycloheptyl]amino]-4-[(1-methylindazol-5-yl)methylene]-1H-imidazol-5-one,
(55) (4Z)-2-(1-Adamantylamino)-4-[(1-methylindazol-5-yl)methylene]-1H-imidazol-5-one,
(56) (4Z)-2-[(3-Hydroxy-1-adamantyl)amino]-4-[(1-methylindazol-5-yl)methylene]-1H-imidazol-5-one,
(57) (4Z)-2-[(3-Methoxy-1-adamantyl)amino]-4-[(1-methylindazol-5-yl)methylene]-1H-imidazol-5-one,
(58) (4Z)-2-[(3-Fluoro-1-adamantyl)amino]-4-[(1-methylindazol-5-yl)methylene]-1H-imidazol-5-one,
(59) (4Z)-4-[(1-Methylindazol-5-yl)methylene]-2-[[2-(trifluoromethyl)phenyl]methylamino]-1H-imidazol-5-one,
(60) (4Z)-2-[[(1S,2S)-2-Hydroxyindan-1-yl]amino]-4-[(1-methylindazol-5-yl)methylene]-1H-imidazol-5-one,
(61) (4Z)-2-[[(1R)-2-Hydroxy-1-phenyl-ethyl]amino]-4-[(1-methylindazol-5-yl)methylene]-1H-imidazol-5-one,
(62) (4Z)-2-[[(1S)-2-Hydroxy-1-phenyl-ethyl]amino]-4-[(1-methylindazol-5-yl)methylene]-1H-imidazol-5-one,
(63) (4Z)-2-[[(1R)-2-Methoxy-1-phenyl-ethyl]amino]-4-[(1-methylindazol-5-yl)methylene]-1H-imidazol-5-one,
(64) (4Z)-2-[[(2R)-2-Hydroxy-2-phenyl-ethyl]amino]-4-[(1-methylindazol-5-yl)methylene]-1H-imidazol-5-one,
(65) (4Z)-2-[[(1R)-2-Amino-1-phenyl-ethyl]amino]-4-[(1-methylindazol-5-yl)methylene]-1H-imidazol-5-one dihydrochloride,
(66) (4Z)-2-[[(1S)-2-Amino-1-phenyl-ethyl]amino]-4-[(1-methylindazol-5-yl)methylene]-1H-imidazol-5-one dihydrochloride,
(67) (4Z)-4-[(1-Methylindazol-5-yl)methylene]-2-[(4-methylthiazol-2-yl)methylamino]-1H-imidazol-5-one,
(68) (4Z)-4-[(1-Methylindazol-5-yl)methylene]-2-(tetrahydropyran-4-ylmethylamino)-1H-imidazol-5-one,
(69) (4Z)-4-[(1-Methylindazol-5-yl)methylene]-2-[4-(4-methylpiperazin-1-yl)anilino]-1H-imidazol-5-one,
(70) (4Z)-4-[(1-Methylindazol-5-yl)methylene]-2-(2-pyridylamino)-1H-imidazol-5-one,
(71) (4Z)-2-[(6,6-Dimethyltetrahydropyran-3-yl)amino]-4-[(1-methylindazol-5-yl)methylene]-1H-imidazol-5-one,
(72) (4Z)-4-[(1-Methylindazol-5-yl)methylene]-2-[[(3S)-tetrahydropyran-3-yl]amino]-1H-imidazol-5-one,
(73) (4Z)-2-[[(3R,4R)-4-Hydroxytetrahydropyran-3-yl]amino]-4-[(1-methylindazol-5-yl)methylene]-1H-imidazol-5-one,
(74) (±)-(4Z)-4-[(1-Methylindazol-5-yl)methylene]-2-(oxepan-3-ylamino)-1H-imidazol-5-one,
(75) (4Z)-4-(1H-Benzimidazol-5-ylmethylene)-2-(cyclohexylamino)-1H-imidazol-5-one,
(76) (4Z)-4-(1H-Benzimidazol-5-ylmethylene)-2-(cycloheptylamino)-1H-imidazol-5-one,
(77) (4Z)-4-(1H-Benzimidazol-5-ylmethylene)-2-(cyclooctylamino)-1H-imidazol-5-one,
(78) (4Z)-4-(1H-Benzimidazol-5-ylmethylene)-2-[[(1R)-1-(hydroxymethyl)-3-methyl-butyl]amino]-1H-imidazol-5-one,
(79) (4Z)-4-(1H-Benzimidazol-5-ylmethylene)-2-[[(1R)-1-(methoxymethyl)-3-methyl-butyl]amino]-1H-imidazol-5-one,
(80) (4Z)-4-(1H-Benzimidazol-5-ylmethylene)-2-[[(1R)-1-(fluoromethyl)-3-methyl-butyl]amino]-1H-imidazol-5-one,
(81) (4Z)-4-(1H-Benzimidazol-5-ylmethylene)-2-[[(1S)-1-(fluoromethyl)-3-methyl-butyl]amino]-1H-imidazol-5-one,
(82) (4Z)-4-(1H-Benzimidazol-5-ylmethylene)-2-[[(1R,2R)-2-methoxycyclopentyl]amino]-1H-imidazol-5-one,
(83) (4Z)-2-(3-Noradamantylamino)-4-(1H-benzimidazol-5-ylmethylene)-1H-imidazol-5-one,
(84) (4Z)-2-(1-Adamantylamino)-4-(1H-benzimidazol-5-ylmethylene)-1H-imidazol-5-one,
(85) (4Z)-4-(1H-Benzimidazol-5-ylmethylene)-2-[(3-hydroxy-1-adamantyl)amino]-1H-imidazol-5-one,
(86) (4Z)-4-(1H-Benzimidazol-5-ylmethylene)-2-[(3-methoxy-1-adamantyl)amino]-1H-imidazol-5-one,
(87) (4Z)-4-(1H-benzimidazol-5-ylmethylene)-2-[(3-fluoro-1-adamantyl)amino]-1H-imidazol-5-one,
(88) (4Z)-4-(1H-Benzimidazol-5-ylmethylene)-2-[[2-(trifluoromethyl)phenyl]methylamino]-1H-imidazol-5-one,
(89) (4Z)-4-(1H-Benzimidazol-5-ylmethylene)-2-[[(1S,2S)-2-hydroxyindan-1-yl]amino]-1H-imidazol-5-one,
(90) (4Z)-4-(1H-Benzimidazol-5-ylmethylene)-2-[[(1R)-2-hydroxy-1-phenyl-ethyl]amino]-1H-imidazol-5-one,
(91) (4Z)-4-(1H-Benzimidazol-5-ylmethylene)-2-[[(1S)-2-hydroxy-1-phenyl-ethyl]amino]-1H-imidazol-5-one,
(92) (4Z)-4-(1H-Benzimidazol-5-ylmethylene)-2-[[(1R)-2-methoxy-1-phenyl-ethyl]amino]-1H-imidazol-5-one,
(93) (4Z)-4-(1H-Benzimidazol-5-ylmethylene)-2-[[(2R)-2-hydroxy-2-phenyl-ethyl]amino]-1H-imidazol-5-one,
(94) (4Z)-4-(1H-Benzimidazol-5-ylmethylene)-2-[[(2S)-2-hydroxy-2-phenyl-ethyl]amino]-1H-imidazol-5-one,
(95) (4Z)-2-[[(1R)-2-Amino-1-phenyl-ethyl]amino]-4-(1H-benzimidazol-5-ylmethylene)-1H-imidazol-5-one dihydrochloride,
(96) (4Z)-2-[[(1S)-2-Amino-1-phenyl-ethyl]amino]-4-(1H-benzimidazol-5-ylmethylene)-1H-imidazol-5-one dihydrochloride,
(97) (4Z)-4-(1H-Benzimidazol-5-ylmethylene)-2-[(4-methylthiazol-2-yl)methylamino]-1H-imidazol-5-one,
(98) (4Z)-4-(1H-Benzimidazol-5-ylmethylene)-2-(tetrahydropyran-4-ylmethylamino)-1H-imidazol-5-one,
(99) (4Z)-4-(1H-Benzimidazol-5-ylmethylene)-2-[4-(4-methylpiperazin-1-yl)anilino]-1H-imidazol-5-one,
(100) (4Z)-4-(1H-Benzimidazol-5-ylmethylene)-2-(2-pyridylamino)-1H-imidazol-5-one,
(101) (4Z)-4-(1H-Benzimidazol-5-ylmethylene)-2-[[(3S)-tetrahydropyran-3-yl]amino]-1H-imidazol-5-one,
(102) (4Z)-4-(1H-Benzimidazol-5-ylmethylene)-2-[[(3R,4R)-4-hydroxytetrahydropyran-3-yl]amino]-1H-imidazol-5-one,
(103) (4Z)-4-(1H-Benzimidazol-5-ylmethylene)-2-[[(3S,4S)-4-hydroxytetrahydropyran-3-yl]amino]-1H-imidazol-5-one,
(104) (4Z)-4-(1H-Benzimidazol-5-ylmethylene)-2-(oxepan-3-ylamino)-1H-imidazol-5-one,
(105) (4Z)-2-(Cyclohexylamino)-4-[(3-methylbenzimidazol-5-yl)methylene]-1H-imidazol-5-one,
(106) (4Z)-2-(Cycloheptylamino)-4-[(3-methylbenzimidazol-5-yl)methylene]-1H-imidazol-5-one,
(107) (4Z)-2-(Cyclooctylamino)-4-[(3-methylbenzimidazol-5-yl)methylene]-1H-imidazol-5-one,
(108) (4Z)-2-[[(1R)-1-(Hydroxymethyl)-3-methyl-butyl]amino]-4-[(3-methylbenzimidazol-5-yl)methylene]-1H-imidazol-5-one,
(109) (4Z)-2-[[(1R)-1-(Methoxymethyl)-3-methyl-butyl]amino]-4-[(3-methylbenzimidazol-5-yl)methylene]-1H-imidazol-5-one,
(110) (4Z)-2-[[(1R)-1-(Fluoromethyl)-3-methyl-butyl]amino]-4-[(3-methylbenzimidazol-5-yl)methylene]-1H-imidazol-5-one,
(111) (4Z)-2-[[(1S)-1-(Fluoromethyl)-3-methyl-butyl]amino]-4-[(3-methylbenzimidazol-5-yl)methylene]-1H-imidazol-5-one,
(112) (4Z)-2-[[(1R,2R)-2-Methoxycyclopentyl]amino]-4-[(3-methylbenzimidazol-5-yl)methylene]-1H-imidazol-5-one,
(113) (4Z)-2-[[(1S,2S)-2-Methoxycyclopentyl]amino]-4-[(3-methylbenzimidazol-5-yl)methylene]-1H-imidazol-5-one,
(114) (4Z)-2-(3-Noradamantylamino)-4-[(3-methylbenzimidazol-5-yl)methylene]-1H-imidazol-5-one,
(115) (4Z)-2-(1-Adamantylamino)-4-[(3-methylbenzimidazol-5-yl)methylene]-1H-imidazol-5-one,
(116) (4Z)-2-[(3-Hydroxy-1-adamantyl)amino]-4-[(3-methylbenzimidazol-5-yl)methylene]-1H-imidazol-5-one,
(117) (4Z)-2-[(3-Methoxy-1-adamantyl)amino]-4-[(3-methylbenzimidazol-5-yl)methylene]-1H-imidazol-5-one,
(118) (4Z)-2-[(3-Fluoro-1-adamantyl)amino]-4-[(3-methylbenzimidazol-5-yl)methylene]-1H-imidazol-5-one,
(119) (4Z)-4-[(3-Methylbenzimidazol-5-yl)methylene]-2-[[2-(trifluoromethyl)phenyl]methylamino]-1H-imidazol-5-one,
(120) (4Z)-2-[[(1S,2S)-2-Hydroxyindan-1-yl]amino]-4-[(3-methylbenzimidazol-5-yl)methylene]-1H-imidazol-5-one,
(121) (4Z)-2-[[(1R)-2-Hydroxy-1-phenyl-ethyl]amino]-4-[(3-methylbenzimidazol-5-yl)methylene]-1H-imidazol-5-one,
(122) (4Z)-2-[[(1S)-2-Hydroxy-1-phenyl-ethyl]amino]-4-[(3-methylbenzimidazol-5-yl)methylene]-1H-imidazol-5-one,
(123) (4Z)-2-[[(1R)-2-Methoxy-1-phenyl-ethyl]amino]-4-[(3-methylbenzimidazol-5-yl)methylene]-1H-imidazol-5-one,
(124) (4Z)-2-[[(2R)-2-Hydroxy-2-phenyl-ethyl]amino]-4-[(3-methylbenzimidazol-5-yl)methylene]-1H-imidazol-5-one,
(125) (4Z)-2-[[(2S)-2-Hydroxy-2-phenyl-ethyl]amino]-4-[(3-methylbenzimidazol-5-yl)methylene]-1H-imidazol-5-one,
(126) (4Z)-2-[[(1R)-2-amino-1-phenyl-ethyl]amino]-4-[(3-methylbenzimidazol-5-yl)methylene]-1H-imidazol-5-one dihydrochloride,
(127) (4Z)-2-[[(1S)-2-amino-1-phenyl-ethyl]amino]-4-[(3-methylbenzimidazol-5-yl)methylene]-1H-imidazol-5-one dihydrochloride,
(128) (4Z)-4-[(3-Methylbenzimidazol-5-yl)methylene]-2-[(4-methylthiazol-2-yl)methylamino]-1H-imidazol-5-one,
(129) (4Z)-4-[(3-Methylbenzimidazol-5-yl)methylene]-2-(tetrahydropyran-4-ylmethylamino)-1H-imidazol-5-one,
(130) (4Z)-4-[(3-Methylbenzimidazol-5-yl)methylene]-2-[4-(4-methylpiperazin-1-yl)anilino]-1H-imidazol-5-one,
(131) (4Z)-4-[(3-Methylbenzimidazol-5-yl)methylene]-2-(2-pyridylamino)-1H-imidazol-5-one,
(132) (5Z)-5-(1,3-Benzoxazol-6-ylmethylene)-2-(cyclohexylamino)-3-methyl-imidazol-4-one,
(133) (5Z)-5-(1,3-Benzoxazol-6-ylmethylene)-2-(cyclooctylamino)-3-methyl-imidazol-4-one,
(134) (5Z)-5-(1,3-Benzoxazol-6-ylmethylene)-2-[[(1R)-1-(methoxymethyl)-3-methyl-butyl]amino]-3-methyl-imidazol-4-one,
(135) (5Z)-5-(1,3-Benzoxazol-6-ylmethylene)-2-[[(1R,2R)-2-methoxycyclopentyl]amino]-3-methyl-imidazol-4-one,
(136) (5Z)-5-(1,3-Benzoxazol-6-ylmethylene)-2-[[(1SR,2S)-2-methoxycyclopentyl]amino]-3-methyl-imidazol-4-one,
(137) (5Z)-2-(3-Noradamantylamino)-5-(1,3-benzoxazol-6-ylmethylene)-3-methyl-imidazol-4-one,
(138) (5Z)-2-(1-Adamantylamino)-5-(1,3-benzoxazol-6-ylmethylene)-3-methyl-imidazol-4-one,
(139) (5Z)-5-(1,3-Benzoxazol-6-ylmethylene)-2-[(3-hydroxy-1-adamantyl)amino]-3-methyl-imidazol-4-one,
(140) (5Z)-5-(1,3-Benzoxazol-6-ylmethylene)-2-[(3-methoxy-1-adamantyl)amino]-3-methyl-imidazol-4-one,
(141) (5Z)-5-(1,3-Benzoxazol-6-ylmethylene)-2-[(3-fluoro-1-adamantyl)amino]-3-methyl-imidazol-4-one,
(142) (5Z)-5-(1,3-Benzoxazol-6-ylmethylene)-3-methyl-2-[[2-(trifluoromethyl)phenyl]methylamino]imidazol-4-one,
(143) (5Z)-5-(1,3-Benzoxazol-6-ylmethylene)-2-[[(1R)-2-methoxy-1-phenyl-ethyl]amino]-3-methyl-imidazol-4-one,
(144) (5Z)-5-(1,3-Benzoxazol-6-ylmethylene)-3-methyl-2-[(4-methylthiazol-2-yl)methylamino]imidazol-4-one,
(145) (5Z)-2-(Cyclohexylamino)-5-(1H-indazol-5-ylmethylene)-3-methyl-imidazol-4-one,
(146) (5Z)-2-(Cycloheptylamino)-5-(1H-indazol-5-ylmethylene)-3-methyl-imidazol-4-one,
(147) (5Z)-2-(Cyclooctylamino)-5-(1H-indazol-5-ylmethylene)-3-methyl-imidazol-4-one,
(148) (5Z)-2-[[(1R)-1-(Hydroxymethyl)-3-methyl-butyl]amino]-5-(1H-indazol-5-ylmethylene)-3-methyl-imidazol-4-one,
(149) (5Z)-5-(1H-Indazol-5-ylmethylene)-2-[[(1R)-1-(methoxymethyl)-3-methyl-butyl]amino]-3-methyl-imidazol-4-one,
(150) (5Z)-5-(1H-Indazol-5-ylmethylene)-2-[[(1R,2R)-2-methoxycyclopentyl]amino]-3-methyl-imidazol-4-one,
(151) (5Z)-5-(1H-Indazol-5-ylmethylene)-2-[[(1S,2S)-2-methoxycyclopentyl]amino]-3-methyl-imidazol-4-one,
(152) (5Z)-2-(3-Nordamantylamino)-5-(1H-indazol-5-ylmethylene)-3-methyl-imidazol-4-one,
(153) (5Z)-2-(1-Adamantylamino)-5-(1H-indazol-5-ylmethylene)-3-methyl-imidazol-4-one,
(154) (5Z)-2-[(3-Hydroxy-1-adamantyl)amino]-5-(1H-indazol-5-ylmethylene)-3-methyl-imidazol-4-one,
(155) (5Z)-5-(1H-Indazol-5-ylmethylene)-2-[(3-methoxy-1-adamantyl)amino]-3-methyl-imidazol-4-one,
(156) (5Z)-2-[(3-Fluoro-1-adamantyl)amino]-5-(1H-indazol-5-ylmethylene)-3-methyl-imidazol-4-one,
(157) (5Z)-5-(1H-Indazol-5-ylmethylene)-3-methyl-2-[[2-(trifluoromethyl)phenyl]methylamino]imidazol-4-one,
(158) (5Z)-2-[[(1S,2S)-2-Hydroxyindan-1-yl]amino]-5-(1H-indazol-5-ylmethylene)-3-methyl-imidazol-4-one,
(159) (5Z)-2-[[(1R)-2-Hydroxy-1-phenyl-ethyl]amino]-5-(1H-indazol-5-ylmethylene)-3-methyl-imidazol-4-one,
(160) (5Z)-2-[[(1S)-2-Hydroxy-1-phenyl-ethyl]amino]-5-(1H-indazol-5-ylmethylene)-3-methyl-imidazol-4-one,
(161) (5Z)-5-(1H-Indazol-5-ylmethylene)-2-[[(1R)-2-methoxy-1-phenyl-ethyl]amino]-3-methyl-imidazol-4-one,
(162) (5Z)-2-[[(2R)-2-Hydroxy-2-phenyl-ethyl]amino]-5-(1H-indazol-5-ylmethylene)-3-methyl-imidazol-4-one,
(163) (5Z)-2-[[(1R)-2-Amino-1-phenyl-ethyl]amino]-5-(1H-indazol-5-ylmethylene)-3-methyl-imidazol-4-one dihydrochloride,
(164) (5Z)-5-(1H-Indazol-5-ylmethylene)-3-methyl-2-[(4-methylthiazol-2-yl)methylamino]imidazol-4-one,
(165) (5Z)-5-(1H-Indazol-5-ylmethylene)-3-methyl-2-(tetrahydropyran-4-ylmethylamino)imidazol-4-one,
(166) (5Z)-5-(1H-Indazol-5-ylmethylene)-3-methyl-2-[4-(4-methylpiperazin-1-yl)anilino]imidazol-4-one,
(167) (5Z)-5-(1H-Indazol-5-ylmethylene)-3-methyl-2-(2-pyridylamino)imidazol-4-one,
(168) (5Z)-5-(1H-Indazol-5-ylmethylene)-3-methyl-2-[[(3S)-tetrahydropyran-3-yl]amino]imidazol-4-one,
(169) (5Z)-2-[[(3R,4R)-4-Hydroxytetrahydropyran-3-yl]amino]-5-(1H-indazol-5-ylmethylene)-3-methyl-imidazol-4-one,
(170) (5Z)-2-(Cyclohexylamino)-3-methyl-5-[(2-methylindazol-5-yl)methylene]imidazol-4-one,
(171) (5Z)-2-(Cycloheptylamino)-3-methyl-5-[(2-methylindazol-5-yl)methylene]imidazol-4-one,
(172) (5Z)-2-(Cyclooctylamino)-3-methyl-5-[(2-methylindazol-5-yl)methylene]imidazol-4-one,
(173) (5Z)-2-[[(1R)-1-(Hydroxymethyl)-3-methyl-butyl]amino]-3-methyl-5-[(2-methylindazol-5-yl)methylene]imidazol-4-one,
(174) (5Z)-2-[[(1R)-1-(Methoxymethyl)-3-methyl-butyl]amino]-3-methyl-5-[(2-methylindazol-5-yl)methylene]imidazol-4-one,
(175) (5Z)-2-[[(1R,2R)-2-Methoxycyclopentyl]amino]-3-methyl-5-[(2-methylindazol-5-yl)methylene]imidazol-4-one,
(176) (5Z)-2-[[(1S,2S)-2-Methoxycyclopentyl]amino]-3-methyl-5-[(2-methylindazol-5-yl)methylene]imidazol-4-one,
(177) (5Z)-2-(3-Noradamantylamino)-3-methyl-5-[(2-methylindazol-5-yl)methylene]imidazol-4-one,
(178) (5Z)-2-(1-Adamantylamino)-3-methyl-5-[(2-methylindazol-5-yl)methylene]imidazol-4-one,
(179) (5Z)-2-[(3-Hydroxy-1-adamantyl)amino]-3-methyl-5-[(2-methylindazol-5-yl)methylene]imidazol-4-one,
(180) (5Z)-2-[(3-Methoxy-1-adamantyl)amino]-3-methyl-5-[(2-methylindazol-5-yl)methylene]imidazol-4-one,
(181) (5Z)-2-[(3-Fluoro-1-adamantyl)amino]-3-methyl-5-[(2-methylindazol-5-yl)methylene]imidazol-4-one,
(182) (5Z)-3-Methyl-5-[(2-methylindazol-5-yl)methylene]-2-[[2-(trifluoromethyl)phenyl]methylamino]imidazol-4-one,
(183) (5Z)-2-[[(1S,2S)-2-Hydroxyindan-1-yl]amino]-3-methyl-5-[(2-methylindazol-5-yl)methylene]imidazol-4-one,
(184) (5Z)-2-[[(1R)-2-Hydroxy-1-phenyl-ethyl]amino]-3-methyl-5-[(2-methylindazol-5-yl)methylene]imidazol-4-one,
(185) (5Z)-2-[[(1S)-2-Hydroxy-1-phenyl-ethyl]amino]-3-methyl-5-[(2-methylindazol-5-yl)methylene]imidazol-4-one,
(186) (5Z)-2-[[(1R)-2-Methoxy-1-phenyl-ethyl]amino]-3-methyl-5-[(2-methylindazol-5-yl)methylene]imidazol-4-one,
(187) (5Z)-2-[[(2R)-2-Hydroxy-2-phenyl-ethyl]amino]-3-methyl-5-[(2-methylindazol-5-yl)methylene]imidazol-4-one,
(188) (5Z)-2-[[(1R)-2-Amino-1-phenyl-ethyl]amino]-3-methyl-5-[(2-methylindazol-5-yl)methylene]imidazol-4-one dihydrochloride,
(189) (5Z)-3-Methyl-5-[(2-methylindazol-5-yl)methylene]-2-[(4-methylthiazol-2-yl)methylamino]imidazol-4-one,
(190) (5Z)-3-Methyl-5-[(2-methylindazol-5-yl)methylene]-2-(tetrahydropyran-4-ylmethylamino)imidazol-4-one,
(191) (5Z)-3-Methyl-5-[(2-methylindazol-5-yl)methylene]-2-[4-(4-methylpiperazin-1-yl)anilino]imidazol-4-one,
(192) (5Z)-3-Methyl-5-[(2-methylindazol-5-yl)methylene]-2-(2-pyridylamino)imidazol-4-one,
(193) (5Z)-3-Methyl-5-[(2-methylindazol-5-yl)methylene]-2-[[(3S)-tetrahydropyran-3-yl]amino]imidazol-4-one,
(194) (5Z)-2-[[(3R,4R)-4-Hydroxytetrahydropyran-3-yl]amino]-3-methyl-5-[(2-methylindazol-5-yl)methylene]imidazol-4-one,
(195) (5Z)-2-(Cyclohexylamino)-3-methyl-5-[(1-methylindazol-5-yl)methylene]imidazol-4-one,
(196) (5Z)-2-(Cycloheptylamino)-3-methyl-5-[(1-methylindazol-5-yl)methylene]imidazol-4-one,
(197) (5Z)-2-(Cyclooctylamino)-3-methyl-5-[(1-methylindazol-5-yl)methylene]imidazol-4-one,
(198) (5Z)-2-[[(1R)-1-(Hydroxymethyl)-3-methyl-butyl]amino]-3-methyl-5-[(1-methylindazol-5-yl)methylene]imidazol-4-one,
(199) (5Z)-2-[[(1R)-1-(Methoxymethyl)-3-methyl-butyl]amino]-3-methyl-5-[(1-methylindazol-5-yl)methylene]imidazol-4-one,
(200) (5Z)-2-[[(1R,2R)-2-Methoxycyclopentyl]amino]-3-methyl-5-[(1-methylindazol-5-yl)methylene]imidazol-4-one,
(201) (5Z)-2-[[(1S,2S)-2-Methoxycyclopentyl]amino]-3-methyl-5-[(1-methylindazol-5-yl)methylene]imidazol-4-one,
(202) (5Z)-2-(3-Noradamantylamino)-3-methyl-5-[(1-methylindazol-5-yl)methylene]imidazol-4-one,
(203) (5Z)-2-(1-Adamantylamino)-3-methyl-5-[(1-methylindazol-5-yl)methylene]imidazol-4-one,
(204) (5Z)-2-[(3-Hydroxy-1-adamantyl)amino]-3-methyl-5-[(1-methylindazol-5-yl)methylene]imidazol-4-one,
(205) (5Z)-2-[(3-Methoxy-1-adamantyl)amino]-3-methyl-5-[(1-methylindazol-5-yl)methylene]imidazol-4-one,
(206) (5Z)-2-[(3-Fluoro-1-adamantyl)amino]-3-methyl-5-[(1-methylindazol-5-yl)methylene]imidazol-4-one,
(207) (5Z)-3-Methyl-5-[(1-methylindazol-5-yl)methylene]-2-[[2-(trifluoromethyl)phenyl]methylamino]imidazol-4-one,
(208) (5Z)-2-[[(1S,2S)-2-Hydroxyindan-1-yl]amino]-3-methyl-5-[(1-methylindazol-5-yl)methylene]imidazol-4-one,
(209) (5Z)-2-[[(1R)-2-Hydroxy-1-phenyl-ethyl]amino]-3-methyl-5-[(1-methylindazol-5-yl)methylene]imidazol-4-one,
(210) (5Z)-2-[[(1R)-2-Hydroxy-1-phenyl-ethyl]amino]-3-methyl-5-[(1-methylindazol-5-yl)methylene]imidazol-4-one,
(211) (5Z)-2-[[(1R)-2-Methoxy-1-phenyl-ethyl]amino]-3-methyl-5-[(1-methylindazol-5-yl)methylene]imidazol-4-one,
(212) (5Z)-2-[[(2R)-2-Hydroxy-2-phenyl-ethyl]amino]-3-methyl-5-[(1-methylindazol-5-yl)methylene]imidazol-4-one,
(213) (5Z)-2-[[(1R)-2-Amino-1-phenyl-ethyl]amino]-3-methyl-5-[(1-methylindazol-5-yl)methylene]imidazol-4-one dihydrochloride,
(214) (5Z)-3-Methyl-5-[(1-methylindazol-5-yl)methylene]-2-[(4-methylthiazol-2-yl)methylamino]imidazol-4-one,
(215) (5Z)-3-Methyl-5-[(1-methylindazol-5-yl)methylene]-2-(tetrahydropyran-4-ylmethylamino)imidazol-4-one,
(216) (5Z)-3-Methyl-5-[(1-methylindazol-5-yl)methylene]-2-[4-(4-methylpiperazin-1-yl)anilino]imidazol-4-one,
(217) (5Z)-3-Methyl-5-[(1-methylindazol-5-yl)methylene]-2-(2-pyridylamino)imidazol-4-one,
(218) (5Z)-3-Methyl-5-[(1-methylindazol-5-yl)methylene]-2-[[(3S)-tetrahydropyran-3-yl]amino]imidazol-4-one,
(219) (5Z)-2-[[(3R,4R)-4-Hydroxytetrahydropyran-3-yl]amino]-3-methyl-5-[(1-methylindazol-5-yl)methylene]imidazol-4-one,
(220) (5Z)-5-(1H-Benzimidazol-5-ylmethylene)-2-(cyclohexylamino)-3-methyl-imidazol-4-one,
(221) (5Z)-5-(1H-Benzimidazol-5-ylmethylene)-2-(cycloheptylamino)-3-methyl-imidazol-4-one,
(222) (5Z)-5-(1H-Benzimidazol-5-ylmethylene)-2-(cyclooctylamino)-3-methyl-imidazol-4-one,
(223) (5Z)-5-(1H-Benzimidazol-5-ylmethylene)-2-[[(1R)-1-(hydroxymethyl)-3-methyl-butyl]amino]-3-methyl-imidazol-4-one,
(224) (5Z)-5-(1H-Benzimidazol-5-ylmethylene)-2-[[(1R)-1-(methoxymethyl)-3-methyl-butyl]amino]-3-methyl-imidazol-4-one,
(225) (5Z)-5-(1H-Benzimidazol-5-ylmethylene)-2-[[(1R,2R)-2-methoxycyclopentyl]amino]-3-methyl-imidazol-4-one,
(226) (5Z)-5-(1H-Benzimidazol-5-ylmethylene)-2-[[(1S,2S)-2-methoxycyclopentyl]amino]-3-methyl-imidazol-4-one,
(227) (5Z)-2-(3-Nordamantylamino)-5-(1H-benzimidazol-5-ylmethylene)-3-methyl-imidazol-4-one,
(228) (5Z)-2-(1-Adamantylamino)-5-(1H-benzimidazol-5-ylmethylene)-3-methyl-imidazol-4-one,
(229) (5Z)-5-(1H-Benzimidazol-5-ylmethylene)-2-[(3-hydroxy-1-adamantyl)amino]-3-methyl-imidazol-4-one,
(230) (5Z)-5-(1H-Benzimidazol-5-ylmethylene)-2-[(3-methoxy-1-adamantyl)amino]-3-methyl-imidazol-4-one,
(231) (5Z)-5-(1H-Benzimidazol-5-ylmethylene)-2-[(3-fluoro-1-adamantyl)amino]-3-methyl-imidazol-4-one,
(232) (5Z)-5-(1H-Benzimidazol-5-ylmethylene)-3-methyl-2-[[2-(trifluoromethyl)phenyl]methylamino]imidazol-4-one,
(233) (5Z)-5-(1H-Benzimidazol-5-ylmethylene)-2-[[(1S,2S)-2-hydroxyindan-1-yl]amino]-3-methyl-imidazol-4-one,
(234) (5Z)-5-(1H-Benzimidazol-5-ylmethylene)-2-[[(1R)-2-hydroxy-1-phenyl-ethyl]amino]-3-methyl-imidazol-4-one,
(235) (5Z)-5-(1H-Benzimidazol-5-ylmethylene)-2-[[(1S)-2-hydroxy-1-phenyl-ethyl]amino]-3-methyl-imidazol-4-one,
(236) (5Z)-5-(1H-Benzimidazol-5-ylmethylene)-2-[[(1R)-2-methoxy-1-phenyl-ethyl]amino]-3-methyl-imidazol-4-one,
(237) (5Z)-5-(1H-Benzimidazol-5-ylmethylene)-2-[[(2R)-2-hydroxy-2-phenyl-ethyl]amino]-3-methyl-imidazol-4-one,
(238) (5Z)-5-(1H-Benzimidazol-5-ylmethylene)-3-methyl-2-[(4-methylthiazol-2-yl)methylamino]imidazol-4-one,
(239) (5Z)-5-(1H-Benzimidazol-5-ylmethylene)-3-methyl-2-(tetrahydropyran-4-ylmethylamino)imidazol-4-one,
(240) (5Z)-5-(1H-Benzimidazol-5-ylmethylene)-3-methyl-2-[4-(4-methylpiperazin-1-yl)anilino]imidazol-4-one,
(241) (5Z)-5-(1H-Benzimidazol-5-ylmethylene)-3-methyl-2-(2-pyridylamino)imidazol-4-one,
(242) (5Z)-5-(1H-Benzimidazol-5-ylmethylene)-3-methyl-2-[[(3S)-tetrahydropyran-3-yl]amino]imidazol-4-one,
(243) (5Z)-5-(1H-Benzimidazol-5-ylmethylene)-2-[[(3R,4R)-4-hydroxytetrahydropyran-3-yl]amino]-3-methyl-imidazol-4-one,
(244) (5Z)-2-(Cyclohexylamino)-3-methyl-5-[(3-methylbenzimidazol-5-yl)methylene]imidazol-4-one,
(245) (5Z)-2-(Cycloheptylamino)-3-methyl-5-[(3-methylbenzimidazol-5-yl)methylene]imidazol-4-one,
(246) (5Z)-2-(Cyclooctylamino)-3-methyl-5-[(3-methylbenzimidazol-5-yl)methylene]imidazol-4-one,
(247) (5Z)-2-[[(1R)-1-(Hydroxymethyl)-3-methyl-butyl]amino]-3-methyl-5-[(3-methylbenzimidazol-5-yl)methylene]imidazol-4-one,
(248) (5Z)-2-[[(1R)-1-(Methoxymethyl)-3-methyl-butyl]amino]-3-methyl-5-[(3-methylbenzimidazol-5-yl)methylene]imidazol-4-one,
(249) (5Z)-2-[[(1R,2R)-2-Methoxycyclopentyl]amino]-3-methyl-5-[(3-methylbenzimidazol-5-yl)methylene]imidazol-4-one,
(250) (5Z)-2-[[(1S,2S)-2-Methoxycyclopentyl]amino]-3-methyl-5-[(3-methylbenzimidazol-5-yl)methylene]imidazol-4-one,
(251) (5Z)-2-(3-Noradamantylamino)-3-methyl-5-[(3-methylbenzimidazol-5-yl)methylene]imidazol-4-one (252) (5Z)-2-(1-Adamantylamino)-3-methyl-5-[(3-methylbenzimidazol-5-yl)methylene]imidazol-4-one,
(253) (5Z)-2-[(3-Hydroxy-1-adamantyl)amino]-3-methyl-5-[(3-methylbenzimidazol-5-yl)methylene]imidazol-4-one,
(254) (5Z)-2-[(3-Methoxy-1-adamantyl)amino]-3-methyl-5-[(3-methylbenzimidazol-5-yl)methylene]imidazol-4-one,
(255) (5Z)-2-[(3-Fluoro-1-adamantyl)amino]-3-methyl-5-[(3-methylbenzimidazol-5-yl)methylene]imidazol-4-one,
(256) (5Z)-3-Methyl-5-[(3-methylbenzimidazol-5-yl)methylene]-2-[[2-(trifluoromethyl)phenyl]methylamino]imidazol-4-one,
(257) (5Z)-2-[[(1R)-2-Hydroxy-1-phenyl-ethyl]amino]-3-methyl-5-[(3-methylbenzimidazol-5-yl)methylene]imidazol-4-one,
(258) (5Z)-2-[[(1S)-2-Hydroxy-1-phenyl-ethyl]amino]-3-methyl-5-[(3-methylbenzimidazol-5-yl)methylene]imidazol-4-one,
(259) (5Z)-2-[[(1R)-2-Methoxy-1-phenyl-ethyl]amino]-3-methyl-5-[(3-methylbenzimidazol-5-yl)methylene]imidazol-4-one,
(260) (5Z)-2-[[(2R)-2-Hydroxy-2-phenyl-ethyl]amino]-3-methyl-5-[(3-methylbenzimidazol-5-yl)methylene]imidazol-4-one,
(261) (5Z)-3-Methyl-5-[(3-methylbenzimidazol-5-yl)methylene]-2-[(4-methylthiazol-2-yl)methylamino]imidazol-4-one,
(262) (5Z)-3-Methyl-5-[(3-methylbenzimidazol-5-yl)methylene]-2-(tetrahydropyran-4-ylmethylamino)imidazol-4-one,
(263) (5Z)-3-Methyl-5-[(3-methylbenzimidazol-5-yl)methylene]-2-[4-(4-methylpiperazin-1-yl)anilino]imidazol-4-one,
(264) (5Z)-3-Methyl-5-[(3-methylbenzimidazol-5-yl)methylene]-2-(2-pyridylamino)imidazol-4-one,
(265) (5Z)-3-Methyl-5-[(3-methylbenzimidazol-5-yl)methylene]-2-[[(3S)-tetrahydropyran-3-yl]amino]imidazol-4-one,
(266) (5Z)-2-[[(3R,4R)-4-Hydroxytetrahydropyran-3-yl]amino]-3-methyl-5-[(3-methylbenzimidazol-5-yl)methylene]imidazol-4-one,
(267) (4Z)-4-(1,2,3-Benzothiadiazol-6-ylmethylene)-2-(cyclohexylamino)-1H-imidazol-5-one,
(268) (4Z)-4-(1,2,3-Benzothiadiazol-6-ylmethylene)-2-(cycloheptylamino)-1H-imidazol-5-one,
(269) (4Z)-4-(1,2,3-Benzothiadiazol-6-ylmethylene)-2-[[(1R)-1-(hydroxymethyl)-3-methyl-butyl]amino]-1H-imidazol-5-one,
(270) (4Z)-2-(1-Adamantylamino)-4-(1,2,3-benzothiadiazol-6-ylmethylene)-1H-imidazol-5-one,
(271) (4Z)-4-(Indazol-5-ylmethylene)-2-[4-(4-methylpiperazin-1-yl)aniline]-1H-imidazol-5-one,
or anyone of their pharmaceutically acceptable salts.

11. A pharmaceutical composition comprising at least one compound as defined in claim 1 or a pharmaceutically acceptable salt thereof.

12. Synthesis process for manufacturing a compound of formula (I) as defined in claim 1 or any of its pharmaceutically acceptable salts, comprising at least a step of coupling a compound of formula (IX) below 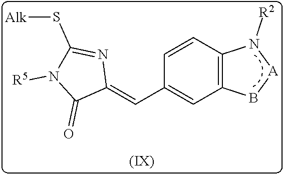
wherein Alk is a (C₁-C₅)alkyl,
with an amine of formula R¹NH₂.

13. Synthesis process for manufacturing a compound of formula (I) as defined in claim 1 or any of its pharmaceutically acceptable salt, comprising at least a step of coupling a compound of formula (II) below 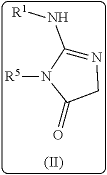
with a compound of formula (III) below

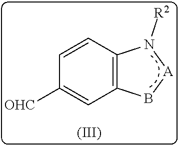

14. A synthetic intermediate of formula (XI) 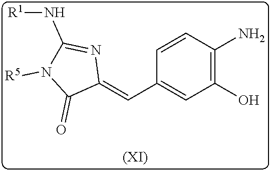
wherein R¹ and R⁵ are as defined in claim 1.

15. (canceled)

16. A therapeutic method for the treatment and/or for the prevention of a disease selected from cognitive deficits and neuroinflammation associated with Down syndrome; Alzheimer's disease and related diseases, dementia and/or tauopathies; as well as other neurodegenerative diseases; CDKL5 Deficiency Disorder; McDermid syndrome; autism; type 1 and type 2 diabetes; abnormal folate and methionine metabolism; tendinopathy and osteoarthritis, Duchenne muscular dystrophy; cancers, viral infections, neuroinflammation; anemia; infections caused by unicellular parasites, cattle diseases due to unicellular pathogens, and for regulating body temperature, in a patient in need thereof comprising at least a step of administering a therapeutically effective amount of a compound of formula (I) according to claim 1 or any of its pharmaceutically acceptable salts.

17. The method according to claim 16 wherein the disease selected from Down syndrome, Alzheimer's disease, dementia, tauopathies, Parkinson's disease, Niemann-Pick Type C Disease, CDKL5 Deficiency Disorder and Phelan-McDermid syndrome and their associated cognitive and motor conditions and type 1 and type 2 diabetes.

18. The method according to claim 16, wherein the disease is selected from brain cancer, glioblastoma, leukemia, megakaryoblastic leukemia and acute lymphoblastic leukemia, head and neck squamous cell carcinoma, pancreatic cancer, pancreatic ductal adenocarcinoma, prostate cancer, gastrointestinal cancer, breast cancer, Triple-negative breast cancer, tissue cancer, liposarcoma, Hedgehog/GLI-dependent cancer, liver cancer, Hepatocellular carcinoma, infections caused by Human immunodeficiency virus type 1, Human cytomegalovirus, Influenza A, Herpes virus, rhesus macaque cytomegalovirus, varicella-zoster virus, herpes simplex virus, Hepatitis C virus, Chikungunya virus, Dengue virus, Influenza virus and Severe acute respiratory syndrome coronavirus, Cytomegalovirus and Human papillomavirus, malaria, Leishmaniasis, Chagas and sleeping sickness.

19. A therapeutic method for the treatment and/or for the prevention of a disease selected from cognitive deficits and neuroinflammation associated with Down syndrome; Alzheimer's disease and related diseases, dementia and/or tauopathies; as well as other neurodegenerative diseases; CDKL5 Deficiency Disorder; McDermid syndrome; autism; type 1 and type 2 diabetes; abnormal folate and methionine metabolism; tendinopathy and osteoarthritis, Duchenne muscular dystrophy; cancers, viral infections, neuroinflammation; anemia; infections caused by unicellular parasites cattle diseases due to unicellular pathogens, and for regulating body temperature, in a patient in need thereof comprising at least a step of administering a therapeutically effective amount of at least any of compounds (1) to (271) as defined in claim 10 or any of its pharmaceutically acceptable salts.

20. The method according to claim 19 wherein the disease is selected from Down syndrome, Alzheimer's disease, dementia, tauopathies, Parkinson's disease, Niemann-Pick Type C Disease, CDKL5 Deficiency Disorder and Phelan-McDermid syndrome and their associated cognitive and motor conditions and type 1 and type 2 diabetes.

21. The method according to claim 19 wherein the disease is selected from brain cancer, glioblastoma, leukemia, megakaryoblastic leukemia and acute lymphoblastic leukemia, head and neck squamous cell carcinoma, pancreatic cancer, pancreatic ductal adenocarcinoma, prostate cancer, gastrointestinal cancer, breast cancer, Triple-negative breast cancer, tissue cancer, liposarcoma, Hedgehog/GLI-dependent cancer, liver cancer, Hepatocellular carcinoma, infections caused by Human immunodeficiency virus type 1, Human cytomegalovirus, Influenza A, Herpes virus, rhesus macaque cytomegalovirus, varicella-zoster virus, herpes simplex virus, Hepatitis C virus, Chikungunya virus, Dengue virus, Influenza virus and Severe acute respiratory syndrome coronavirus, Cytomegalovirus and Human papillomavirus, malaria, Leishmaniasis, Chagas and sleeping sickness.

22. A pharmaceutical composition comprising at least one compound selected from (1) to (271) as defined in claim 10 or a pharmaceutically acceptable salt thereof.
